(19) United States
(12) Patent Application Publication
(10) Pub. No.: US 2019/0194179 A1
(43) Pub. Date: Jun. 27, 2019

(54) ANTIBIOTIC COMPOUNDS

(71) Applicant: DISCUVA LTD., Cambridge (GB)

(72) Inventors: Paul MEO, Cambridge (GB); Nawaz KHAN, Cambridge (GB)

(73) Assignee: DISCUVA LTD., Cambridge (GB)

(21) Appl. No.: 16/327,286

(22) PCT Filed: Aug. 22, 2017
(86) PCT No.: PCT/GB2017/052478

§ 371 Date: Feb. 21, 2019

(30) Foreign Application Priority Data
Aug. 22, 2016 (GB) 1614314.1

(51) Int. Cl. C07D 413/12; C07D 413/14; C07D 417/12; A61P 31/04

(52) U.S. Cl. CPC C07D 413/12; A61P 31/04; C07D 417/12; C07D 413/14

(57) ABSTRACT

The present invention relates to antibiotic compounds of formula (I), to compositions containing these compounds and to methods of treating bacterial diseases and infections using the compounds. The compounds find application in the treatment of infection with, and diseases caused by, Gram-positive and/or Gram-negative bacteria, and in particular in the treatment of infection with, and diseases caused by, Neisseria gonorrhoeae.

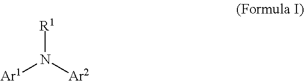

### FIELD OF THE INVENTION

The present invention relates to a new class of antibiotic compounds as defined herein, to compositions containing these compounds and to methods of treating bacterial diseases and infections using the compounds. The compounds find application in the treatment of infection with, and diseases caused by, Gram-positive and/or Gram-negative bacteria, and in particular in the treatment of infection with, and diseases caused by, Neisseria gonorrhoeae.

### BACKGROUND TO THE INVENTION

There is an urgent need for new antibiotics to counter the emergence of new bacterial pathogens and resistance to existing antibacterial drugs. For example, Neisseria gonorrhoeae is evolving into a superbug with resistance to previously and currently recommended antimicrobials for the treatment of gonorrhoea, and is now a major public health concern globally. Given the global nature of gonorrhoea, the high rate of usage of antimicrobials, suboptimal control and monitoring of antimicrobial resistance and the extraordinary capacity of the gonococci to develop and retain resistance, there is a risk that the severe complications of gonorrhoea will emerge as a silent epidemic (Unemo and Schafer (2014) Clin Microbiol Rev. 27 (3): 587-613).

Accordingly, there exists a need for new agents for the treatment of bacterial infection, for example in the treatment of Gram-negative infections, including in particular infection with Neisseria gonorrhoeae.

### SUMMARY OF THE INVENTION

Therefore, in a first aspect of the present invention, there is provided a compound of general formula (I), or a pharmaceutically acceptable salt, derivative, hydrate, solvate, complex, isomer, tautomer, bioisostere, N-oxide, ester, prodrug, isotope or protected form thereof:

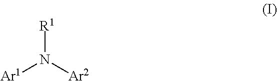

wherein Ar⁰ has the formula (A1)

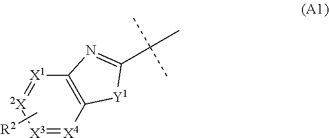

X¹, X², X³, and X⁴ are each independently selected from N and CH;

Y¹ is selected from O and NR³,

R¹ is selected from hydrogen and C₁₋₄alkyl;

R² is one or more optional substituents each independently selected from halogen, cyano, hydroxyl, hydroxylC₁₋₄alkyl, C₁₋₄alkyl, haloC₁₋₄alkyl, C₁₋₄alkoxy, haloC₁₋₄alkyloxy, —C₁₋₄alkylC₁₋₄alkoxy, C₁₋₄alkoxyC₁₋₄alkoxy, NR^{4A}R^{4B}, NO₂, —CONR^{4A}R^{4B}, —C₁₋₄alkylNR^{4A}R^{4B}, —C₁₋₄alkoxyNR^{4A}R^{4B}, C₃₋₇cycloalkyl, morpholinyl, C₂₋₄alkynyl and —CO₂R⁴ wherein

R³ is hydrogen or C₁₋₄alkyl,

R⁴ is hydrogen or C₁₋₄alkyl,

R^{4A} and R^{4B} are each independently selected from hydrogen, C₁₋₄alkyl, —C₁₋₄alkylC₁₋₄alkoxy, and COR⁴, or

R^{4A} and R^{4B}, together with the nitrogen atom to which they are attached, join together to form a cyclic amino group, wherein the cyclic amino group is optionally substituted with oxo;

Ar² is a ring system selected from Groups (i), (ii), and (iii), wherein:

Group (i) is a 5-membered heteroaryl ring system selected from any one of (IIa) to (IIm):

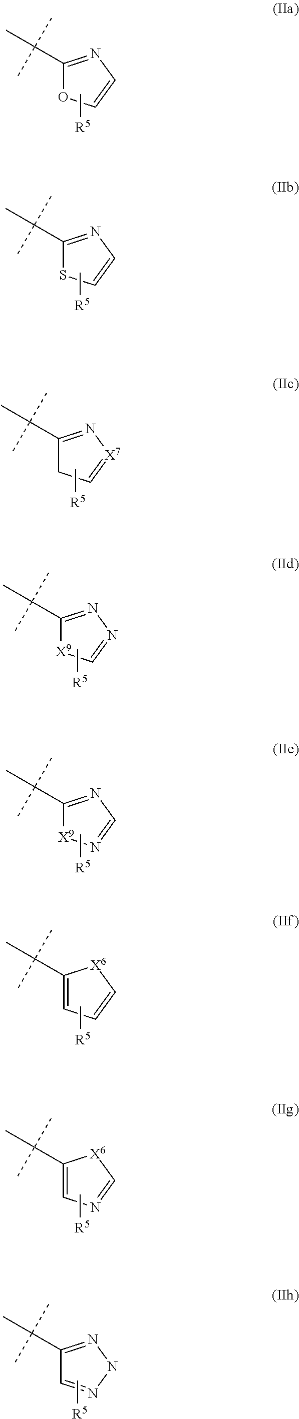

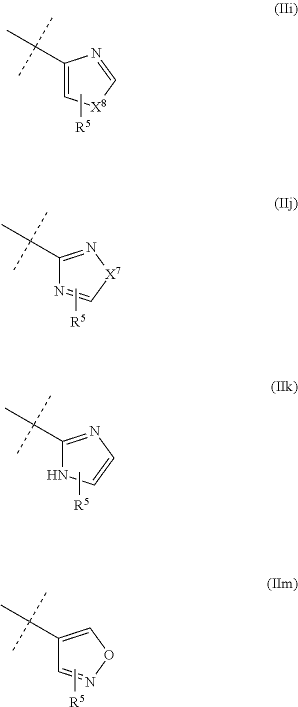

wherein X⁶, X⁷, X⁸, and X⁹ are each independently selected from O, S, and NH, and

R⁵ is one or more optional substituents each independently selected from halogen, cyano, C₁₋₄alkyl, haloC₁₋₄alkyl, C₁₋₄alkoxy, —C₁₋₄alkylC₁₋₄alkoxy, —CO₂R⁶, and -L-Q wherein:

L is a linker group selected from a direct bond, C₁₋₃alkylene and —CO—; and

Q is a group selected from NR^{5A}R^{5B}, C₃cycloalkyl and 4-7 membered heterocyclyl; and wherein the 4-7 membered heterocyclyl ring is optionally substituted with one or more substituents selected from halogen, cyano, C₁₋₄alkyl, C₁₋₄alkoxy and CO₂R⁶;

R^{5A} and R^{5B} are each independently selected from hydrogen, C₁₋₄alkyl, C₃₋₇ cycloalkyl, COR⁷, —C₁₋₄alkyl-NR⁸R⁹, —C₁₋₄alkylC₁₋₄alkoxy, phenyl and 5 or 6-membered heteroaryl wherein the phenyl or 5 or 6-membered heteroaryl rings are optionally substituted with one or more substituents selected from halogen and C₁₋₄alkyl; or

R^{5A} and R^{5B}, together with the nitrogen atom to which they are attached, join together to form a cyclic amino group, which cyclic amino group is optionally substituted with one or more groups selected from halogen, C₁₋₄alkyl, C₁₋₄alkoxy, cyano, and CO₂R⁶,

R⁶ is hydrogen, C₁₋₄alkyl or an alkali metal;

R⁷ is C₁₋₄alkyl

R⁸ and R⁹ are each independently selected from hydrogen and C₁₋₄alkyl; Group (ii) is a 5,6-fused bicyclic heteroaryl ring system having the formula (III):

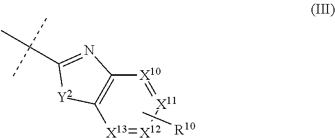

wherein Y² is selected from O and NR^{5C};

R^{5C} is hydrogen or C₁₋₄alkyl,

X¹⁰, X¹¹, X¹², and X¹³ are each independently selected from N and CH;

R¹⁰ is one or more optional substituents each independently selected from halogen, cyano, C₁₋₄alkyl, haloC₁₋₄alkyl, C₁₋₄alkoxy, and —CO₂R⁴;

Group (iii) is a fused 5,6-fused bicyclic ring system having the formula (IVa) or (IVb)

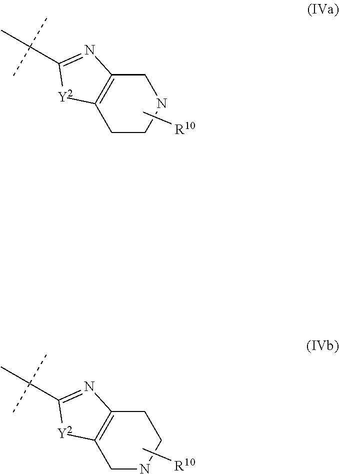

wherein Y² is selected from O and NR^{5C}; and

R¹⁰ is one or more optional substituents each independently selected from halogen, cyano, C₁₋₄alkyl, haloC₁₋₄alkyl, C₁₋₄alkoxy, and —CO₂R⁴;

PROVIDED THAT the compound of formula (I) is other than:

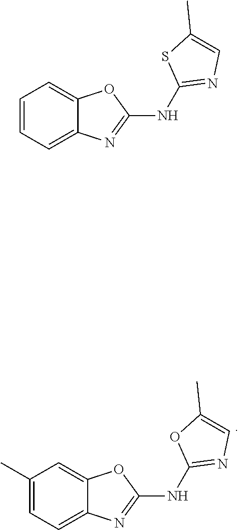 [SMILES: Cc1cnc(Nc2nc3ccccc3o2)s1 ; Cc1ccc2nc(Nc3ncc(C)o3)oc2c1]

In another aspect, there is provided a compound as defined above, or a pharmaceutically acceptable salt, derivative, hydrate, solvate, complex, isomer, tautomer, bioisostere, N-oxide, ester, prodrug, isotope or protected form thereof, for use in a method of treatment of an infection with, or a disease caused by, a bacterium.

In another aspect, there is provided a compound as defined above, or a pharmaceutically acceptable salt, derivative, hydrate, solvate, complex, isomer, tautomer, bioisostere, N-oxide, ester, prodrug, isotope or protected form thereof, together with a pharmaceutically acceptable excipient or carrier.

In another aspect, there is provided the use of a compound as defined above, or a pharmaceutically acceptable salt, derivative, hydrate, solvate, complex, isomer, tautomer, bioisostere, N-oxide, ester, prodrug, isotope or protected form thereof, for the manufacture of a medicament for use in the treatment of an infection with, or a disease caused by, a bacterium.

In another aspect, there is provided a method of treating an infection with, or disease caused by, a bacterium in a subject in need thereof, comprising administering to said subject an effective amount of a compound as defined above, or a pharmaceutically acceptable salt, derivative, hydrate, solvate, complex, isomer, tautomer, bioisostere, N-oxide, ester, prodrug, isotope or protected form thereof.

In another aspect there is provided a bactericidal or bacteriostatic composition comprising a compound or composition as defined above.

In certain embodiments, the compounds of the invention have bactericidal and/or bacteriostatic activity against Neisseria gonorrhoeae, and may be used in the treatment or prophylaxis of an infection with, or a disease caused by, Neisseria gonorrhoeae.

Other aspects and embodiments of the invention are as defined in the claims attached hereto.

### DETAILED DESCRIPTION OF THE INVENTION

All publications, patents, patent applications and other references mentioned herein are hereby incorporated by reference in their entireties for all purposes as if each individual publication, patent or patent application were specifically and individually indicated to be incorporated by reference and the content thereof recited in full.

Definitions and general preferences Where used herein and unless specifically indicated otherwise, the following terms are intended to have the following meanings in addition to any broader (or narrower) meanings the terms might enjoy in the art: Unless otherwise required by context, the use herein of the singular is to be read to include the plural and vice versa. The term “a” or “an” used in relation to an entity is to be read to refer to one or more of that entity. As such, the terms “a” (or “an”), “one or more,” and “at least one” are used interchangeably herein.

As used herein, the term “comprise,” or variations thereof such as “comprises” or “comprising,” are to be read to indicate the inclusion of any recited integer (e.g. a feature, element, characteristic, property, method/process step or limitation) or group of integers (e.g. features, element, characteristics, properties, method/process steps or limitations) but not the exclusion of any other integer or group of integers. Thus, as used herein the term “comprising” is inclusive or open-ended and does not exclude additional, unrecited integers or method/process steps.

As used herein, the term “consisting” is used to indicate the presence of the recited integer (e.g. a feature, element, characteristic, property, method/process step or limitation) or group of integers (e.g. features, element, characteristics, properties, method/process steps or limitations) alone.

As used herein, the term “disease” is used to define any abnormal condition that impairs physiological function and is associated with specific symptoms. The term is used broadly to encompass any disorder, illness, abnormality, pathology, sickness, condition or syndrome in which physiological function is impaired irrespective of the nature of the aetiology (or indeed whether the aetiological basis for the disease is established). It therefore encompasses conditions arising from trauma, injury, surgery, radiological ablation, poisoning or nutritional deficiencies.

As used herein, the term “bacterial disease” refers to any disease that involves (e.g. is caused, exacerbated, associated with or characterized by the presence of) a bacterium residing and/or replicating in the body and/or cells of a subject. The term therefore includes diseases caused or exacerbated by bacterial toxins (which may also be referred to herein as “bacterial intoxication”).

As used herein, the term “bacterial infection” is used to define a condition in which a subject is infected with a bacterium. The infection may be symptomatic or asymptomatic. In the former case, the subject may be identified as infected on the basis of established diagnostic criteria. In the latter case, the subject may be identified as infected on the basis of various tests, including for example biochemical tests, serological tests, microbiological culture and/or microscopy.

Thus, the invention finds application in the treatment of subjects in which bacterial infection (e.g. by Neisseria gonorrhoeae) has been diagnosed or detected.

As used herein, the term “treatment” or “treating” refers to an intervention (e.g. the administration of an agent to a subject) which cures, ameliorates or lessens the symptoms of a disease or removes (or lessens the impact of) its cause(s) (for example, the causative bacterium). In this case, the term is used synonymously with the term “therapy”. Thus, the treatment of infection according to the invention may be characterized by the (direct or indirect) bacteriostatic and/or bactericidal action of the compounds of the invention. Thus, the compounds of the invention find application in methods of killing, or preventing the growth of, bacterial cells.

Additionally, the terms “treatment” or “treating” refers to an intervention (e.g. the administration of an agent to a subject) which prevents or delays the onset or progression of a disease or reduces (or eradicates) its incidence within a treated population. In this case, the term treatment is used synonymously with the term “prophylaxis”.

The term “subject” (which is to be read to include “individual”, “animal”, “patient” or “mammal” where context permits) defines any subject, particularly a mammalian subject, for whom treatment is indicated. Mammalian subjects include, but are not limited to, humans, domestic animals, farm animals, zoo animals, sport animals, pet animals such as dogs, cats, guinea pigs, rabbits, rats, mice, horses, cattle, cows; primates such as apes, monkeys, orangutans, and chimpanzees; canids such as dogs and wolves; felids such as cats, lions, and tigers; equids such as horses, donkeys, and zebras; food animals such as cows, pigs, and sheep; ungulates such as deer and giraffes; rodents such as mice, rats, hamsters and guinea pigs; and so on. In preferred embodiments, the subject is a human, for example an infant human or a geriatric human.

The terms Gram-negative bacterium and Gram-positive bacterium are terms of art defining two distinct classes of bacteria on the basis of certain cell wall staining characteristics.

The term low G+C Gram-positive bacterium is a term of art defining a particular subclass class of evolutionarily related bacteria within the Gram-positives on the basis of the composition of the bases in the DNA. The subclass includes Streptococcus spp., Staphylococcus spp., Listeria spp., Bacillus spp., Clostridium spp., Enterococcus spp. and Lactobacillus spp.).

The term high G+C Gram-positive bacterium is a term of art defining a particular subclass class of evolutionarily related bacteria within the Gram-positives on the basis of the composition of the bases in the DNA. The subclass includes actinomycetes (actinobacteria) including Actinomyces spp., Arthrobacter spp., Corynebacterium spp., Frankia spp., Micrococcus spp., Micromonospora spp., Mycobacterium spp., Nocardia spp., Propionibacterium spp. and Streptomyces spp.

As used herein, the term “combination”, as applied to two or more compounds and/or agents (also referred to herein as the components), is intended to define material in which the two or more compounds/agents are associated. The terms “combined” and “combining” in this context are to be interpreted accordingly.

The association of the two or more compounds/agents in a combination may be physical or non-physical. Examples of physically associated combined compounds/agents include:
compositions (e.g. unitary formulations) comprising the two or more compounds/agents in admixture (for example within the same unit dose);
compositions comprising material in which the two or more compounds/agents are chemically/physicochemically linked (for example by crosslinking, molecular agglomeration or binding to a common vehicle moiety);
compositions comprising material in which the two or more compounds/agents are chemically/physicochemically co-packaged (for example, disposed on or within lipid vesicles, particles (e.g. micro- or nanoparticles) or emulsion droplets);
pharmaceutical kits, pharmaceutical packs or patient packs in which the two or more compounds/agents are co-packaged or co-presented (e.g. as part of an array of unit doses);

Examples of non-physically associated combined compounds/agents include:
material (e.g. a non-unitary formulation) comprising at least one of the two or more compounds/agents together with instructions for the extemporaneous association of the at least one compound/agent to form a physical association of the two or more compounds/agents;
material (e.g. a non-unitary formulation) comprising at least one of the two or more compounds/agents together with instructions for combination therapy with the two or more compounds/agents;
material comprising at least one of the two or more compounds/agents together with instructions for administration to a patient population in which the other(s) of the two or more compounds/agents have been (or are being) administered;
material comprising at least one of the two or more compounds/agents in an amount or in a form which is specifically adapted for use in combination with the other(s) of the two or more compounds/agents.

As used herein, the term “combination therapy” is intended to define therapies which comprise the use of a combination of two or more compounds/agents (as defined above). Thus, references to “combination therapy”, “combinations” and the use of compounds/agents “in combination” in this application may refer to compounds/agents that are administered as part of the same overall treatment regimen. As such, the posology of each of the two or more compounds/agents may differ: each may be administered at the same time or at different times. It will therefore be appreciated that the compounds/agents of the combination may be administered sequentially (e.g. before or after) or simultaneously, either in the same pharmaceutical formulation (i.e. together), or in different pharmaceutical formulations (i.e. separately). Simultaneously in the same formulation is as a unitary formulation whereas simultaneously in different pharmaceutical formulations is non-unitary. Each of the two or more compounds/agents in a combination therapy may also be administered via a different route and/or according to a different dosing regimen/duration.

As used herein, the term “pharmaceutical kit” defines an array of one or more unit doses of a pharmaceutical composition together with dosing means (e.g. measuring device) and/or delivery means (e.g. inhaler or syringe), optionally all contained within common outer packaging. In pharmaceutical kits comprising a combination of two or more compounds/agents, the individual compounds/agents may unitary or non-unitary formulations. The unit dose(s) may be contained within a blister pack. The pharmaceutical kit may optionally further comprise instructions for use.

As used herein, the term “pharmaceutical pack” defines an array of one or more unit doses of a pharmaceutical composition, optionally contained within common outer packaging. In pharmaceutical packs comprising a combination of two or more compounds/agents, the individual compounds/agents may unitary or non-unitary formulations. The unit dose(s) may be contained within a blister pack. The pharmaceutical pack may optionally further comprise instructions for use.

As used herein, the term “patient pack” defines a package, prescribed to a patient, which contains pharmaceutical compositions for the whole course of treatment. Patient packs usually contain one or more blister pack(s). Patient packs have an advantage over traditional prescriptions, where a pharmacist divides a patient's supply of a pharmaceutical from a bulk supply, in that the patient always has access to the package insert contained in the patient pack, normally missing in patient prescriptions. The inclusion of a package insert has been shown to improve patient compliance with the physician's instructions. The combinations of the invention may produce a therapeutically efficacious effect relative to the therapeutic effect of the individual compounds/agents when administered separately.

As used herein, an effective amount or a therapeutically effective amount of a compound defines an amount that can be administered to a subject without excessive toxicity, irritation, allergic response, or other problem or complication, commensurate with a reasonable benefit/risk ratio, but one that is sufficient to provide the desired effect, e.g. the treatment or prophylaxis manifested by a permanent or temporary improvement in the subject's condition. The amount will vary from subject to subject, depending on the age and general condition of the individual, mode of administration and other factors. Thus, while it is not possible to specify an exact effective amount, those skilled in the art will be able to determine an appropriate “effective” amount in any individual case using routine experimentation and background general knowledge. A therapeutic result in this context includes eradication or lessening of symptoms, reduced pain or discomfort, prolonged survival, improved mobility and other markers of clinical improvement. A therapeutic result need not be a complete cure.

As used herein, a “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

The term “adjunctive agent” as used herein is intended to define any compound or composition which yields an efficacious combination (as herein defined) when combined with a compound of the invention. The adjunctive agent or treatment may therefore contribute to efficacy (for example, by producing a synergistic or additive effect or by potentiating the activity of the compound of the invention).

The term “efficacious” includes advantageous effects such as additivity, synergism, reduced side effects, reduced toxicity or improved performance or activity. Advantageously, an efficacious effect may allow for lower doses of each or either component to be administered to a patient, thereby decreasing the toxicity, whilst producing and/or maintaining the same therapeutic effect. A synergistic effect in the present context refers to a therapeutic effect produced by the combination which is larger than the sum of the therapeutic effects of the components of the combination when presented individually. An additive effect in the present context refers to a therapeutic effect produced by the combination which is larger than the therapeutic effect of any of the components of the combination when presented individually.

The term “adjunctive” as applied to the use of the compounds and compositions of the invention in therapy or prophylaxis defines uses in which the materials are administered together with one or more other drugs, interventions, regimens or treatments (such as surgery and/or irradiation). Such adjunctive therapies may comprise the concurrent, separate or sequential administration/application of the materials of the invention and the other treatment(s). Thus, in some embodiments, adjunctive use of the materials of the invention is reflected in the formulation of the pharmaceutical compositions of the invention. For example, adjunctive use may be reflected in a specific unit dosage, or in formulations in which the compound of the invention is present in admixture with the other drug(s) with which it is to be used adjunctively (or else physically associated with the other drug(s) within a single unit dose). In other embodiments, adjunctive use of the compounds or compositions of the invention may be reflected in the composition of the pharmaceutical kits of the invention, wherein the compound of the invention is co-packaged (e.g. as part of an array of unit doses) with the other drug(s) with which it is to be used adjunctively. In yet other embodiments, adjunctive use of the compounds of the invention may be reflected in the content of the information and/or instructions co-packaged with the compound relating to formulation and/or posology.

The term pharmaceutically acceptable salt as applied to the compounds of the invention defines any non-toxic organic or inorganic acid addition salt of the free base which are suitable for use in contact with the tissues of humans and lower animals without undue toxicity, irritation, allergic response and which are commensurate with a reasonable benefit/risk ratio. Suitable pharmaceutically acceptable salts are well known in the art. Examples are the salts with inorganic acids (for example hydrochloric, hydrobromic, sulphuric and phosphoric acids), organic carboxylic acids (for example acetic, propionic, glycolic, lactic, pyruvic, malonic, succinic, fumaric, malic, tartaric, citric, ascorbic, maleic, hydroxymaleic, dihydroxymaleic, benzoic, phenylacetic, 4-aminobenzoic, 4-hydroxybenzoic, anthranilic, cinnamic, salicylic, 2-phenoxybenzoic, 2-acetoxybenzoic and mandelic acid) and organic sulfonic acids (for example methanesulfonic acid and p-toluenesulfonic acid).

The term pharmaceutically acceptable derivative as applied to the compounds of the invention define compounds which are obtained (or obtainable) by chemical derivatization of the parent compounds of the invention. The pharmaceutically acceptable derivatives are therefore suitable for administration to or use in contact with mammalian tissues without undue toxicity, irritation or allergic response (i.e. commensurate with a reasonable benefit/risk ratio). Preferred derivatives are those obtained (or obtainable) by alkylation, esterification or acylation of the parent compounds of the invention. The derivatives may be active per se, or may be inactive until processed in vivo. In the latter case, the derivatives of the invention act as prodrugs. Particularly preferred prodrugs are ester derivatives which are esterified at one or more of the free hydroxyls and which are activated by hydrolysis in vivo. Other preferred prodrugs are covalently bonded compounds which release the active parent drug according to general formula (I) after cleavage of the covalent bond(s) in vivo.

In its broadest aspect, the present invention contemplates all optical isomers, racemic forms and diastereoisomers of the compounds described herein. Those skilled in the art will appreciate that, owing to the asymmetrically substituted carbon atoms present in the compounds of the invention, the compounds may be produced in optically active and racemic forms. If a chiral centre or another form of isomeric centre is present in a compound of the present invention, all forms of such isomer or isomers, including enantiomers and diastereoisomers, are intended to be covered herein. Compounds of the invention containing a chiral centre (or multiple chiral centres) may be used as a racemic mixture, an enantiomerically enriched mixture, or the racemic mixture may be separated using well-known techniques and an individual enantiomer may be used alone. Thus, references to particular compounds of the present invention encompass the products as a mixture of diastereoisomers, as individual diastereoisomers, as a mixture of enantiomers as well as in the form of individual enantiomers.

Therefore, the present invention contemplates all optical isomers and racemic forms thereof of the compounds of the invention, and unless indicated otherwise (e.g. by use of dash-wedge structural formulae) the compounds shown herein are intended to encompass all possible optical isomers of the compounds so depicted. In cases where the stereochemical form of the compound is important for pharmaceutical utility, the invention contemplates use of an isolated eutomer.

The term bioisostere (or simply isostere) is a term of art used to define drug analogues in which one or more atoms (or groups of atoms) have been substituted with replacement atoms (or groups of atoms) having similar steric and/or electronic features to those atoms which they replace. The substitution of a hydrogen atom or a hydroxyl group with a fluorine atom is a commonly employed bioisosteric replacement. Sila-substitution (C/Si-exchange) is a relatively recent technique for producing isosteres. This approach involves the replacement of one or more specific carbon atoms in a compound with silicon (for a review, see Tacke and Zilch (1986) Endeavour, New Series 10: 191-197). The sila-substituted isosteres (silicon isosteres) may exhibit improved pharmacological properties, and may for example be better tolerated, have a longer half-life or exhibit increased potency (see for example Englebienne (2005) Med. Chem., 1 (3): 215-226). Similarly, replacement of an atom by one of its isotopes, for example hydrogen by deuterium, may also lead to improved pharmacological properties, for example leading to longer half-life (see for example Kushner et al (1999) Can J Physiol Pharmacol. 77 (2):79-88). In its broadest aspect, the present invention contemplates all bioisosteres (and specifically, all silicon Bioisosteres, and all deuterium Bioisosteres) of the compounds of the invention.

All references to particular chemical compounds herein are to be interpreted as covering the compounds per se, and also, where appropriate, pharmaceutically acceptable salts, derivatives, hydrates, solvates, complexes, isomers, tautomers, bioisosteres, N-oxides, esters, prodrugs, isotopes or protected forms thereof.

The term “C₁₋₄-alkyl” denotes a straight or branched alkyl group having from 1 to 4 carbon atoms. For parts of the range C₁₋₄-alkyl all subgroups thereof are contemplated such as C₁₋₃-alkyl, C₁₋₂-alkyl, C₂₋₄-alkyl, C₂₋₃-alkyl and C₃₋₄-alkyl. Examples of said C₁₋₄-alkyl include methyl, ethyl, n-propyl, isopropyl, n-butyl, isobutyl, sec-butyl and tert-butyl.

The term “C₁₋₄-alkylene” denotes a straight or branched divalent saturated hydrocarbon chain having from 1 to 4 carbon atoms. The C₁₋₄-alkylene chain may be attached to the rest of the molecule and to the radical group through one carbon within the chain or through any two carbons within the chain. Examples of C₁₋₄-alkylene radicals include methylene [—CH₂—], 1,2-ethylene [—CH₂—CH₂—], 1,1-ethylene [—CH(CH₃)—], 1,2-propylene [—CH₂—CH(CH₃)—] and 1,3-propylene [—CH₂—CH₂—CH₂—]. When referring to a “C₁₋₄-alkylene” radical, all subgroups thereof are contemplated, such as C₁₋₂-alkylene, C₁₋₃-alkylene, C₂₋₃-alkylene, or C₃₋₄-alkylene.

The term “C₂₋₄alkynyl” denotes a straight or branched monovalent saturated hydrocarbon chain having 2 to 4 carbon atoms and comprising at least one carbon-carbon triple bond. The C₂₋₄alkynyl chain may be attached to the rest of the molecule through a carbon within the chain. Examples of said C₂₋₄alkynyl include ethynyl, propargyl, but-1-ynyl and but-2-ynyl. When referring to a “C₂₋₄alkynyl”, all subgroups thereof are contemplated, such as C₂₋₃alkynyl and C₃₋₄alkynyl.

The term “C₁₋₄-alkoxy” refers to a straight or branched C₁₋₄-alkyl group which is attached to the remainder of the molecule through an oxygen atom. For parts of the range C₁₋₄-alkoxy, all subgroups thereof are contemplated such as C₁₋₃-alkoxy, C₁₋₂-alkoxy, C₂₋₄-alkoxy, C₂₋₃-alkoxy and C₃₋₄-alkoxy. Examples of said C₁₋₄-alkoxy include methoxy, ethoxy, n-propoxy, isopropoxy, n-butoxy, isobutoxy, sec-butoxy and tert-butoxy.

The term “halo-C₁₋₄-alkyl” denotes a straight or branched C₁₋₄-alkyl group that has one or more hydrogen atoms thereof replaced with halogen. Examples of said halo-C₁₋₄-alkyl include fluoro-C₁₋₄-alkyl such as fluoromethyl, trifluoromethyl, or 2-fluoroethyl, and chloro-C₁₋₄-alkyl such as trichloromethyl.

The term “halo-C₁₋₄-alkoxy” denotes a straight or branched C₁₋₄-alkyl group that has one or more hydrogen atoms thereof replaced with halogen and is connected to the rest of the molecule through an oxygen atom. Examples of said halo-C₁₋₄-alkyl include fluoro-C₁₋₄-alkyl such as fluoromethyl, trifluoromethyl, or 2-fluoroethyl, and chloro-C₁₋₄-alkyl such as trichloromethyl.

The term “C₁₋₄-alkyl-X”, wherein X is a substituent means that a single X substituent is connected to any carbon atom of C₁₋₄-alkyl. Said C₁₋₄-alkyl-X may be attached to the rest of the molecule through a carbon atom of the C₁₋₄alkyl. The substituent X can be any substituent, such as —NR^{4A}R^{4B}, —C₁₋₄-alkoxy, and C₃₋₇-cycloalkyl. Examples of “C₁₋₄-alkyl-X” groups include —CH₂—NR^{4A}R^{4B}, —CH₂CH₂—NR^{4A}R^{4B}, —CH₂CH(NR^{4A}R^{4B})CH₃—, —CH₂CH₂OCH₃, and —C(H)(OCH₃)CH₃.

“Halogen” refers to fluorine, chlorine, bromine or iodine, preferably fluorine and chlorine, most preferably fluorine.

“Hydroxy” and “Hydroxyl” refer to the —OH radical.

The term “hydroxylC₁₋₄alkyl” denotes a straight or branched C₁₋₄alkyl group that has one or more hydrogen atoms replaced with hydroxy and is attached to the rest of the molecule through a carbon atom of the C₁₋₄alkyl group. Examples of said hydroxylC₁₋₄alkyl include —CH₂OH, —CH₂CH₂OH, —CH(OH)CH₃ and CH₂CH₂CH₂OH.

“Cyano” refers to the —CN radical.

“Oxo” refers to the carbonyl group ═O.

“Alkali metal” refers to elements occupying Group 1 of the periodic table. Examples of said alkali metals include lithium, sodium and potassium.

“Optional” or “optionally” means that the subsequently described event or circumstance may but need not occur, and that the description includes instances where the event or circumstance occurs and instances in which it does not.

The term “C₃₋₇-cycloalkyl” refers to a monocyclic saturated or partially unsaturated hydrocarbon ring system having from 3 to 7 carbon atoms. Examples of said C₃₋₇-cycloalkyl include cyclopropyl, cyclobutyl, cyclopentyl, cyclohexyl, cyclohexenyl, cycloheptyl, and cycloheptenyl. For parts of the range “C₃₋₇-cycloalkyl” all subgroups thereof are contemplated such as C₃₋₇-cycloalkyl, C₃₋₆-cycloalkyl, C₃₋₅-cycloalkyl, C₃₋₄-cycloalkyl, C₄₋₇-cycloalkyl, C₄₋₆-cycloalkyl, C₄₋₅-cycloalkyl, C₅₋₇-cycloalkyl, C₅₋₆-cycloalkyl, and C₆₋₇-cycloalkyl.

The terms “heterocyclyl” and “heterocyclic ring” denote a non-aromatic, fully saturated or partially unsaturated, preferably fully saturated, monocyclic ring system having from 4 to 7 ring atoms, especially 5 or 6 ring atoms, in which one or more of the ring atoms are other than carbon, such as nitrogen, sulphur or oxygen. The said ring system may be attached to the rest of the molecule through either a heteroatom or a carbon atom of the ring system. Examples of heterocyclic groups include but are not limited to piperidinyl, morpholinyl, homomorpholinyl, azepanyl, piperazinyl, oxo-piperazinyl, diazepinyl, tertahydropyridinyl, tetrahydropyranyl, pyrrolidinyl, tertrahydrofuranyl, and dihydropyrrolyl.

The terms “heteroaryl” and “heteroaromatic ring” denote a monocyclic heteroaromatic ring comprising 5 to 6 ring atoms in which one or more of the ring atoms are other than carbon, such as nitrogen, sulphur or oxygen. Typically the heteroaryl ring will contain up to 4 heteroatoms, more typically up to 3 heteroatoms, more usually up to 2, for example a single heteroatom. The said heteroaromatic ring may be attached to the rest of the molecule through either a heteratom or a carbon atom of the ring system. Examples of heteroaryl groups include but are not limited to furyl, pyrrolyl, thienyl, oxazolyl, isoxazolyl, oxadiazolyl, imidazolyl, oxatriazoly, thiazolyl, isothiazolyl, tetrazolyl, pyrazolyl, pyridinyl, pyrimidinyl, pyridazinyl, pyrazinyl, triazinyl and thiadiazolyl. In some embodiments, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general the number of basic nitrogen atoms present in the heteroaryl group, including any amino group substituents of the ring, will be less than five.

The terms “unsaturated” and “partially saturated” refer to rings wherein the ring structure(s) contains atoms sharing more than one valence bond i.e. the ring contains at least one multiple bond e.g. a C═C, C═C or N═C bond. The term “fully saturated” refers to rings where there are no multiple bonds between ring atoms. Saturated carbocyclic groups include cycloalkyl groups as defined below. Partially saturated carbocyclic groups include cycloalkene groups as defined below.

Examples of monocyclic non-aromatic heterocyclic groups include 5-, 6-, and 7-membered monocyclic heterocyclic groups. The monocyclic non-aromatic heterocyclic groups may be attached to the rest of the molecule through either a heteroatom or a carbon atom of the heterocyclic group. Particular examples include morpholine, piperidine (e.g. 1-piperidinyl, 2-piperidinyl, 3-piperidinyl and 4-piperidinyl), pyrrolidine (e.g. 1-pyrrolidinyl, 2-pyrrolidinyl and 3-pyrrolidinyl), pyrrolidone, pyran (2H-pyran or 4H-pyran), dihydrothiophene, dihydropyran, dihydrofuran, dihydrothiazole, tetrahydrofuran, tetrahydrothiophene, dioxane, tetrahydropyran (e.g. 4-tetrahydro pyranyl), imidazoline, imidazolidinone, oxazoline, thiazoline, 2-pyrazoline, pyrazolidine, piperazine, and N-alkyl piperazines such as N-methyl piperazine. Further examples include thiomorpholine and its S-oxide and S,S-dioxide (particularly thiomorpholine). Still further examples include azetidine, piperidone, piperazone, and N-alkyl piperidines such as N-methyl piperidine.

The term “cyclic amino group” refers to a non-aromatic, fully saturated or partially unsaturated, preferably fully saturated, monocyclic ring system having from 4 to 7 ring atoms, especially 5 or 6 ring atoms, in which one of the ring atoms is nitrogen and the group is attached to the rest of the molecule via this nitrogen atom. In such cyclic amino groups, one or more of the remaining ring atoms may be other than carbon, such as nitrogen, sulphur or oxygen. Examples of such cyclic amino groups include piperidine (1-piperidinyl), pyrrolidine (1-pyrrolidinyl), pyrrolidone, morpholine or piperazine.

Embodiments of the compounds of general formula (I) are described below.

The Group R¹

R¹ is selected from hydrogen (i.e. H) and C₁₋₄alkyl such as methyl, ethyl, and isopropyl. In an embodiment, R¹ is hydrogen (i.e. H).

The Group Ar¹

Ar¹ has the formula (A1)

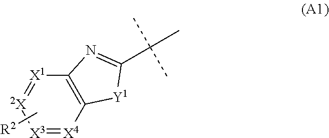

X¹, X², X³, and X⁴ are each independently selected from N and CH;

Y¹ is selected from O and NR³.

R³ is hydrogen or C₁₋₄alkyl such as methyl, ethyl, n-propyl, iso-propyl, sec-butyl, or tert-butyl. In an embodiment R³ is hydrogen (i.e. H) or methyl.

R² is one or more optional substituents on the 6-membered ring of Ar¹. The R² subsituent(s) is (are) optional, meaning that it (they) may be present or not. In an embodiment, R² is absent, meaning that the 6-membered ring system of A1 is unsubstituted. Each R² substituent, when present, is independently selected from halogen such as fluoro, chloro, bromo or iodo, hydroxyl, cyano, hydroxylC₁₋₄alkyl such as —CH₂OH, C₁₋₄alkyl such as methyl, ethyl, n-propyl, iso-propyl, sec-butyl, or tert-butyl, haloC₁₋₄alkyl such as trifluoromethyl or difluoromethyl, C₁₋₄alkoxy such as methoxy, ethoxy or isopropoxy, haloC₁₋₄alkyloxy such as trifluoromethoxy, —C₁₋₄ alkylC₁₋₄alkoxy such as —CH₂CH₂OCH₃, C₁₋₄alkoxyC₁₋₄alkoxy such as —OCH₂CH₂OCH₃, —NR^{4A}R^{4B} such as —N(CH₃)₂, —NH(CH₃) or —NHCOCH₃, —CONR^{4A}R^{4B} such as CON(CH₃)₂ or CONHCH₃, —C₁₋₄alkylNR^{4A}R^{4B} such as —CH₂CH₂N(CH₃)₂, —C₁₋₄alkoxyNR^{4A}R^{4B} such as OCH₂CH₂N(CH₃)₂, NO₂, morpholinyl (—NH(CH₂CH₂)₂₀), C₃₋₇ cycloalkyl such as cyclopropyl, cyclobutyl, cyclopentyl or cyclohexyl, alkynyl such as —CCH, and —CO₂R⁴ such as CO₂H, CO₂CH₃, or CO₂CH₂CH₃ wherein R⁴ is hydrogen or C₁₋₄alkyl.

R^{4A} and R^{4B} are each independently selected from hydrogen, C₁₋₄alkyl, —C₁₋₄alkylC₁₋₄alkoxy, and COR⁴, or

R^{4A} and R^{4B}, together with the nitrogen atom to which they are attached, join together to form a cyclic amino group such as a pyrrolidine ring, wherein the cyclic amino group is optionally substituted with oxo;

In an embodiment, R² is one or more substituents each independently selected from fluoro, chloro, methyl, ethyl, iso-propyl, cyclopropyl, methoxy, trifluoromethyl, trifluoromethyloxy (—OCF₃), —NR^{4A}R^{4B}, CO₂H and CO₂CH₃. In embodiments having two or more R² substituents on the 6-membered ring, the R² substituents may be the same or different.

In an embodiment, one or two of the ring atoms X¹⁻⁴ are N (i.e. a nitrogen atom), and the remaining X¹⁻⁴ ring atoms are independently selected from CH and CR².

Y¹ can be an oxygen (i.e. O) atom. Embodiments having one or more optional R² substituents include:

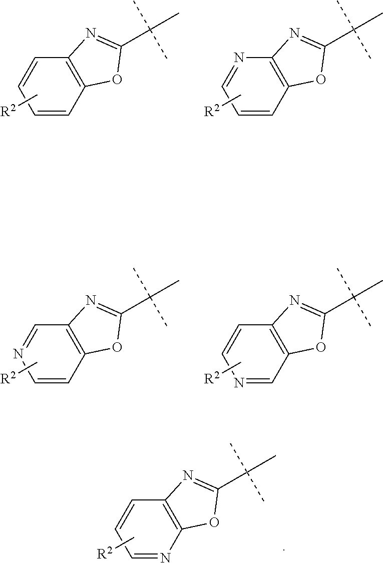

Further more specific embodiments of Ar¹ having Y¹═O include:

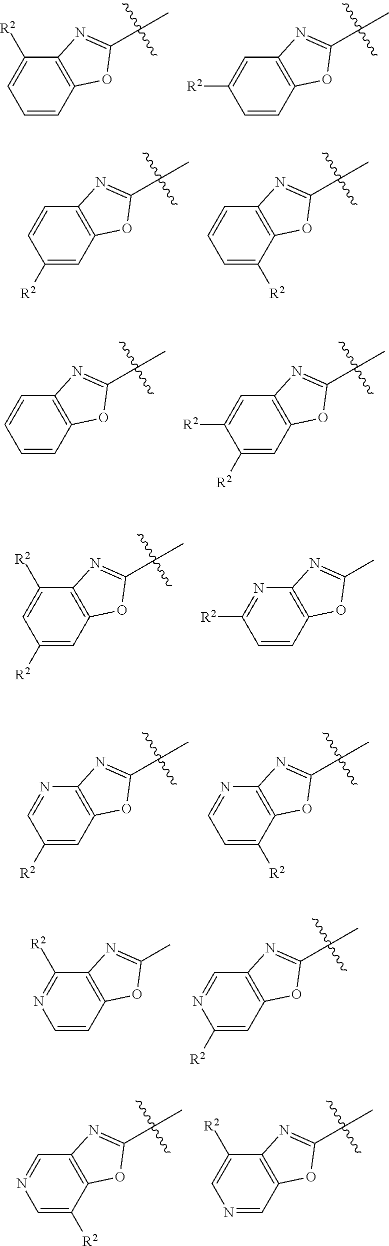

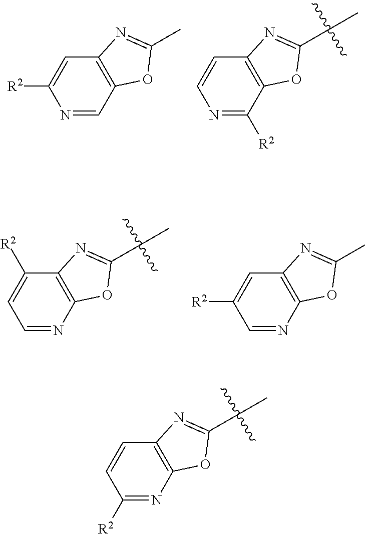

wherein R² is a substituent as defined above.

Yet further embodiments of Ar¹ having Y¹═O include:

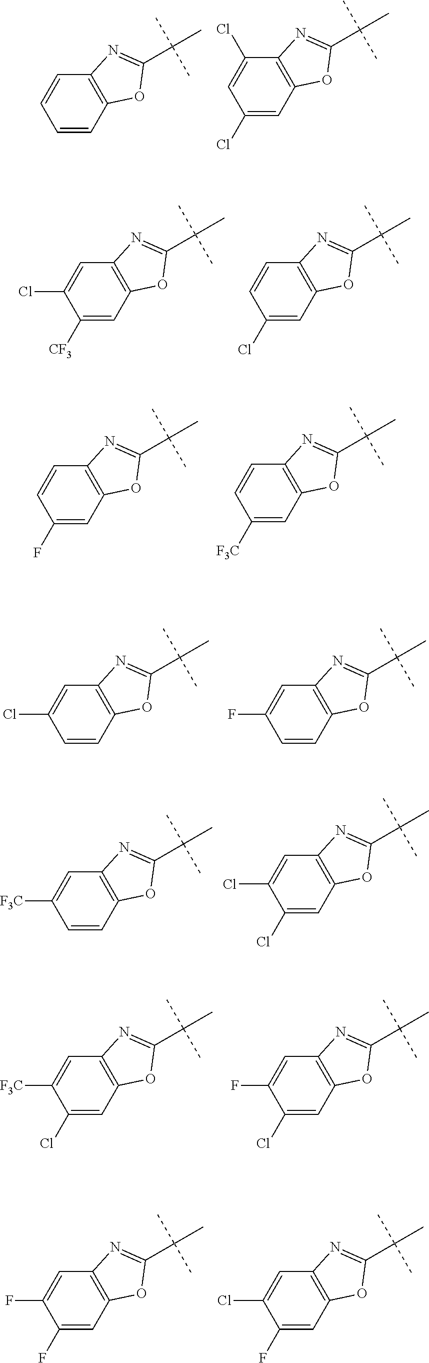

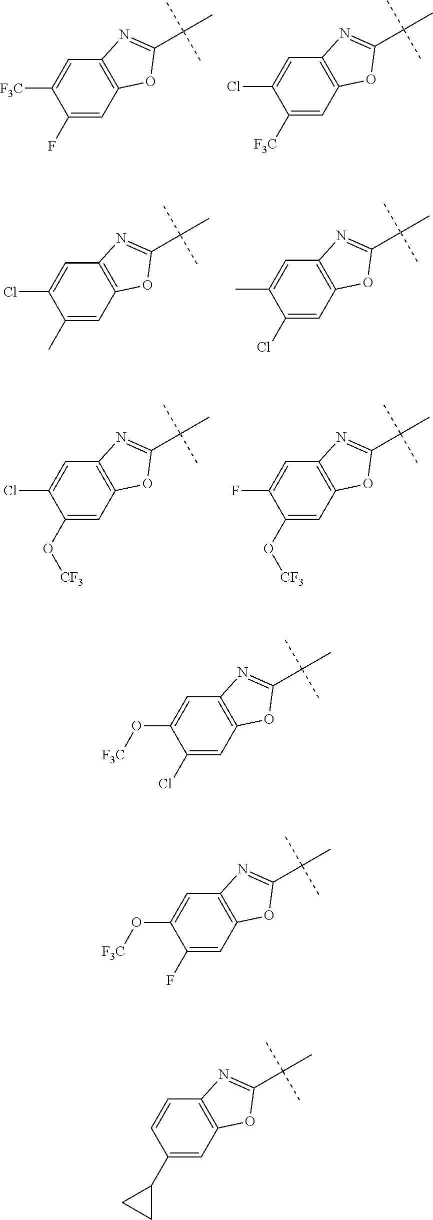

Embodiments having Y¹═NR³ (i.e. a nitrogen atom substituted with R³) include:

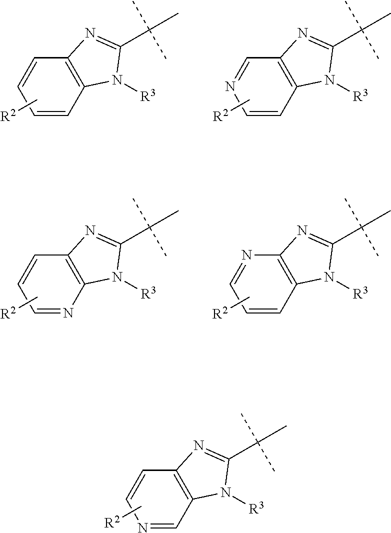

wherein R² is one or more optional substituents as defined above, and R³ is as defined above.

Further embodiments having Y¹═NR³ include:

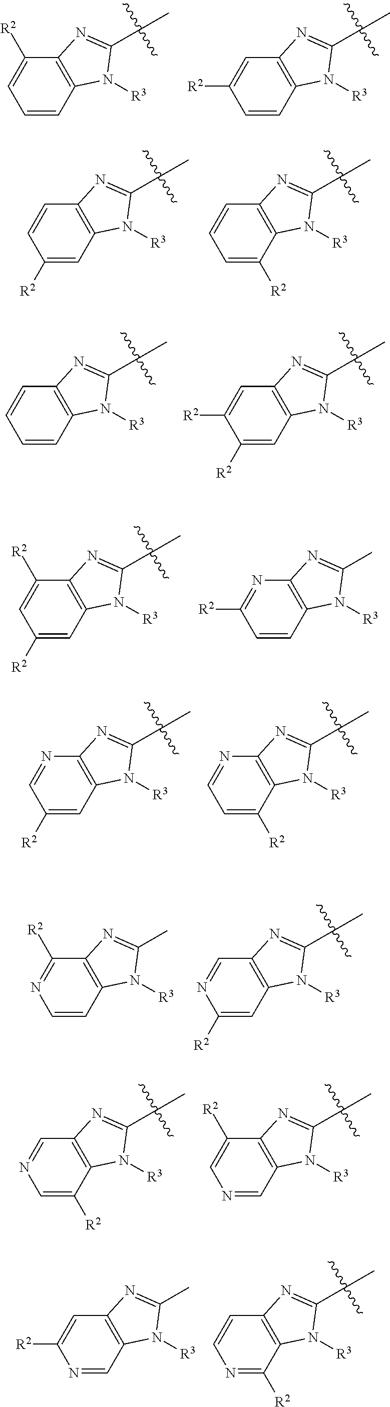

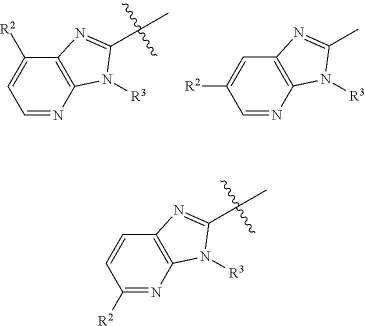

wherein R² is a substituent as defined above, and R³ is as defined above.

The Group Ar²

Ar² is a ring system selected from Group (i), Group (ii), and Group (iii), wherein: Group (i) is a 5-membered heteroaryl ring system selected from any one of (IIa) to (IIm):

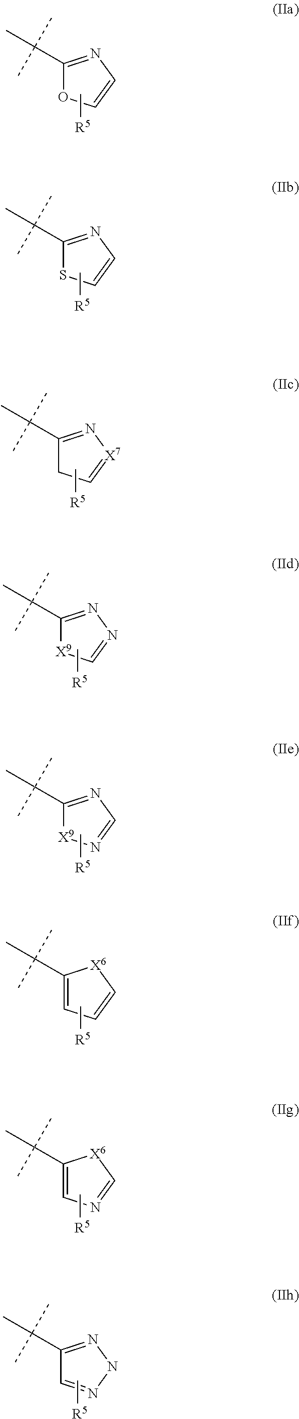

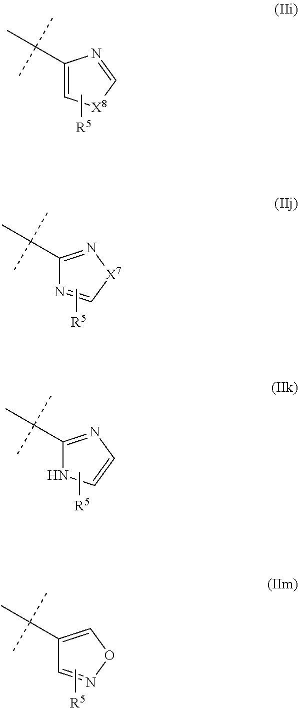

wherein X⁶, X⁷, X⁸, and X⁹ are each independently selected from O, S, and NH.

The R⁵ subsituent(s) is (are) optional, meaning that it (they) may be present or not. In an embodiment, R⁵ is absent, meaning that the Ar² ring is unsubstituted. When present, R⁵ can be connected to any suitable carbon or nitrogen Ar² ring atom. In embodiments having two or more R⁵ substituents on the Ar² ring, the R⁵ substituents can be the same or different. R⁵, when present, is one or more substituents each independently selected from halogen such as fluoro, chloro, bromo or iodo, cyano, C₁₋₄alkyl such as methyl, ethyl, n-propyl, iso-propyl, sec-butyl, or tert-butyl, haloC₁₋₄alkyl such as trifluoromethyl, C₁₋₄alkoxy such as methoxy, ethoxy or isopropoxy, —C₁₋₄alkylC₁₋₄alkoxy such as —CH₂CH₂OCH₃, —CO₂R⁶ such as CO₂H, CO₂CH₃ or CO₂CH₂CH₃, and -L-Q wherein:

L is a linker group selected from a direct bond, C₁₋₃alkylene such as methylene, ethylene or propylene and —CO— (a carbonyl group); and

Q is a group selected from NR^{5A}R^{5B}, C₃cycloalkyl (cyclopropyl) and 4-7 membered heterocyclyl such as pyrrolidinyl, piperidinyl, morpholinyl, or piperazinyl, and wherein the 4-7 membered heterocyclyl ring is optionally substituted with one or more substituents selected from halogen such as fluoro, chloro, bromo or iodo, cyano, C₁₋₄alkyl such as methyl, ethyl, n-propyl, iso-propyl, sec-butyl, or tert-butyl, C₁₋₄alkoxy such as methoxy, ethoxy or isopropoxy and CO₂R⁶ such as CO₂H, CO₂CH₃ or CO₂CH₂CH₃;

R^{5A} and R^{5B} are each independently selected from hydrogen, C₁₋₄alkyl such as methyl, ethyl, n-propyl, iso-propyl, sec-butyl, or tert-butyl, C₃₋₇cycloalkyl such as cyclopropyl, cyclobutyl, cyclopentyl or cyclohexyl, COR⁷ such as CO₂CH₃ or CO₂CH₂CH₃, —C₁₋₄alkyl-NR⁸R⁹ such as —CH₂NHCH₃, —CH₂N(CH₃)₂ or —CH₂CH₂N(CH₃)₂, —C₁₋₄alkylC₁₋₄alkoxy such as —CH₂CH₂OCH₃, phenyl and 5 or 6-membered heteroaryl pyridyl, pyrimidinyl, pyridazinyl, imidazolyl, or pyrazolyl wherein the phenyl or 5 or 6-membered heteroaryl rings are optionally substituted with one or more substituents selected from halogen such as fluoro, chloro, bromo or iodo, and C₁₋₄alkyl such as methyl, ethyl, n-propyl, iso-propyl, sec-butyl, or tert-butyl; or

R^{5A} and R^{5B}, together with the nitrogen atom to which they are attached, join together to form a cyclic amino group such as pyrrolidinyl, piperidinyl, morpholinyl, or piperazinyl, which cyclic amino group is optionally substituted with one or more groups selected from halogen such as fluoro, chloro, bromo or iodo, C₁₋₄alkyl such as methyl, ethyl, n-propyl, iso-propyl, sec-butyl, or tert-butyl, C₁₋₄alkoxy such as methoxy, ethoxy or isopropoxy, cyano, and CO₂R⁶ such as CO₂H, CO₂CH₃ or CO₂CH₂CH₃, R⁶ is hydrogen, C₁₋₄alkyl such as methyl, ethyl, n-propyl, iso-propyl, sec-butyl, or tert-butyl or an alkali metal such as sodium or potassium;

R⁷ is C₁₋₄alkyl such as methyl, ethyl, n-propyl, iso-propyl, sec-butyl, or tert-butyl

R⁸ and R⁹ are each independently selected from hydrogen and C₁₋₄alkyl such as methyl, ethyl, n-propyl, iso-propyl, sec-butyl, or tert-butyl;

In an embodiment, R⁵ is independently selected from any one of fluoro, chloro, methyl, isopropyl, tert-butyl, trifluoromethyl, cyclopropyl, CO₂Et, —NR^{5A}R^{5B}, —CONR^{5A}R^{5B}, —CH₂NR^{5A}R^{5B}, and a ring system selected from pyrrolidinyl, morpholinyl, piperidinyl and piperazinyl, any of which rings is optionally substituted with one or more groups selected from fluoro, chloro, methyl, methoxy, cyano, and CO₂^{t}Bu, and wherein R^{5A} and R^{5B} are each independently selected from hydrogen, methyl, ethyl, isopropyl, cyclopropyl, —COCH₃, —CH₂CH₂N(CH₃)₂, —CH₂CH₂OCH₃, phenyl, and pyridyl, either of which phenyl, and pyridyl rings is optionally substituted with one or more groups selected from fluoro, chloro, and methyl; or R^{5A} and R^{5B} which together with the nitrogen atom to which they are attached form a cyclic amino group selected from pyrrolidinyl, morpholinyl, piperidinyl, piperazinyl, any of which rings is optionally substituted with one or more groups selected from fluoro, methyl, methoxy, cyano, and CO₂^{t}Bu.

In an embodiment, R⁵ is independently selected from any one of fluoro, chloro, methyl, isopropyl, tert-butyl, trifluoromethyl, cyclopropyl, CO₂Et, —NR^{5A}R^{5B}, —CONR^{5A}R^{5B}, —CH₂NR^{5A}R^{5B}, and a ring system selected from pyrrolidinyl, morpholinyl, piperidinyl and piperazinyl, any of which rings is optionally substituted with one or more groups selected from fluoro, chloro, methyl, methoxy, cyano, and CO₂^{t}Bu; wherein R^{5A} and R^{5B} are as defined in the preceding paragraph.

In an embodiment, Ar² is selected from the following ring systems:

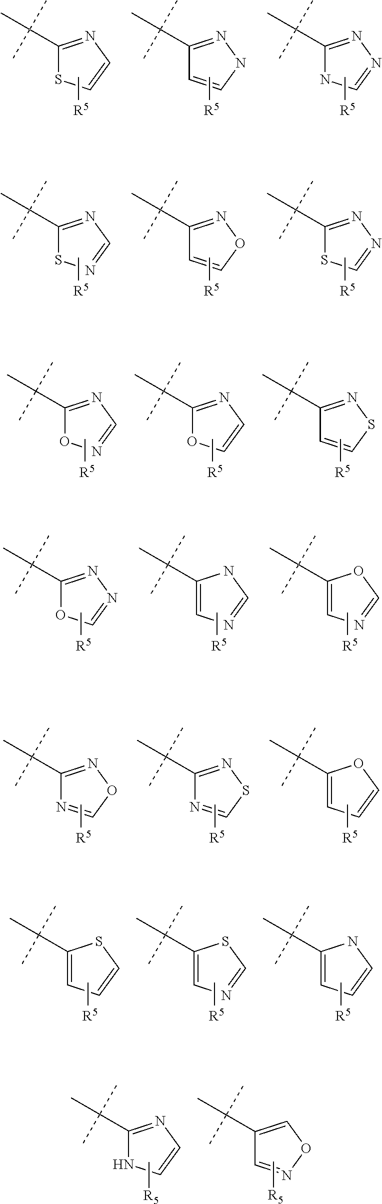

wherein R⁵ is one or more optional substituents as defined above.

In an embodiment, Ar² is selected from the following ring systems:

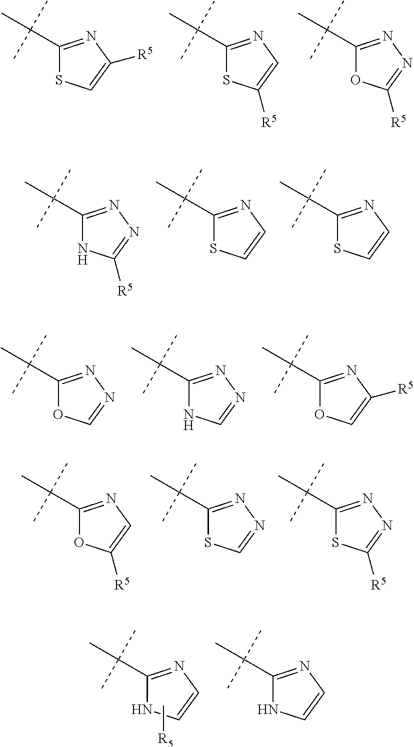

wherein R is a substituent as defined above.

In an embodiment, Ar² is the following ring system:

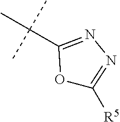

wherein R⁵ is a substituent as defined above.

In an embodiment, Ar² is the following ring system:

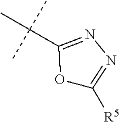

wherein R⁵ is C₁₋₄alkyl such as methyl, isopropyl, tert-butyl, cyclopropyl, —CONR^{5A}R^{5B} or —CH₂NR^{5A}R^{5B}.

In an embodiment, Ar² is the following ring system:

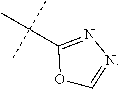

Group (ii) is a 5,6-fused bicyclic heteroaryl ring system having the formula (III):

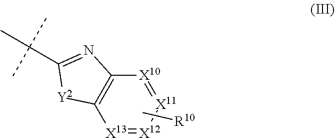

wherein Y² is selected from O and NR^{5C}

R^{5C} is hydrogen or C₁₋₄alkyl such as methyl, ethyl, n-propyl, iso-propyl, sec-butyl, or tert-butyl. In an embodiment is R^{5C} is hydrogen (i.e. H). In an alternative embodiment, R^{5C} is methyl.

X¹⁰, X¹¹, X¹², and X¹³ are each independently selected from N and CH;

R¹⁰ is one or more optional substituents each independently selected from halogen such as fluoro, chloro, bromo or iodo, cyano, C₁₋₄alkyl such as methyl, ethyl, n-propyl, iso-propyl, sec-butyl, or tert-butyl, haloC₁₋₄alkyl such as trifluoromethyl, C₁₋₄ alkoxy such as methoxy, ethoxy or isopropoxy, and —CO₂R⁴ such as CO₂CH₃, or CO₂CH₂CH₃ wherein R⁴ is C₁₋₄alkyl. In an embodiment, R¹⁰ is independently selected from any one of fluoro, chloro, methyl, trifluoromethyl, and CO₂CH₃.

In an embodiment, Ar² is selected from any one of formula (IIIa), (IIIb), and (IIIc):

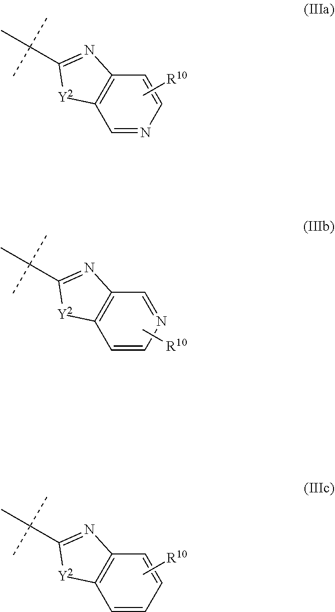

wherein Y² is selected from O and NR^{5C}; and R¹⁰ is as defined above.

Group (iii) is a fused 5,6-fused bicyclic ring system having the formula (IVa) or (IVb)

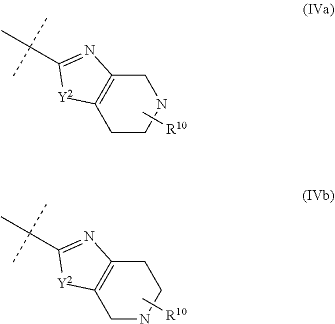

wherein Y² is selected from O and NR^{5C}; and

R¹⁰ is one or more optional substituents each independently selected from halogen such as fluoro, chloro, bromo or iodo, cyano, C₁₋₄alkyl such as methyl, ethyl, n-propyl, iso-propyl, sec-butyl, or tert-butyl, haloC₁₋₄alkyl such as trifluoromethyl, C₁₋₄alkoxy such as methoxy, ethoxy or isopropoxy, and —CO₂R⁴ such as CO₂CH₃, or CO₂CH₂CH₃ wherein R⁴ is C₁₋₄alkyl. In an embodiment, R¹⁰ is independently selected from any one of fluoro, chloro, methyl, trifluoromethyl, and CO₂CH₃. The R¹⁰ substituent may be present on the nitrogen atom of the 6-membered ring and/or on one or more carbon atoms in the 6-membered ring.

In an embodiment, the compound of formula (I) is one of the examples, and pharmaceutically acceptable salts thereof.

In the following embodiments, the compounds of formula (I) have been found to have surprisingly high anti-bacterial activity as well as advantageous pharmacokinetic properties such as high plasma binding and low toxicity levels.

In particular, such affects are most pronounced when the compound of formula (I) has Ar¹ wherein Y¹ is O and as exemplified in the following embodiments and Ar² is selected from amongst Group (i), in particular oxadiazole as exemplified in the following embodiments, wherein R⁵ is selected from methyl, isopropyl, tert-butyl, cyclopropyl, —CONR^{5A}R^{5B} and —CH₂NR^{5A}R^{5B}, and most preferably, wherein R⁵ is absent such that the Ar² ring is unsubstituted. Such compounds may be utilised in the treatment of any bacterial disease. In particular, such compounds are used in the treatment or prophylaxis of infection or intoxication with, or a disease caused by, Neisseria gonorrhoeae.

In a particular embodiment, the compound of formula (I) has Ar¹=(A1) wherein Y¹ is O, R¹ is H and Ar² is selected from amongst Group (i).

In a particular embodiment, the compound of formula (I) has Ar¹=(A1), wherein Y¹ is O, R¹ is H and Ar² is selected from one of the following groups:

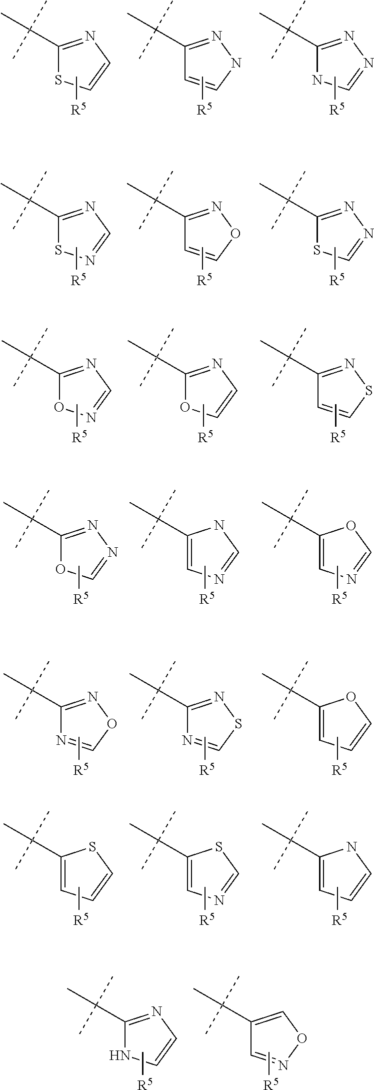

wherein R⁵ is as defined above.

In a particular embodiment, the compound of formula (I) has Ar¹=(A1), wherein Y¹ is O, R¹ is H and Ar² is selected from one of the following groups:

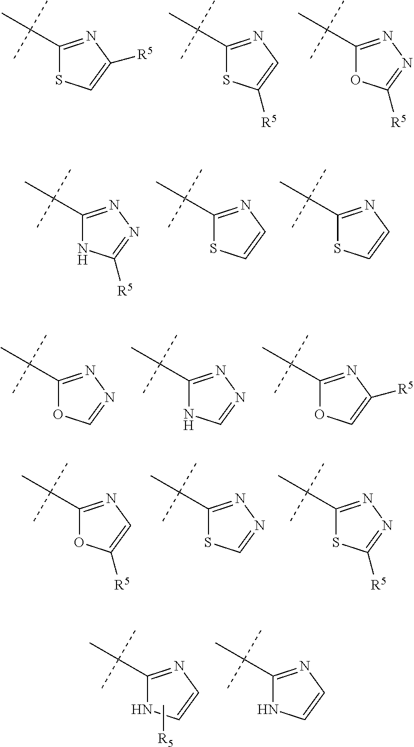

wherein R⁵ is as defined above.

In a particular embodiment, the compound of formula (I) has Ar¹=(A1), wherein Y¹ is O, R, is H and Ar² is the following group:

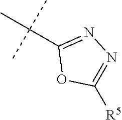

wherein R⁵ is as defined above.

In a particular embodiment, the compound of formula (I) has Ar¹=(A1), wherein Y¹ is O, R¹ is H and Ar² is the following group:

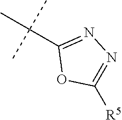

wherein R⁵ is C₁₋₄alkyl such as methyl, isopropyl, tert-butyl, cyclopropyl, —CONR^{5A}R^{5B} or —CH₂NR^{5A}R^{5B}.

In a particular embodiment, the compound of formula (I) has Ar¹=(A1), wherein Y¹ is O, R¹ is H and Ar² is the following group:

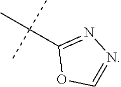

In a particular embodiment, the compound of formula (I) has Ar² selected from amongst Group (i), R¹ is H and Ar¹ is selected from one of the following groups:

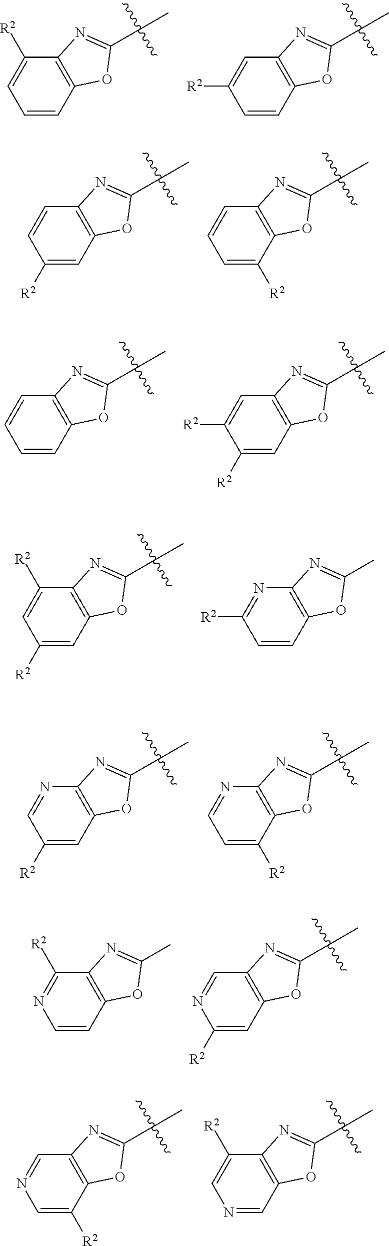

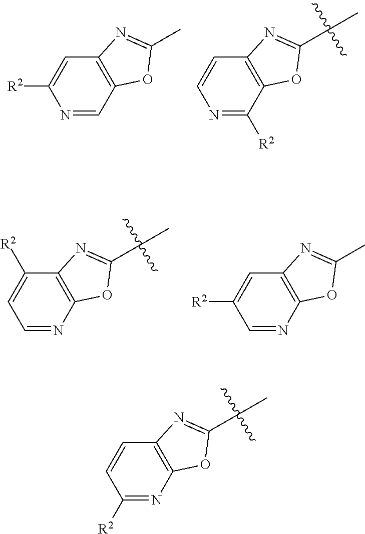

wherein R² is as defined above.

In a particular embodiment, the compound of formula (I) has Ar² selected from amongst Group (i), R¹ is H and Ar¹ is selected from the following groups:

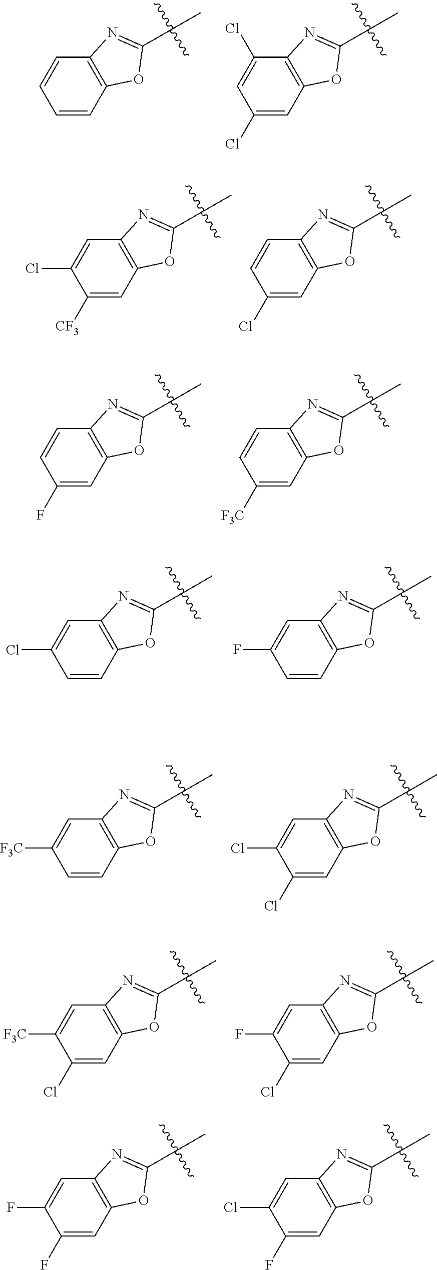

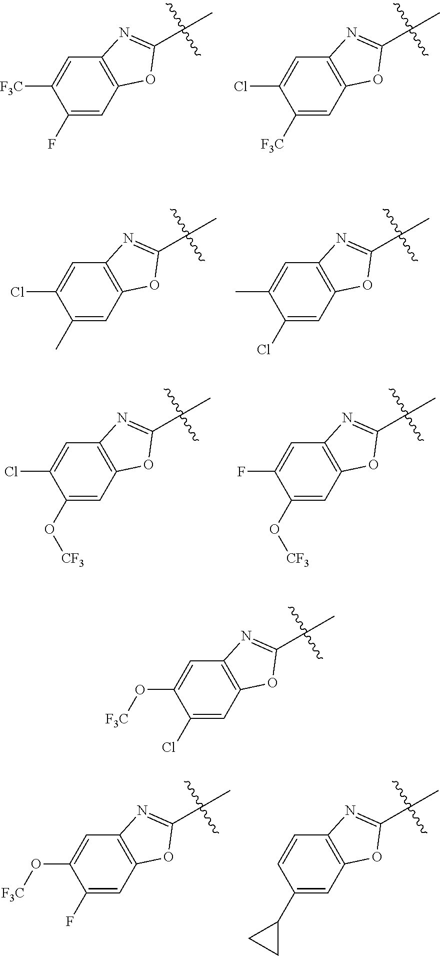

In a particular embodiment, Ar¹ is selected from one of the following groups:

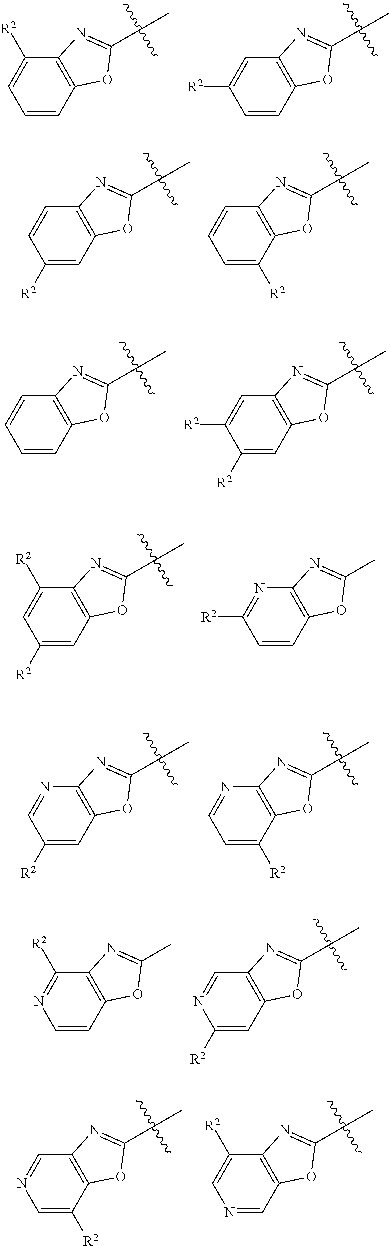

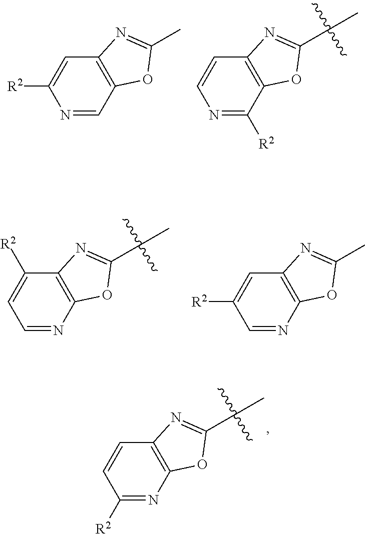

R¹ is H and Ar² is selected from one of the following groups:

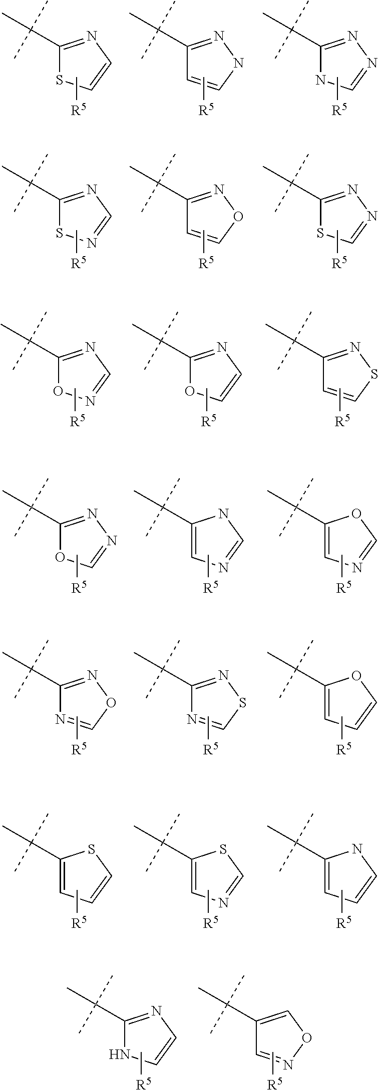

wherein R² and R⁵ are as defined above.

In a particular embodiment, Ar¹ is selected from one of the following groups:

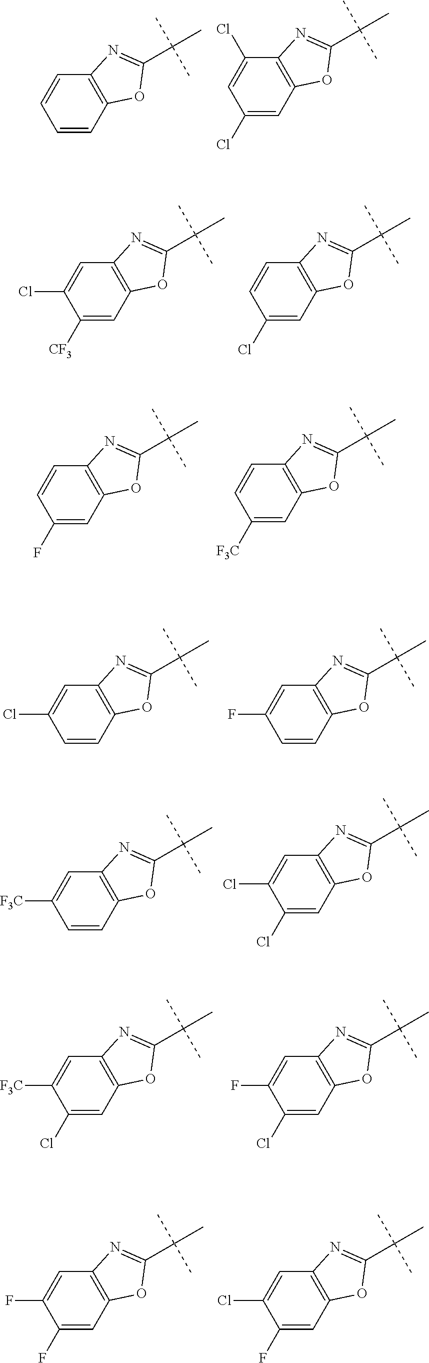

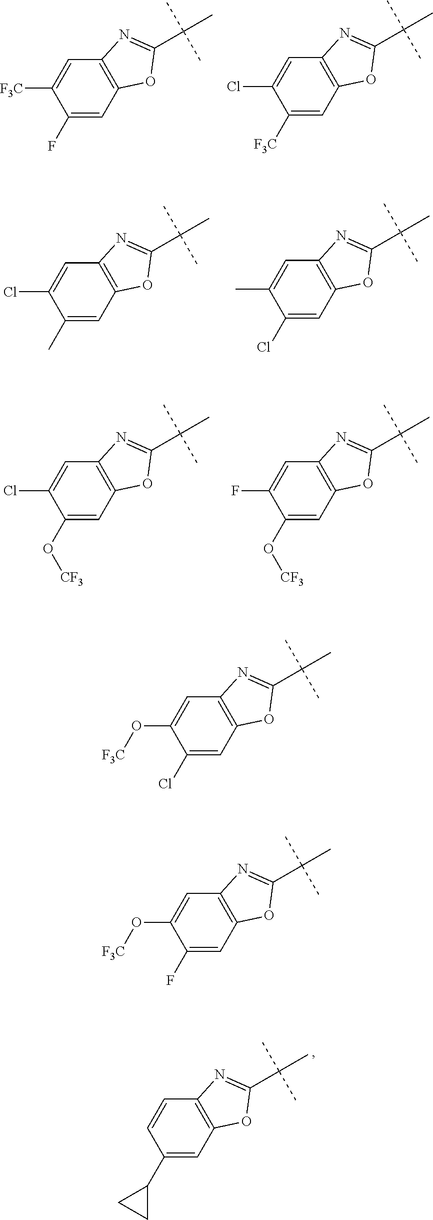

R¹ is H and Ar² is selected from one of the following groups:

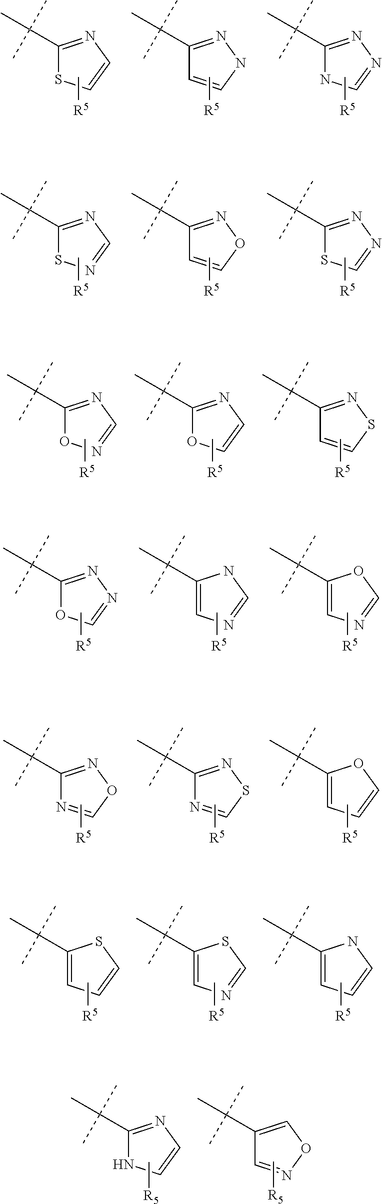

wherein R⁵ is as defined above.

In a particular embodiment, Ar¹ is selected from any one of the following groups:

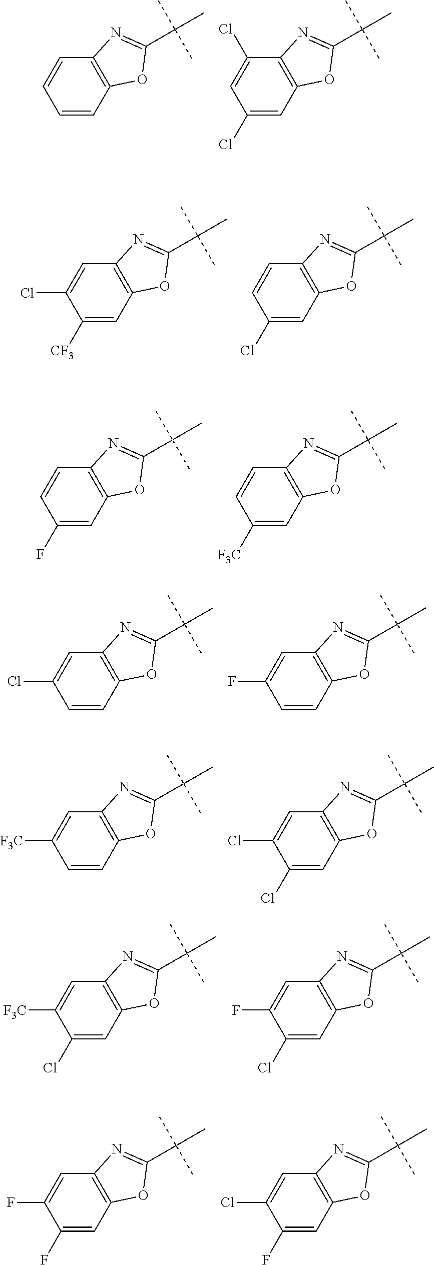

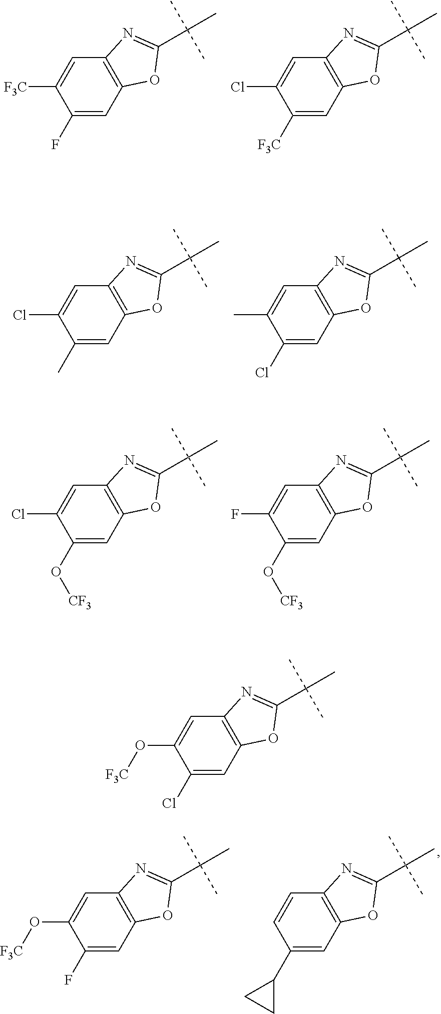

R¹ is H and Ar² is selected from any of the following groups:

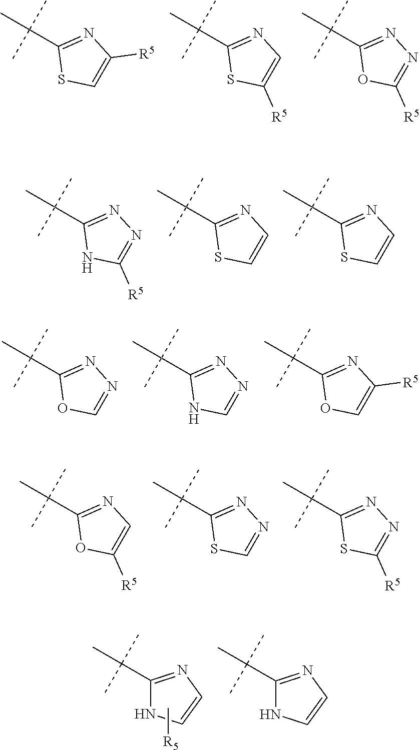

wherein R⁵ is as defined above.

In a particular embodiment, Ar¹ is selected from any one of the following groups:

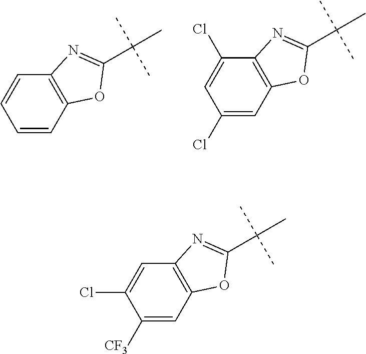

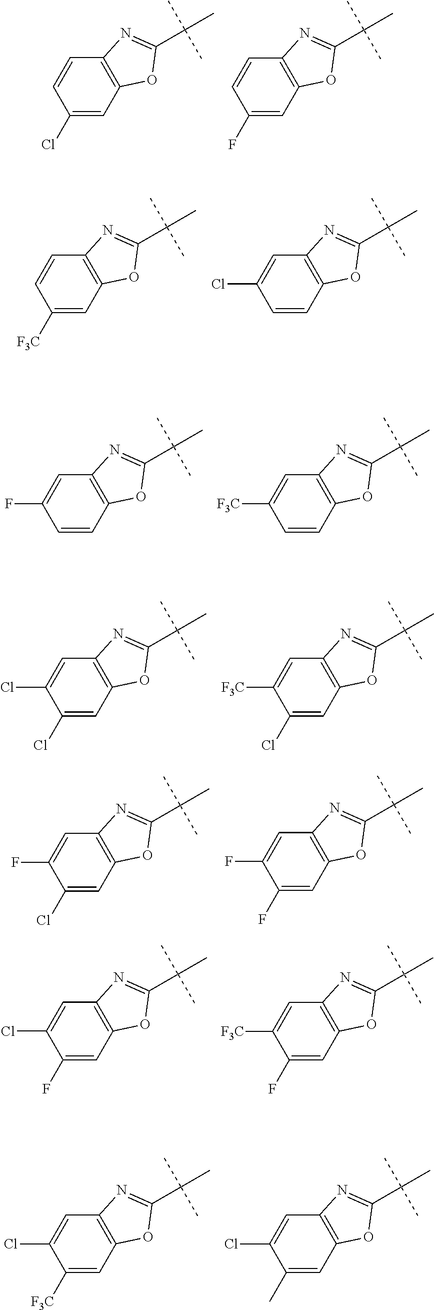

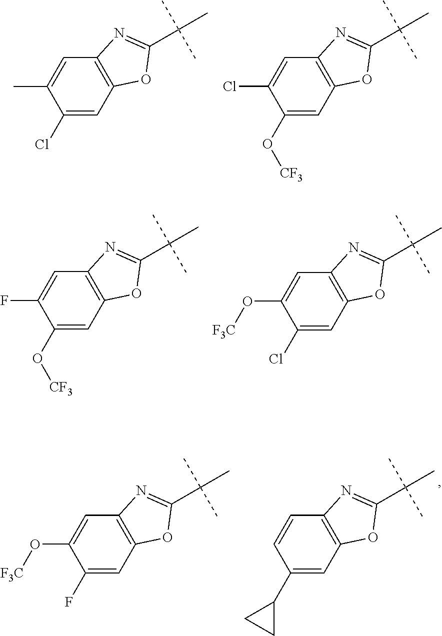

R¹ is H and Ar² is the following group:

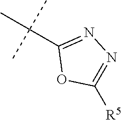

wherein R⁵ is as defined above.

In a particular embodiment, Ar¹ is selected from any one of the following groups:

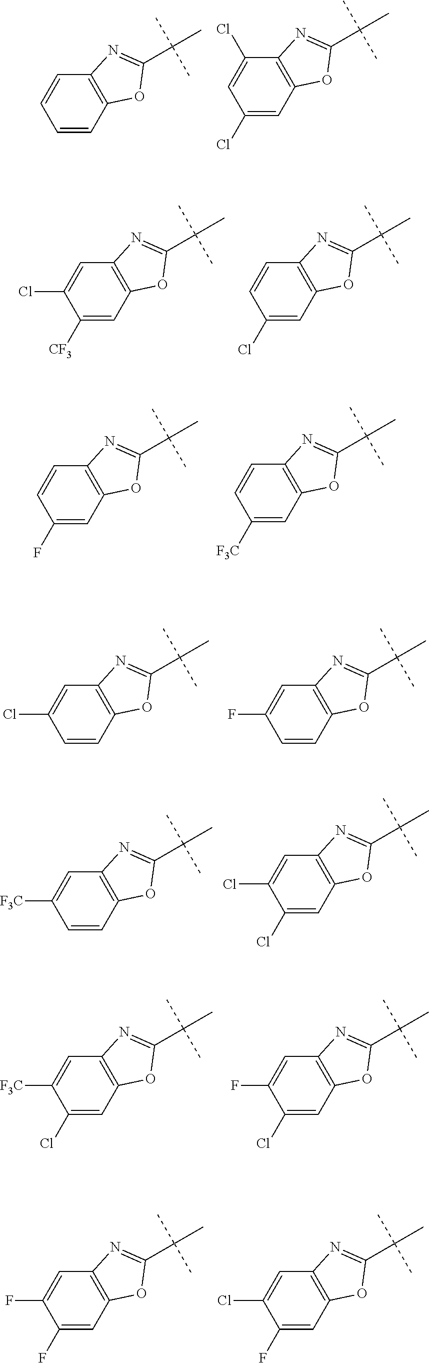

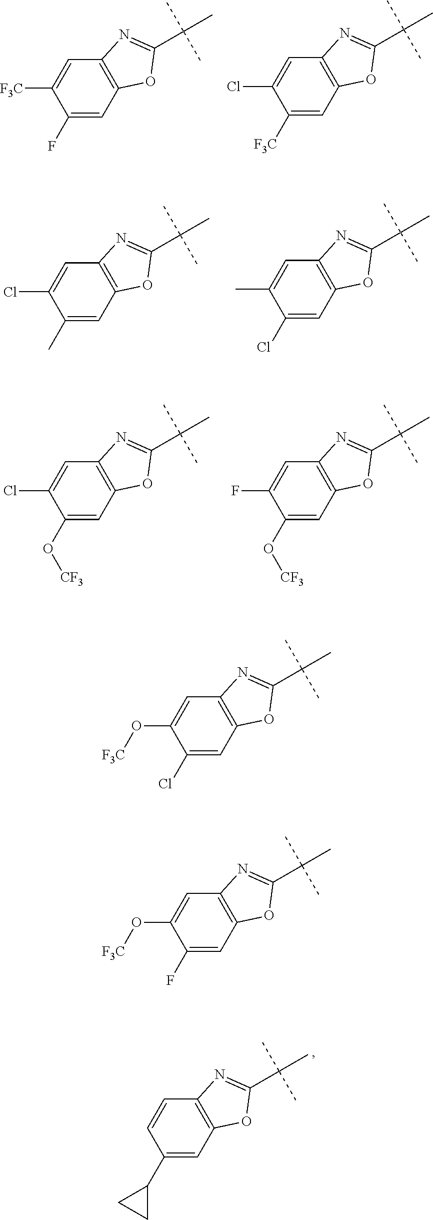

R¹ is H and Ar² is the following group:

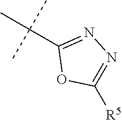

wherein R⁵ is C₁₋₄alkyl such as methyl, ethyl, n-propyl, isopropyl, tert-butyl, cyclopropyl, —CONR^{5A}R^{5B} Or —CH₂NR^{5A}R^{5B}.

In a particular embodiment, Ar¹ is selected from any one of the following groups:

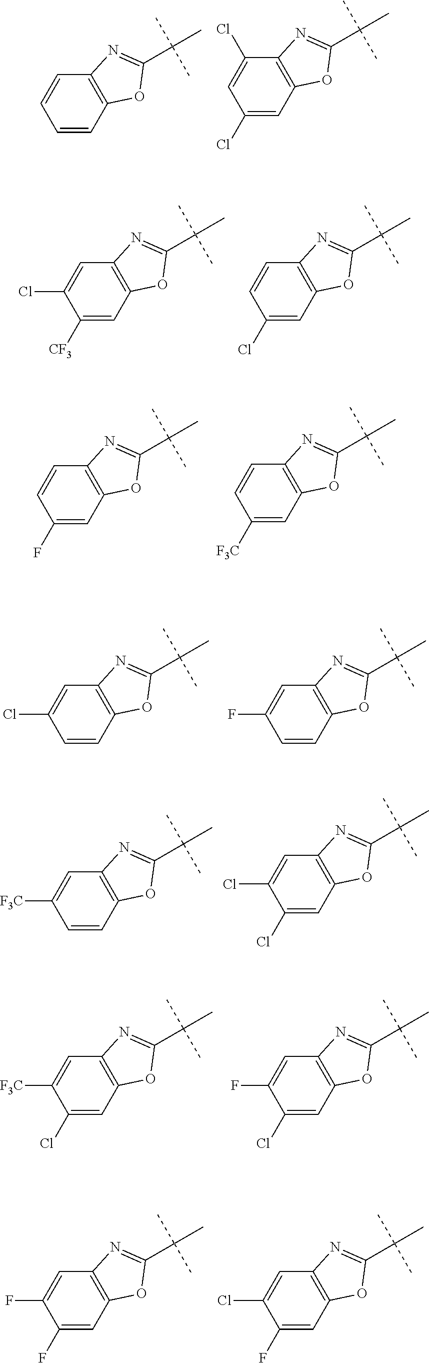

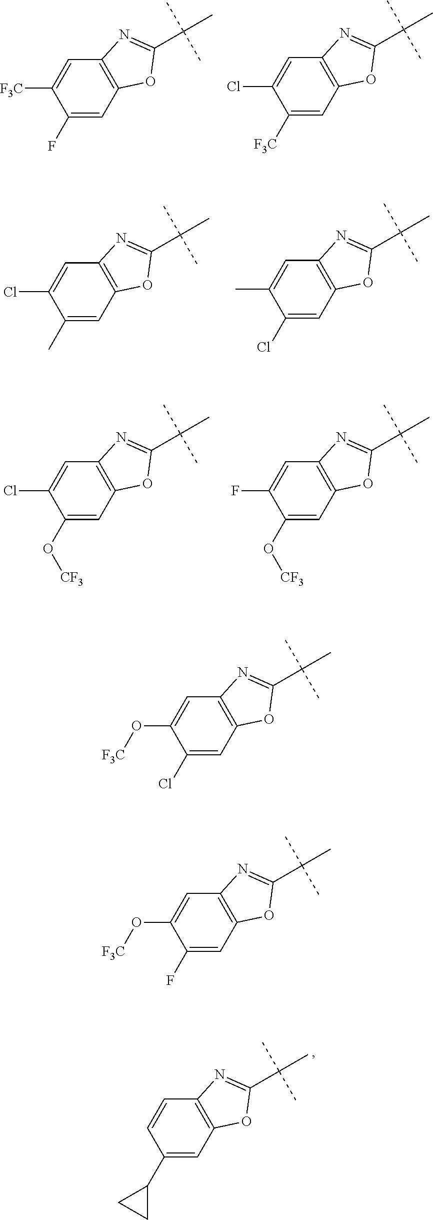

R¹ is H wherein Ar² is the following group:

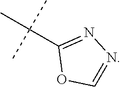

Biological Activity of the Compounds of the Invention

The compounds of the invention may exhibit: (a) broad spectrum antibacterial activity (i.e. against Gram-positive and Gram-negative bacteria); (b) narrow spectrum activity (i.e. against Gram positive or Gram negative bacteria); or (c) specific activity (i.e. against a single bacterial species).

Medical Applications

The compounds of the invention find application in the treatment of a wide range of diseases. Thus, the invention contemplates the compounds as described herein for use in medicine (e.g. for use in treatment or prophylaxis), methods of medical treatment or prophylaxis involving the administration of the compounds as described herein as well as pharmaceutical compositions comprising the compounds as described herein.

The compounds of the invention find particular application in the medical applications are described in more detail below:

(a) Treatment of Bacterial Disease and Infection

The invention finds broad application in the treatment of any bacterial infection or disease, including Gram-positive and Gram-negative infections and diseases. Gram-positive infections and diseases which may be targeted by the invention include those involving high G+C and low G+C Gram-positive bacteria.

Examples of bacteria which may be targeted by the compounds of the invention include but are not limited to: Helicobacter pylori, Borelia burgdorferi, Legionella pneumophilia, Mycobacterium spp (e.g. M. tuberculosis, M. leprae, M. avium, M. intracellulare, M. kansaii and M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus viridans, Streptococcus faecalis, Streptococcus bovis, any anaerobic species of the genus Streptococcus, Streptococcus pneumoniae, Campylobacter spp., Enterococcus spp., Haemophilus influenzae, Bacillus anthracis, Corynebacterium spp. (including C. diphtheriae), Erysipelothrix rhusiopathiae, Clostridium perfringens, Clostridium tetani, Enterobacter aerogenes, Klebsiella spp (including K. pneumoniae), Pasteurella multocida, Bacteroides spp., Fusobacterium nucleatum, Streptobacillus monilijormis, Treponema pallidium, Treponema pertenue, Leptospira spp., Rickettsia spp. and Actinomyces spp. (including A. israelii).

(b) Exemplary Bacterial Targets of the Compounds of the Invention

The compounds of the present invention may have antibacterial (e.g. bacteriostatic or bactericidal) activity against any bacterium.

Thus, the compounds of the invention may target: (a) Gram-positive, Gram-negative and/or Gram-variable bacteria; (b) spore-forming bacteria; (c) non-spore forming bacteria; (d) filamentous bacteria; (e) intracellular bacteria; (f) obligate aerobes; (g) obligate anaerobes; (h) facultative anaerobes; (i) microaerophilic bacteria and/or (f) opportunistic bacterial pathogens.

In certain embodiments, the compounds of the invention target one or more bacteria of the following genera: Acinetobacter (e.g. A. baumannii); Aeromonas (e.g. A. hydrophila); Bacillus (e.g. B. anthracis); Bacteroides (e.g. B. fragilis); Bordetella (e.g. B. pertussis); Borrelia (e.g. B. burgdorferi); Brucella (e.g. B. abortus, B. canis, B. melitensis and B. suis); Burkholderia (e.g. B. cepacia complex); Campylobacter (e.g. C. jejuni); Chlamydia (e.g. C. trachomatis, C. suis and C. muridarum); Chlamydophila (e.g. (e.g. C. pneumoniae, C. pecorum, C. psittaci, C. abortus, C. felis and C. caviae); Citrobacter (e.g. C. freundi); Clostridium (e.g. C. botulinum, C. difficile, C. perfringens and C. tetani); Corynebacterium (e.g. C. diphteriae and C. glutamicum); Enterobacter (e.g. E. cloacae and E. aerogenes); Enterococcus (e.g. E. faecalis and E. faecium); Escherichia (e.g. E. coli); Flavobacterium; Francisella (e.g. F. tularensis); Fusobacterium (e.g. F. necrophorum); Haemophilus (e.g. H. somnus, H. influenzae and H. parainfluenzae); Helicobacter (e.g. H. pylon); Klebsiella (e.g. K. oxytoca and K. pneumoniae), Legionella (e.g. L. pneumophila); Leptospira (e.g. L. interrogans); Listeria (e.g. L. monocytogenes); Moraxella (e.g. M. catarrhalis); Morganella (e.g. M. morgani); Mycobacterium (e.g. M. leprae and M. tuberculosis); Mycoplasma (e.g. M. pneumoniae); Neisseria (e.g. N. gonorrhoeae and N. meningitidis); Pasteurella (e.g. P. multocida); Peptostreptococcus; Prevotella; Proteus (e.g. P. mirabilis and P. vulgaris), Pseudomonas (e.g. P. aeruginosa); Rickettsia (e.g. R. rickettsii); Salmonella (e.g. S. typhi and S. typhimurium); Serratia (e.g. S. marcesens); Shigella (e.g. S. flexnaria, S. dysenteriae and S. sonnei); Staphylococcus (e.g. S. aureus, S. haemolyticus, S. intermedius, S. epidermidis and S. saprophyticus); Stenotrophomonas (e.g. S. maltophila); Streptococcus (e.g. S. agalactiae, S. mutans, S. pneumoniae and S. pyogenes); Treponema (e.g. T. pallidum); Vibrio (e.g. V. cholerae) and Yersinia (e.g. Y. pestis).

The compounds of the invention may be used to target multi-drug resistant bacteria, including, but not limited to penicillin-resistant, methicillin-resistant, quinolone-resistant, macrolide-resistant, and/or vancomycin-resistant bacterial strains, including for example penicillin-, methicillin-, macrolide-, vancomycin-, and/or quinolone-resistant Streptococcus pneumoniae; penicillin-, methicillin-, macrolide-, vancomycin-, and/or quinolone-resistant Staphylococcus aureus; penicillin-, methicillin-, macrolide-, vancomycin-, and/or quinolone-resistant Streptococcus pyogenes; and penicillin-, methicillin-, macrolide-, vancomycin-, and/or quinolone-resistant enterococci.

Thus, the compounds of the invention may also be used to target MRSA, for example selected from any of C-MSRA1, C-MRSA2, C-MRSA3, C-MSRA4, Belgian MRSA, Swiss MRSA and any of the EMRSA strains.

The compounds of the invention may be used to target high G+C Gram-positive bacteria. The term “high G+C Gram-positive bacteria” is a term of art defining a particular class of evolutionarily related bacteria. The class includes Micrococcus spp. (e.g. M. luteus), Mycobacterium spp. (for example a fast- or slow-growing mycobacterium, e.g. M. tuberculosis, M. leprae, M. smegmatis or M. bovis), Streptomyces spp. (e.g. S. rimosus and S. coelicolor) and Corynebacterium spp. (e.g. C. glutamicum).

The compounds of the invention may be used to target low G+C Gram-positive bacteria. The term “low G+C Gram-positive bacteria” is a term of art defining a particular class of evolutionarily related bacteria. The class includes members of the Firmicutes phylum, including for example Staphylococcus spp. and Bacillus spp.

(c) Exemplary Target Bacterial Diseases

Any bacterial disease may be treated using the compounds of the invention.

Preferred is the treatment or prophylaxis of infection or intoxication with, or a disease caused by, a bacterium selected from: Staphylococcus aureus; Enterococcus faecalis, Enterococcus faecium and Neisseria gonorrhoeae.

Particularly preferred is the treatment or prophylaxis of infection or intoxication with, or a disease caused by, Neisseria gonorrhoeae.

Thus, the compounds of the invention find application in the treatment or prophylaxis of a bacterial disease selected from: anthrax (e.g. cutaneous anthrax, pulmonary anthrax and gastrointestinal anthrax); bacterial pneumonia; whooping cough; Lyme disease; brucellosis; acute enteritis; botulism; tetanus; diphtheria; tularemia; Lemierre's syndrome; Legionnaire's Disease; leprosy (Hansen's disease); tuberculosis, meningitis, syphilis, gas gangrene, scarlet fever, erysipelas, rheumatic fever, streptococcal pharyngitis, toxic shock syndrome, listeriosis, Whipple's disease, erythrasma, nocardiosis, maduromycosis, Ghon's complex, Pott's disease, Rich focus, scrofula, Bazin disease, lupus vulgaris, Lady Windermere syndrome, Buruli ulcer, yaws, relapsing fever, trench mouth, rat-bite fever, leptospirosis, mycoplasmal pneumonia, ureaplasmal infection, psittacosis, chlamydia, lymphogranuloma venereum, trachoma, rickettsioses, typhus, spotted fever, Rocky Mountain spotted fever, Boutonneuse fever, Rickettsial pox, ehrlichiosis (including human granulocytic ehrlichiosis and human monocytic ehrlichiosis), Q fever, bartonella, orientia, bacillary angiomatosis, Waterhouse-Friderichsen syndrome, gonorrhoea, burkholderiales, glanders, melioidosis, pertussis, typhoid fever, paratyphoid fever, salmonellosis, rhinoscleroma, donovanosis, shigellosis, pasteurellosis, Brazilian purpuric fever, chancroid, actinobacillosis, cholera, campylobacteriosis, bronchitis, sinusitis, laryngitis, otitis media, bronchitis, C. difficile colitis, cervicitis, endocarditis, gonococcal urethritis, inhalation anthrax, intra-abdominal infections, meningitis, osteomyelitis, otitis media, pharyngitis, pneumonia, prostatitis, bronchitis, C. difficile colitis, cervicitis, septicemia, skin and soft tissue infections, urinary tract infections, sepsis (including catheter-related sepsis), hospital-acquired pneumonia (HAP), gynecological infection, respiratory tract infection (RTI), sexually transmitted disease, urinary tract infection, acute exacerbation of chronic bronchitis (ACEB), acute otitis media, acute sinusitis, an infection caused by drug resistant bacteria, skin and skin structure infection, febrile neutropenia, gonococcal cervicitis, upper and lower respiratory tract infections, skin and soft tissue infections, hospital-acquired lung infections, bone and joint infections, respiratory tract infections, acute bacterial otitis media, pyelonephritis, intra-abdominal infections, deep-seated abcesses, central nervous system infections, bacteremia, wound infections, peritonitis, infections after burn, urogenital tract infections, gastro-intestinal tract infections, pelvic inflammatory disease; intravascular infections and plague.

The compounds of the invention may be used to treat multi-drug resistant bacterial infections, including infections caused by penicillin-resistant, methicillin-resistant, quinolone-resistant, macrolide-resistant, and/or vancomycin-resistant bacterial strains. The multi-drug resistant bacterial infections to be treated using the methods of the invention include, for example, infections by penicillin-, methicillin-, macrolide-, vancomycin-, and/or quinolone-resistant Streptococcus pneumoniae; penicillin-, methicillin-, macrolide-, vancomycin-, and/or quinolone-resistant Staphylococcus aureus; penicillin-, methicillin-, macrolide-, vancomycin-, and/or quinolone-resistant Streptococcus pyogenes; and penicillin-, methicillin-, macrolide-, vancomycin-, and/or quinolone-resistant enterococci.

The compounds of the invention may also be used to treat diseases arising from infection with MRSA, for example selected from any of C-MSRA1, C-MRSA2, C-MRSA3, C-MSRA4, Belgian MRSA, Swiss MRSA and any of the EMRSA strains. Accordingly, the invention therefore finds utility in the treatment or prophylaxis of infections mediated by drug-resistant bacteria and in the treatment or prophylaxis of nosocomial infections.

The compounds of the invention may also be used to treat mycobacterial diseases. The term “mycobacterial disease” defines any disease, disorder, pathology, symptom, clinical condition or syndrome in which bacteria of the genus Mycobacterium (i.e. mycobacteria) act as aetiological agents or in which infection with mycobacteria is implicated, detected or involved. Any mycobacterial infection may be treated, including those in which bacteria of the Mycobacterium avium complex (MAC) is involved. This term defines a class of genetically-related bacteria belonging to the genus Mycobacterium and includes Mycobacterium avium subspecies avium (MAA), Mycobacterium avium subspecies hominis (MAH), and Mycobacterium avium subspecies paratuberculosis (MAP) together with the genetically distinct Mycobacterium avium intracellulare (MAI).

The term therefore includes the various forms of tuberculosis (TB), leprosy, paediatric lymphadenitis and mycobacterial skin ulcers. The term therefore covers mycobacterial conditions arising from or associated with infection by nontuberculous mycobacteria as well as tuberculous mycobacteria.

Thus, the invention finds particular application in the treatment and prophylaxis of a mycobacterial condition selected from:
AIDS-related mycobacterial infection
Mycobacterial infection in immunocompromised patients (e.g. attendant on malignancy, receipt of an organ transplant, immunoablation or administration of steroids)
Pulmonary TB
Extra-pulmonary TB (including but not limited to miliary TB, central nervous system TB, pleural TB, pericardital TB, genitourinary TB, gastrointestinal TB, peritonital TB and TB of the bones and joints).
Latent (persistent or asymptomatic) mycobacterial infection
Active mycobacterial disease
MDR-TB (multidrug resistant TB)
XDR-TB (Extensive Drug Resistant TB or Extreme Drug Resistance TB): this is a recently recognized class of MDR-TB that displays resistance to three or more of the six principal classes of second-line drugs.

The compounds of the invention may therefore be used in combination with one or more additional compounds useful for the treatment of TB. Examples of such compounds include but are not limited to, isoniazid, rifamycin and derivatives thereof, pyrazinamide, ethambutol, cycloserine, ethionamide, streptomycin, amikacin, kanamycin, capreomycin, p-aminosalicylic acid, and fluoroquinolones such as levofloxacin, moxifloxacin or gatifloxacin. Examples of rifamycin derivatives include rifampin, rifabutin and rifapentine.

Other infections which may be treated according to the invention include those involving

Corynebacterium spp. (including Corynebacterium diphtheriae), Tropherymawhippelii,

Nocardia spp. (including Nocardia asteroides and Nocardia brasiliensis), Streptomyces spp. (including Streptomyces griseus, Streptomyces paraguayensis and Streptomyces somaliensis), Actinomadura spp., Nocardiopsis spp., Rhodococcus spp., Gordona spp., Tsukamurella spp. and Oerskovia spp. as well as other pathogenic organisms from the group referred to as high G+C Gram-positive bacteria. Other infections which may be treated include those involving pathogenic low G+C Gram-positive bacteria.

(d) Treatment of Bacterial Intoxication

The bacterial disease or infection may involve intoxication with one or more bacterial toxins, including for example endotoxins, exotoxins and/or toxic enzymes.

Thus, the compounds of the invention find application in the treatment of bacterial intoxication. In such embodiments, preferred is the treatment of intoxication with bacterial endotoxins, exotoxins and/or toxic enzymes, for example with endotoxins, exotoxins and/or toxic enzymes produced by the bacteria described in the preceding section.

Adjunctive Agents for Use in the Combinations of the Invention

(a) General

In addition to the compound of the invention, the invention also contemplates the use of one or more of the following adjunctive agents as further components of the invention.

Thus, the invention provides compositions comprising the compound of the invention in combination with one or more adjunctive agents selected from those described below.

(b) Antiviral Adjunctive Agents

The combinations preferably further comprise one or more auxiliary antiviral agent(s). Such auxiliary antiviral agents may be selected from one or more of: (a) viral enzyme inhibitors (for example selected from (i) protease inhibitors, (ii) helicase inhibitors and (iii) polymerase inhibitors); (b) nucleoside/nucleotide reverse transcriptase inhibitors; (c) non-nucleoside reverse transcriptase inhibitors; (d) integrase inhibitors; (e) maturation inhibitors; (f) cytokines or cytokine stimulatory factors; (g) viral entry inhibitors, for example selected from: (i) an attachment inhibitor; (ii) a co-receptor binding inhibitor; and (iii) a membrane fusion inhibitor.

(c) Antibacterial Adjunctive Agents

The compounds of the invention may be used in combination with various antibacterial agents, including, but not limited to one or more antibiotic(s) selected from the following:
Aminoglycosides (for example amikacin, gentamicin, kanamycin, neomycin, netilmicin, streptomycin, tobramycin and paromomycin).
Ansamycins (for example geldanamycin and herbimycin).
Carbacephems (for example loracarbef).
Carbapenems (for example ertapenem, doripenem, imipenem/cilastatin and meropenem)
Cephalosporins (first generation), including for example cefadroxil, cefazolin, cefalotin/cefalothin and cephalexin).
Cephalosporins (second generation), including for example cefaclor, cefamandole, cefoxitin, cefprozil and cefuroxime.
Cephalosporins (third generation), including for example cefixime, cefdinir, cefditoren, cefoperazone, cefotaxime, cefpodoxime, ceftazidime, ceftibuten, ceftizoxime, ceftriaxone and cefdinir.
Cephalosporins (fourth generation), including for example cefepime.
Glycopeptides (for example vancomycin and teicoplanin).
Macrolides (for example azithromycin, clarithromycin, dirithromycin, erythromycin, roxithromycin, troleandomycin, telithromycin and spectinomycin).
Monobactams (for example aztreonam).
Penicillins (for example amoxicillin, ampicillin, azlocillin, carbenicillin, cloxacillin, dicloxacillin, flucloxacillin, mezlocillin, nafcillin, penicillin, piperacillin and ticarcillin).
Polypeptides (for example bacitracin, polymixin B and colistin).
Quinolones (for example ciprofloxacin, enoxacin, gatifloxacin, levofloxacin, lomefloxacin, moxifloxacin, norfloxacin, ofloxacin and trovafloxacin).
Sulfonamides (for example mafenide, prontosil, sulfacetamide, sulfamethizole, sulfanilimide, sulfasalazine, sulfisoxazole, trimethoprim, trimethoprim-sulfamethoxazole (co-trimoxazole, TMP-SMX)).
Tetracyclines (for example demeclocycline, doxycycline, minocycline, oxytetracycline and tetracycline).
Aminocoumarins (for example novobiocin, albamycin, coumermycin and clorobiocin).
Oxazolidinones (for example linezolid and AZD2563).
Lipopeptides (for example daptomycin).
Streptogramins (for example quinupristin/dalfopristin).
Glycylcyclines (for example tigecycline).
Lantibiotics (for example Type A Lantibiotics (such as nisin, subtilin, epidermin, mutacin II, mutacin I & III) and Type B Lantibiotics (such as mersacidin, actagardine and cinnamycin).

Other suitable antibiotics useful as adjunctive agents include one or more antibiotic(s) selected from the following: arsphenamine, chloramphenicol, clindamycin, lincoamycin, ethambutol, fosfomycin, fusidic acid, furazolidone, isoniazid, linezolid, metronidazole, mupirocin, nitrofurantoin, platensimycin, pyrazinamide, quinupristin/dalfopristin, rifampin/rifampicin and tinidazole.

Thus, the compounds of the invention may be used in combination with one or more antibiotics selected from: penicillin, cloxacillin, dicloxacillin, methicillin, nafcillin, oxacillin, ampicillin, amoxicillin, bacampicillin, capreomycin, cycloserine, azlocillin, carbenicillin, mezlocillin, piperacillin, ticarcillin, azithromycin, clarithromycin, clindamycin, erythromycin, lincomycin, demeclocycline, doxycycline, ethambutol, ethionamide, minocycline, oxytetracycline, tetracycline, quinolone, cinoxacin, nalidixic acid, fluoroquinolones (for example levofloxacin, moxafloxacin and gatifloxacin, ciprofloxacin, enoxacin, grepafloxacin), kanamycin, levofloxacin, lomefloxacin, norfloxacin, ofloxacin, p-aminosalicylic acid, sparfloxacin, trovafloxacin, bacitracin, colistin, polymyxin B, sulfonamide, trimethoprim-sulfamethoxazole, co-amoxyclav, cephalothin, cefuroxime, ceftriaxone, vancomycin, gentamicin, amikacin, metronidazole, chloramphenicol, streptomycin, nitrofurantoin, co-trimoxazole, rifamycin and derivatives thereof (for example rifampicin, rifabutin and rifapentine), isoniazid, pyrazinamide, kirromycin, thiostrepton, micrococcin, fusidic acid, thiolactomycin and fosmidomycin.

Other suitable antibacterial adjunctive agents may be selected from those listed in the table below:

| Compound | Class |
|---|---|
| DU-6859 | Fluoroquinolone |
| Erythromycin stinoprate | Macrolide |
| Oritavancin | Glycopeptide |
| Telavancin | Glycopeptide |
| Dalbavancin | Glycopeptide |
| Ceftobiprole medocaril | Cephalosporin |
| Tebipenem pivoxil | Carbapenem |
| Iclaprim | DHFR |
| OPT-80 | Difimicin |
| Ceftaroline fosamil | Cephalosporin |
| RX-3341 | Fluoroquinolone |
| Cethromycin | Ketolide |
| TD-1792 | Glycopeptide-β-lactam dimer |
| EDP-420 | Macrolide |
| RX-1741 | Oxazolidinone |
| MK-2764 | Glycycline |
| Nemonoxacin | Fluoroquinolone |
| Flopristin + Linopristin | Streptogramin |
| Tomopenem | Carbapenem |
| Ramoplanin | Glycolipodepsipeptide |
| Linezolid | Oxazolidinone |
| Cefditoren pivoxil | Cephalosporin |
| Ertapenem | Carbapenem |
| Gemifloxacin | Fluoroquinolone |
| Daptomycin | Lipopetide |
| Telithromycin | Lipopetide |
| Tigecyline | Glycylcycline |

(d) Antifungal Adjunctive Agents

The compounds of the invention may be used in combination with various antifungal agents (antimycotics).

(e) Antiprotozoal Adjunctive Agents

The compounds of the invention may be used in combination with various antiprotozoal agents, including but not limited to, chloroquine, doxycycline, mefloquine, metronidazole, eplornithine, furazolidone, hydroxychloroquine, iodoquinol, pentamidine, mebendazole, piperazine, halofantrine, primaquine, pyrimethamine sulfadoxine, doxycycline, clindamycin, quinine sulfate, quinidine gluconate, quinine dihydrochloride, hydroxychloroquine sulfate, proguanil, quinine, clindamycin, atovaquone, azithromycin, suramin, melarsoprol, eflornithine, nifurtimox, amphotericin B, sodium stibogluconate, pentamidine isethionate, trimethoprim-sulfamethoxazole, pyrimethamine and sulfadiazine.

(f) Other Adjunctive Agents

The compounds of the invention may be co-administered with a variety of other co-therapeutic agents which treat or prevent side effects arising from the antiinfective treatment and/or presenting as sequelae of the infection. Adjunctive agents of this type may or may not have antiinfective activity and include, for example, PPIs and H2RAs (as hereinbefore described).

Thus, the compounds of the invention may be used adjunctively with PPIs including, but are not limited to, omeprazole (Losec, Prilosec, Zegerid), lansoprazole (Prevacid, Zoton, Inhibitol), esomeprazole (Nexium), pantoprazole (Protonix, Somac, Pantoloc, Pantozol, Zurcal, Pan) and rabeprazole (Rabecid, Aciphex, Pariet, Rabeloc).

The compounds of the invention may also be used adjunctively with H2RAs including, but are not limited to, cimetidine (Tagamet), ranitidine (Zinetac, Zantac), famotidine, (Pepcidine, Pepcid), roxatidine (Roxit) and nizatidine (Tazac, Axid).

The compounds of the invention may be used adjunctively with triple therapy with PPIs or H2RAs together with a combination of two antibiotics (including, but not limited to, antibiotics selected from metronidazole, amoxicillin, levofloxacin and clarithromycin).

Various probiotics may be used as adjunctive agents, including for example Saccharomyces boulardii or Lactobacillus acidophilus cells. Probiotics are mono or mixed cultures of live microorganisms which are proposed to help re-establish the natural gut microflora. In addition, such microorganisms may act to stimulate the patient's immune system and to elicit production of enzymes that degrade the bacterial toxins. Particular microorganisms of interest are, but not limited to, Saccharomyces spp. (for example Saccharomyces boulardii and Saccharomyces cerevisiae) and Lactobacillus spp. (for example Lactobacillus rhamnosus, Lactobacillus casei, Lactobaccillus acidophilus, Lactobacillus bulgaris and Lactobacillus plantarum). Any other common probiotic composition or microorganism that is a normal member of the human intestinal tract may also be considered.

Pre-biotics, agents aimed at stimulating the growth of the intestinal flora, may also be used as adjunctive agents. For example, the use of oligofructose has been shown to increase levels of Bifidobacterium spp. and reduce subsequent relapse rates in patients.

Other approaches aimed at reestablishing the normal enteric flora include faecal biotherapy and faecal enemas prepared from the stools of healthy individuals which contain the normal microorganisms of the gut. Faecal bacteriotherapy may therefore also be used adjunctively with the compounds of the invention.

The compounds of the invention may be used adjunctively with various immunoglobulins.

Agents aimed at reducing diarrhoea may be of benefit when trying to increase levels of an antimicrobial agent at the site of infection and/or when trying to increase the length of time an antibacterial agent is in contact with the enteric pathogen. Such agents may include, but are not limited to, loperamide (Lopex, Imodium, Dimor, Pepto) diphenoxylate (Lomotil, Co-phenotrope) difenoxin (Motofen), and racecadotril. Thus, the compounds of the invention may be used adjunctively with various anti-diarrhoeal agents, including any of those listed above.

Co-therapeutic agents which treat or prevent any of the following side effects may be used as part of the same treatment regimen as the compounds of the invention: (a) lipodystrophy and wasting; (b) facial lipoatrophy; (c) hyperlipidemia; (d) fatigue; (e) anaemia; (f) peripheral neuropathy; (g) nausea; (h) diarrhoea; (i) hepatotoxicity; (j) osteopenia; (k) dehydration and (I) osteoporosis.

The treatment or prophylaxis may comprise the administration of a compound as defined herein as an adjunctive to one or more of the following treatments or interventions:
(a) Cancer therapy;
(b) AIDS therapy;
(c) Immunosuppressive interventions;
(d) Post-transplantation graft/implant management;
(e) Onychomycotic nail surgery or debridement;
(f) Topical antimycotic therapy (for example with an antimycotic agent selected from azoles, allylamines (e.g. terbinafine) or a morpholine (e.g. amorolfine);
(g) Systemic antimycotic therapy;
(h) Antibacterial therapy;
(i) Antiviral therapy;
(j) Anti-inflammation therapy (e.g. with steroids);
(k) Analgesic administration;
(l) Antipruritic administration;
(m) Probiotic administration;
(n) Faecal bacteriotherapy; or
(o) Skin grafting.

Thus, the invention may comprise the treatment or prophylaxis of a patient population in which one or more of the treatment or interventions (a) to (o) are being (or have been) carried out.

(g) Adjunctive Treatments

The treatment or prophylaxis may comprise the administration of a compound as defined herein as an adjunctive to one or more of the following treatments or interventions:
1. Cancer therapy;
2. Immunosuppressive interventions;
3. Immunostimulatory interventions;
4. Post-transplantation graft/implant management;
5. Onychomycotic nail surgery or debridement;
6. Anti-inflammation therapy (e.g. with steroids);
7. Analgesic administration;
8. Antipruritic administration;
9. Surgery;
10. Cell or tissue ablation;
11. Radiotherapy;
12. Cryotherapy;
13. Faecal transplantation therapy (faecal bacteriotherapy);
14. Probiotic therapy; or
15. Skin grafting.

Thus, the invention may comprise the treatment or prophylaxis of a patient population in which one or more of the treatment or interventions (1) to (15) are being (or have been) carried out.

Posology

The compounds of the present invention can be administered by oral or parenteral routes, including intravenous, intramuscular, intraperitoneal, subcutaneous, transdermal, airway (aerosol), rectal, vaginal and topical (including buccal and sublingual) administration.

The amount of the compound administered can vary widely according to the particular dosage unit employed, the period of treatment, the age and sex of the patient treated, the nature and extent of the disorder treated, and the particular compound selected.

In general, the effective amount of the compound administered will generally range from about 0.01 mg/kg to 10000 mg/kg daily. A unit dosage may contain from 0.05 to 500 mg of the compound, and can be taken one or more times per day. The compound can be administered with a pharmaceutical carrier using conventional dosage unit forms either orally, parenterally or topically, as described below.

The preferred route of administration is oral administration. In general a suitable dose will be in the range of 0.01 to 500 mg per kilogram body weight of the recipient per day, preferably in the range of 0.1 to 1000 mg per kilogram body weight per day and most preferably in the range 1 to 5 mg per kilogram body weight per day.

The desired dose is preferably presented as a single dose for daily administration.

However, two, three, four, five or six or more sub-doses administered at appropriate intervals throughout the day may also be employed. These sub-doses may be administered in unit dosage forms, for example, containing 0.001 to 100 mg, preferably 0.01 to 10 mg, and most preferably 0.5 to 1.0 mg of active ingredient per unit dosage form.

In determining an effective amount or dose, a number of factors are considered by the attending physician, including, but not limited to, the potency and duration of action of the compounds used, the nature and severity of the illness to be treated, as well as the sex, age, weight, general health and individual responsiveness of the patient to be treated, and other relevant circumstances. Those skilled in the art will appreciate that dosages can also be determined with guidance from Goodman & Goldman's The Pharmacological Basis of Therapeutics, Ninth Edition (1996), Appendix II, pp. 1707-1711.

The amount of the compound that can be combined with carrier materials to produce a single dosage form varies depending upon the subject to be treated and the particular mode of administration. For example, a formulation intended for oral administration to humans can contain about 0.5 mg to about 7 g of active agent compounded optionally with an appropriate and convenient amount of carrier material which can vary from about 5 to about 95 percent of the total composition. Dosage unit forms for the compounds of the invention generally contain about 1 mg to about 500 mg of the active ingredient, for example 5 mg, 10 mg, 20 mg, 25 mg, 50 mg, 100 mg, 200 mg, 300 mg, 400 mg, 500 mg, 600 mg, 800 mg or 1000 mg.

The effectiveness of a particular dosage of the compound of the invention can be determined by monitoring the effect of a given dosage on the progression of the disease or its prevention.

Formulation

The compound of the invention may take any form. It may be synthetic, purified or isolated from natural sources using techniques described in the art.

Illustrative pharmaceutically acceptable salts are prepared from formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, 3-hydroxybutyric, galactaric and galacturonic acids.

Suitable pharmaceutically-acceptable base addition salts include metallic ion salts and organic ion salts. Metallic ion salts include, but are not limited to, appropriate alkali metal (group Ia) salts, alkaline earth metal (group IIa) salts and other physiologically acceptable metal ions. Such salts can be made from the ions of aluminium, calcium, lithium, magnesium, potassium, sodium and zinc. Organic salts can be made from tertiary amines and quaternary ammonium salts, including in part, trimethylamine, diethylamine, N, N′-dibenzylethylenediamine, chloroprocaine, choline, diethanolamine, ethylenediamine, meglumine (N-methylglucamine) and procaine. All of the above salts can be prepared by those skilled in the art by conventional means from the corresponding compound.

Pharmaceutical compositions can include stabilizers, antioxidants, colorants and diluents. Pharmaceutically acceptable carriers and additives are chosen such that side effects from the pharmaceutical compound are minimized and the performance of the compound is not compromised to such an extent that treatment is ineffective.

The pharmaceutical compositions may be administered enterally and/or parenterally. Oral (intra-gastric) is a typical route of administration. Pharmaceutically acceptable carriers can be in solid dosage forms, including tablets, capsules, pills and granules, which can be prepared with coatings and shells, such as enteric coatings and others well known in the art. Liquid dosage forms for oral administration include pharmaceutically acceptable emulsions, solutions, suspensions, syrups and elixirs. Parenteral administration includes subcutaneous, intramuscular, intradermal, intravenous, and other routes known in the art. Enteral administration includes solution, tablets, sustained release capsules, enteric coated capsules, and syrups. When administered, the pharmaceutical composition can be at or near body temperature.

Compositions intended for oral use can be prepared according to any method known in the art for the manufacture of pharmaceutical compositions and such compositions can contain one or more agents selected from the group consisting of sweetening agents, flavouring agents, colouring agents and preserving agents in order to provide pharmaceutically elegant and palatable preparations. Tablets contain the active ingredient in admixture with non-toxic pharmaceutically acceptable excipients, which are suitable for the manufacture of tablets. These excipients may be, for example, inert diluents, such as calcium carbonate, sodium carbonate, lactose, calcium phosphate or sodium phosphate, granulating and disintegrating agents, for example, maize starch, or alginic acid, binding agents, for example starch, gelatin or acacia, and lubricating agents, for example magnesium stearate, stearic acid, or talc. Tablets can be uncoated or they can be coated by known techniques, for example to delay disintegration and absorption in the gastrointestinal tract and thereby provide sustained action over a longer period. For example, a time delay material such as glyceryl monostearate or glyceryl distearate can be employed. Formulations for oral use can also be presented as hard gelatin capsules wherein the active ingredients are mixed with an inert solid diluent, for example, calcium carbonate, calcium phosphate or kaolin, or as soft gelatin capsules wherein the active ingredients are present as such, or mixed with water or an oil medium, for example, peanut oil, liquid paraffin or olive oil.

Aqueous suspensions can be produced that contain the active materials in a mixture with excipients suitable for the manufacture of aqueous suspensions. Such excipients include suspending agents, for example, sodium carboxymethylcellulose, methylcellulose, hydroxypropylmethylcellulose, sodium alginate, polyvinylpyrrolidone, gum tragacanth and gum acacia; dispersing or wetting agents can be naturally-occurring phosphatides, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyoxyethylene sorbitan monooleate. Aqueous suspensions can also contain one or more preservatives, for example, ethyl or N-propyl p-hydroxybenzoate, one or more colouring agents, one or more flavouring—agents, or one or more sweetening agents, such as sucrose or saccharin. Suitable aqueous vehicles include Ringer's solution and isotonic sodium chloride. Aqueous suspensions according to the invention may include suspending agents such as cellulose derivatives, sodium alginate, polyvinylpyrrolidone and gum tragacanth, and a wetting agent such as lecithin. Suitable preservatives for aqueous suspensions include ethyl and N-propyl p-hydroxybenzoate.

Oily suspensions may be formulated by suspending the active ingredients in an omega-3 fatty acid, a vegetable oil, for example, arachis oil, olive oil, sesame oil or coconut oil, or in a mineral oil such as liquid paraffin. The oily suspensions can contain a thickening agent, for example beeswax, hard paraffin or cetyl alcohol.

Sweetening agents, such as those set forth above, and flavouring agents can be added to provide a palatable oral preparation. These compositions can be preserved by addition of an antioxidant such as ascorbic acid.

Dispersible powders and granules suitable for preparation of an aqueous suspension by addition of water provide the active ingredient in admixture with a dispersing or wetting agent, a suspending agent and one or more preservatives. Suitable dispersing or wetting agents and suspending agents are exemplified by those already mentioned above. Additional excipients, for example sweetening, flavouring and colouring agents, can also be present.

Syrups and elixirs containing the compound of the invention can be formulated with sweetening agents, for example glycerol, sorbitol, or sucrose. Such formulations can also contain a demulcent, a preservative and flavouring and colouring agents.

The compound of the invention can be administered parenterally, for example subcutaneously, intravenously, or intramuscularly, or by infusion techniques, in the form of sterile injectable aqueous or oleaginous suspensions. Such suspensions can be formulated according to known art using suitable dispersing or wetting agents and suspending agents such as those mentioned above or other acceptable agents. A sterile injectable preparation can be a sterile injectable solution or suspension in a non-toxic parenterally acceptable diluent or solvent, for example a solution in 1,3-butanediol. Among acceptable vehicles and solvents that can be employed are water, Ringer's solution and isotonic sodium chloride solution. In addition, sterile fixed oils are conventionally employed as a solvent or suspending medium. For this purpose, any bland fixed oil may be employed, including synthetic mono- or diglycerides. In addition, omega-3 polyunsaturated fatty acids can find use in preparation of injectables. Administration can also be by inhalation, in the form of aerosols or solutions for nebulizers, or rectally, in the form of suppositories prepared by mixing the drug with a suitable non-irritating excipient which is solid at ordinary temperature, but liquid at rectal” temperature and will therefore, melt in the rectum to release the drug. Such materials are cocoa butter and polyethylene glycols. Also encompassed by the present invention is buccal and sub-lingual administration, including administration in the form of lozenges, pastilles or a chewable gum comprising the compounds set forth herein. The compounds can be deposited in a flavoured base, usually sucrose, and acacia or tragacanth.

Other methods for administration of the compounds of the invention include dermal patches that release the medicaments directly into and/or through a subject's skin.

Topical delivery systems are also encompassed by the present invention and include ointments, powders, sprays, creams, jellies, collyriums, solutions or suspensions.

Compositions of the present invention can optionally be supplemented with additional agents such as, for example, viscosity enhancers, preservatives, surfactants and penetration enhancers. Viscosity-building agents include, for example, polyvinyl alcohol, polyvinyl pyrrolidone, methylcellulose, hydroxypropylmethylcellulose, hydroxyethylcellulose, carboxymethylcellulose, hydroxypropylcellulose or other agents known to those skilled in the art. Such agents are typically employed at a level of about 0.01% to about 2% by weight of a pharmaceutical composition.

Preservatives are optionally employed to prevent microbial growth prior to or during use. Suitable preservatives include polyquaternium-1, benzalkonium chloride, thimerosal, chlorobutanol, methylparaben, propylparaben, phenylethyl alcohol, edetate disodium, sorbic acid, or other agents known to those skilled in the art.

Typically, such preservatives are employed at a level of about 0.001% to about 1.0% by weight of a pharmaceutical composition.

Solubility of components of the present compositions can be enhanced by a surfactant or other appropriate cosolvent in the composition. Such cosolvents include polysorbates 20, 60 and 80, polyoxyethylene/polyoxypropylene surfactants (e. g., Pluronic F-68, F-84 and P-103), cyclodextrin, or other agents known to those skilled in the art. Typically, such cosolvents are employed at a level of about 0.01% to about 2% by weight of a pharmaceutical composition.

Pharmaceutically acceptable excipients and carriers encompass all the foregoing and the like. The above considerations concerning effective formulations and administration procedures are well known in the art and are described in standard textbooks. See for example Remington: The Science and Practice of Pharmacy, 20th Edition (Lippincott, Williams and Wilkins), 2000; Lieberman et al., ed; Pharmaceutical Dosage Forms, Marcel Decker, New York, N.Y. (1980) and Kibbe et al., ed., Handbook of Pharmaceutical Excipients (3rd Edition), American Pharmaceutical Association, Washington (1999). Thus, in embodiments where the compound of the invention is formulated together with a pharmaceutically acceptable excipient, any suitable excipient may be used, including for example inert diluents, disintegrating agents, binding agents, lubricating agents, sweetening agents, flavouring agents, colouring agents and preservatives. Suitable inert diluents include sodium and calcium carbonate, sodium and calcium phosphate, and lactose, while cornstarch and alginic acid are suitable disintegrating agents.

Binding agents may include starch and gelatin, while the lubricating agent, if present, will generally be magnesium stearate, stearic acid or talc. The pharmaceutical compositions may take any suitable form, and include for example tablets, elixirs, capsules, solutions, suspensions, powders, granules, nail lacquers, varnishes and veneers, skin patches and aerosols.

The pharmaceutical composition may take the form of a kit of parts, which kit may comprise the composition of the invention together with instructions for use and/or a plurality of different components in unit dosage form.

For oral administration the compound of the invention can be formulated into solid or liquid preparations such as capsules, pills, tablets, troches, lozenges, melts, powders, granules, solutions, suspensions, dispersions or emulsions (which solutions, suspensions dispersions or emulsions may be aqueous or non-aqueous). The solid unit dosage forms can be a capsule which can be of the ordinary hard- or soft-shelled gelatin type containing, for example, surfactants, lubricants, and inert fillers such as lactose, sucrose, calcium phosphate, and cornstarch. Tablets for oral use may include the compound of the invention, either alone or together with pharmaceutically acceptable excipients, such as inert diluents, disintegrating agents, binding agents, lubricating agents, sweetening agents, flavouring agents, colouring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid or talc. If desired, the tablets may be coated with a material such as glyceryl monostearate or glyceryl distearate, to delay absorption in the gastrointestinal tract. Capsules for oral use include hard gelatin capsules in which the compound of the invention is mixed with a solid diluent, and soft gelatin capsules wherein the active ingredient is mixed with water or an oil such as peanut oil, liquid paraffin or olive oil. Formulations for rectal administration may be presented as a suppository with a suitable base comprising for example cocoa butter or a salicylate. Formulations suitable for vaginal administration may be presented as pessaries, tampons, creams, gels, pastes, foams or spray formulations containing in addition to the active ingredient such carriers as are known in the art to be appropriate. For intramuscular, intraperitoneal, subcutaneous and intravenous use, the compounds of the invention will generally be provided in sterile aqueous solutions or suspensions, buffered to an appropriate pH and isotonicity.

The compounds of the invention may also be presented as liposome formulations.

In another embodiment, the compounds of the invention are tableted with conventional tablet bases such as lactose, sucrose, and cornstarch in combination with binders such as acacia, cornstarch, or gelatin, disintegrating agents intended to assist the break-up and dissolution of the tablet following administration such as potato starch, alginic acid, corn starch, and guar gum, lubricants intended to improve the flow of tablet granulations and to prevent the adhesion of tablet material to the surfaces of the tablet dies and punches, for example, talc, stearic acid, or magnesium, calcium, or zinc stearate, dyes, colouring agents, and flavouring agents intended to enhance the aesthetic qualities of the tablets and make them more acceptable to the patient.

Suitable excipients for use in oral liquid dosage forms include diluents such as water and alcohols, for example, ethanol, benzyl alcohol, and the polyethylene alcohols, either with or without the addition of a pharmaceutically acceptably surfactant, suspending agent or emulsifying agent.

The compounds of the invention may also be administered parenterally, that is, subcutaneously, intravenously, intramuscularly, or interperitoneally. In such embodiments, the compound is provided as injectable doses in a physiologically acceptable diluent together with a pharmaceutical carrier (which can be a sterile liquid or mixture of liquids). Suitable liquids include water, saline, aqueous dextrose and related compound solutions, an alcohol (such as ethanol, isopropanol, or hexadecyl alcohol), glycols (such as propylene glycol or polyethylene glycol), glycerol ketals (such as 2,2-dimethyl-1,3-dioxolane-4-methanol), ethers (such as poly(ethylene-glycol) 400), an oil, a fatty acid, a fatty acid ester or glyceride, or an acetylated fatty acid glyceride with or without the addition of a pharmaceutically acceptable surfactant (such as a soap or a detergent), suspending agent (such as pectin, carbomers, methylcellulose, hydroxypropylmethylcellulose, or carboxymethylcellulose), or emulsifying agent and other pharmaceutically adjuvants. Suitable oils which can be used in the parenteral formulations of this invention are those of petroleum, animal, vegetable, or synthetic origin, for example, peanut oil, soybean oil, sesame oil, cottonseed oil, corn oil, olive oil, petrolatum, and mineral oil.

Suitable fatty acids include oleic acid, stearic acid, and isostearic acid. Suitable fatty acid esters are, for example, ethyl oleate and isopropyl myristate. Suitable soaps include fatty alkali metal, ammonium, and triethanolamine salts and suitable detergents include cationic detergents, for example, dimethyl dialkyl ammonium halides, alkyl pyridinium halides, and alkylamines acetates; anionic detergents, for example, alkyl, aryl, and olefin sulphonates, alkyl, olefin, ether, and monoglyceride sulphates, and sulphosuccinates; nonionic detergents, for example, fatty amine oxides, fatty acid alkanolamides, and polyoxyethylenepolypropylene copolymers; and amphoteric detergents, for example, alkyl-beta-aminopropionates, and 2-alkylimidazoline quarternary ammonium salts, as well as mixtures.

The parenteral compositions of this invention will typically contain from about 0.5 to about 25% by weight of the compound of the invention in solution. Preservatives and buffers may also be used. In order to minimize or eliminate irritation at the site of injection, such compositions may contain a non-ionic surfactant having a hydrophile-lipophile balance (HLB) of from about 12 to about 17. The quantity of surfactant in such formulations ranges from about 5 to about 15% by weight. The surfactant can be a single component having the above HLB or can be a mixture of two or more components having the desired HLB. Illustrative of surfactants used in parenteral formulations are the class of polyethylene sorbitan fatty acid esters, for example, sorbitan monooleate and the high molecular weight adducts of ethylene oxide with a hydrophobic base, formed by the condensation of propylene oxide with propylene glycol.

The compounds of the invention may also be administered topically, and when done so the carrier may suitably comprise a solution, ointment or gel base. The base, for example, may comprise one or more of the following: petrolatum, lanolin, polyethylene glycols, bee wax, mineral oil, diluents such as water and alcohol, and emulsifiers and stabilizers. Topical formulations may contain a concentration of the compound from about 0.1 to about 10% w/v (weight per unit volume).

When used adjunctively, the compounds of the invention may be formulated for use with one or more other drug(s). In particular, the compounds of the invention may be used in combination with analgesics, anti-inflammatories (e.g. steroids), immunomodulatory agents and anti-spasmodics.

Thus, adjunctive use may be reflected in a specific unit dosage designed to be compatible (or to synergize) with the other drug(s), or in formulations in which the compound is admixed with one or more anti-inflammatories, cytokines or immunosuppressive agents (or else physically associated with the other drug(s) within a single unit dose). Adjunctive uses may also be reflected in the composition of the pharmaceutical kits of the invention, in which the compound of the invention is co-packaged (e.g. as part of an array of unit doses) with the antimicrobial agents and/or anti-inflammatories. Adjunctive use may also be reflected in information and/or instructions relating to the co-administration of the compound with antimicrobial agents and/or anti-inflammatories.

### EXEMPLIFICATION

The invention will now be described with reference to specific Examples. These are merely exemplary and for illustrative purposes only: they are not intended to be limiting in any way to the scope of the monopoly claimed or to the invention described. These examples constitute the best mode currently contemplated for practicing the invention.

The following abbreviations have been used:
Ac acetyl
Ac₂O acetic anhydride
AcOH acetic acid
aq aqueous
Ar aryl
Boc tert-butoxycarbonyl
nBuLi N-butyllithium
calcd calculated
CDI carbonyldiimidazole
conc concentrated
d day
DCE dichloroethane
DCM dichloromethane
DIBALH diisobutylaluminium hydride
DIPEA diisopropylethylamine
DMAP 4-dimethylaminopyridine
DMF dimethylformamide
EDC 1-ethyl-3-(3-dimethylaminopropyl)carbodiimide hydrochloride
ES+ electrospray ionization
EtOAc ethyl acetate
EtOH ethanol
Ex Example
h hour(s)
H BTU O-benzotriazole-N,N,N′,N′-tetramethyl-uronium-hexafluoro-phosphate
HOBt 1-hydroxybenzotriazole hydrate
HPLC High Performance Liquid Chromatography
HRMS High-Resolution Mass Spectrometry
Int Intermediate
LCMS Liquid Chromatography Mass Spectrometry
LDA lithium diisopropylamide
M molar
Me methyl
mCPBA meta-chloroperbenzoic acid
MeCN acetonitrile
MeOH methanol
min minute(s)
Ms methanesulfonate
MS Mass Spectrometry
NaBH(OAc)₃ sodium triacetoxyborohydride
NIS N-iodosuccinimide
   NMP N-methylpyrrolidone
   Rf Retention time
   RT room temperature
   sat saturated
   SCX Strong Cation Exchange
   SM starting material
   TFA trifluoroacetic acid
   TH F tetrahydrofuran

### EXAMPLES AND INTERMEDIATE COMPOUNDS

### Experimental Methods

Reactions were conducted at room temperature unless otherwise specified. Microwave reactions were performed with a CEM Discover microwave reactor using process vials fitted with aluminium caps and septa. Preparative flash chromatography was performed using silica gel (100-200 mesh).

Prep HPLC was performed using one of the following methods: Instrument—Agilent-1260 infinity; Column: Sunfire C8 (19×250) mm, 5μ or Sunfire C18 (19×250) mm, 5μ; Solvents: solvent A=5 mM Ammonium acetate in water; solvent B=acetonitrile/solvent A=0.1% TFA; solvent B=acetonitrile/; Detection wavelength 214 nm. Instrument—Waters 2767 autoprep with 2998 detector; Column: X TERRA C18 (19×250) mm, 10μ or Sunfire C18 (19×250) mm, 10μ; Solvents: solvent A=5 mM Ammonium acetate in water; solvent B=acetonitrile/solvent A=acetonitrile; solvent B=0.1% TFA in Water; Detection wavelength 214 nm. The purest fractions were collected, concentrated and dried under vacuum. Compounds were typically dried in a vacuum oven at 40° C. prior to purity analysis. Compound analysis was performed by Waters Acquity UPLC, Waters 3100 PDA Detector, SQD; Column: Acquity BEH C-18, 1.7 micron, 2.1×100 mm; Gradient [time (min)/solvent B in A (%)]:0.00/10, 1.00/10, 2.00/15, 4.50/55, 6.00/90, 8.00/90, 9.00/10, 10.00/10; Solvents: solvent A=5 mM ammonium acetate in water; solvent B=acetonitrile; Injection volume 1 μL; Detection wavelength 214 nm; Column temperature 30° C.; Flow rate 0.3 mL/min or Waters Acquity UPLC, Waters 3100 PDA Detector, SQD; Column: Acquity HSS-T3, 1.8 micron, 2.1×100 mm; Gradient [time (min)/solvent B in A (%)]: 0.00/10, 1.00/10, 2.00/15, 4.50/55, 6.00/90, 8.00/90, 9.00/10, 10.00/10; Solvents: solvent A=0.1% trifluoroacetic acid in water; solvent B=acetonitrile; Injection volume 1 μL; Detection wavelength 214 nm; Column temperature 30° C.; Flow rate 0.3 mL/min.

400 MHz 1H nuclear magnetic resonance spectra (NMR) were recorded on an Avance Bruker AV400 spectrometer. In the NMR spectra the chemical shifts (b) are expressed in ppm relative to the residual solvent peak. Abbreviations have the following significances: b=broad signal, s=singlet, d=doublet, t=triplet, dd=doublet of doublets, ddd=doublet of double doublets. Abbreviations may be compounded and other patterns are unabbreviated.

The compounds prepared were named using ChemBioDraw Ultra 13.0 by CambridgeSoft.

In the absence of intermediate synthesis, the compounds are commercially available.

### Antibiotic Compounds

### Examples and Intermediate Compounds

These are summarized below:

### Synthetic Route 1

### N-Cyclopropyl-2-((5-(trifluoromethyl)benzo[d]oxazol-2-yl)amino)thiazole-4-carboxamide (Example 1)

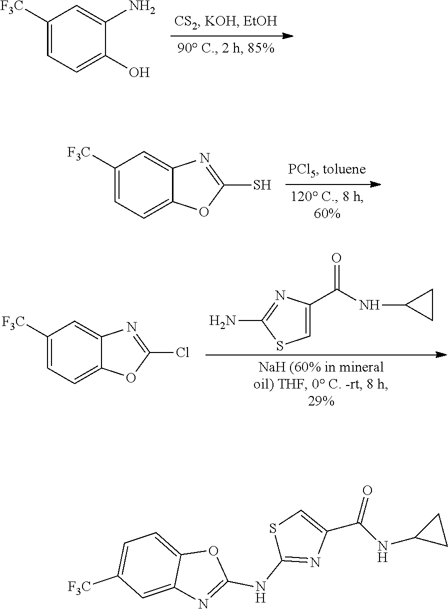 [SMILES: Nc1cc(C(F)(F)F)ccc1O ; FC(F)(F)c1ccc2oc(S)nc2c1 ; FC(F)(F)c1ccc2oc(Cl)nc2c1 ; Nc1nc(C(=O)NC2CC2)cs1 ; O=C(NC1CC1)c1csc(Nc2nc3cc(C(F)(F)F)ccc3o2)n1]

### 5-(Trifluoromethyl)benzo[d]oxazole-2-thiol

To a solution of KOH (4.75 g, 84.8 mmol) in EtOH (100 mL) were added 2-amino-4-(trifluoromethyl)phenol (5 g, 28.25 mmol) and CS₂ (5.11 mL, 84.8 mmol) at rt. The reaction mixture was refluxed overnight. The TLC showed the reaction to be complete. The solvent was removed under reduced pressure to give crude residue. The residue was acidified with 1N HCl (100 mL) and extracted with EtOAc (3×100 mL). The organic layer was washed with brine (100 mL), dried (Na₂SO₄), filtered and concentrated under reduced pressure to afford 5-(trifluoromethyl)benzo[d]oxazole-2-thiol as an off white solid. Yield: 5.2 g (85%); ¹H NMR (400 MHz, DMSO-d₆): δ 14.25 (bs, 1H), 7.72 (d, J=8.4 Hz, 1H), 7.64 (d, J=8.4 Hz, 1H), 7.51 (s, 1H); MS (ESI−) for CHNOS m/z 217.94 [M−H]⁺.

### 2-Chloro-5-(trifluoromethyl)benzo[d]oxazole

To a solution of 5-(trifluoromethyl)benzo[d]oxazole-2-thiol (5 g, 22.8 mmol) in toluene (50 mL) was added PCl₅ (47.4 g, 2.28 mmol) portion wise at rt. The reaction mixture was heated at 120° C. overnight. The TLC showed reaction to be complete. The reaction mixture was concentrated under reduced pressure to dryness. The residue was dissolved in Et₂O. The insoluble solid was filtered. The filtrate was concentrated under reduced pressure. The crude residue was purified by column chromatography using silica gel (100-200 mesh), eluting with hexane to 5% EtOAc in hexane to afford 2-chloro-5-(trifluoromethyl)benzo[d]oxazole as an orange solid. Yield: 2.4 g (48%); ¹H NMR (400 MHz, DMSO-d₆): δ8.08 (s, 1H), 7.89 (d, J=8.0 Hz, 1H), 7.70 (d, J=8.0 Hz, 1H).

### N-Cyclopropyl-2-((5-(trifluoromethyl)benzo[d]oxazol-2-yl)amino)thiazole-4-carboxamide

To a solution of 2-amino-N-cyclopropylthiazole-4-carboxamide (415 mg, 2.30 mmol) in dry THF (30 mL) at 0° C. was added sodium hydride (60% in mineral oil, 170 mg, 2.30 mmol). The resulted mixture was stirred at 0° C. for 15 min. 2-Chloro-5-(trifluoromethyl)benzo[d]oxazole (500 mg, 2.30 mmol) was added to reaction mixture and was stirred at rt for 8 h. The TLC showed reaction to be complete. The reaction mixture was quenched with sat. aq. NH₄Cl solution (20 mL) and extracted with EtOAc (3×20 mL). The organic layer was washed with brine (20 mL), dried (Na₂SO₄), filtered and concentrated under reduce pressure. The residue was triturated with Et₂O (25 mL) and dried under vacuum to give N-cyclopropyl-2-((5-(trifluoromethyl)benzo[d]oxazol-2-yl)amino)thiazole-4-carboxamide as a yellow solid. Yield: 240 mg (29%); ¹H NMR (400 MHz, DMSO-d₆): δ 13.11 (bs, 1H), 8.29 (bs, 1H), 7.79 (s, 1H), 7.70-7.73 (m, 2H), 7.53 (d, J=8.4 Hz, 1H), 2.79-2.84 (m, 1H), 0.71-0.75 (m, 2H), 0.56-0.60 (m, 2H); MS (ESI+) for CHNOS m/z 369.14 [M+H]⁺.

### Intermediate 1

### 2-Amino-N-cyclopropylthiazole-4-carboxamide

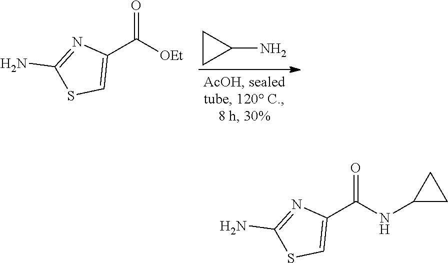

A mixture of ethyl 2-aminothiazole-4-carboxylate (10 g, 58.0 mmol) and cyclopropylamine (100 ml) in AcOH (10 mL) was heated at 80° C. in a sealed tube overnight. The TLC showed reaction to be complete. The reaction was cooled to rt and quenched into ice-water. The solid precipitated was and filtered and dried under vacuum. The obtained solid was triturated with Et₂O (200 ml) to give 2-amino-N-cyclopropylthiazole-4-carboxamide as an off white solid. Yield: 3.2 g (30%); ¹H NMR (400 MHz, DMSO-d₆): δ 7.68 (bs, 1H), 7.16 (s, 1H), 7.03 (bs, 2H), 2.76-2.77 (m, 1H), 0.66 (bs, 2H), 0.55 (bs, 2H); MS (ESI+) for CHNOS m/z 184.0 [M+H]⁺.

The following intermediates were prepared in a similar manner to 2-amino-N-cyclopropylthiazole-4-carboxamide following intermediate synthesis 1.

| | | | | Spectral Data |
|---|---|---|---|---|
| Name | Int | Structure | Yield | 1H NMR & LCMS |
| 2-Amino-N- isopropylthiazole- 4- carboxamide | 2 | 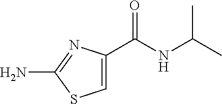 | 55% | ¹H NMR (400 MHz, DMSO-d₆): δ 7.34 (bs, 1H), 7.15 (s, 1H), 7.10 (bs, 2H), 3.93-4.05 (m, 1H), 1.04-1.26 (m, 6H). |
| 5-Amino-N- cyclopropyl- 1,3,4- oxadiazole-2- carboxamide | 3 | 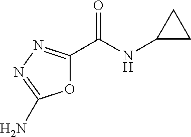 [SMILES: Nc1nnc(C(=O)NC2CC2)o1] | 47% | ¹H NMR (400 MHz, DMSO-d₆): δ 8.92 (bs, 1H), 7.50 (bs, 2H), 2.76- 2.79 (m, 1H), 0.60-0.68 (m, 4H). |

### Intermediate 4

### tert-Butyl (4-(methylcarbamoyl)thiazol-2-yl)carbamate

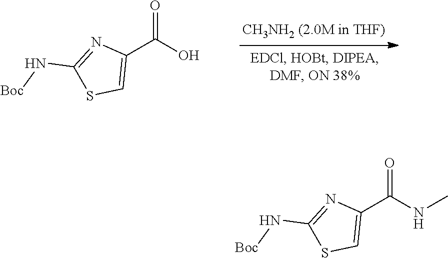 [SMILES: CC(C)(C)OC(=O)Nc1nc(C(=O)O)cs1 ; CNC(=O)c1csc(NC(=O)OC(C)(C)C)n1]

To a solution of 2-((tert-butoxycarbonyl)amino)thiazole-4-carboxylic acid (3 g, 12.2 mmol) in DMF (30 mL) was added EDCl (3.6 g, 18.7 mmol), HOBt (2.5 g, 18.7 mmol) and DIPEA (6.6 mL) at rt. The resulting reaction mixture was stirred at rt for 0.5 h and methylamine (2M in THF, 12.3 mL, 24.4 mmol) was added at rt. The reaction mixture was stirred overnight at rt. The TLC showed the reaction to be complete. The reaction mixture was poured into ice-cold water (50 mL) and extracted with EtOAc (3×50 mL). The organic layer was washed subsequently with 1 N HCl (50 mL), aq sat. NaHCO₃ (50 mL), water (50 mL) and brine (50 mL). The organics were dried (Na₂SO₄) and concentrated under reduced pressure to afford tert-butyl (4-(methylcarbamoyl)thiazol-2-yl)carbamate as a yellow solid. Yield: 1.2 g (38%); ¹H NMR (400 MHz, DMSO-d₆): δ 11.61 (bs, 1H), 7.72 (bs, 1H), 7.69 (s, 1H), 2.77 (bs, 3H), 1.49 (s, 9H); MS (ESI+) for CHNOS m/z 258.08 [M+H]⁺.

The following intermediates were prepared in a similar manner to tert-butyl (4-(methylcarbamoyl) thiazol-2-yl) carbamate.

| | | | | Spectral Data |
|---|---|---|---|---|
| Name | Int | Structure | Yield | 1H NMR & LCMS |
| tert-Butyl (4- (ethylcarbamoyl) thiazol-2- yl)carbamate | 5 | 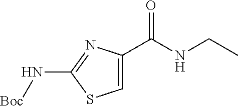 [SMILES: CCNC(=O)c1csc(NC(=O)OC(C)(C)C)n1] | 45% | MS (ESI+) for CHNOS m/z 272.08 [M + H]⁺; ¹H NMR (400 MHz, DMSO-d₆): δ 11.61 (bs, 1H), 7.65-7.75 (m, 2H), 3.28 (q, J = 7.2 Hz, 2H), 1.49 (s, 9H), 1.18 (t, J = 7.2 Hz, 3H). |
| tert-Butyl (4- (phenylcarba- moyl)thiazol-2- yl)carbamate | 6 | 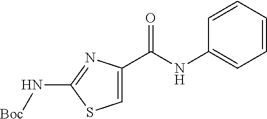 [SMILES: CC(C)(C)OC(=O)Nc1nc(C(=O)Nc2ccccc2)cs1] | 50% | MS (ESI+) for CHNOS m/z 320.09 [M + H]⁺; ¹H NMR (400 MHz, DMSO-d₆): δ 11.73 (bs, 1H), 9.65 (s, 1H), 7.94 (d, J = 9.4 Hz, 1H), 7.71 (d, J = 8.0 Hz, 2H), 7.30-7.40 (m, 2H), 6.97-7.01 (m, 1H), 1.50 (s, 9H). |
| tert-Butyl (4-((3- fluorophenyl)car- bamoyl)thiazol- 2-yl)carbamate | 7 | 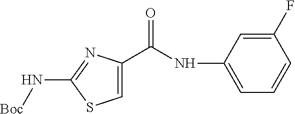 [SMILES: CC(C)(C)OC(=O)Nc1nc(C(=O)Nc2cccc(F)c2)cs1] | 63% | MS (ESI+) for CHNOS m/z 338.06 [M + H]⁺; ¹H NMR (400 MHz, DMSO-d₆): δ 11.73 (bs, 1H), 9.93 (s, 1H), 7.96 (s, 1H), 7.73 (d, J = 11.6 Hz, 1H), 7.49 (d, J = 8.0 Hz, 1H), 7.35-7.41 (m, 1H), 6.91-6.95 (m, 1H), 1.50 (s, 9H). |
| tert-Butyl (4-((3- chlorophenyl)car- bamoyl)thiazol- 2-yl)carbamate | 8 | 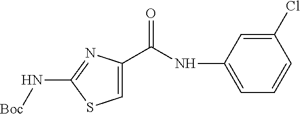 [SMILES: CC(C)(C)OC(=O)Nc1nc(C(=O)Nc2cccc(Cl)c2)cs1] | 40% | MS (ESI+) for CHNOS m/z 354.21 [M + H]⁺; ¹H NMR (400 MHz, DMSO-d₆): δ 11.72 (bs, 1H), 9.94 (s, 1H), 7.96 (bs, 2H), 7.62 (d, J = 8.4 Hz, 1H), 7.35-7.39 (m, 1H), 7.16 (d, J = 7.6 Hz, 1H), 1.50 (s, 9H). |
| tert-Butyl (4- carbamoylthiazol- 2- yl)carbamate | 9 | 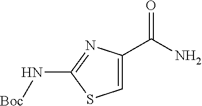 [SMILES: CC(C)(C)OC(=O)Nc1nc(C(N)=O)cs1] | 57% | MS (ESI+) for CHNOS m/z 244.16 [M + H]⁺; ¹H NMR (400 MHz, DMSO-d₆): δ 11.61 (bs, 1H), 7.72 (s, 1H), 7.51 (s, 1H), 7.12 (s, 1H), 1.49 (s, 9H). |
| tert-Butyl (4- (cyclopropylcar- bamoyl)oxazol- 2-yl)carbamate | 10 | 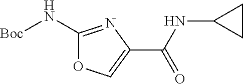 [SMILES: CC(C)(C)OC(=O)Nc1nc(C(=O)NC2CC2)co1] | 45% | MS (ESI+) for CHNOS m/z 268.28 [M + H]⁺; ¹H NMR (400 MHz, DMSO-d₆): δ 10.72 (bs, 1H), 8.27 (s, 1H), 7.95 (s, 1H), 2.74- 2.77 (m, 1H), 1.45 (s, 9H), |
| | | | | 0.58-0.66 (m, 4H). |
| tert-Butyl (4- (cyclopropylcar- bamoyl)-5- methylthiazol-2- yl)carbamate | 11 | 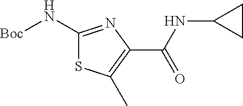 [SMILES: Cc1sc(NC(=O)OC(C)(C)C)nc1C(=O)NC1CC1] | 79% | MS (ESI+) for CHNOS m/z 298.27 [M + H]⁺; ¹H NMR (400 MHz, DMSO-d₆): δ 11.36 (bs, 1H), 7.42 (s, 1H), 2.73-2.76 (m, 1H), 2.58 (s, 3H), 1.47 (s, 9H), 0.67-0.70 (m, 2H), 0.50- |
| | | | | 0.57 (m, 2H). |
| tert-Butyl (5- (cyclopropylcar- bamoyl)thiazol- 2-yl)carbamate | 12 | 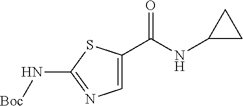 [SMILES: CC(C)(C)OC(=O)Nc1ncc(C(=O)NC2CC2)s1] | 36% | MS (ESI+) for CHNOS m/z 284.23 [M + H]⁺; ¹H NMR (400 MHz, DMSO-d₆): δ 11.68 (bs, 1H), 8.39 (bs, 1H), 7.90 (s, 1H), 2.65- 2.73 (m, 1H), 1.48 (s, 9H), 0.60-0.66 (m, 2H), 0.47- 0.53 (m, 2H). |

### Intermediate 13

### 2-Amino-N-methylthiazole-4-carboxamide

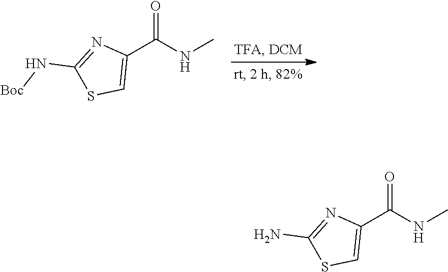

To a solution of tert-butyl (4-(methylcarbamoyl)thiazol-2-yl)carbamate (1.2 g, 4.6 mmol) in DCM (35 mL) was added TFA (12 mL) dropwise at rt. The reaction mixture was stirred at rt for 3 h. The TLC showed the reaction to be complete. The solvent was removed under reduced pressure. The residue obtained was basified to pH 8 with aq. NaHCO₃ solution and extracted with EtOAc (3×50 mL). The organic layer was washed with brine (50 mL), dried (Na₂SO₄), filtered and concentrated under vacuum to afford 2-amino-N-methylthiazole-4-carboxamide as a yellow solid. Yield: 600 mg (82%); ¹H NMR (400 MHz, DMSO-d₆): δ 7.74 (bs, 1H), 7.14 (s, 1H), 7.03 (bs, 2H), 2.71 (bs, 3H); MS (ESI+) for CHNOS m/z 158.04 [M+H]⁺.

The following intermediates were prepared in a similar manner to 2-amino-N-methylthiazole-4-carboxamide.

| | | | | Spectral Data |
|---|---|---|---|---|
| Name | Int | Structure | Yield | 1H NMR & LCMS |
| 2-Amino-N- ethylthiazole- 4-carboxamide | 14 | 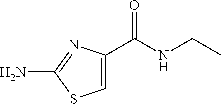 | 79% | MS (ESI+) for CHNOS m/z 171.92 [M + H]⁺; ¹H NMR (400 MHz, DMSO- d₆): δ 7.72 (bs, 1H), 7.14 (s, 1H), 7.05 (bs, 2H), 3.23 (q, J = 6.8 Hz, 2H), 1.06 (t, J = 6.8 Hz, 3H). |
| 2-Amino-N- phenylthiazole- 4-carboxamide | 15 | 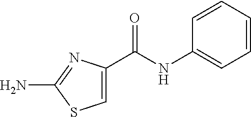 [SMILES: Nc1nc(C(=O)Nc2ccccc2)cs1] | 84% | MS (ESI+) for CHNOS m/z 220.16 [M + H]⁺; ¹H NMR (400 MHz, DMSO- d₆): δ 9.61 (bs, 1H), 7.74 (d, J = 7.6 Hz, 2H), 7.29- 7.40 (m, 3H), 7.21 (bs, 2H), 7.05-7.09 (m, 1H). |
| 2-Amino-N-(3- fluorophenyl)thi- azole-4- carboxamide | 16 | 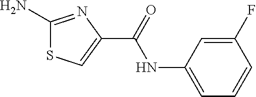 [SMILES: Nc1nc(C(=O)Nc2cccc(F)c2)cs1] | 81% | MS (ESI+) for CHNOS m/z 238.19 [M + H]⁺; ¹H NMR (400 MHz, DMSO- d₆): δ 9.98 (bs, 1H), 7.74 (d, J = 11.6 Hz, 1H), 7.55 (d, J = 8.0 Hz, 1H), 7.46 (s, 1H), 7.33-7.38 (m, 3H), 6.89-6.93 (m, 1H). |
| 2-Amino-N-(3- chlorophenyl)thi- azole-4- carboxamide | 17 | 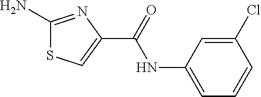 [SMILES: Nc1nc(C(=O)Nc2cccc(Cl)c2)cs1] | 76% | MS (ESI+) for CHNOS m/z 254.07 [M + H]⁺; ¹H NMR (400 MHz, DMSO- d₆): δ 9.89 (bs, 1H), 7.99 (s, 1H), 7.67 (d, J = 7.6 Hz, 1H), 7.41 (s, 1H), 7.32-7.36 (m, 1H), 7.12- 7.17 (m, 3H). |
| 2- Aminothiazole- 4-carboxamide | 18 | 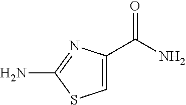 | 74% | MS (ESI+) for CHNOS m/z 144.18 [M + H]⁺; ¹H NMR (400 MHz, DMSO- d₆): δ 7.35 (bs, 1H), 7.17 (s, 1H), 7.05 (bs, 3H). |
| 2-Amino-N- cyclopropyloxa- zole-4- carboxamide | 19 | 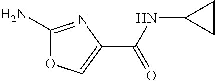 [SMILES: Nc1nc(C(=O)NC2CC2)co1] | 64% | MS (ESI+) for CHNOS m/z 167.98 [M + H]⁺; ¹H NMR (400 MHz, DMSO- d₆): δ 7.80 (s, 1H), 7.69 (bs, 1H), 6.76 (bs, 2H), 2.74-2.77 (m, 1H), 0.55- 0.66 (m, 4H). |
| 2-Amino-N- cyclopropyl-5- methylthiazole- 4-carboxamide | 20 | 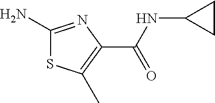 | 60% | MS (ESI+) for CHNOS m/z 198.03 [M + H]⁺; ¹H NMR (400 MHz, DMSO- d₆): δ 7.55 (bs, 1H), 6.80 (bs, 2H), 2.71-2.77 (m, 1H), 2.49 (s, 3H), 0.61- 0.68 (m, 2H), 0.49-0.56 (m, 2H). |
| 2-Amino-N- cyclopropylthia- zole-5- carboxamide | 21 | 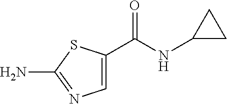 [SMILES: Nc1ncc(C(=O)NC2CC2)s1] | 56% | MS (ESI+) for CHNOS m/z 184.0 [M + H]⁺; ¹H NMR (400 MHz, DMSO-d₆): δ 8.06 (bs, 1H), 7.55 (s, 1H), 7.41 (bs, 2H), 2.65- 2.71 (m, 1H), 0.60-0.66 (m, 2H), 0.47-0.54 (m, 2H). |

### Intermediate 22

### 5-Morpholinothiazol-2-amine

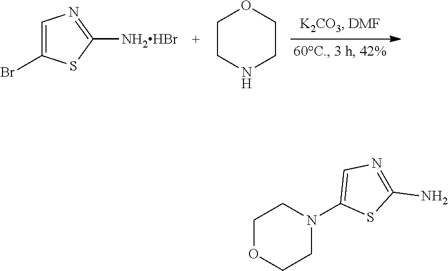

To a mixture of 5-bromothiazol-2-amine hydrobromide (1 g, 3.85 mmol) and K₂CO₃ (2.1 g, 15.2 mmol) in DMF (10 mL) was added morpholine (0.67 mL, 7.7 mmol) at rt under N₂ atmosphere. The reaction mixture was heated at 60° C. for 3 h. The TLC showed reaction to be complete. The reaction mixture was allowed to cool to rt, poured into ice-cold H₂O (50 mL) and extracted with EtOAc (3×50 mL). The organic layer was washed with brine (50 mL), dried (Na₂SO₄), filtered and concentrated under vacuum to afford 5-morpholinothiazol-2-amine as off white solid. Yield: 300 mg (42%); ¹H NMR (400 MHz, DMSO-d₆): δ6.46 (bs, 2H), 6.28 (s, 1H), 3.65 (t, J=4.5 Hz, 4H), 2.79 (t, J=4.5 Hz, 4H); MS (ESI+) for CHNOS m/z 186.05 [M+H]⁺.

The following intermediates were prepared in a similar manner to 5-morpholinothiazol-2-amine.

| | | | | Spectral Data |
|---|---|---|---|---|
| Name | Int | Structure | Yield | 1H NMR & LCMS |
| 5-(Piperidin-1- yl)thiazol-2- amine | 23 | 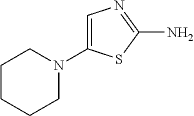 [SMILES: Nc1ncc(N2CCCCC2)s1] | 35% | MS (ESI+) for CHNOS m/z 184.08 [M − H]⁺; ¹H NMR (400 MHz, DMSO- d₆): δ 6.39 (bs, 2H), 6.23 (s, 1H), 2.77 (t, J = 5.0 Hz, 4H), 1.1.54-1.59 (m, 4H), 1.42-1.50 (m, 2H). |
| tert-Butyl 4-(2- aminothiazol- 5- yl)piperazine- 1-carboxylate | 24 | 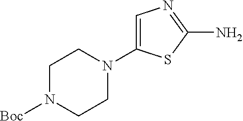 [SMILES: CC(C)(C)OC(=O)N1CCN(c2cnc(N)s2)CC1] | 59% | MS (ESI+) for CHNOS m/z 285.20 [M + H]⁺; ¹H NMR (400 MHz, DMSO- d₆): δ 6.50 (bs, 2H), 5.76 (s, 1H), 3.38 (t, J = 4.8 Hz, 4H), 2.75 (t, J = 4.8 Hz, 4H), 1.40 (s, 9H). |
| 5-(4- Methylpiperazin- 1-yl)thiazol- 2-amine | 25 | 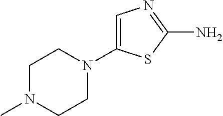 | 32% | MS (ESI+) for CHNOS m/z 199.12 [M + H]⁺; ¹H NMR (400 MHz, DMSO- d₆): δ 6.42 (s, 2H), 6.23 (s, 1H), 2.79-2.82 (m, 4H), 2.39 (bs, 4H), 2.19 (S, 3H). |

### Intermediate 26

### 3-Methyl-1,2,4-oxadiazol-5-amine

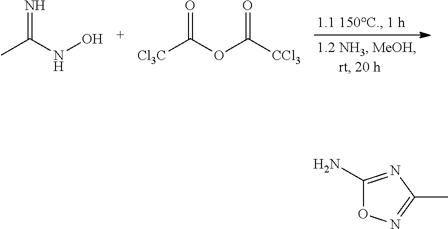

A mixture of N-hydroxyacetimidamide (1.1 g, 14.8 mmol) and trichloroacetic anhydride (6 mL) was heated at 150° C. for 1 h. TLC showed reaction to be complete. The reaction mixture was cooled to rt, poured into water (20 mL) and extracted with Et₂O (3×25 mL). The organic layer was washed with brine (25 mL), dried (Na₂SO₄) and concentrated under reduced pressure to obtain residue. The residue was taken in MeOH (10 mL) and purged NH₃ (g) for 0.5 h at −40° C. The reaction mixture was stirred at rt for 16 h. The TLC showed reaction to be complete. The reaction mixture was concentrated under reduced pressure and triturated with Et₂O (25 mL) to afford 3-methyl-1,2,4-oxadiazol-5-amine as an orange solid. Yield: 540 mg (38%); ¹H NMR (400 MHz, DMSO-d₆): δ 7.62 (s, 2H), 2.05 (s, 3H); MS (ESI+) for CHNOS m/z 98.9[M+H]⁺.

### Intermediate 27

### 2-Amino-5-chloro-4-(trifluoromethyl)phenol

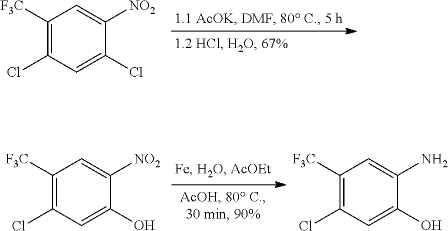 [SMILES: O=[N+]([O-])c1cc(C(F)(F)F)c(Cl)cc1Cl ; O=[N+]([O-])c1cc(C(F)(F)F)c(Cl)cc1O ; Nc1cc(C(F)(F)F)c(Cl)cc1O]

### 5-Chloro-2-nitro-4-(trifluoromethyl)phenol

To a solution of 1,5-dichloro-2-nitro-4-(trifluoromethyl)benzene (4 g, 15.4 mmol) in DMF (20 mL) was added potassium acetate (1.7 g, 16.9 mmol) portionwise. The reaction was stirred at 60° C. for 1 h and at 80° C. for 3 h. To this reaction mixture potassium acetate (1.7 g, 16.9 mmol) was added and it was stirred at 80° C. for 1 h. The TLC showed reaction to be complete. The reaction mixture was cooled to rt, 1N HCl (100 mL) was added and extracted with EtOAc (3×100 mL). The organic layer was washed with water (100 mL), brine (100 mL), dried (Na₂SO₄), filtered and concentrated under reduced pressure. The crude residue was purified by column chromatography using silica gel (100-200 mesh), eluting with hexane to 5% EtOAc in hexane to afford 5-chloro-2-nitro-4-(trifluoromethyl)phenol as a yellow solid. Yield: 2.5 g (67%); ¹H NMR (400 MHz, CDCl₃): 510.81 (s, 1H), 8.49 (s, 1H), 7.31 (s, 1H); MS (ESI+) for CHNOS m/z 240.11 [M−H]⁺.

### 2-Amino-5-chloro-4-(trifluoromethyl)phenol

To a suspension of Fe (2.9 g, 51.8 mmol) in AcOH (10 mL) and H₂O (15 mL) at 80° C. was added 5-chloro-2-nitro-4-(trifluoromethyl)phenol (2.5 g, 10.3 mmol) in EtOAc (5.0 mL) dropwise. The reaction mixture was heated at 80° C. for 30 min. The TLC showed reaction to be complete. The reaction mixture was cooled to rt, H₂O (50 mL) was added and extracted with EtOAc (3×50 mL). The organic layer was washed with water (100 mL), brine (100 mL), dried (Na₂SO₄), filtered and concentrated under reduced pressure to afford 2-amino-5-chloro-4-(trifluoromethyl)phenol as a white solid. Yield: 2.0 g (90%); MS (ESI+) for CHNOS m/z 210.12 [M−H]⁺.

### Intermediate 28

### 3-Amino-4-hydroxybenzonitrile

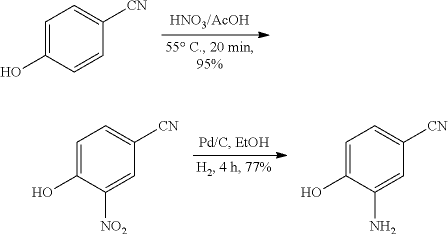 [SMILES: N#Cc1ccc(O)cc1 ; N#Cc1ccc(O)c([N+](=O)[O-])c1 ; N#Cc1ccc(O)c(N)c1]

### 4-Hydroxy-3-nitrobenzonitrile

To a mixture of HNO₃ (2.7 mL, 63.0 mmol) and AcOH (5 mL) was added 4-hydroxybenzonitrile (5 g, 42 mmol) in AcOH (5 mL) dropwise at 40° C. The reaction mixture was heated at 55° C. for 20 min. The TLC showed reaction to be complete. The reaction mixture was poured into ice-water (100 mL). The precipitated solid was filtered, washed with water (200 mL) and dried under vacuum to afford 4-hydroxy-3-nitrobenzonitrile as a yellow solid. Yield: 2.5 g (67%); ¹H NMR (400 MHz, DMSO-d₆): δ12.34 (bs, 1H), 8.43 (s, 1H), 7.94 (d, J=10.5 Hz, 1H), 7.24 (d, J=8.7 Hz, 1H); MS (ESI+) for CHNOS m/z 163.03 [M+H]⁺.

### 3-Amino-4-hydroxybenzonitrile

To a solution of 4-hydroxy-3-nitrobenzonitrile (5 g, 30.4 mmol) in EtOH (100 mL) was added 10% Pd/C (4 g). The reaction mixture was stirred at rt under H₂ balloon atmosphere for 4 h. The TLC showed reaction to be complete. The reaction mixture was passed through a pad of celite. The celite was washed with EtOH (100 mL). The filtrate was concentrated under reduced pressure to afford 3-amino-4-hydroxybenzonitrile as a black solid. Yield: 2.5 g (67%); ¹H NMR (400 MHz, DMSO-d₆): δ 9.02 (bs, 1H), 6.81-6.86 (m, 1H), 6.73 (d, J=7.8 Hz, 1H), 6.49 (d, J=7.8 Hz, 1H), 6.38 (bs, 1H), 6.17 (d, J=6.5 Hz, 1H); MS (ESI+) for CHNOS m/z 165.10 [M+H]⁺.

The following intermediate was prepared in a similar manner to 4-hydroxy-3-nitrobenzonitrile.

| | | | | Spectral Data |
|---|---|---|---|---|
| Name | Int | Structure | Yield | 1H NMR & LCMS |
| 3- Hydroxy- 4-nitro- benzo- nitrile | 29 | 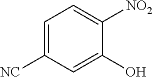 [SMILES: N#Cc1ccc([N+](=O)[O-])c(O)c1] | 18% | MS (ESI−) for CHNOS m/z 163.12 [M − H]⁺; ¹H NMR (400 MHz, DMSO- d₆): δ 11.84 (s, 1H), |
| | | | | 8.01 (d, J = 8.4 Hz, |
| | | | | 1H), 7.50 (s, 1H), |
| | | | | 7.41-7.45 (m, 1H). |

The following intermediate was prepared in a similar manner to 3-amino-4-hydroxybenzonitrile

| | | | | Spectral Data |
|---|---|---|---|---|
| Name | Int | Structure | Yield | 1H NMR & LCMS |
| 4-Amino-3- hydroxybenzo- nitrile | 30 | 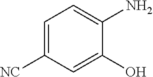 | 60% | MS (ESI+) for CHNOS m/z 134.97 [M + H]⁺; ¹H NMR (400 MHz, DMSO-d₆): δ 9.77 (bs, 1H), 6.98 (dd, J = 8.2 Hz, 1.6 Hz, 1H), 6.86 (d, J = 1.6 Hz, 1H), 6.62 (d, J = 8.2 Hz, 1H), 5.56 (bs, 2H). |

### Intermediate 31

### 2-Amino-3,5-dichlorophenol

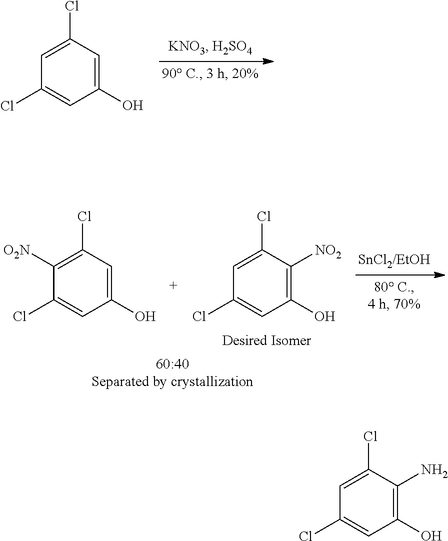 [SMILES: Oc1cc(Cl)cc(Cl)c1 ; O=[N+]([O-])c1c(Cl)cc(O)cc1Cl ; O=[N+]([O-])c1c(O)cc(Cl)cc1Cl ; Nc1c(O)cc(Cl)cc1Cl]

### 3,5-Dichloro-2-nitrophenol

To a solution of 3,5-dichlorophenol (10 g, 6.17 mmol) in H₂O (30 mL) were added potassium nitrate (0.93 g, 9.21 mmol) and 1.0 mL of H₂SO₄ (diluted with 5 mL H₂O).

The reaction mixture was stirred at 90° C. for 3 h. The TLC showed reaction to be complete. The resulting solution was cooled, neutralized with sodium bicarbonate solution (5% w/v) and extracted with EtOAc (3×100 mL). The organic layer was washed with water (50 mL), brine (50 mL), dried (Na₂SO₄), filtered and concentrated under reduced pressure. The crude residue was purified by column chromatography using silica gel (100-200 mesh), eluting with hexane to 15% EtOAc in hexane to give a mixture of regioisomers, 3,5-dichloro-2-nitrophenol and 3,5-dichloro-4-nitrophenol as a brown liquid. The hexane was added to brown liquid slowly and precipitated solid was filtered. The filtrate was concentrated under reduced pressure to afford desired 3,5-dichloro-2-nitrophenol as a orange oil. Yield: 2.5 g (20%); ¹H NMR (400 MHz; MeOD): δ 12.02 (bs, 1H), 7.30 (d, J=1.7 Hz, 1H), 7.07 (d, J=1.7 Hz, 1H); (MS (ESI+) for CHNOS m/z 206.06 [M−H]⁺.

### 2-Amino-3,5-dichlorophenol

A mixture of 3,5-dichloro-2-nitrophenol (2.5 g, 12.1 mmol) and SnCl₂ (3 g, 12.1 mmol) in EtOH (30 mL) was stirred at 90° C. for 4 h. The TLC showed reaction to be complete. The solvent was removed under reduced pressure. The residue was dissolved in EtOAc (50 mL) and washed with aq. NaHCO₃ (50 mL). The organic layer was washed brine (50 mL), dried (Na₂SO₄), filtered and concentrated under reduced pressure 2-amino-3,5-dichlorophenol as a off-white solid. Yield: 1.5 g (70%); ¹H NMR (400 MHz; DMSO-d₆): 56.79 (d, J=2.0 Hz, 1H), 6.64 (d, J=2.0 Hz, 1H), 4.78 (bs, 2H); (MS (ESI+) for CHNOS m/z 176.07 [M−H]⁺.

### Intermediate 32

### 2-Amino-5-chloro-3-methyl phenol

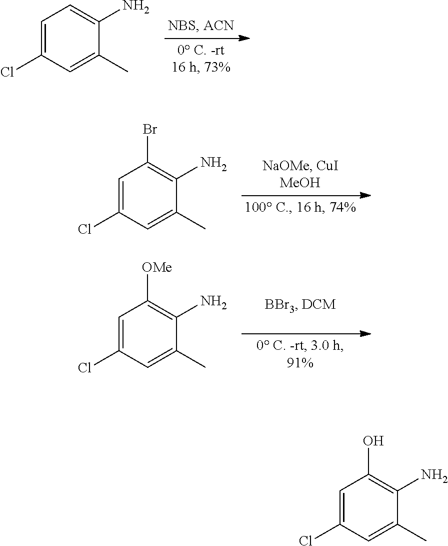

### 2-Bromo-4-chloro-6-methylaniline

To a solution of 4-chloro-2-methylaniline (15 g, 106.38 mmol) in ACN (150 mL) was added NBS (20.8 g, 110 mmol) at 0° C. slowly. The reaction mixture was stirred at rt 16 h. The TLC showed reaction to be complete. The reaction mixture was diluted with H₂O (200 mL) and extracted with ethyl acetate (3×200 mL). The organic layer was washed with saturated aq NaHCO₃ solution (200 mL). The organic layer was washed brine (200 mL), dried (Na₂SO₄), filtered and concentrated under reduced pressure to give the residue. The residue was purified by column chromatography using silica gel (100-200 mesh), eluting with 5% EtOAc in hexane to afford 2-bromo-4-chloro-6-methylaniline as a light brown solid. Yield: 17.1 g (73%); ¹H NMR (400 MHz, CDCl₃): δ7.29 (d, J=1.9 Hz, 1H), 7.26 (s, 1H), 6.99 (bs, 1H), 3.90 (bs, 2H), 2.19 (s, 3H).

### 4-Chloro-2-methoxy-6-methylaniline

To a solution of 2-bromo-4-chloro-6-methylaniline (5.0 g, 22.8 mmol) and CuI (4.78 g, 25 mmol) in MeOH (50 mL) was added sodium methoxide solution (25% in MeOH, 25 mL) slowly at rt. The mixture was stirred at 100° C. for 16 h. The TLC showed reaction to be complete. The solvent was evaporated under reduced pressure. The residue was diluted with aq. Saturated NH₄Cl solution (100 mL) and extracted with EtOAc (2×100 mL). The organic layer was washed with brine (100 mL), dried (Na₂SO₄), filtered and concentrated under reduced pressure. The residue was purified by column chromatography using silica gel (100-200 mesh), eluting with 5% EtOAc in hexane to afford 4-chloro-2-methoxy-6-methylaniline as dark brown liquid. Yield: 2.9 g (74%); (MS (ESI+) for CHNOS m/z 172.07 [M+H]⁺. ¹H NMR (400 MHz, DMSO-d₆): δ 6.72 (d, J=1.4 Hz, 1H), 6.65 (s, 1H), 4.53 (bs, 2H), 3.77 (s, 3H), 2.06 (s, 3H).

### 2-Amino-5-chloro-3-methyl phenol

To a solution of 4-chloro-2-methoxy-6-methylaniline (2.7 g, 15.7 mmol) in DCM (50 mL) was added BBr₃ (19.7 g, 78 mmol) at 0° C. slowly. The reaction mixture was stirred at rt for 3 h. The TLC showed reaction to be complete. The reaction mixture was neutralized with aq. NaHCO₃ solution (50 mL) at 0° C. and extracted with DCM (3×100 mL). The organic layer was washed with brine (50 mL), dried (Na₂SO₄), filtered and concentrated under reduced pressure to afford 2-amino-5-chloro-3-methylphenol as a brown solid. Yield: 2.27 g (91%); MS (ESI+) for CHNOS m/z 156.15 [M+H]⁺; ¹H NMR (400 MHz, DMSO-d₆): δ 9.46 (bs, 1H), 6.54 (s, 1H), 6.50 (s, 1H), 4.32 (bs, 2H), 2.03 (s, 3H).

### Intermediate 33

### 2-Amino-4,5-dichlorophenol

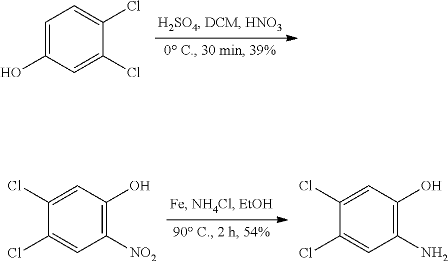 [SMILES: Oc1ccc(Cl)c(Cl)c1 ; O=[N+]([O-])c1cc(Cl)c(Cl)cc1O ; Nc1cc(Cl)c(Cl)cc1O]

### 4,5-Dichloro-2-nitrophenol

To a solution of 3,4-dichlorophenol (3 g, 18.41 mmol) and concentrated H₂SO₄ (1.56 mL, 27.6 mmol) in DCM (50 mL) at 0° C. was added fuming HNO₃ (1.2 mL, 18.41 mmol) dropwise. The reaction mixture was stirred at 0° C. for 30 minutes. The TLC showed reaction to be complete. Reaction was cooled to room temperature, quenched with ice-cold water (25 mL) and extracted with DCM (3×25 mL). The organics were dried (Na₂SO₄), filtered and concentrated under reduced pressure. The residue was purified by column chromatography using silica gel (100-200 mesh), eluting with 2% EtOAc in hexane to afford 4,5-dichloro-2-nitrophenol as yellow solid. Yield: 1.5 g (39%); (MS (ESI−) for CHNOS m/z 205.9 [M−H]⁻. ¹H NMR (400 MHz, CDCl₃): δ 10.46 (bs, 1H), 8.23 (s, 1H), 7.33 (s, 1H).

### 2-Amino-4,5-dichlorophenol

To a solution of 4,5-dichloro-2-nitrophenol (1.5 g, 7.21 mmol) in EtOH (20 mL) were added NH₄Cl (1.93 g, 36.1 mmol), Fe powder (2.0 g, 36.1 mmol) and H₂O (5.0 mL). The reaction mixture was stirred at 90° C. for 2 h. The TLC showed reaction to be complete. Reaction mixture was cooled to room temperature and filtered through a celite bed. The filtrate was concentrated, diluted with H₂O (25 mL) and extracted with EtOAc (3×50 mL). The organics were dried (Na₂SO₄), filtered and concentrated under reduced pressure The residue was purified by column chromatography using silica gel (100-200 mesh), eluting with 25% EtOAc in hexane to afford 2-amino-4,5-dichlorophenol as yellow solid. Yield: 700 mg (54%); (MS (ESI−) for CHNOS m/z 176.13 [M−H]⁻. ¹H NMR (400 MHz, DMSO-d₆): δ 9.73 (bs, 1H), 6.71-6.74 (m, 2H), 4.95 (bs, 2H).

The following intermediates were prepared in a similar manner to 5-(trifluoromethyl)benzo[d]oxazole-2-thiol.

| | | | | Spectral Data |
|---|---|---|---|---|
| Name | Int | Structure | Yield | 1H NMR & LCMS |
| Benzo[d]oxazole- 2-thiol | 34 | 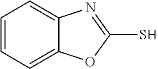 | 70% | MS (ESI−) for CHNOS m/z 149.87 [M − H]⁺; ¹H NMR (400 MHz, DMSO-d₆): δ 11.0 (bs, 1H), 7.37 (d, J = 8.4 Hz, 1H), 7.20-7.32 (m, |
| | | | | 3H). |
| 5- Methylbenzo[d] oxazole-2-thiol | 35 | 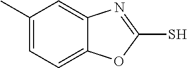 | 80% | MS (ESI+) for CHNOS m/z 166.10 [M + H]⁺; ¹H NMR (400 MHz, DMSO-d₆): δ 13.79 (bs, 1H), 7.38 (d, J = 8.4 Hz, 1H), 7.03-7.07 (m, |
| | | | | 2H), 2.36 (s, 3H). |
| 5- Chlorobenzo[d] oxazole-2-thiol | 36 | 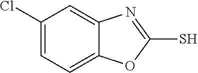 [SMILES: Sc1nc2cc(Cl)ccc2o1] | 76% | MS (ESI−) for CHNOS m/z 184.09 [M − H]⁺; ¹H NMR (400 MHz, DMSO-d₆): δ 14.05 (bs, 1H), 7.53 (d, J = 8.4 Hz, 1H), 7.29-7.33 (m, |
| | | | | 2H). |
| 5- Fluorobenzo[d] oxazole-2-thiol | 37 | 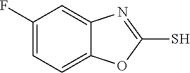 [SMILES: Fc1ccc2oc(S)nc2c1] | 84% | ¹H NMR (400 MHz, DMSO- d₆): δ 9.74 (bs, 1H), 7.29 (s, 1H), 6.91-7.01 (m, 2H). |
| 6- Fluorobenzo[d] oxazole-2-thiol | 38 | 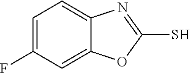 [SMILES: Fc1ccc2nc(S)oc2c1] | 80% | MS (ESI−) for CHNOS m/z 167.8 [M − H]⁺; ¹H NMR (400 MHz, DMSO-d₆): δ 14.57 (bs, 1H), 7.57 (d, J = 8.4 Hz, 1H), 7.20-7.26 (m, 1H), |
| | | | | 7.12-7.19 (m, 1H). |
| 6- Chlorobenzo[d] oxazole-2-thiol | 39 | 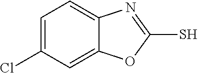 [SMILES: Sc1nc2ccc(Cl)cc2o1] | 89% | MS (ESI+) for CHNOS m/z 185.97 [M + H]⁺; ¹H NMR (400 MHz, DMSO-d₆): δ 14.02 (bs, 1H), 7.73 (s, 1H), 7.34 (d, J = 8.4 Hz, |
| | | | | 1H), 7.23 (d, J = 8.4 Hz, |
| | | | | 1H). |
| 6- (Trifluoromethyl) benzo[d]oxazole- 2-thiol | 40 | 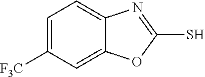 [SMILES: FC(F)(F)c1ccc2nc(S)oc2c1] | 80% | MS (ESI+) for CHNOS m/z 218.11 [M − H]⁺; ¹H NMR (400 MHz, DMSO-d₆): δ 14.12 (bs, 1H), 7.97 (s, 1H), 7.64 (d, J = 7.2 Hz, |
| | | | | 1H), 7.40 (d, J = 8.0 Hz, |
| | | | | 1H). |
| 6-Chloro-5- (trifluoromethyl) benzo[d]oxazole- 2-thiol | 41 | 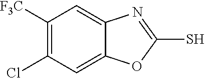 [SMILES: FC(F)(F)c1cc2nc(S)oc2cc1Cl] | 91% | MS (ESI+) for CHNOS m/z 254.03 [M − H]⁺; ¹H NMR (400 MHz, CDCl₃): δ 10.67 (bs, 1H), 7.56 (s, 2H). |
| 2- Mercaptobenzo [d]oxazole-5- carbonitrile | 42 | 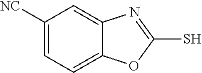 [SMILES: N#Cc1ccc2oc(S)nc2c1] | 65% | MS (ESI+) for CHNOS m/z 175.03 [M − H]. |
| 2- Mercaptobenzo [d]oxazole-6- carbonitrile | 43 | 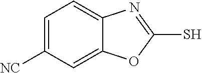 [SMILES: N#Cc1ccc2nc(S)oc2c1] | 87% | MS (ESI+) for CHNOS m/z 174.96 [M − H]⁺; ¹H NMR (400 MHz, DMSO-d₆): δ 14.33 (bs, 1H), 8.11 (s, 1H), 7.75 (d, J = 8.4 Hz, |
| | | | | 1H), 7.38 (d, J = 8.4 Hz, |
| | | | | 1H). |
| 5- (Methylsulfonyl) benzo[d]oxazole- 2-thiol | 44 | 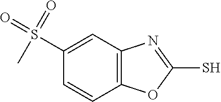 | 95% | MS (ESI+) for CHNOS m/z 227.99 [M − H]⁺; ¹H NMR (400 MHz, DMSO-d₆): δ 14.33 (bs, 1H), 7.81-7.85 (m, 1H), 7.73-7.77 (m, 1H), 7.67 (d, J = 1.4 Hz, 1H), 3.16 (s, 3H). |
| 7- Chlorobenzo[d] oxazole-2-thiol | 45 | 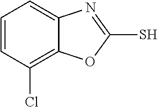 [SMILES: Sc1nc2cccc(Cl)c2o1] | 80% | MS (ESI+) for CHNOS m/z 183.97 [M − H]⁺; ¹H NMR (400 MHz, DMSO-d₆): δ 14.16 (bs, 1H), 7.26-7.36 (m, 2H), 7.20 ((d, J = 7.6 Hz, 1H). |
| 4- (trifluoromethyl) benzo[d]oxazole- 2-thiol | 46 | 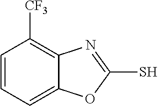 [SMILES: FC(F)(F)c1cccc2oc(S)nc12] | 94% | MS (ESI−) for CHNOS m/z 218.02 [M − H]⁺; ¹H NMR (400 MHz, DMSO-d₆): δ 14.46 (bs, 1H), 7.81 (d, J = 8.1 Hz, 1H), 7.57-7.62 (m, 1H), 7.39-7.46 (m, 1H). |
| 4,6- Dichlorobenzo[d] oxazole-2-thiol | 47 | 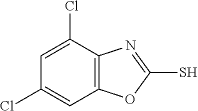 [SMILES: Sc1nc2c(Cl)cc(Cl)cc2o1] | 19% | MS (ESI−) for CHNOS m/z 218.02 [M − H]⁺; ¹H NMR (400 MHz, DMSO-d₆): δ 7.20 (s, 1H), 7.07 (s, 1H). |
| 6-Chloro-4- methylbenzo[d] oxazole-2-thiol | 48 | 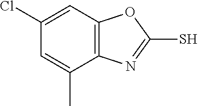 | 85% | (MS (ESI+) for CHNOS m/z 198.10 [M + H]⁺; ¹H NMR (400 MHz, DMSO- d₆): δ 7.23 (s, 1H), 6.98 (s, 1H), 2.31 (s, 3H). |
| Methyl 2- mercaptobenzo [d]oxazole-5- carboxylate | 49 | 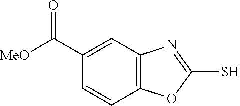 [SMILES: COC(=O)c1ccc2oc(S)nc2c1] | 74% | MS (ESI−) for CHNOS m/z 207.96 [M − H]^{+; 1}H NMR (400 MHz, DMSO-d₆): δ 14.13 (bs, 1H), 7.88 (d, J = 8.4 Hz, 1H), 7.68 (s, 1H), 7.62 (d, J = 8.4 Hz, 1H), 3.87 (s, 3H). |
| 5,6- diFluorobenzo[d] oxazole-2-thiol | 50 | 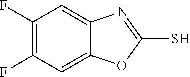 [SMILES: Fc1cc2nc(S)oc2cc1F] | 77% | MS (ESI−) for CHNOS m/z 186.17 [M − H]^{-; 1}H NMR (400 MHz, DMSO-d₆): δ 14.13 (bs, 1H), 7.83-7.87 (m, 1H), 7.39-7.44 (m, |
| | | | | 1H). |
| 6-Chloro-5- fluorobenzo[d] oxazole-2-thiol | 51 | 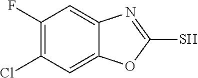 [SMILES: Fc1cc2nc(S)oc2cc1Cl] | 80% | MS (ESI−) for CHNOS m/z 202.11 [M − H]^{-; 1}H NMR (400 MHz, DMSO-d₆): δ 14.17 (bs, 1H), 7.89-7.91 (m, 1H), 7.36-7.39 (m, 1H). |
| 5-Chloro-6- (trifluoromethyl) benzo[d]oxazole- 2-thiol | 52 | 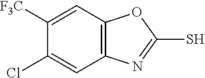 [SMILES: FC(F)(F)c1cc2oc(S)nc2cc1Cl] | 78% | MS (ESI−) for CHNOS m/z 252.17 [M − ]−^{; 1}H NMR (400 MHz, DMSO-d₆): δ 14.15 (bs, 1H), 7.55 (s, 1H), 7.28 (s, 1H). |
| 5,6- Dichlorobenzo[d] oxazole-2-thiol | 53 | 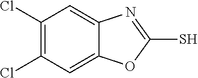 [SMILES: Sc1nc2cc(Cl)c(Cl)cc2o1] | 68% | MS (ESI−) for CHNOS m/z 217.94 [M − H]^{+; 1}HNMR (400 MHz, DMSO-d₆): δ 14.17 (bs, 1H), 7.95 (s, 1H), 7.49 (s, 1H). |

The following intermediates were prepared in a similar manner to 2-Chloro-5-(trifluoromethyl)benzo[d]oxazole.

| | | | | Spectral Data |
|---|---|---|---|---|
| Name | Int | Structure | Yield | 1H NMR & LCMS |
| 2- Chlorobenzo[d] oxazole | 54 | 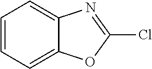 | 60% | Crude data showed product. Proceeded further without purification. |
| 2- Chloro-5- methylbenzo[d] oxazole | 55 | 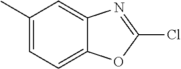 | 55% | Crude data showed product. Proceeded further without purification. |
| 2,5- Dichlorobenzo [d]oxazole | 56 | 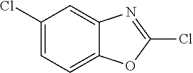 [SMILES: Clc1ccc2oc(Cl)nc2c1] | 50% | Crude data showed product. Proceeded further without purification. |
| 2-Chloro-5- fluorobenzo[d] oxazole | 57 | 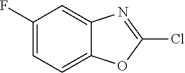 [SMILES: Fc1ccc2oc(Cl)nc2c1] | 62% | Crude data showed product. Proceeded further without purification. |
| 2-Chloro-6- fluorobenzo[d] oxazole | 58 | 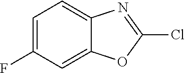 [SMILES: Fc1ccc2nc(Cl)oc2c1] | 60% | Crude data showed product. Proceeded further without purification. |
| 2,6- Dichlorobenzo [d]oxaole | 59 | 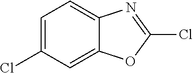 [SMILES: Clc1ccc2nc(Cl)oc2c1] | 60% | Crude data showed product. Proceeded further without purification. |
| 2-Chloro-6- (trifluoromethyl) benzo[d]oxa- zole | 60 | 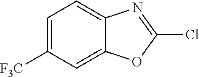 [SMILES: FC(F)(F)c1ccc2nc(Cl)oc2c1] | 26% | Crude data showed product. Proceeded further without purification. |
| 2,6-Dichloro- 5- (trifluorometh- yl)benzo[d]oxa- zole | 61 | 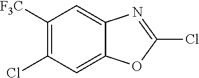 [SMILES: FC(F)(F)c1cc2nc(Cl)oc2cc1Cl] | 60% | Crude data showed product. Proceeded further without purification. |
| 2- Chlorobenzo[d] oxazole-5- carbonitrile | 62 | 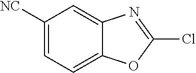 [SMILES: N#Cc1ccc2oc(Cl)nc2c1] | 50% | Crude data showed product. Proceeded further without purification. |
| 2- Chlorobenzo[d] oxazole-6- carbonitrile | 63 | 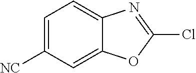 [SMILES: N#Cc1ccc2nc(Cl)oc2c1] | 62% | Crude data showed product. Proceeded further without purification. |
| 2-Chloro-5- (methylsulfonyl) benzo[d]oxa- zole | 64 | 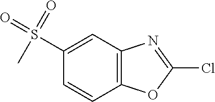 | 55% | Crude data showed product. Proceeded further without purification. |
| 2,7- Dichlorobenzo [d]oxazole | 65 | 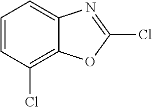 [SMILES: Clc1nc2cccc(Cl)c2o1] | 52% | Crude data showed product. Proceeded further without purification. |
| 2-Chloro-4- (trifluorometh- yl)benzo[d]oxa- zole | 66 | 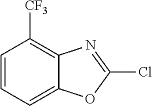 [SMILES: FC(F)(F)c1cccc2oc(Cl)nc12] | 45% | Crude data showed product. Proceeded further without purification. |
| 2,4,6- Trichlorobenzo [d]oxazole | 67 | 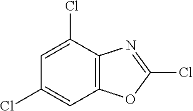 [SMILES: Clc1cc(Cl)c2nc(Cl)oc2c1] | 57% | Crude data showed product. Proceeded further without purification. |
| 2,5,6- trichlorobenzo [d]oxazole | 68 | 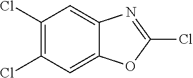 [SMILES: Clc1nc2cc(Cl)c(Cl)cc2o1] | 54% | Crude data showed product. Proceeded further without purification. |

### Intermediate 69

### 2,6-Dichloro-4-methylbenzo[d]oxazole

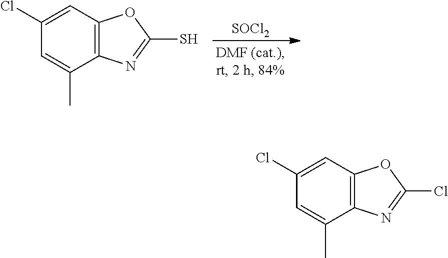

### 2,6-Dichloro-4-methylbenzo[d]oxazole

To a solution of 6-chloro-4-methylbenzo[d]oxazole-2-thiol (1.3 g, 6.5 mmol) in DCM (50 mL) were added DMF (0.5 mL) and SOCl₂ (12 mL) slowly at 0° C. The mixture was stirred at rt for 2 h. The TLC showed reaction to be complete. The solvent was evaporated under reduced pressure. The residue was diluted ice-water (20 mL) and extracted with EtOAc (3×25 mL). The organic layer was washed with brine (50 mL), dried (Na₂SO₄), filtered and concentrated under reduced pressure to afford 2,6-dichloro-4-methylbenzo[d]oxazole as a light brown solid. Yield: 1.1 g (85%); ¹H NMR (400 MHz, DMSO-d₆): δ 7.80 (s, 1H), 7.35 (s, 1H), 2.48 (s, 3H).

The following intermediates were prepared in a similar manner to 2,6-Dichloro-4-methylbenzo[d]oxazole.

| | | | | Spectral Data |
|---|---|---|---|---|
| Name | Int | Structure | Yield | 1H NMR & LCMS |
| Methyl 2- chlorobenzo[d] oxazole-5- carboxylate | 70 | 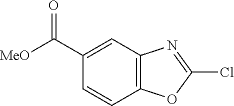 [SMILES: COC(=O)c1ccc2oc(Cl)nc2c1] | 60% | Crude data showed product. Proceeded further without purification. |
| 2-Chloro-5,6- difluorobenzo [d]oxazole | 71 | 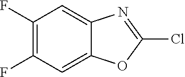 [SMILES: Fc1cc2nc(Cl)oc2cc1F] | 52% | Crude data showed product. Proceeded further without purification. |
| 2,6-diChloro- 5- fluorobenzo[d] oxazole | 72 | 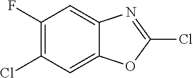 [SMILES: Fc1cc2nc(Cl)oc2cc1Cl] | 55% | Crude data showed product. Proceeded further without purification. |
| 2,5-dichloro-6- (trifluorometh- yl)benzo[d]oxa- ole | 73 | 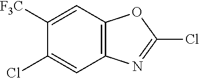 [SMILES: FC(F)(F)c1cc2oc(Cl)nc2cc1Cl] | 45% | Crude data showed product. Proceeded further without purification. |

### Intermediate 74

### N-Cyclopropyl-2-(methylamino)thiazole-4-carboxamide

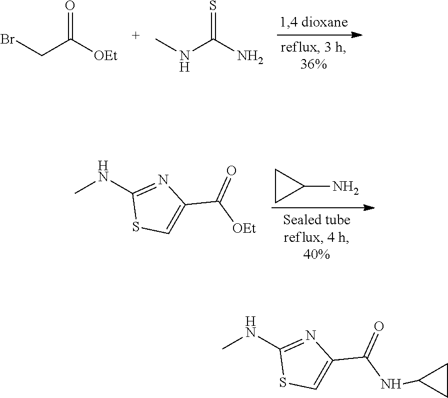

### Ethyl 2-(methylamino)thiazole-4-carboxylate

A mixture of ethyl 2-bromoacetate (6 g, 30.0 mmol) and 1-methylthiourea (2.92 g, 0.030) in 1,4 dioxane was stirred at 90° C. for 3 h. The TLC showed reaction to be complete. Solvent was removed under reduced pressure. The residue was diluted with H₂O (100 mL) and extracted with EtOAc (3×100 mL). The organic layer was washed with brine (50 mL), dried (Na₂SO₄), filtered and concentrated under reduced pressure. The residue was triturated with Et₂O (25 mL) to afford ethyl 2-(methylamino)thiazole-4-carboxylate as an off white solid. Yield: 2.3 g (40%); ¹H NMR (400 MHz, DMSO-d₆): δ 7.72 (bs, 1H), 7.51 (s, 1H), 4.22 (q, J=6.9 Hz, 2H), 2.82 (d, J=4.5 Hz, 3H), 1.26 (t, J=6.9 Hz, 3H); MS (ESI+) for CHNOS m/z 187.15 [M+H]⁺.

### N-Cyclopropyl-2-(methylamino)thiazole-4-carboxamide

A mixture of ethyl 2-(methylamino)thiazole-4-carboxylate (3 g, 10 mmmol) and cyclopropanamine (15 mL) in acetic acid (2 mL) taken in sealed tube. The reaction mixture was stirred at 120° C. for 4 h. The TLC showed reaction to be complete. Reaction mixture was allowed to cool to room temperature and quenched with ice-water (100 mL). The precipitated solid was filtered, washed with water (100 mL) followed by Et₂O (100 ml), dried under vacuum to afford N-cyclopropyl-2-(methylamino)thiazole-4-carboxamide as an off white solid. Yield: 600 mg (28%); ¹H NMR (400 MHz, DMSO-d₆): δ7.74 (q, J=4.3 Hz, 1H), 7.56 (bs, 1H), 7.19 (s, 1H), 2.84 (d, J=4.3 Hz, 3H), 2.74-2.77 (m, 1H), 0.64-0.68 (m, 2H), 0.48-0.58 (m, 2H); MS (ESI+) for CHNOS m/z 198.10 [M+H]⁺.

The following examples were prepared in a similar manner to N-cyclopropyl-2-((5-(trifluoromethyl)benzo[d]oxazol-2-yl)amino) thiazole-4-carboxamide following synthetic route 1.

| | | | | Spectral Data |
|---|---|---|---|---|
| Name | Ex | Structure | Yield | 1H NMR & LCMS |
| N-Methyl-2-((5- (trifluoromethyl) benzo[d]oxazol- 2- yl)amino)thiazole- 4- carboxamide | 2 | 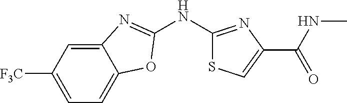 [SMILES: CNC(=O)c1csc(Nc2nc3cc(C(F)(F)F)ccc3o2)n1] | 28% | MS (ESI+) for CHNOS m/z 343.23 [M + H]⁺; LC purity 98.9% (Ret. Time- 5.73 min); ¹H NMR (400 MHz, DMSO-d₆): δ 13.10 (bs, 1H), 8.28 |
| | | | | (bs, 1H), 7.82 (s, 1H), |
| | | | | 7.70-7.76 (m, 2H), |
| | | | | 7.56 (d, J = 8.4 Hz, |
| | | | | 1H), 2.79 (d, J = 4.7 |
| | | | | Hz, 3H). |
| N-Ethyl-2-((5- (trifluoromethyl) benzo[d]oxazol- 2- yl)amino)thiazole- 4- carboxamide | 3 | 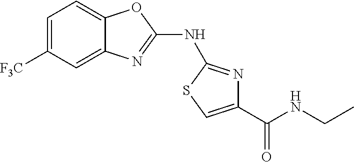 [SMILES: CCNC(=O)c1csc(Nc2nc3cc(C(F)(F)F)ccc3o2)n1] | 21% | MS (ESI+) for CHNOS m/z 357.12 [M + H]⁺; LC purity 98.7% (Ret. Time- 5.97 min); ¹H NMR (400 MHz, DMSO-d₆): δ 13.16 (bs, 1H), 8.25 (bs, 1H), 7.70-7.86 (m, 3H), 7.56 (d, J = 8.0 Hz, 1H), 3.28 (q, J = 7.2 Hz, 2H), 1.12 |
| | | | | (t, J = 7.2 Hz, 3H). |
| N-Isopropyl-2- ((5- (trifluoromethyl) benzo[d]oxazol- 2- yl)amino)thiazole- 4- carboxamide | 4 | 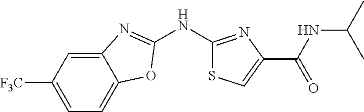 [SMILES: CC(C)NC(=O)c1csc(Nc2nc3cc(C(F)(F)F)ccc3o2)n1] | 17% | MS (ESI+) for CHNOS m/z 371.31 [M + H]⁺; LC purity 97.4% (Ret. Time- 6.17 min); ¹H NMR (400 MHz, DMSO-d₆): δ 13.18 (bs, 1H), 8.0 (bs, 1H), 7.85 (s, 1H), |
| | | | | 7.75 (s, 1H), 7.72 (d, J = |
| | | | | 8.2 Hz, 1H), 7.53 (d, |
| | | | | J = 8.4 Hz, 1H), 3.98- |
| | | | | 4.08 (m, 1H), 1.17 (d, |
| | | | | J = 6.4 Hz, 6H). |
| N-Phenyl-2- ((5- (trifluoromethyl) benzo[d]oxazol- 2- yl)amino)thiazole- 4- carboxamide | 5 | 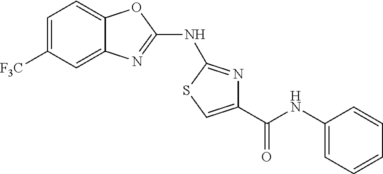 [SMILES: O=C(Nc1ccccc1)c1csc(Nc2nc3cc(C(F)(F)F)ccc3o2)n1] | 9% | MS (ESI+) for CHNOS m/z 405.92 [M + H]⁺; LC purity 96.8% (Ret. Time- 6.66 min); ¹H NMR (400 MHz, DMSO-d₆): δ 13.36 (bs, 1H), 9.99 (bs, 1H), 8.03 (s, 1H), 7.71-7.94 (m, 4H), 7.58 (d, J = 8.4 Hz, 1H), 7.34-7.40 (m, |
| | | | | 2H), 7.12-7.15 (m, |
| | | | | 1H). |
| N-(Thiazol-2- yl)-5- (trifluoromethyl) benzo[d]oxazol- 2-amine | 6 | 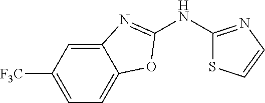 [SMILES: FC(F)(F)c1ccc2oc(Nc3nccs3)nc2c1] | 17% | MS (ESI+) for CHNOS m/z 286.23 [M + H]⁺; LC purity 99.9% (Ret. Time- 5.68 min); ¹H NMR (400 MHz, DMSO-d₆): δ 13.0 (bs, 1H), 7.77 |
| | | | | (s, 1H), 7.65 (d, J = |
| | | | | 8.4 Hz, 1H), 7.47 (d, |
| | | | | J = 8.4 Hz, 1H), 7.42 |
| | | | | (d, J = 4.4 Hz, 1H), |
| | | | | 7.04 (d, J = 4.4 Hz, |
| | | | | 1H). |
| N-(4- Methylthiazol- 2-yl)-5- (trifluoromethyl) benzo[d]oxazol- 2-amine | 7 | 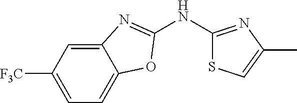 [SMILES: Cc1csc(Nc2nc3cc(C(F)(F)F)ccc3o2)n1] | 22% | MS (ESI+) for CHNOS m/z 300.28 [M + H]⁺; LC purity 99.1% (Ret. Time- 5.92 min); ¹H NMR (400 MHz, DMSO-d₆): δ 12.91 (bs, 1H), 7.74 |
| | | | | (s, 1H), 7.63 (d, J = |
| | | | | 8.4 Hz, 1H), 7.46 (d, |
| | | | | J = 8.4 Hz, 1H), 6.60 |
| | | | | (s, 1H), 2.20 (s, 3H). |
| Ethyl 2-((5- (trifluoromethyl) benzo[d]oxazol- 2- yl)amino)thiazole- 4- carboxylate | 8 | 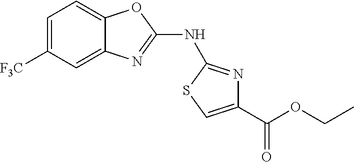 [SMILES: CCOC(=O)c1csc(Nc2nc3cc(C(F)(F)F)ccc3o2)n1] | 7% | MS (ESI+) for CHNOS m/z 358.27 [M + H]⁺; LC purity 98.3% (Ret. Time- 6.66 min); ¹H NMR (400 MHz, DMSO-d₆): δ 13.57 (bs, 1H), 8.07 (s, 1H), 7.89 (s, 1H), 7.78 (d, J = 8.0 Hz, 1H), 7.57 (d, J = 8.0 |
| | | | | Hz, 1H), 4.29 (q, J = |
| | | | | 6.8 Hz, 2H), 1.31 (t, J = |
| | | | | 6.8 Hz, 3H). |
| N-(3- Fluorophenyl)- 2-((5- (trifluoromethyl) benzo[d]oxazol- 2- yl)amino)thiazole- 4- carboxamide | 9 | 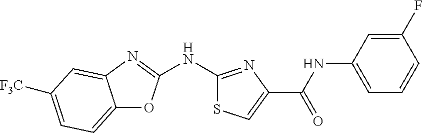 [SMILES: O=C(Nc1cccc(F)c1)c1csc(Nc2nc3cc(C(F)(F)F)ccc3o2)n1] | 7% | MS (ESI+) for CHNOS m/z 423.07 [M + H]⁺; LC purity 96.4% (Ret. Time- 6.77 min); ¹H NMR (400 MHz, DMSO-d₆): δ 13.22 (bs, 1H), 10.30 (bs, 1H), 8.04 (s, 1H), 7.84 (s, 1H), |
| | | | | 7.76 (d, J = 8.0 Hz, |
| | | | | 2H), 7.55-7.58 (m, |
| | | | | 2H), 7.38-7.44 (m, |
| | | | | 1H), 6.94-6.98 (m, |
| | | | | 1H). |
| N-(3- Chlorophenyl)- 2-((5- (trifluoromethyl) benzo[d]oxazol- 2- yl)amino)thiazole- 4- carboxamide | 10 | 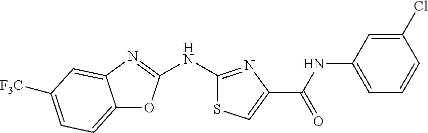 [SMILES: O=C(Nc1cccc(Cl)c1)c1csc(Nc2nc3cc(C(F)(F)F)ccc3o2)n1] | 12% | MS (ESI+) for CHNOS m/z 439.07 [M + H]⁺; LC purity 95.3% (Ret. Time- 6.99 min); ¹H NMR (400 MHz, DMSO-d₆): δ 10.30 (bs, 1H), 8.06 (s, 1H), 7.98 (s, 1H), 7.86 (s, 1H), 7.78 (d, |
| | | | | J = 8.4 Hz, 1H), 7.68 |
| | | | | (d, J = 8.4 Hz, 1H), |
| | | | | 7.58 (d, J = 8.4 Hz, |
| | | | | 1H), 7.38-7.42 (m, |
| | | | | 2H), 7.19 (d, J = 7.6 |
| | | | | Hz, 1H). |
| N-(Isoxazol-3- yl)-5- (trifluoromethyl) benzo[d]oxazol- 2-amine | 11 | 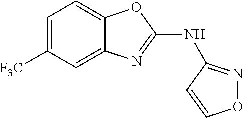 [SMILES: FC(F)(F)c1ccc2oc(Nc3ccon3)nc2c1] | 16% | MS (ESI+) for CHNOS m/z 270.06 [M + H]⁺; LC purity 99.5% (Ret. Time- 5.98 min); ¹H NMR (400 MHz, DMSO-d₆): δ 12.02 (bs, 1H), 8.89 (s, 1H), 7.83 (bs, 1H), |
| | | | | 7.76 (d, J = 8.4 Hz, |
| | | | | 1H), 7.54 (d, J = 8.4 |
| | | | | Hz, 1H), 7.08 (bs, |
| | | | | 1H). |
| N-(1-Methyl- 1H-1,2,3- triazol-4-yl)-5- (trifluoromethyl) benzo[d]oxazol- 2-amine | 12 | 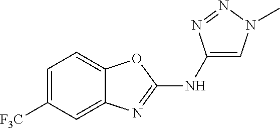 [SMILES: Cn1cc(Nc2nc3cc(C(F)(F)F)ccc3o2)nn1] | 21% | MS (ESI+) for CHNOS m/z 284.07 [M + H]⁺; LC purity 99.7% (Ret. Time- 5.17 min); ¹H NNMR (400 MHz, DMSO-d₆): δ 11.68 (bs, 1H), 8.23 (s, 1H), 7.68-7.75 (m, |
| | | | | 2H), 7.49 (d, J = 8.4 |
| | | | | Hz, 1H), 4.09 (s, 3H). |
| N-(4-(tert- butyl)thiazol-2- yl)-5- (trifluoromethyl) benzo[d]oxazol- 2-amine | 13 | 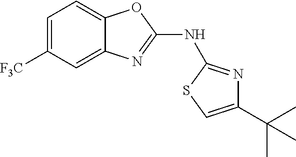 [SMILES: CC(C)(C)c1csc(Nc2nc3cc(C(F)(F)F)ccc3o2)n1] | 15% | MS (ESI+) for CHNOS m/z 342.13 [M + H]⁺; LC purity 98.8% (Ret. Time- 6.90 min); ¹H NMR (400 MHz, DMSO-d₆): δ 12.97 (bs, 1H), 7.76 (s, 1H), 7.64 (d, J = 8.0 Hz, 1H), 7.46 (d, J = 8.0 Hz, 1H), 6.57 |
| | | | | (s, 1H), 1.28 (s, 9H). |
| N-(1,3,4- Thiadiazol-2- yl)-5- (trifluoromethyl) benzo[d]oxazol- 2-amine | 14 | 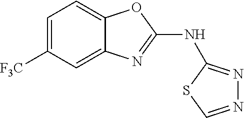 [SMILES: FC(F)(F)c1ccc2oc(Nc3nncs3)nc2c1] | 14% | MS (ESI+) for CHNOS m/z 287.06 [M + H]⁺; LC purity 99.6% (Ret. Time- 4.75 min); ¹H NMR (400 MHz, DMSO-d₆): δ 14.53 (bs, 1H), 8.94 (s, 1H), 7.85 (s, 1H), |
| | | | | 7.72 (d, J = 8.0 Hz, |
| | | | | 1H), 7.53 (d, J = 8.0 |
| | | | | Hz, 1H). |
| 2- (Benzo[d]oxazol- 2-ylamino)- N- cyclopropylthia- zole-4- carboxamide | 15 | 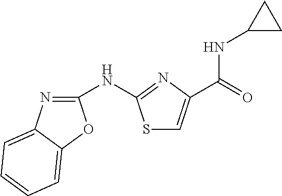 [SMILES: O=C(NC1CC1)c1csc(Nc2nc3ccccc3o2)n1] | 24% | MS (ESI+) for CHNOS m/z 301.28 [M + H]⁺; LC purity 99.9% (Ret. Time- 5.29 min); ¹H NMR (400 MHz, DMSO-d₆): δ 12.80 (bs, 1H), 8.24 (bs, 1H), 7.73 (s, 1H), 7.40-7.52 (m, 2H), 7.20-7.30 (m, 2H), 2.78-2.82 (m, 1H), 0.71-0.74 (m, 2H), 0.55-0.61 (m, 2H). |
| N-Cyclopropyl- 2-((5- methylbenzo[d] oxazol-2- yl)amino)thiazole- 4- carboxamide | 16 | 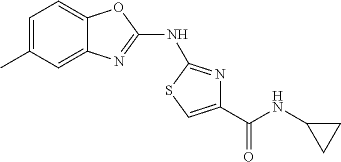 [SMILES: Cc1ccc2oc(Nc3nc(C(=O)NC4CC4)cs3)nc2c1] | 24% | MS (ESI+) for CHNOS m/z 315.30 [M + H]⁺; LC purity 97.0% (Ret. Time- 5.12 min); ¹H NMR (400 MHz, DMSO-d₆): δ 12.87 (bs, 1H), 8.60 (bs, 1H), 7.97 (s, 1H), 7.40 (d, J = 7.6 Hz, 1H), 7.29 (s, 1H), 7.01 |
| | | | | (d, J = 7.6 Hz, 1H), |
| | | | | 2.76-2.85 (m, 1H), |
| | | | | 0.71-0.74 (m, 2H), |
| | | | | 0.55-0.61 (m, 2H). |
| 2-((5- Chlorobenzo[d] oxazol-2- yl)amino)-N- cyclopropylthia- zole-4- carboxamide | 17 | 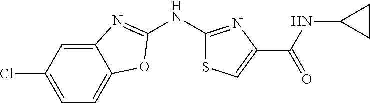 [SMILES: O=C(NC1CC1)c1csc(Nc2nc3cc(Cl)ccc3o2)n1] | 23% | MS (ESI+) for CHNOS m/z 335.27 [M + H]⁺; LC purity 99.8% (Ret. Time- 5.81 min); ¹H NMR (400 MHz, DMSO-d₆): δ 13.05 (bs, 1H), 8.30 |
| | | | | (bs, 1H), 7.73 (s, 1H), |
| | | | | 7.53-7.55 (m, 2H), |
| | | | | 7.22 (d, J = 8.8 Hz, |
| | | | | 1H), 2.77-2.84 (m, |
| | | | | 1H), 0.70-0.75 (m, |
| | | | | 2H), 0.55-0.61 (m, |
| | | | | 2H). |
| N-Cyclopropyl- 2-((5- fluorobenzo[d] oxazol-2- yl)amino)thiazole- 4- carboxamide | 18 | 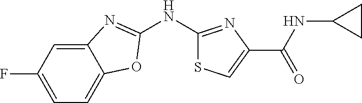 [SMILES: O=C(NC1CC1)c1csc(Nc2nc3cc(F)ccc3o2)n1] | 20% | MS (ESI+) for CHNOS m/z 319.28 [M + H]⁺; LC purity 95.7% (Ret. Time- 4.74 min); ¹H NMR (400 MHz, DMSO-d₆): δ 12.91 (bs, 1H), 8.21 |
| | | | | (bs, 1H), 7.70 (s, 1H), |
| | | | | 7.49-7.54 (m, 1H), |
| | | | | 7.32 (d, J = 7.6 Hz, |
| | | | | 1H), 6.95-7.02 (m, |
| | | | | 1H), 2.76-2.84 (m, |
| | | | | 1H), 0.68-0.75 (m, |
| | | | | 2H), 0.55-0.61 (m, |
| | | | | 2H). |
| N-Cyclopropyl- 2-((6- fluorobenzo[d] oxazol-2- yl)amino)thiazole- 4- carboxamide | 19 | 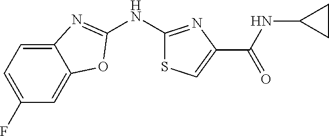 [SMILES: O=C(NC1CC1)c1csc(Nc2nc3ccc(F)cc3o2)n1] | 27% | MS (ESI+) for CHNOS m/z 319.29 [M + H]⁺; LC purity 98.9% (Ret. Time- 4.78 min); ¹H NMR (400 MHz, DMSO-d₆): δ 12.71 (bs, 1H), 8.23 (bs, 1H), 7.69 (s, 1H), 7.51-7.61 (m, 1H), |
| | | | | 7.43-7.48 (m, 1H), |
| | | | | 7.06-7.14 (m, 1H), |
| | | | | 2.76-2.85 (m, 1H), |
| | | | | 0.69-0.75 (m, 2H), |
| | | | | 0.55-0.61 (m, 2H). |
| 2-((6- Chlorobenzo[d] oxazol-2- yl)amino)-N- cyclopropylthia- zole-4- carboxamide | 20 | 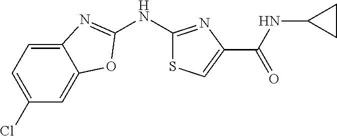 [SMILES: O=C(NC1CC1)c1csc(Nc2nc3ccc(Cl)cc3o2)n1] | 24% | MS (ESI+) for CHNOS m/z 335.25 [M + H]⁺; LC purity 96.8% (Ret. Time- 5.83 min); ¹H NMR (400 MHz, DMSO-d₆): δ 12.99 (bs, 1H), 8.29 (bs, 1H), 7.73 (bs, 2H), |
| | | | | 7.48 (d, J = 8.0 Hz, |
| | | | | 1H), 7.30 (d, J = 8.0 |
| | | | | Hz, 1H), 2.76-2.84 (m, |
| | | | | 1H), 0.68-0.74 (m, |
| | | | | 2H), 0.55-0.61 (m, |
| | | | | 2H). |
| 2-((5- (Trifluorometh- yl)benzo[d]oxa- zol-2- yl)amino)thia- zole-4- carboxamide | 21 | 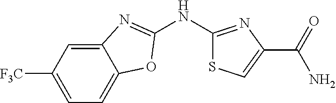 [SMILES: NC(=O)c1csc(Nc2nc3cc(C(F)(F)F)ccc3o2)n1] | 5% | MS (ESI+) for CHNOS m/z 329.35 [M + H]⁺; LC purity 92.4% (Ret. Time- 4.82 min); ¹H NMR (400 MHz, DMSO-d₆): δ 13.11 (bs, 1H), |
| | | | | 7.58-7.84 (m, 6H). |
| N-(5-Methyl- 1,3,4- oxadiazol-2-yl)- 5- (trifluoromethyl) benzo[d]oxa- zol-2-amine | 22 | 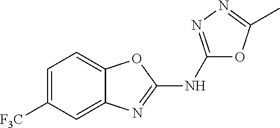 [SMILES: Cc1nnc(Nc2nc3cc(C(F)(F)F)ccc3o2)o1] | 6% | MS (ESI+) for CHNOS m/z 285.38 [M + H]⁺; LC purity 96.2% (Ret. Time- 4.43 min); ¹H NMR (400 MHz, DMSO-d₆): δ 7.67-7.73 (m, 2H), 7.55 (d, J = 8.3 Hz, 1H), 2.43 (s, 3H). |
| N-(1,2,4- Thiadiazol-5- yl)-5- (trifluoromethyl) benzo[d]oxa- zol-2-amine | 23 | 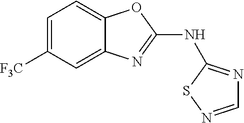 [SMILES: FC(F)(F)c1ccc2oc(Nc3ncns3)nc2c1] | 11% | MS (ESI+) for CHNOS m/z 287.21 [M + H]⁺; LC purity 96.8% (Ret. Time- 4.66 min); ¹H NMR (400 MHz, DMSO-d₆): δ 14.15 (bs, 1H), 8.53 (s, 1H), 7.94 (s, 1H), |
| | | | | 7.80 (d, J = 8.4 Hz, |
| | | | | 1H), 7.59 (d, J = 8.4 |
| | | | | Hz, 1H). |
| N-Cyclopropyl- 2-((5- (trifluoromethyl) benzo[d]oxa- zol-2- yl)amino)oxa- zole-4- carboxamide | 24 | 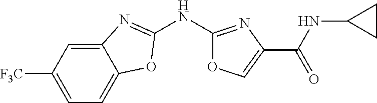 [SMILES: O=C(NC1CC1)c1coc(Nc2nc3cc(C(F)(F)F)ccc3o2)n1] | 8% | MS (ESI+) for CHNOS m/z 353.12 [M + H]⁺; LC purity 97.9% (Ret. Time- 5.89 min); ¹H NMR (400 MHz, DMSO-d₆): δ 12.55 (bs, 1H), 8.33 (bs, 1H), 8.23 (s, 1H), |
| | | | | 7.75 (d, J = 8.4 Hz, |
| | | | | 1H), 7.61-7.66 (m, |
| | | | | 2H), 2.76-2.83 (m, |
| | | | | 1H), 0.74-0.81 (m, |
| | | | | 2H), 0.49-0.65 (m, |
| | | | | 2H). |
| N-Cyclopropyl- 5-methyl-2-((5- (trifluoromethyl) benzo[d]oxazol- 2- yl)amino)thia- zole-4- carboxamide | 25 | 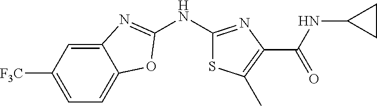 [SMILES: Cc1sc(Nc2nc3cc(C(F)(F)F)ccc3o2)nc1C(=O)NC1CC1] | 4% | MS (ESI+) for CHNOS m/z 383.37 [M + H]⁺; LC purity 96.6% (Ret. Time- 5.75 min); ¹H NMR (400 MHz, DMSO-d₆): δ 12.77 (bs, 1H), 7.98 (s, 1H), 7.76 (s, 1H), |
| | | | | 7.72 (d, J = 8.4 Hz, |
| | | | | 1H), 7.53 (d, J = 8.4 |
| | | | | Hz, 1H), 2.77-2.82 (m, |
| | | | | 1H), 2.61 (s, 3H), |
| | | | | 0.68-0.77 (m, 2H), |
| | | | | 0.55-0.66 (m, 2H). |
| N-Cyclopropyl- 2-((6- (trifluoromethyl) benzo[d]oxazol- 2- yl)amino)thia- zole-4- carboxamide | 26 | 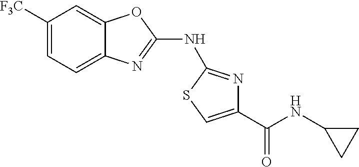 [SMILES: O=C(NC1CC1)c1csc(Nc2nc3ccc(C(F)(F)F)cc3o2)n1] | 38% | MS (ESI+) for CHNOS m/z 369.31 [M + H]⁺; LC purity 99.4% (Ret. Time- 5.14 min); ¹H NMR (400 MHz, DMSO-d₆): δ 13.16 (bs, 1H), 8.46 (bs, 1H), 7.97 (s, 1H), 7.75 (s, 1H), 7.58-7.63 (m, 2H), 2.79-2.82 (m, |
| | | | | 1H), 0.67-0.76 (m, |
| | | | | 2H), 0.57-0.60 (m, |
| | | | | 2H). |
| N-(5- Morpholinothia- zol-2-yl)-6- (trifluoromethyl) benzo[d]oxazol- 2-amine | 27 | 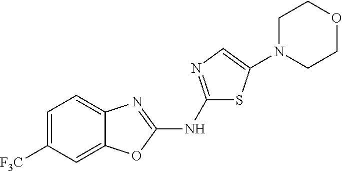 [SMILES: FC(F)(F)c1ccc2nc(Nc3ncc(N4CCOCC4)s3)oc2c1] | 13% | MS (ESI+) for CHNOS m/z 371.30 [M + H]⁺; LC purity 96.1% (Ret. Time- 5.88 min); ¹H NMR (400 MHz, DMSO-d₆): δ 12.55 (bs, 1H), 7.80 (s, 1H), 7.48-7.54 (m, 2H), 6.66 (s, 1H), 3.72 (t, J = 4.3 Hz, 4H), 3.00 (t, J = 4.3 Hz, |
| | | | | 4H). |
| N-(5-(Piperidin- 1-yl)thiazol-2- yl)-6- (trifluoromethyl) benzo[d]oxa- zol-2-amine | 28 | 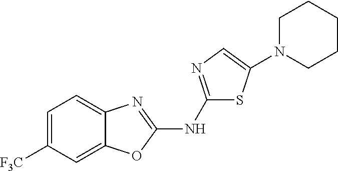 [SMILES: FC(F)(F)c1ccc2nc(Nc3ncc(N4CCCCC4)s3)oc2c1] | 10% | MS (ESI+) for CHNOS m/z 369.40 [M + H]⁺; LC purity 96.1% (Ret. Time- 6.62 min); ¹H NMR (400 MHz, DMSO-d₆): δ 7.74 (bs, 1H), 7.47 (bs, 2H), 6.55 (bs, 1H), 2.95-3.01 (m, 4H), 1.60-1.63 (m, 4H), |
| | | | | 1.48-1.52 (m, 2H). |
| tert-Butyl 4-(2- ((6- (trifluoromethyl) benzo[d]oxazol- 2- yl)amino)thia- zol-5- yl)piperazine- 1-carboxylate | 29 | 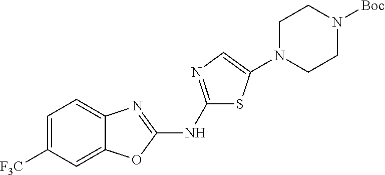 [SMILES: CC(C)(C)OC(=O)N1CCN(c2cnc(Nc3nc4ccc(C(F)(F)F)cc4o3)s2)CC1] | 15% | MS (ESI+) for CHNOS m/z 470.34 [M + H]⁺; LC purity 98.7% (Ret. Time- 5.78 min); ¹H NMR (400 MHz, DMSO-d₆): δ 12.54 (bs, 1H), 7.81 (s, 1H), 7.50-7.55 (m, 2H), 6.68 (s, 1H), 3.46 (bs, 4H), 2.98 (bs, 4H), 1.42 (s, 9H). |
| N-Cyclopropyl- 5-((6- (trifluoromethyl) benzo[d]oxazol- 2-yl)amino)- 1,3,4- oxadiazol-2- carboxamide | 30 | 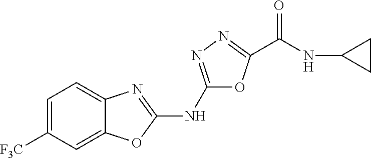 [SMILES: O=C(NC1CC1)c1nnc(Nc2nc3ccc(C(F)(F)F)cc3o2)o1] | 41% | MS (ESI+) for CHNOS m/z 354.28 [M + H]⁺; LC purity 98.6% (Ret. Time- 4.43 min); ¹H NMR (400 MHz, DMSO-d₆): δ 8.80 (bs, 1H), 7.52 (s, 1H), 7.35 (d, J = 8.0 Hz, 1H), 7.29 (d, J = 8.0 Hz, 1H), 2.78- 2.84 (m, 1H), 0.62- |
| | | | | 0.70 (m, 4H). |
| N-(3-Methyl- 1,2,4- oxadiazol-5-yl)- 5- (trifluoromethyl) benzo[d]oxa- zol-2-amine | 31 | 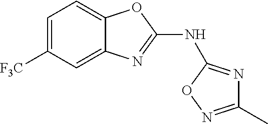 [SMILES: Cc1noc(Nc2nc3cc(C(F)(F)F)ccc3o2)n1] | 4% | MS (ESI+) for CHNOS m/z 285.25 [M + H]⁺; LC purity 98.9% (Ret. Time- 4.20 min); ¹H NMR (400 MHz, DMSO-d₆): δ 7.73-7.80 (m, 2H), 7.64 (d, J = 8.2 Hz, |
| | | | | 1H), 2.32 (s, 3H). |
| N-(5-(4- Methylpiperazin- 1-yl)thiazol- 2-yl)-5- (trifluoromethyl) benzo[d]oxa- zol-2-amine | 32 | 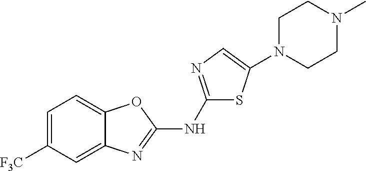 [SMILES: CN1CCN(c2cnc(Nc3nc4cc(C(F)(F)F)ccc4o3)s2)CC1] | 2% | MS (ESI+) for CHNOS m/z 383.96 [M + H]⁺; LC purity 9.62% (Ret. Time- 4.88 min); ¹H NMR (400 MHz, DMSO-d₆): δ 12.43 (bs, 1H), 7.69 (s, 1H), 7.60 (bs, 1H), 7.34 (bs, 1H), 6.61 (s, 1H), 3.02 (bs, 4H), 2.49 (bs, 4H), 2.24 (s, |
| | | | | 3H). |
| 2-((6- Chlorobenzo[d] oxazol-2- yl)amino)thia- zole-4- carboxamide | 33 | 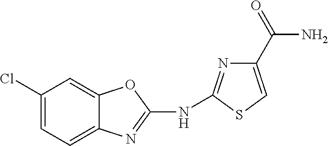 [SMILES: NC(=O)c1csc(Nc2nc3ccc(Cl)cc3o2)n1] | 3% | MS (ESI+) for CHNOS m/z 295.01 [M + H]⁺; LC purity 96.3% (Ret. Time- 4.75 min); ¹H NMR (400 MHz, DMSO-d₆): δ 12.91 (bs, 1H), 8.10 (bs, 1H), 7.63-7.77 (m, 3H). |
| 2-((6-Chloro-5- (trifluoromethyl) benzo[d]oxa- zol-2- yl)amino)thia- zole-4- carboxamide | 34 | 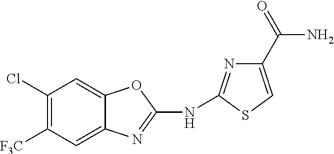 [SMILES: NC(=O)c1csc(Nc2nc3cc(C(F)(F)F)c(Cl)cc3o2)n1] | 12% | MS (ESI+) for CHNOS m/z 263.04 [M + H]⁺; LC purity 99.4% (Ret. Time- 5.87 min); ¹H NMR (400 MHz, DMSO-d₆): δ 8.0 (s, 1H), 7.90 (bs, 2H), 7.78 (s, 1H), 7.69 (s, 1H). |
| N-(5-(4- Methylpiperazin- 1-yl)thiazol- 2-yl)-6- (trifluoromethyl) benzo[d]oxazol- 2-amine | 35 | 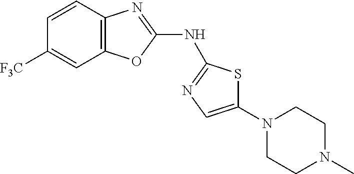 [SMILES: CN1CCN(c2cnc(Nc3nc4ccc(C(F)(F)F)cc4o3)s2)CC1] | 15% | MS (ESI+) for CHNOS m/z 384.08 [M + H]⁺; LC purity 98.8% (Ret. Time- 4.86 min); ¹H NMR (400 MHz, DMSO-d₆): δ 12.38 (bs, 1H), 7.80 (s, 1H), 7.51 (s, 2H), 6.60 (s, 1H), 3.02 (bs, 4H), 2.46 (bs, 4H), 2.22 (bs, 3H). |
| 2-((7- Chlorobenzo[d] oxazol-2- yl)amino)thia- zole-4- carboxamide | 36 | 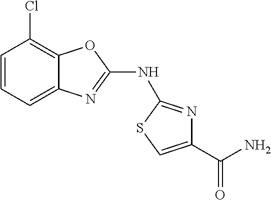 [SMILES: NC(=O)c1csc(Nc2nc3cccc(Cl)c3o2)n1] | 2% | MS (ESI+) for CHNOS m/z 294.98 [M + H]⁺; LC purity 99.4% (Ret. Time- 5.31 min); ¹H NMR (400 MHz, DMSO-d₆): δ 12.94 (bs, 1H), 8.01 (bs, 1H), 7.76 (s, 1H), 7.66 (s, 1H), 7.45 (s, 1H), 7.25 (bs, 2H). |
| N-Cyclopropyl- 2-((5- (trifluoromethyl) benzo[d]oxazol- 2- yl)amino)thia- zole-5- carboxamide | 37 | 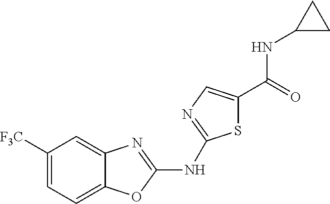 [SMILES: O=C(NC1CC1)c1cnc(Nc2nc3cc(C(F)(F)F)ccc3o2)s1] | 15% | MS (ESI+) for CHNOS m/z 369.33 [M + H]⁺; LC purity 97.1% (Ret. Time- 4.99 min); ¹H NMR (400 MHz, DMSO-d₆): δ 13.22 (bs, 1H), 8.49, (bs, 1H), 8.01 (s, 1H), 7.90 (s, 1H), 7.70 (d, J = 8.2 Hz, 1H), 7.52 (d, J = 8.2 Hz, 1H), 2.76- 2.79 (m, 1H), 0.69- |
| | | | | 0.73 (m, 2H), 0.53- |
| | | | | 0.60 (m, 2H). |
| N-(1-Methyl- 1H-pyrazol-3- yl)-5- (trifluoromethyl) benzo[d]oxa- zol-2-amine | 38 | 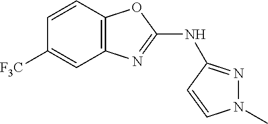 [SMILES: Cn1ccc(Nc2nc3cc(C(F)(F)F)ccc3o2)n1] | 14% | MS (ESI+) for CHNOS m/z 283.20 [M + H]⁺; LC purity 96.3% (Ret. Time- 5.85 min); ¹H NMR (400 MHz, DMSO-d₆): δ 11.20 (bs, 1H), 7.71 (s, 1H), 7.63-7.70 (m, 2H), |
| | | | | 7.45 (d, J = 8.2 Hz, |
| | | | | 1H), 6.56 (d, J = 1.8 |
| | | | | Hz, 1H), 3.78 (s, 3H). |
| 7-Chloro-N-(5- methyl-1,3,4- oxadiazol-2- yl)benzo[d]oxa- zol-2-amine | 39 | 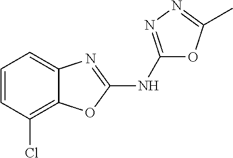 [SMILES: Cc1nnc(Nc2nc3cccc(Cl)c3o2)o1] | 3% | MS (ESI+) for CHNOS m/z 251.12 [M + H]⁺; LC purity 97.1% (Ret. Time- 4.26 min); ¹H NMR (400 MHz, DMSO-d₆): δ 7.08 (bs, 1H), 7.91- 7.98 (m, 1H), 6.83 (d, J = 7.6 Hz, 1H), 2.28 (s, 3H) |
| 4,6-Dichloro-N- (5-methyl- 1,3,4- oxadiazol-2- yl)benzo[d]oxa- zol-2-amine | 40 | 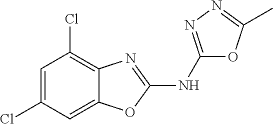 [SMILES: Cc1nnc(Nc2nc3c(Cl)cc(Cl)cc3o2)o1] | 24% | MS (ESI+) for CHNOS m/z 285.11 [M + H]⁺; LC purity 99.9% (Ret. Time- 4.39 min); ¹H NMR (400 MHz, DMSO-d₆): δ 7.66 (d, J = 1.3 Hz, 1H), 7.43 (d, J = 1.3 Hz, 1H), 2.43 (s, 3H). |
| N-(4-methyl- 4H-1,2,4- triazol-3-yl)-5- (trifluoromethyl) benzo[d]oxa- zol-2-amine | 41 | 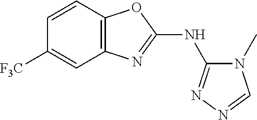 [SMILES: Cn1cnnc1Nc1nc2cc(C(F)(F)F)ccc2o1] | 3% | MS (ESI+) for CHNOS m/z 284.19 [M + H]⁺; LC purity 99.4% (Ret. Time- 5.38 min); ¹H NMR (400 MHz, DMSO-d₆): δ 8.06 (s, 1H), 7.59-7.63 (m, |
| | | | | 2H), 7.50 (d, J = 8.3 |
| | | | | Hz, 1H), 3.68 (s, 3H). |
| 6-Chloro-N-(5- methylisoxazol- 3-yl)-5- (trifluoromethyl) benzo[d]oxa- zol-2-amine | 42 | 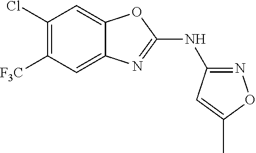 [SMILES: Cc1cc(Nc2nc3cc(C(F)(F)F)c(Cl)cc3o2)no1] | 49% | MS (ESI+) for CHNOS m/z 318.31 [M + H]⁺; LC purity 99.3% (Ret. Time- 6.55); ¹H NMR (400 MHz, DMSO-d₆): δ 12.01 (bs, 1H), 8.03 (s, 1H), 7.87 (s, 1H), 6.75 (s, 1H), 2.42 (s, 3H). |
| 6-Chloro-N- (4H-1,2,4- triazol-3-yl)-5- (trifluoromethyl) benzo[d]oxa- zol-2-amine | 43 | 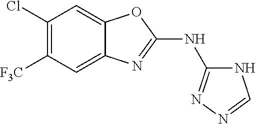 [SMILES: FC(F)(F)c1cc2nc(Nc3nnc[nH]3)oc2cc1Cl] | 28% | MS (ESI+) for CHNOS m/z 304.30 [M + H]⁺; LC purity 99.4% (Ret. Time- 5.02); ¹H NMR (400 MHz, DMSO-d_{6 +} d- TFA): δ 8.72 (s, 1H), 7.89 (s, 1H), 7.74 (s, |
| | | | | 1H). |
| Methyl 2-((5- methyl-1,3,4- oxadiazol-2- yl)amino)benzo [d]oxazole-5- carboxylate | 44 | 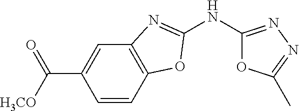 [SMILES: COC(=O)c1ccc2oc(Nc3nnc(C)o3)nc2c1] | 12% | MS (ESI+) for CHNOS m/z 277.21 [M + H]⁺; LC purity 97.7% (Ret. Time- 3.81 min); ¹H NMR (400 MHz, DMSO-d₆): δ 7.96 (d, J = 1.6 Hz, 1H), 7.85 (dd, J = 1.6, |
| | | | | 8.4 Hz, 1H), 7.61 (d, |
| | | | | J = 8.4 Hz, 1H), 3.87 |
| | | | | (s, 3H), 2.43 (s, 3H). |
| 4,6-Dichloro-N- (1,3,4- oxadiazol-2- yl)benzo[d]oxa- zol-2-amine | 45 | 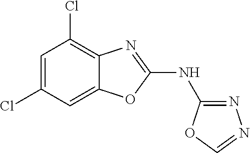 [SMILES: Clc1cc(Cl)c2nc(Nc3nnco3)oc2c1] | 12% | MS (ESI+) for CHNOS m/z 270.96 [M + H]⁺; LC purity 99.7% (Ret. Time- 3.03 min); ¹H NMR (400 MHz, DMSO-d₆ + D₂O): δ 8.51 (s, 1H), 7.42 (s, 1H), 7.25 (s, 1H). |
| 6-Chloro-N- (isoxazol-3- yl)benzo[d]oxa- zol-2-amine | 46 | 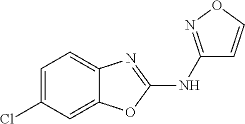 [SMILES: Clc1ccc2nc(Nc3ccon3)oc2c1] | 14% | MS (ESI+) for CHNOS m/z 235.99 [M + H]⁺; LC purity 99.3% (Ret. Time- 5.78 min); ¹H NMR (400 MHz, DMSO-d₆): δ 11.85 (bs, 1H), 8.86 (d, J = 1.4 Hz, 1H), 7.74 (d, J = 1.4, Hz, |
| | | | | 1H), 7.46 (d, J = 8.4 |
| | | | | Hz, 1H), 7.30 (dd, J = |
| | | | | 1.4, 8.4 Hz, 1H), 7.06 |
| | | | | (s, 1H). |
| 6-Chloro-5- fluoro-N-(1,3,4- oxadiazol-2- yl)benzo[d]oxa- zol-2-amine | 47 | 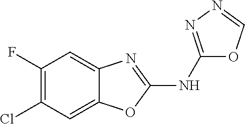 [SMILES: Fc1cc2nc(Nc3nnco3)oc2cc1Cl] | 2% | MS (ESI+) for CHNOS m/z 254.99 [M + H]⁺; LC purity 98.4% (Ret. Time- 3.97 min); ¹H NMR (400 MHz, DMSO-d₆): δ 12.89 (bs, 1H), 8.85 (s, 1H), 7.90 (s, 1H), |
| | | | | 7.41 (s, 1H). |
| 5,6-diFluoro-N- (1,3,4- oxadiazol-2- yl)benzo[d]oxa- zol-2-amine | 48 | 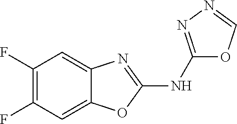 [SMILES: Fc1cc2nc(Nc3nnco3)oc2cc1F] | 4% | MS (ESI+) for CHNOS m/z 239.05 [M + H]⁺; LC purity 99.6% (Ret. Time- 4.59 min); ¹H NMR (400 MHz, DMSO-d₆): δ 8.84 (s, 1H), 7.83- 7.89 (m, 1H), 7.42- |
| | | | | 7.50 (m, 1H). |

### Synthetic Route 2

### N-(5-(Piperazin-1-yl)thiazol-2-yl)-6-(trifluoromethyl)benzo[d]oxazol-2-amine hydrochloride (Example 49)

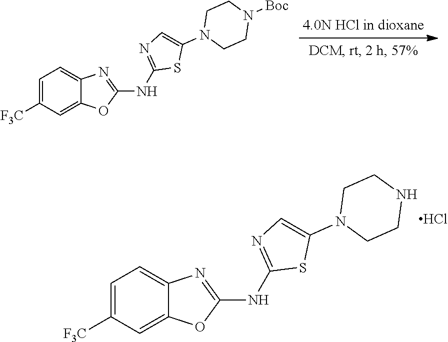 [SMILES: CC(C)(C)OC(=O)N1CCN(c2cnc(Nc3nc4ccc(C(F)(F)F)cc4o3)s2)CC1 ; FC(F)(F)c1ccc2nc(Nc3ncc(N4CCNCC4)s3)oc2c1 ; Cl]

To a solution of tert-butyl 4-(2-((6-(trifluoromethyl)benzo[d]oxazol-2-yl)amino)thiazol-5-yl)piperazine-1-carboxylate (200 mg, 0.40 mmol) in CH₂Cl₂ (4 mL) was added 4 N HCl in 1,4-dioxane (4 mL) and stirred at rt for 2 h. The TLC showed reaction to be complete. The reaction mixture was concentrated under reduced pressure. The residue was triturated with Et₂O (10 mL), filtered and dried under vacuum to afford N-(5-(piperazin-1-yl)thiazol-2-yl)-6-(trifluoromethyl)benzo[d]oxazol-2-amine hydrochloride an off white solid. Yield: 90 mg (57%); ¹H NMR (400 MHz, DMSO-d₆): δ 9.18 (bs, 2H), 7.84 (s, 1H), 7.50-7.57 (m, 2H), 6.81 (s, 1H), 3.25 (bs, 8H); MS (ESI+) for CHNOS m/z 370.38 [M+H]⁺.

### Intermediate 75

### 5-Amino-2-(trifluoromethyl)pyridin-4-ol

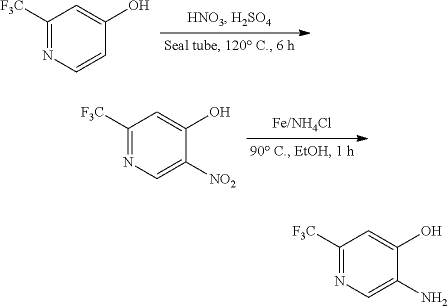

### 5-Nitro-2-(trifluoromethyl)pyridin-4-ol

To a cooled solution of 2-(trifluoromethyl)pyridin-4-ol (1.95 g, 11.9 mmol) in concentrated H₂SO₄ (4.8 mL) in sealed tube was added fuming HNO₃ (12 mL) dropwise. The reaction mixture was stirred at 120° C. for 6 h. The TLC showed reaction to be complete. Reaction was cooled to room temperature, quenched with ice-cold water and extracted with EtOAc (3×100 mL). The organics were dried (Na₂SO₄), filtered and concentrated under reduced pressure to afford 5-nitro-2-(trifluoromethyl)pyridin-4-ol as brown solid. Yield: 2.2 g (crude); MS (ESI+) for CHNOS m/z 209.20 [M+H]⁺.

### 5-Amino-2-(trifluoromethyl)pyridin-4-ol

To a solution of 5-nitro-2-(trifluoromethyl)pyridin-4-ol (2.2 g, 10.5 mmol) were added ammonium chloride (2.9 g, 52.8 mmol), Fe powder (2.9 g, 52.8 mmol) and water (3 mL). The reaction mixture was stirred at 90° C. for 1 h. The TLC showed reaction to be complete. Reaction mixture was cooled to room temperature and filtered through a celite bed. The filtrate was concentrated, diluted with water (25 mL) and extracted with EtOAc (3×50 mL). The organics were dried (Na₂SO₄), filtered and concentrated under reduced pressure to afford 5-amino-2-(trifluoromethyl)pyridin-4-ol as a brown liquid. Yield: 890 mg (crude); MS (ESI+) for CHNOS m/z 179.01 [M+H]⁺.

### Intermediate 76

### 6-(Trifluoromethyl)pyridine-3,4-diamine

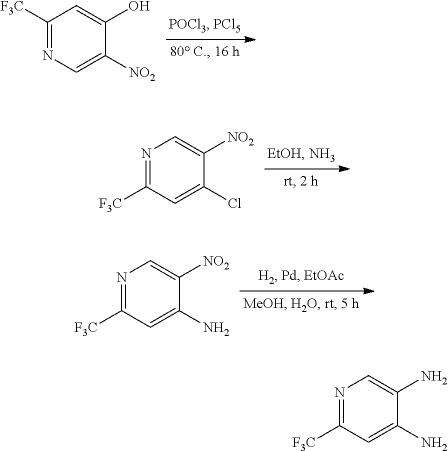 [SMILES: O=[N+]([O-])c1cnc(C(F)(F)F)cc1O ; O=[N+]([O-])c1cnc(C(F)(F)F)cc1Cl ; Nc1cc(C(F)(F)F)ncc1[N+](=O)[O-] ; Nc1cnc(C(F)(F)F)cc1N]

### 4-Chloro-5-nitro-2-(trifluoromethyl)pyridine

To a stirred solution of 5-nitro-2-(trifluoromethyl)pyridin-4-ol (3.9 g, 0.014 mol), PCl₅ (4.5 g, 0.021 mol) and POCl₃ (2 mL, 0.02 mol) was heated to 80° C. for 16 h. The TLC showed reaction to be complete. The reaction mixture was cooled to room temperature, diluted with DCM and washed with water (100 mL), saturated NaHCO₃ solution (100 mL) and brine (100 mL). The organics were dried (Na₂SO₄), filtered and concentrated under reduced pressure to afford 4-chloro-5-nitro-2-(trifluoromethyl)pyridine as yellow oil. Yield: 3 g (94%); ¹H NMR (400 MHz; DMSO-d₆): δ 9.42 (s, 1H), 8.56 (s, 1H); MS (ESI+) for CHNOS m/z 227.34 [M+H]⁺.

### 5-Nitro-2-(trifluoromethyl)pyridin-4-amine

To a stirred solution of 4-chloro-5-nitro-2-(trifluoromethyl)pyridine (1 g, 4.42 mmol) in ethanol (7 mL) in sealed tube, NH₃ gas was purged at −78° C. for 15 min. The reaction mixture was stirred at room temperature for 2 h. The TLC showed reaction to be complete. The reaction mixture was evaporated under reduced pressure to afford 5-nitro-2-(trifluoromethyl)pyridin-4-amine as yellow solid. Yield: 1 g (crude); ¹H NMR (400 MHz; DMSO-d₆): δ 9.02 (s, 1H), 7.39 (s, 1H); MS (ESI+) for CHNOS m/z 208.20 [M+H]⁺.

### 6-(Trifluoromethyl)pyridine-3,4-diamine

To a stirred solution of 5-nitro-2-(trifluoromethyl)pyridin-4-amine (1 g, 4.83 mmol) in MeOH/EtOAc (1.5:1), Pd—C was added then the reaction mixture was stirred at room temperature for 5 h at hydrogen atmosphere. The TLC showed reaction to be complete. The reaction mixture was cooled to room temperature and filtered through a celite bed and washed with methanol (50 mL). The methanol layer was evaporated under vacuum to afford 6-(trifluoromethyl)pyridine-3,4-diamine as a red liquid. Yield: 700 mg (81%); ¹H NMR (400 MHz; DMSO-d₆): δ 7.69 (s, 1H), 6.82 (s, 1H), 5.73 (bs, 2H), 5.08 (bs, 2H); MS (ESI+) for CHNOS m/z 178.03 [M+H]⁺.

### Intermediate 77

### Ethyl methyl-L-prolinate

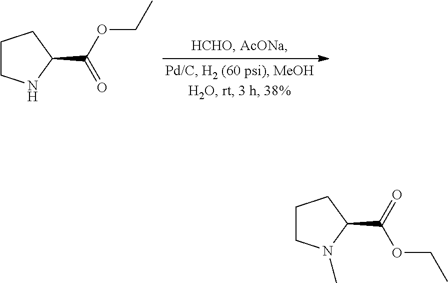

To a solution of ethyl L-prolinate (5 g, 3.49 mmol) in EtOH (30 mL) were added AcONa (2.8 g, 3.49 mmol), formaldehyde (37% in H₂O, 10 mL), Pd—C (1 g) at rt in the Parr reactor. The reaction mixture was stirred under H₂ atmosphere (60 psi) at rt for 3 h. The TLC showed reaction to be complete. The reaction mixture was filtered through celite bed and washed with EtOH (100 mL). The filtrate was concentrated under reduced pressure. The residue was acidified with 1N HCl (100 mL) and extracted with Et₂O (200 mL). The aqueous layer was basified to pH 12 with K₂CO₃ and extracted with CH₂Cl₂ (3×50 mL). The organics were dried (Na₂SO₄), filtered and concentrated under reduced pressure to afford ethyl methyl-L-prolinate as colourless oil. Yield: 2.1 g (38%); ¹H NMR (400 MHz; DMSO-d₆): δ 4.08 (q, J=7.1 Hz, 2H), 3.61 (bs, 1H), 2.80-2.91 (m 2H), 2.22 (s, 3H), 1.65-2.15 (m, 4H), 1.30 (t, J=7.1 Hz, 3H); MS (ESI+) for CHNOS m/z 144.20 [M+H]⁺.

The following intermediate was prepared in a similar manner to ethyl methyl-L-prolinate.

| | | | | Spectral Data |
|---|---|---|---|---|
| Name | Int | Structure | Yield | 1H NMR & LCMS |
| Ethyl methyl-D- prolinate | 78 | 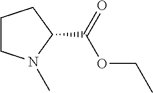 | 37% | MS (ESI+) for CHNOS m/z 126.21 [M + H]⁺; ¹H NMR (400 MHz, DMSO-d₆): δ 6.80 (bs, 2H), 1.90-2.05 (m, 1H), 0.90- 1.08 (m, 2H), 0.75-0.90 (m, 2H). |

### Intermediate 79

### 1-Methyl-1H-pyrazole-5-carbohydrazide

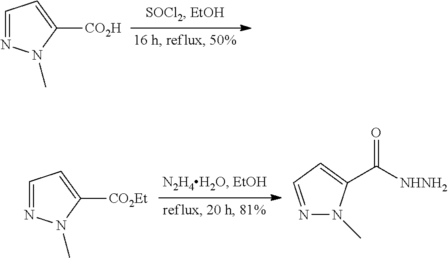

### Ethyl 1-methyl-1H-pyrazole-5-carboxylate

To a stirred solution of 1-methyl-1H-pyrazole-5-carboxylic acid (5 g, 37.0 mmol) in EtOH (30 mL) was added SOCl₂ (4.35 mL, 58.0 mmol). The reaction mixture was stirred at 80° C. for 18 h. The TLC showed reaction to be complete. The reaction mixture was concentrated under reduced pressure. The residue was basified by aq NaHCO₃ (100 mL) and extracted with EtOAC (3×50 mL). The organics were dried (Na₂SO₄), filtered and concentrated under reduced pressure to afford ethyl 1-methyl-1H-pyrazole-5-carboxylate as pale yellow oil. Yield: 3.0 g (50%); ¹H NMR (400 MHz, DMSO-d₆): δ 7.53 (d, J=1.9 Hz, 1H), 6.86 (d, J=1.9 Hz, 1H), 4.30 (q, J=7.1 Hz, 2H), 4.08 (s, 3H) 1.30 (t, J=7.1 Hz, 3H); MS (ESI+) for CHNOS m/z 155.22[M+H]⁺.

### 1-Methyl-1H-pyrazole-5-carbohydrazide

To a solution of ethyl 1-methyl-1H-pyrazole-5-carboxylate (3 g, 19.4 mmol) in EtOH (20 mL) was added hydrazine hydrate (10 mL, 194 mmol) at rt. The reaction mixture was stirred at 90° C. for 14 h. The TLC showed reaction to be complete. The reaction mixture was concentrated under reduced pressure. The residue was triturated with Et₂O (50 mL), dried under vacuum to afford 1-Methyl-1H-pyrazole-5-carbohydrazide as off white solid. Yield: 3 g (50%); ¹H NMR (400 MHz, DMSO-d₆): δ 9.74 (bs, 1H), 7.43 (d, J=1.7 Hz, 1H), 6.78 (d, J=1.9 Hz, 1H), 4.50 (bs, 2H), 4.04 (s, 3H); MS (ESI+) for CHNOS m/z 141.16 [M+H]⁺.

The following intermediate was prepared in a similar manner to 1-methyl-1H-pyrazole-5-carbohydrazide.

| | | | | Spectral Data |
|---|---|---|---|---|
| Name | Int | Structure | Yield | 1H NMR & LCMS |
| 3- Methylisonicotino hydrazide | 80 | 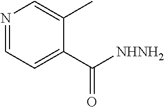 | 54% | MS (ESI+) for CHNOS m/z 151.99 [M + H]⁺; ¹H NMR (400 MHz, DMSO-d₆): δ 9.63 (bs, 1H), , 8.45 (s, 1H), 8.44 (d, J = 4.5 Hz, 1H), 7.24 (d, J = 8.2 Hz, 1H), 4.53 (bs, 2H), 2.50 (s, 3H). |
| (S)-1- Methylpyrrolidine- 2- carbohydrazide | 81 | 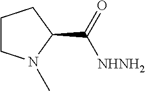 | 60% | MS (ESI+) for CHNOS m/z 144.22 [M + H]⁺. Crude data showed product. Proceeded further without purification. |
| (R)-1- Methylpyrrolidine- 2- carbohydrazide | 82 | 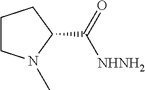 | 55% | MS (ESI+) for CHNOS m/z 144.22 [M + H]⁺. Crude data showed product. Proceeded further without purification. |
| tert-Butyl (S)-2- (hydrazinecarbonyl) pyrrolidine-1- carboxylate | 83 | 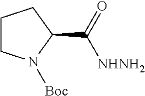 [SMILES: CC(C)(C)OC(=O)N1CCC[C@H]1C(=O)NN] | 55% | MS (ESI+) for CHNOS m/z 230.31 [M + H]⁺. Crude data showed product. Proceeded further without purification. |
| tert-Butyl (R)-2- (hydrazinecarbonyl) pyrrolidine-1- carboxylate | 84 | 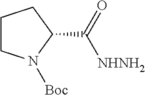 [SMILES: CC(C)(C)OC(=O)N1CCC[C@@H]1C(=O)NN] | 50% | MS (ESI+) for CHNOS m/z 230.31 [M + H]⁺. Crude data showed product. Proceeded further without purification. |

### Intermediate 85

### 5-Cyclopropyl-1,3,4-oxadiazol-2-amine

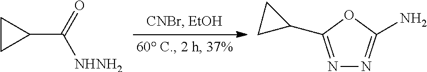 [SMILES: NNC(=O)C1CC1 ; Nc1nnc(C2CC2)o1]

To a solution of cyclopropanecarbohydrazide (2.0 g, 19.9 mmol) in EtOH (75 mL) was added cynaogen bromide (4.2 g, 39.6 mmol) at rt. The reaction mixture was heated at 60° C. for 2 h. The TLC showed reaction to be complete. The reaction mixture was poured in sat NaHCO₃ solution (100 mL) and extracted with EtOAc (3×100 mL). The organics layer was washed with brine (100 mL), dried (Na₂SO₄), filtered and concentrated under reduced pressure. The residue was triturated with DCM (50 mL) followed by Et2O (25 mL), dried under vacuum to afford 5-cyclopropyl-1,3,4-oxadiazol-2-amine as an off white solid Yield: 926 mg (37%); MS (ESI+) for CHNOS m/z 126.21 [M+H]; ¹H NMR (400 MHz, DMSO-d₆): δ6.80 (bs, 2H), 1.90-2.05 (m, 1H), 0.90-1.08 (m, 2H), 0.75-0.90 (m, 2H)

The following intermediates were prepared in a similar manner to 5-cyclopropyl-1,3,4-oxadiazol-2-amine.

| | | | | Spectral Data |
|---|---|---|---|---|
| Name | Int | Structure | Yield | 1H NMR & LCMS |
| 5-Methyl-1,3,4- oxadiazol-2- amine | 86 | 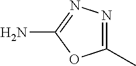 | 20% | MS (ESI+) for CHNOS m/z 100.11 [M + H]⁺; ¹H NMR (400 MHz, DMSO-d₆): δ 6.80 (s, 2H), 2.32 (s, 3H). |
| 5- Chlorobenzo[d] oxazol-2-amine | 87 | 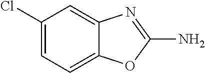 [SMILES: Nc1nc2cc(Cl)ccc2o1] | 78% | MS (ESI+) for CHNOS m/z 167.18 [M − H]⁺; ¹H NMR (400 MHz, DMSO-d₆): δ 7.55 (s, 2H), 7.49 (s, 1H), 7.10-7.19 (m, 2H). |
| 5-Isopropyl- 1,3,4-oxadiazol- 2-amine | 88 | 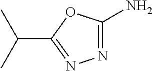 | 20% | ¹H NMR (400 MHz, DMSO- d₆): δ 6.83 (s, 2H), 2.91-3.01 (m, 1H), 1.21 (d, J = 6.9 Hz, 6H). |
| Oxazolo[4,5-c] pyridin-2- amine | 89 | 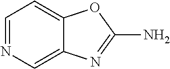 | 46% | MS (ESI+) for CHNOS m/z 135.95 [M + H]⁺; ¹H NMR (400 MHz, DMSO-d₆): δ 8.46 (s, 1H), 8.19 (d, J = 5.2 Hz, 1H), 7.74 (bs, 2H), 7.43 (d, J = |
| | | | | 5.2 Hz, 1H). |
| Oxazolo[5,4- c]pyridin-2- amine | 90 | 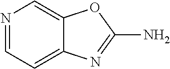 | 25% | MS (ESI+) for CHNOS m/z 163.22 [M + H]⁺; ¹H NMR (400 MHz, DMSO-d₆): δ 8.52 (s, 1H), 8.22 (d, J = 5.0 Hz, 1H), 7.99 (bs, 2H), 7.22 (d, J = |
| | | | | 5.0 Hz, 1H). |
| 5- (trifluoromethyl) benzo[d]oxazol- 2-amine | 91 | 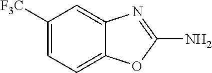 [SMILES: Nc1nc2cc(C(F)(F)F)ccc2o1] | 32% | MS (ESI+) for CHNOS m/z 203.0 [M + H]⁺. |
| (S)-5-(1- Methylpyrrolidin- 2-yl)-1,3,4- oxadiazol-2- amine | 92 | 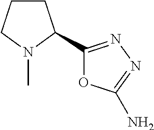 | 55% | MS (ESI+) for CHNOS m/z 169.22 [M + H]⁺. Crude data showed product. Proceeded further without purification. |
| (R)-5-(1- methylpyrrolidin- 2-yl)-1,3,4- oxadiazol-2- amine | 93 | 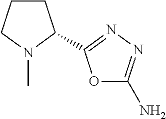 | 45% | MS (ESI+) for CHNOS m/z 169.22 [M + H]⁺. Crude data showed product. Proceeded further without purification. |

### Intermediate 94

### 1-Tosyl-1H-benzo[d]imidazol-2-amine

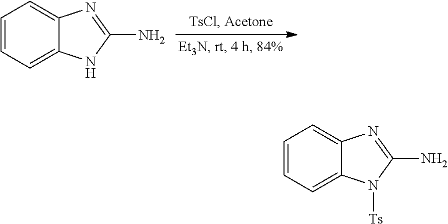 [SMILES: Nc1nc2ccccc2[nH]1 ; Cc1ccc(S(=O)(=O)n2c(N)nc3ccccc32)cc1]

To a solution of 1H-benzo[d]imidazol-2-amine (5 g, 37.5 mmol) in acetone (50 mL) were added triethylamine (15.8 mmol, 112.7 mmol) and Tscl (8.5 g, 45.1 mmol) in acetone (25 mL) slowly. The reaction mixture was stirred at rt for 4 h. The TLC showed reaction to be completed. The solvent was removed under reduced pressure. The residue was added to H₂O (50 mL) and extracted with EtOAc (3×50 mL). The organics layer was washed with brine (100 mL), dried (Na₂SO₄), filtered and concentrated under reduced pressure. The residue was triturated with DCM (100 mL), dried under vacuum to afford 1-tosyl-1H-benzo[d]imidazol-2-amine as a brown solid. Yield: 9 g (84%); ¹H NMR (400 MHz, DMSO-d₆): δ 10.14 (bs, 1H), 7.93 (d, J=8.3 Hz, 2H), 7.66 (d, J=8.0 Hz, 1H), 7.45 (d, J=8.3 Hz, 2H), 7.30 (bs, 2H), 7.09-7.15 (m, 2H), 6.99-7.06 (m, 1H), 2.35 (s, 3H); MS (ESI+) for CHNOS m/z 288.09 [M+H]⁺.

The following intermediate was prepared in a similar manner to 1-tosyl-1H-benzo[d]imidazol-2-amine.

| | | | | Spectral Data |
|---|---|---|---|---|
| Name | Int | Structure | Yield | 1H NMR & LCMS |
| 5-Chloro-1- tosyl-1H- benzo[d]imidazol- 2-amine | 95 | 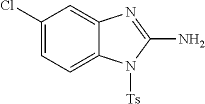 [SMILES: Cc1ccc(S(=O)(=O)n2c(N)nc3cc(Cl)ccc32)cc1] | 61% | MS (ESI+) for CHNOS m/z 322.29 [M + H]⁺; ¹H NMR (400 MHz, DMSO-d6): δ 7.89- 7.97 (m, 2H) 7.63 (d, J = 8.8 Hz, 1H), 7.42-7.49 (m, 2H), 7.34 (bs, 1H), 7.21 (bs, 1H), 7.12- 7.27 (m, 1H), 7.01-7.05 (m, |
| | | | | 1H), 2.36 (s, 3H). |

### Intermediate 96

### Dimethyl benzo[d]oxazol-2-ylcarbonimidodithioate

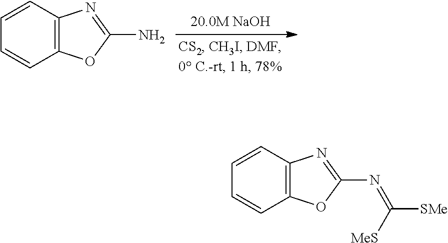 [SMILES: Nc1nc2ccccc2o1 ; CSC(=Nc1nc2ccccc2o1)SC]

To a suspension of benzo[d]oxazol-2-amine (5.0 g, 37.3 mmol) in DMF (50 mL) was added 20.0 M NaOH (1.86 mL, 37.3 mmol) at 0° C. The reaction mixture was stirred for 10 min and CS₂ (6.32 mL, 93.2 mmol) was added dropwise at 0° C. and the reaction mixture was further stirred for 10 min at 0° C. An additional portion of 20.0 M NaOH (1.86 mL, 37.3 mmol) was added at 0° C. and reaction mixture was again stirred for 10 min at 0° C. Finally, CH₃I (5.84 mL, 93.2 mmol) was added dropwise at 0° C. The reaction mixture was stirred at rt for 30 min. The TLC showed reaction to be complete. The mixture was poured into ice-water (100 mL) and the precipitated solid was filtered, washed with water (50 mL) followed by hexane (30 mL) and dried under reduced pressure to obtain dimethyl benzo[d]oxazol-2-ylcarbonimidodithioate as an off white solid. Yield: 6.92 g (78%). MS (ESI+) for CHNOS m/z 239.03 [M+H]⁺; ¹H NMR (400 MHz, DMSO-d₆): δ 7.64 (bs, 2H), 7.32 (bs, 2H), 2.67 (bs, 6H).

The following intermediates were prepared in a similar manner to dimethyl benzo[d]oxazol-2-ylcarbonimidodithioate.

| | | | | Spectral Data |
|---|---|---|---|---|
| Name | Int | Structure | Yield | 1H NMR & LCMS |
| Dimethyl (5- methyl-1,3,4- oxadiazol- 2-yl) carbonimidodithioate | 97 | 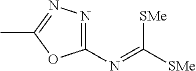 | 43% | MS (ESI+) for CHNOS m/z 204.21 [M + H]⁺; ¹H NMR (400 MHz, DMSO- d₆): δ 2.62 (bs, 6H), 2.44 (s, 3H). |
| Dimethyl (5- chlorobenzo[d] oxazol-2-yl) carbonimidodithioate | 98 | 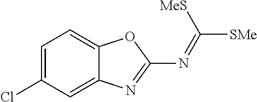 [SMILES: CSC(=Nc1nc2cc(Cl)ccc2o1)SC] | 50% | MS (ESI+) for CHNOS m/z 273.13 [M + H]⁺; ¹H NMR (400 MHz, DMSO- d₆): δ 7.75 (s, 1H), 7.65 (d, J = 8.6 Hz, 1H), 7.34- 7.38 (m, 1H), 2.67 (s, 6H). |
| Dimethyl (5- cyclopropyl-1,3,4- oxadiazol-2- yl) carbonimidodithioate | 99 | 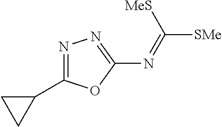 [SMILES: CSC(=Nc1nnc(C2CC2)o1)SC] | 60% | MS (ESI+) for CHNOS m/z 230.19 [M + H]⁺; ¹H NMR (400 MHz, DMSO- d₆): δ 2.56-2.80 (bs, 6H), 2.12-2.20 (m, 1H), 1.09- 1.18 (m, 2H), 0.90-1.07 (m, 2H). |
| Ethyl 5- ((bis(methylthio) methylene)amino)- 1,3,4-oxadiazole- 2-carboxylate | 100 | 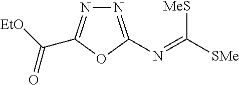 [SMILES: CCOC(=O)c1nnc(N=C(SC)SC)o1] | 25% | MS (ESI+) for CHNOS m/z 262.21 [M + H]⁺; ¹H NMR (400 MHz, DMSO- d₆): δ 4.40 (q, J = 7.1 Hz, 2H), 2.68 (s, 6H), 1.31 (t, J = 7.1 Hz, 3H). |
| Dimethyl (5- (trifluoromethyl) benzo[d]oxazol- 2-yl) carbonimidodithioate | 101 | 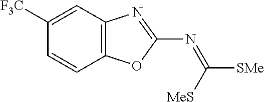 [SMILES: CSC(=Nc1nc2cc(C(F)(F)F)ccc2o1)SC] | 13% | MS (ESI+) for CHNOS m/z 306.91 [M + H]⁺. |
| Dimethyl (1-tosyl- 1H- benzo[d]imidazol- 2-yl) carbonimidodithioate | 102 | 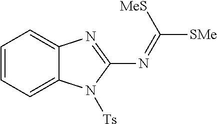 [SMILES: CSC(=Nc1nc2ccccc2n1S(=O)(=O)c1ccc(C)cc1)SC] | 35% | MS (ESI+) for CHNOS m/z 280.97 [M + H]⁺; ¹H NMR (400 MHz, DMSO- d₆): δ 7.96 (d, J = 8.2 Hz, 2H), 7.89-7.93 (m, 1H), 7.55-7.69 (m, 1H), 7.46 (d, J = 8.2 Hz, 2H), 7.29- 7.34 (m, 2H), 2.67 (s, 6H), |
| | | | | 2.35 (s, 3H). |
| Dimethyl (5- chloro-1-tosyl-1H- benzo[d]imidazol- 2-yl) carbonimidodithioate | 103 | 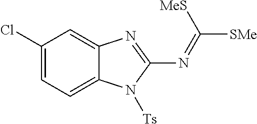 [SMILES: CSC(=Nc1nc2cc(Cl)ccc2n1S(=O)(=O)c1ccc(C)cc1)SC] | 10% | MS (ESI+) for CHNOS m/z 426.12 [M + H]⁺; ¹H NMR (400 MHz, DMSO- d₆): δ 8.01 (d, J = 8.8 Hz, 1H), 7.82 (d, J = 8.4 Hz, 2H), 7.66 (d, J = 2.0 Hz, 1H), 7.36-7.47 (m, 3H), 2.62 (s, 6H), 2.36 (s, 3H). |

### Intermediate 104

### N¹-Methyl-5-(trifluoromethyl)benzene-1,2-diamine

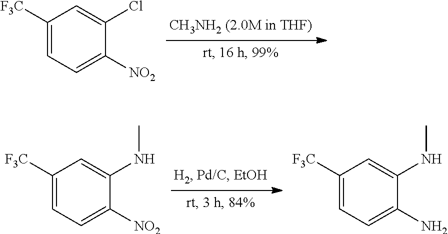

### N-Methyl-2-nitro-5-(trifluoromethyl)aniline

A mixture of 2-chloro-1-nitro-4-(trifluoromethyl)benzene (3 g, 13.3 mmol) and methylamine (2 M in THF) was stirred at rt for 16 h. The TLC showed reaction to be complete. The reaction mixture was diluted with H₂O (25 mL) and extracted with EtOAc (3×25 mL). The organics were washed with brine (50 mL), dried (Na₂SO₄), filtered and concentrated under reduced pressure to afford N-methyl-2-nitro-5-(trifluoromethyl)aniline as a yellow solid. Yield: 2.9 g (99%); ¹H NMR (400 MHz, CDCl₃): δ8.29 (d, J=8.8 Hz, 1H), 8.08 (bs, 1H), 7.09 (s, 1H), 6.88 (d, J=8.8 Hz, 1H), 3.06 (d, J=5.1 Hz, 1H); MS (ESI+) for CHNOS m/z 221.2 [M+H]⁺.

### N¹-Methyl-5-(trifluoromethyl)benzene-1,2-diamine

To a stirred solution of N-methyl-2-nitro-5-(trifluoromethyl)aniline (1.5 g, 6.31 mmol) in EtOH (20 mL) was added 10% Pd/C (700 mg) at room temperature. The reaction mixture was stirred at room temperature for 5 h under H₂ atmosphere (1 atm). The TLC showed reaction to be complete. The mixture was filtered through a celite bed and washed with EtOH (50 mL). The filtrate was evaporated under vacuum to afford N-methyl-5-(trifluoromethyl)benzene-1,2-diamine as brown liquid. Yield: 1.1 g (84%); ¹H NMR (400 MHz, CDCl₃): δ6.73 (d, J=7.7 Hz, 1H), 6.59 (d, J=7.7 Hz, 1H), 6.50 (s, 1H), 5.14 (bs, 2H), 4.97 (d, J=4.5 Hz, 1H), 2.74 (d, J=4.5 Hz, 3H); MS (ESI+) for CHNOS m/z 191.17 [M+H]⁺.

The following intermediate was prepared in a similar manner to 1 N¹-methyl-5-(trifluoromethyl)benzene-1,2-diamine.

| | | | | Spectral Data |
|---|---|---|---|---|
| Name | Int | Structure | Yield | 1H NMR & LCMS |
| N¹-Methyl-4- (trifluoromethyl) benzene- 1,2-diamine | 105 | 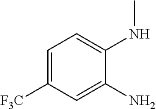 | 97% | MS (ESI+) for CHNOS m/z 190.64 [M + H]⁺; ¹H NMR (400 MHz, DMSO-d₆): δ 6.82 (d, J = 8.2 Hz, 1H), 6.78 (s, 1H), 6.42 (d, J = 8.2 Hz, 1H), 5.22 (d, J = 5.5 Hz, 1H), 4.86 (bs, 2H), 2.75 (d, J = 5.5 Hz, 3H). |

### Intermediate 106

### 2-Amino-5-chlorobenzenethiol

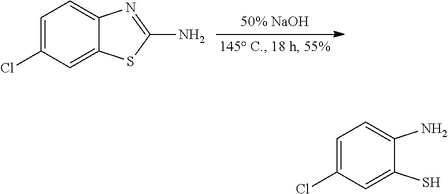 [SMILES: Nc1nc2ccc(Cl)cc2s1 ; Nc1ccc(Cl)cc1S]

A mixture of 6-chlorobenzo[d]thiazol-2-amine 8 g, 43.4 mmol) in 50% aq NaOH solution (120 mL) was stirred at 145° C. for 18 h. The TLC showed reaction to be complete. The reaction mixture was cooled to rt, diluted with H₂O (50 mL) and filtered. The filtrate was cooled to 0° C. and pH was adjusted to 6-7 with glacial acetic acid. The mixture was extracted with Et₂O (3×100 mL). The organic layer was washed with brine (100 mL), dried (Na₂SO₄), filtered and concentrated under reduced pressure to obtain compound as brown liquid as a dimer (2,2′-disulfanediylbis(4-chloroaniline). MS (ESI−) for CHNOS m/z 315.11 [M−H]⁺.

### Intermediate 107

### 2-Amino-5-chloro-4-(trifluoromethyl)phenol

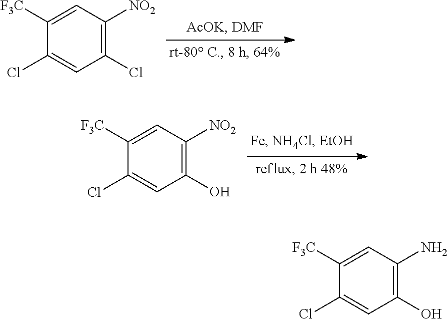 [SMILES: O=[N+]([O-])c1cc(C(F)(F)F)c(Cl)cc1Cl ; O=[N+]([O-])c1cc(C(F)(F)F)c(Cl)cc1O ; Nc1cc(C(F)(F)F)c(Cl)cc1O]

### 5-Chloro-2-nitro-4-(trifluoromethyl)phenol

To a solution of 1,5-dichloro-2-nitro-4-(trifluoromethyl)benzene (5 g, 19.2 mmol) in DMF (30 mL) was added potassium acetate (4.2 g, 42.4 mmol) at rt. The reaction mixture was stirred at 80° C. for 3 h. The TLC showed reaction to be complete. The reaction mixture was cooled to rt, quenched with 1N HCl and extracted with EtOAc (3×100 mL). The organics were dried (Na₂SO₄), filtered and concentrated under reduced pressure. The residue was purified by column chromatography using silica gel (100-200 mesh), eluting with 5% EtOAc in hexane to afford 5-chloro-2-nitro-4-(trifluoromethyl)phenol as an off white solid. Yield: 3 g (64%); ¹H NMR (400 MHz, DMSO-d₆): δ 12.36 (bs, 1H), 8.30 (s, 1H), 7.37 (s, 1H); MS (ESI−) for CHNOS m/z 240.11 [M−H]⁺.

### 2-Amino-5-chloro-4-(trifluoromethyl)phenol

To a mixture of 5-chloro-2-nitro-4-(trifluoromethyl)phenol (500 mg, 2.1 mmol) in EtOH (5 mL) and H₂O (5 mL) were added Fe powder (576 mg, 10.5 mmol) and ammonium chloride (553 mg 10.5 mmol) at rt. The reaction mixture was stirred at 90° C. for 2 h. The TLC showed reaction to be complete. The mixture was cooled to rt and filtered through celite pad. The filtrate was concentrated. The residue was diluted with H₂O (20 ml) and extracted with EtOAc (3×25 mL). The organics were dried (Na₂SO₄), filtered and concentrated under reduced pressure to afford 2-amino-5-chloro-4-(trifluoromethyl)phenol as a colourless liquid. Yield: 210 mg (48%); ¹H NMR (400 MHz, CDCl₃): δ 7.02 (s, 1H), 6.83 (s, 1H); MS (ESI−) for CHNOS m/z 210.13 [M−H]⁺.

### Intermediate 108

### 5-Chloro-N-methyl-2-nitro-4-(trifluoromethyl)aniline

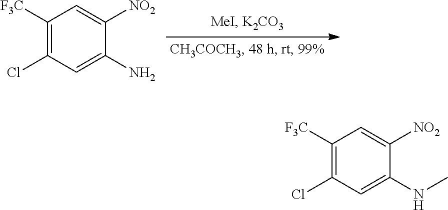 [SMILES: Nc1cc(Cl)c(C(F)(F)F)cc1[N+](=O)[O-] ; CNc1cc(Cl)c(C(F)(F)F)cc1[N+](=O)[O-]]

To a solution of 5-chloro-2-nitro-4-(trifluoromethyl)aniline (2 g, 8.31 mmol) in acetone (50 mL) were added K₂CO₃ (3.45 g, 24.94 mmol) and MeI (11.8 g, 83.14 mmol) at rt. The reaction mixture was stirred at rt for 48 h. The TLC showed reaction to be complete. The reaction was diluted with H₂O (50 mL) and extracted with EtOAc (3×50 mL). The organic layer was washed with brine (50 mL), dried (Na₂SO₄), filtered and concentrated under reduced pressure to afford 5-chloro-N-methyl-2-nitro-4-(trifluoromethyl)aniline as a yellow solid. Yield: 2 g (95%); ¹H NMR (400 MHz, DMSO-d₆): δ 8.63 (d, J=2.7 Hz, 1H), 8.35 (s, 1H), 7.27 (s, 1H), 3.01 (d, J=4.9 Hz, 3H); MS (ESI−) for CHNOS m/z 253.13 [M−H]⁺.

The following intermediate was prepared in a similar manner to 2-amino-5-chloro-4-(trifluoromethyl)phenol.

| | | | | Spectral Data |
|---|---|---|---|---|
| Name | Int | Structure | Yield | 1H NMR & LCMS |
| 3-Amino-5- chloro- pyridin- 4-ol | 109 | 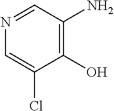 | Used crude | MS (ESI+) for CHNOS m/z 145.06 [M + H]⁺. |
| 5-Chloro- N¹- methyl-4- (trifluoro- methyl) | 110 | 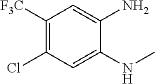 | 52% | MS (ESI+) for CHNOS m/z 225.01 [M + H]⁺. |
| benzene- | | | | |
| 1,2- | | | | |
| diamine | | | | |

### Intermediate 111

### 4-Chloro-5-(trifluoromethyl)benzene-1,2-diamine

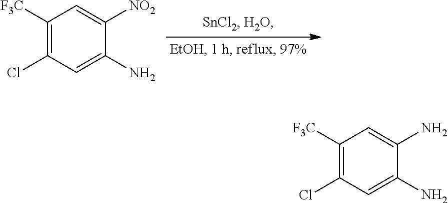 [SMILES: Nc1cc(Cl)c(C(F)(F)F)cc1[N+](=O)[O-] ; Nc1cc(Cl)c(C(F)(F)F)cc1N]

To a solution of 5-chloro-2-nitro-4-(trifluoromethyl)aniline (2 g, 8.31 mmol) in EtOH:H₂O (5:1, 10 mL) was added SnCl₂ (4.73 g, 24.9 mmol) at rt. The reaction mixture was stirred at 80° C. for 3 h. The TLC showed reaction to be complete. The reaction mixture was filtered through celite bed and concentrated under reduced pressure to afford 4-chloro-5-(trifluoromethyl)benzene-1,2-diamine as yellow semi solid. Yield: 1.7 g (97%); ¹H NMR (400 MHz, DMSO-d₆): δ 6.95 (s, 1H), 6.88 (s, 1H), 4.68-5.08 (bs, 4H), MS (ESI−) for CHNOS m/z 209.15 [M−H]⁺.

### Intermediate 112

### 2,6-Dichloro-1-methyl-5-(trifluoromethyl)-1H-benzo[d]imidazole

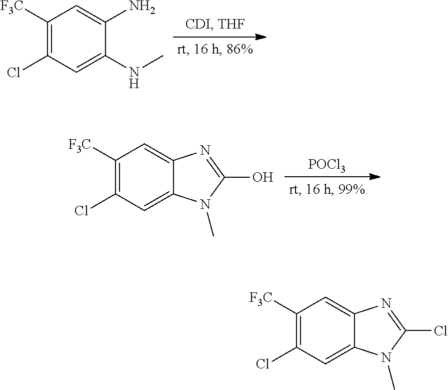 [SMILES: CNc1cc(Cl)c(C(F)(F)F)cc1N ; Cn1c(O)nc2cc(C(F)(F)F)c(Cl)cc21 ; "POCl3" ; Cn1c(Cl)nc2cc(C(F)(F)F)c(Cl)cc21]

### 6-Chloro-1-methyl-5-(trifluoromethyl)-1H-benzo[d]imidazol-2-ol

To a solution of 5-chloro-N¹-methyl-4-(trifluoromethyl)benzene-1,2-diamine (1 g, 4.45 mmol) in THF (50 mL) was added CDI (3.61 g, 22.3 mmol) at rt. The reaction mixture was stirred at rt for 16 h. The TLC showed reaction to be complete. The reaction was diluted with H₂O (50 mL) and extracted with EtOAc (3×50 mL). The organic layer was washed with brine (50 mL), dried (Na₂SO₄), filtered and concentrated under reduced pressure to afford 6-chloro-1-methyl-5-(trifluoromethyl)-1H-benzo[d]imidazol-2-ol as brown solid. Yield: 900 mg (86%); ¹H NMR (400 MHz, DMSO-d₆): δ 11.31 (bs, 1H), 7.51 (s, 1H), 7.27 (s, 1H), 3.31 (s, 3H); MS (ESI−) for CHNOS m/z 249.15 [M−H]⁺.

### 2,6-Dichloro-1-methyl-5-(trifluoromethyl)-1H-benzo[d]imidazole

A solution of 6-chloro-1-methyl-5-(trifluoromethyl)-1H-benzo[d]imidazol-2-ol (500 mg, 2.0 mmol) in POCl₃ (20 mL) was heated at 80° C. for 16 h. The TLC showed reaction to be complete. The solvent was removed under reduced pressure. The residue was diluted with ice-cold water (50 mL) and extracted with EtOAc (3×50 mL). The organic layer was washed with brine (50 mL), dried (Na₂SO₄), filtered and concentrated under reduced pressure to afford 2,6-dichloro-1-methyl-5-(trifluoromethyl)-1H-benzo[d]imidazole. Yield: 500 mg (93%); MS (ESI+) for CHNOS m/z 269.0 [M+H]⁺.

The following intermediate was prepared in a similar manner to 6-chloro-1-methyl-5-(trifluoromethyl)-1H-benzo[d]imidazol-2-ol.

| | | | | Spectral Data |
|---|---|---|---|---|
| Name | Int | Structure | Yield | 1H NMR & LCMS |
| 6-Chloro-5- (trifluoromethyl)-1H- benzo[d]imidazol- 2-ol | 113 | 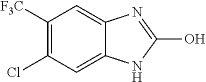 [SMILES: Oc1nc2cc(C(F)(F)F)c(Cl)cc2[nH]1] | 20% | MS (ESI−) for CHNOS m/z 235.14 [M − H]⁺; ¹H NMR (400 MHz, DMSO- d₆): δ 11.22 (bs, 1H), 11.10 (bs, 1H), 7.24 (s, 1H), 7.18 (s, 1H). |

The following intermediate was prepared in a similar manner to 2,6-dichloro-5-(trifluoromethyl)-1H-benzo[d]imidazole.

| | | | | Spectral Data |
|---|---|---|---|---|
| Name | Int | Structure | Yield | 1H NMR & LCMS |
| 2,6-Dichloro-5- (trifluoromethyl)- 1H-benzo [d]imidazole | 114 | 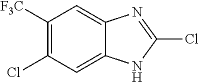 [SMILES: FC(F)(F)c1cc2nc(Cl)[nH]c2cc1Cl] | 66% | MS (ESI−) for CHNOS m/z 253.13 [M − H]⁺; ¹H NMR (400 MHz, DMSO- d₆): δ]⁺ δ 13.98 (bs, 1H), 8.01 (s, 1H), 7.89 (s, 1H). |

### Intermediate 115

### N¹,3-Dimethylbenzene-1,2-diamine

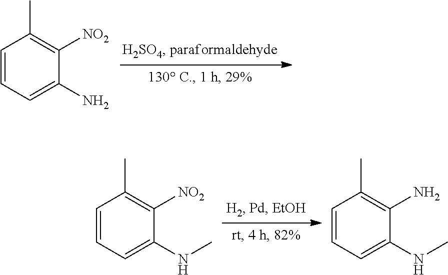

### N,3-Dimethyl-2-nitroaniline

To a solution of 3-methyl-2-nitroaniline (500 mg, 3.28 mmol) in H₂SO₄ (2 mL) was added paraformaldehye (400 mg, 13.3 mmol) slowly at rt. The reaction mixture was stirred at 80° C. for 1 h. The TLC showed reaction to be complete. The reaction was poured in H₂O (50 mL) and extracted with EtOAc (3×50 mL). The organic layer was washed with brine (50 mL), dried (Na₂SO₄), filtered and concentrated under reduced pressure. The residue was purified by column chromatography using silica gel (100-200 mesh), eluting with 2% EtOAc in hexane to afford N,3-dimethyl-2-nitroaniline as a yellow solid. Yield: 160 mg (29%); ¹H NMR (400 MHz, DMSO-d₆): δ 7.21-7.29 (m, 1H), 6.67 (d, J=8.4 Hz, 1H), 6.54 (d, J=8.4 Hz, 1H), 2.93 (s, 1H), 2.48 (s, 2H).

### N¹,3-Dimethylbenzene-1,2-diamine

To a solution of N,3-dimethyl-2-nitroaniline (160 mg, 0.96 mmol) in MeOH (10 mL), 10% Pd—C (160 mg) was added. The reaction mixture was stirred at rt under H₂ balloon atmosphere for 2 h. TLC showed the reaction to be complete and filtered through a celite bed and washed with MeOH (50 mL). The filtrate was evaporated under vacuum to afford N¹,3-dimethylbenzene-1,2-diamine as a red liquid. Yield: 100 mg (82%); ¹H NMR (400 MHz; DMSO-d₆): δ 6.70-6.81 (m, 1H), 6.58-6.66 (m, 2H), 2.87 (s, 3H), 2.21 (s, 3H); MS (ESI+) for CHNOS m/z 137.01 [M+H]⁺.

### Synthetic Route 3

### 5-Chloro-N-(5-methyl-1,3,4-oxadiazol-2-yl)benzo[d]oxazol-2-amine (Example 50)

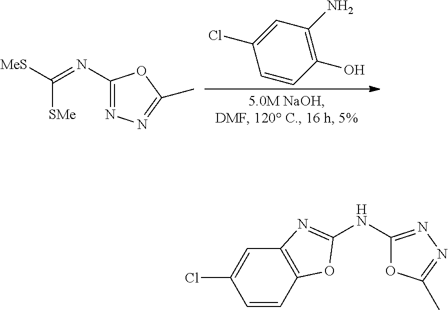 [SMILES: CSC(=Nc1nnc(C)o1)SC ; Nc1cc(Cl)ccc1O ; Cc1nnc(Nc2nc3cc(Cl)ccc3o2)o1]

### 5-Chloro-N-(5-methyl-1,3,4-oxadiazol-2-yl)benzo[d]oxazol-2-amine

To a solution of 2-amino-4-chlorophenol (500 mg, 3.49 mmol) in DMF (10 mL) was added 5.0 N NaOH solution (1.4 mL, 6.96 mmol) at rt. The reaction mixture was stirred at rt for 20 min and dimethyl (5-methyl-1,3,4-oxadiazol-2-yl)carbonimidodithioate (708 mg, 3.49 mmol) was added to it at rt. The reaction mixture was stirred at 120° C. for 16 h. TLC showed the reaction to be complete. The reaction mixture was allowed to cool to rt, poured into ice-water (50 mL), acidified to pH 4-5 with 1.0N HCl and extracted with EtOAc (3×50 mL). The organics were washed with ice-cold water (2×50 mL), brine (100 mL), dried (Na₂SO₄), filtered and concentrated under reduced pressure. The residue was triturated with DCM (5.0 mL) followed by Et₂O (10 mL) and dried under reduced pressure to afford 5-chloro-N-(5-methyl-1,3,4-oxadiazol-2-yl)benzo[d]oxazol-2-amine as off white solid Yield: 43 mg (5%); MS (ESI+) for CHNOS m/z 250.98 [M+H]⁺; LC purity 99.4% (Ret. Time—5.41 min); ¹H NMR (400 MHz, DMSO-d₆): δ 12.40 (bs, 1H), 7.53 (d, J=8.6 Hz, 1H), 7.44 (s, 1H), 7.22-7.27 (m, 1H), 2.42 (S, 3H).

The following examples were prepared in a similar manner to 5-chloro-N-(5-methyl-1,3,4-oxadiazol-2-yl)benzo[d]oxazol-2-amine using synthetic route 3.

| | | | | Spectral Data |
|---|---|---|---|---|
| Name | Ex | Structure | Yield | 1H NMR & LCMS |
| N-(5- Cyclopropyl- 1,3,4- oxadiazol-2- yl)-6- (trifluoromethyl) oxazolo[4,5- c]pyridin-2- amine | 51 | 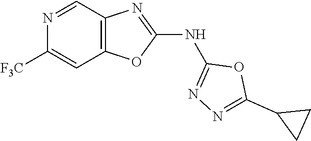 [SMILES: FC(F)(F)c1cc2oc(Nc3nnc(C4CC4)o3)nc2cn1] | 13% | MS (ESI+) for CHNOS m/z 312.26 [M + H]⁺; LC purity 96.9% (Ret. Time- 5.77 min); ¹H NMR (400 MHz, DMSO-d₆): δ 8.44 (s, 1H), 7.69 (s, 1H), 2.01 (bs, 1H), 0.98 (bs, 2H), 0.87 (bs, 2H). |
| Ethyl 5-((5- (trifluoromethyl) benzo[d]oxazol- 2-yl)amino)- 1,3,4- oxadiazole-2- carboxylate | 52 | 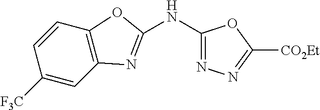 [SMILES: CCOC(=O)c1nnc(Nc2nc3cc(C(F)(F)F)ccc3o2)o1] | 24% | MS (ESI+) for CHNOS m/z 343.30 [M + H]⁺; LC purity 98.42% (Ret. Time- 1.79 min); ¹H NMR (400 MHz, DMSO-d₆): δ 7.50 (s, 1H), 7.40 (d, J = 8.0 Hz, 1H), 7.24 (d, J = 8.0 Hz, 1H), 4.34 (q, J = 7.1 |
| | | | | Hz, 2H), 1.32 (t, J = 7.1 |
| | | | | Hz, 3H). |
| N-(5-Methyl- 1,3,4- oxadiazol-2- yl)-6- (trifluoromethyl) oxazolo[4,5- c]pyridin-2- amine | 53 | 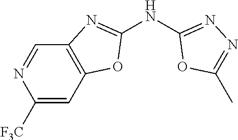 [SMILES: Cc1nnc(Nc2nc3cnc(C(F)(F)F)cc3o2)o1] | 6% | MS (ESI+) for CHNOS m/z 286.09 [M + H]⁺; LC purity 96.6% (Ret. Time- 5.11 min); ¹H NMR (400 MHz, DMSO-d₆): δ 8.81 (s, 1H), 8.69 (s, 1H), 2.39 (s, 3H). |
| 6-Fluoro-N-(5- methyl-1,3,4- oxadiazol-2- yl)benzo[d] oxazol-2-amine | 54 | 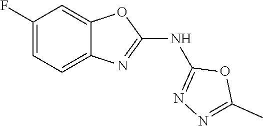 [SMILES: Cc1nnc(Nc2nc3ccc(F)cc3o2)o1] | 13% | MS (ESI+) for CHNOS m/z 235.05 [M + H]⁺; LC purity 99.2% (Ret. Time- 4.02 min); ¹H NMR (400 MHz, DMSO-d₆): δ 7.53 (d, J = 7.4 Hz, 1H), 7.40 (bs, 1H). 7.12 (bs, 1H), 2.41 (s, 3H). |
| Ethyl 5-((1- methyl-5- (trifluoromethyl)- 1H- benzo[d]imidazol- 2-yl)amino)- 1,3,4- oxadiazole-2- carboxylate | 55 | 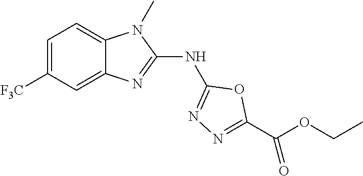 [SMILES: CCOC(=O)c1nnc(Nc2nc3cc(C(F)(F)F)ccc3n2C)o1] | 31% | MS (ESI+) for CHNOS m/z 356.15 [M + H]⁺; LC purity 99.0% (Ret. Time- 5.91 min); ¹H NMR (400 MHz, DMSO-d₆): δ 12.29 (bs, 1H), 7.88 (s, 1H), 7.58-7.69 (m, 2H), 4.38 (q, J = 7.1 Hz, 2H), 3.64 (s, 3H), 1.34 (t, J = 7.1 Hz, 3H). |
| Ethyl 5-((5- (trifluoromethyl)- 1H- benzo[d]imidazol- 2-yl)amino)- 1,3,4- oxadiazole-2- carboxylate | 56 | 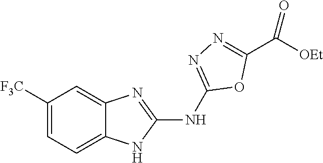 [SMILES: CCOC(=O)c1nnc(Nc2nc3cc(C(F)(F)F)ccc3[nH]2)o1] | 57% | MS (ESI+) for CHNOS m/z 342.11 [M + H]⁺; LC purity 98.2% (Ret. Time- 5.36 min); ¹H NMR (400 MHz, DMSO-d₆): δ 12.50 (bs, 2H), 7.71 (s, 1H), 7.46-7.57 (m, 2H), 4.38 (q, J = 7.1 Hz, 2H), 1.34 (t, J = 7.1 Hz, 3H). |
| 7-Chloro-N- (1,3,4- oxadiazol-2- yl)benzo[d] oxazol-2-amine | 57 | 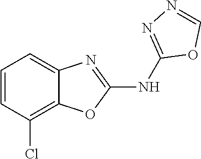 [SMILES: Clc1cccc2nc(Nc3nnco3)oc12] | 35% | MS (ESI+) for CHNOS m/z 237.11 [M + H]⁺; LC purity 99% (Ret. Time- 3.88 min); ¹H NMR (400 MHz, DMSO-d₆): δ 8.86 (s, 1H), 7.36-7.41 (m, 1H), 7.25-7.35 (m, 2H). |
| N-(5-Methyl- 1,3,4- oxadiazol-2- yl)oxazolo[4,5- c]pyridin-2- amine | 58 | 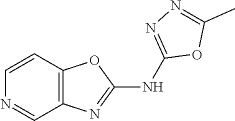 [SMILES: Cc1nnc(Nc2nc3cnccc3o2)o1] | 17% | MS (ESI+) for CHNOS m/z 218.14 [M + H]⁺; LC purity 97.4% (Ret. Time- 5.51 min); ¹H NMR (400 MHz, DMSO-d₆): δ 8.87 (s, 1H), 8.57 (d, J = 5.9 Hz, 1H), 7.91 d, J = 5.9 Hz, 1H), 2.43 (s, 3H). |
| N-(5- (triFluoromethyl) benzo[d] oxazol-2- yl)oxazolo[5,4- c]pyridin-2- amine | 59 | 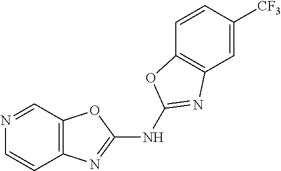 [SMILES: FC(F)(F)c1ccc2oc(Nc3nc4ccncc4o3)nc2c1] | 27% | MS (ESI+) for CHNOS m/z 321.24 [M + H]⁺; LC purity 96.2% (Ret. Time- 4.80 min); ¹H NMR (400 MHz, DMSO-d₆): δ 8.51 (s, 1H), 8.20 (s, 1H), 7.42- 7.74 (m, 2H), 7.20-7.42 (m, 2H). |
| N-(5- (triFluoromethyl) benzo[d] oxazol-2- yl)oxazolo[4,5- c]pyridin-2- amine | 60 | 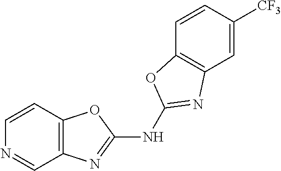 [SMILES: FC(F)(F)c1ccc2oc(Nc3nc4cnccc4o3)nc2c1] | 47% | MS (ESI+) for CHNOS m/z 321.12 [M + H]+; LC purity 94.4%; 1H NMR (400 MHz, DMSO-d6): δ 8.51 (s, 1H), 8.13 (d, J = 5.0 Hz, 1H), 7.95 (s, 1H), 7.58 (bs, 1H), 7.42 (d, J = 7.8 Hz, 1H), 7.23-7.38 (m, 2H). |
| Ethyl 5-((6- chlorobenzo[d] oxazol-2- yl)amino)- 1,3,4- oxadiazole-2- carboxylate | 61 | 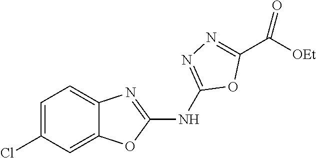 [SMILES: CCOC(=O)c1nnc(Nc2nc3ccc(Cl)cc3o2)o1] | 28% | MS (ESI+) for CHNOS m/z 309.17 [M + H]+; LC purity 93% (Ret. Time- 1.61 min); 1H NMR (400 MHz, DMSO-d6): δ 7.34 (s, 1H), 7.22 (d, J = 8.2 Hz, 1H), 7.07 (d, J = 8.2 Hz, 1H), 4.34 (q, J = 6.9 Hz, 2H), 1.31 (t, J = 6.9 Hz, 3H). |
| 4-Fluoro-N-(5- methyl-1,3,4- oxadiazol-2- yl)benzo[d] oxazol-2-amine | 62 | 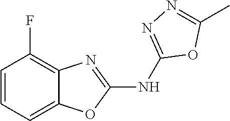 [SMILES: Cc1nnc(Nc2nc3c(F)cccc3o2)o1] | 8% | MS (ESI+) for CHNOS m/z 235.20 [M + H]+; LC purity 98.2% (Ret. Time- 3.53 min); 1H NMR (400 MHz, DMSO-d6): δ 8.52 (s, 1H), 6.98 (d, J = 5.8 Hz, 1H), 6.77-6.85 (m, 2H), 2.28 (s, 3H). |
| 6-Fluoro-N- (1,3,4- oxadiazol-2- yl)benzo[d] oxazol-2-amine | 63 | 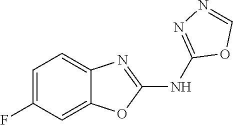 [SMILES: Fc1ccc2nc(Nc3nnco3)oc2c1] | 9% | MS (ESI+) for CHNOS m/z 221.02 [M + H]⁺; LC purity 99.8% (Ret. Time- 3.38); ¹H NMR (400 MHz, DMSO-d₆): δ 12.39 (bs, 1H), 8.83 (s, 1H), 7.58 (dd, J = 2.0, 8.4 Hz, 1H), 7.36-7.47 (m, 1H), 7.11- 7.21 (m, 1H). |
| 7-Chloro-N-(5- methyl-1,3,4- oxadiazol-2- yl)oxazolo[4,5- c]pyridin-2- amine | 64 | 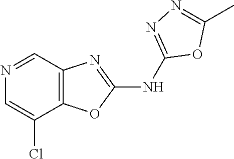 [SMILES: Cc1nnc(Nc2nc3cncc(Cl)c3o2)o1] | 17% | MS (ESI+) for CHNOS m/z 252.22 [M + H]⁺; LC purity 95.2% (Ret. Time- 4.25); ¹H NMR (400 MHz, DMSO-d₆): δ 8.64 (s, 1H), 8.43 (s, 1H), 2.43 (s, 3H). |
| Ethyl 5-((6- chloro-5- (trifluoromethyl) benzo[d] oxazol-2-yl) amino)-1,3,4- oxadiazole-2- carboxylate | 65 | 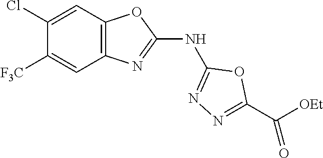 [SMILES: CCOC(=O)c1nnc(Nc2nc3cc(C(F)(F)F)c(Cl)cc3o2)o1] | 34% | MS (ESI+) for CHNOS m/z 377.14 [M + H]+; LC purity 96%) 1H NMR (400 MHz, DMSO-d6): δ 7.68 (s, 1H), 7.63 (s, 1H), 4.34 (q, J = 7.1 Hz, 2H), 1.32 (q, J = 7.1 Hz, 3H). |
| 5-Fluoro-N- (1,3,4- oxadiazol-2- yl)benzo[d] oxazol-2-amine | 66 | 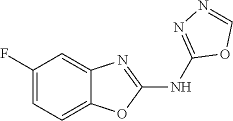 [SMILES: Fc1ccc2oc(Nc3nnco3)nc2c1] | 2% | MS (ESI+) for CHNOS m/z 221.02 [M + H]⁺; LC purity 97.2% (Ret. Time- 4.47); ¹H NMR (400 MHz, DMSO-d₆): δ 12.68 (bs, 1H), 8.85 (s, 1H), 7.47- 7.62 (m, 1H), 7.21-7.29 (m, 1H). 7.01-7.11 (m, 1H). |

### Synthetic Route 4

### N-(1,3,4-Oxadiazol-2-yl)-5-(trifluoromethyl)benzo[d]oxazol-2-amine (Example 67)

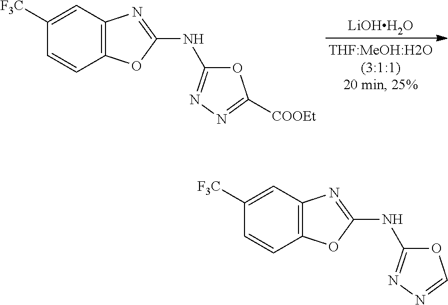 [SMILES: CCOC(=O)c1nnc(Nc2nc3cc(C(F)(F)F)ccc3o2)o1 ; "LiOH·H2O" ; FC(F)(F)c1ccc2oc(Nc3nnco3)nc2c1]

To a stirred solution of ethyl 5-((5-(trifluoromethyl)benzo[d]oxazol-2-yl)amino)-1,3,4-oxadiazole-2-carboxylate (100 mg, 2.9 mmol) in THF:MeOH:H₂O (3:1:1, 5.0 mL) was added LiOH.H₂O (25 mg, 0.58 mmol) at rt. The reaction mixture was stirred for 20 min at rt. The TLC showed reaction to be complete. The reaction mixture was concentrated under reduced pressure. The residue was acidified to pH=2 by 1N HCl. The solid precipitated was filtered, triturated with Et₂O (5 mL) and dried under vacuum to afford N-(1,3,4-oxadiazol-2-yl)-5-(trifluoromethyl)benzo[d]oxazol-2-amine as off white solid. Yield: 20 mg (25%); ¹H NMR (400 MHz, DMSO-d₆): δ 8.87 (s, 1H), 7.66-7.82 (m, 2H), 7.61 (d, J=7.8 Hz, 1H); MS (ESI+) for CHNOS m/z 271.04 [M+H]⁺.

The following examples were prepared in a similar manner to N-(1,3,4-oxadiazol-2-yl)-5-(trifluoromethyl)benzo[d]oxazol-2-amine following synthetic route 4.

| | | | | Spectral Data |
|---|---|---|---|---|
| Name | Ex | Structure | Yield | 1H NMR & LCMS |
| N-(5- (Trifluoromethyl)- 1H-benzo[d] imidazol-2-yl)- 1,3,4- oxadiazol-2- amine | 68 | 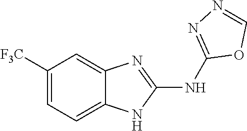 [SMILES: FC(F)(F)c1ccc2[nH]c(Nc3nnco3)nc2c1] | 31% | MS (ESI+) for CHNOS m/z 270.07 [M + H]⁺; LC purity 97% (Ret. Time-5.06 min); ¹H NMR (400 MHz, DMSO- d₆): δ 12.18 (bs, 2H), 8.65 (s, 1H), 7.65 (s, 1H), 7.47 (s, 2H). |
| N-(1-methyl- 5-(trifluoromethyl)- 1H-benzo[d] imidazol-2-yl)- 1,3,4-oxadiazol- 2-amine | 69 | 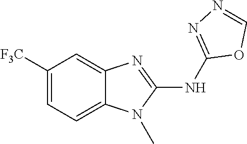 [SMILES: Cn1c(Nc2nnco2)nc2cc(C(F)(F)F)ccc21] | 50% | MS (ESI+) for CHNOS m/z 284.11 [M + H]⁺; LC purity 97.7% (Ret. Time-5.62 min); ¹H NMR (400 MHz, DMSO-d₆): δ 12.09 (bs, 1H), 8.71 (s, 1H), 7.84 (s, 1H), 7.53-7.61 (m, 2H), 3.59 (s, 3H). |

### Synthetic Route 5

### N-(5-(morpholinomethyl)-1,3,4-oxadiazol-2-yl)-5-(trifluoromethyl)benzo[d]oxazol-2-amine (Example 70)

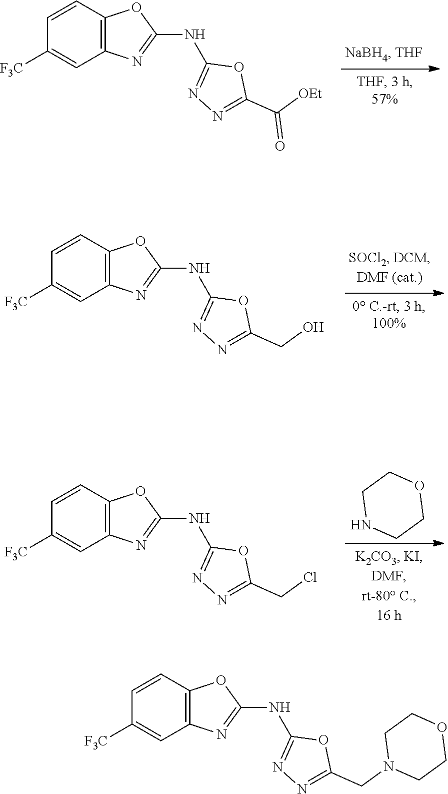 [SMILES: CCOC(=O)c1nnc(Nc2nc3cc(C(F)(F)F)ccc3o2)o1 ; OCc1nnc(Nc2nc3cc(C(F)(F)F)ccc3o2)o1 ; FC(F)(F)c1ccc2oc(Nc3nnc(CCl)o3)nc2c1 ; C1COCCN1 ; FC(F)(F)c1ccc2oc(Nc3nnc(CN4CCOCC4)o3)nc2c1]

### (5-((5-(Trifluoromethyl)benzo[d]oxazol-2-yl)amino)-1,3,4-oxadiazol-2-yl)methanol

To a stirred solution of ethyl 5-((5-(trifluoromethyl)benzo[d]oxazol-2-yl)amino)-1,3,4-oxadiazole-2-carboxylate (2 g, 5.84 mmol) was added sodium borohydride (700 mg, 17.5 mmol) portionwise at 0° C. under N₂ atmosphere. The reaction was stirred at rt for 3 h. The TLC showed reaction to be complete. The solvent was removed under reduced pressure. The residue was dissolved in 5% MeOH in EtOAC (100 mL) and washed with saturated NH₄Cl solution (100 mL). The aqueous layer was extracted with 5% MeOH in EtOAC (3×50 mL). The organics layer was washed with brine (20 mL), dried (Na₂SO₄), filtered and concentrated under reduced pressure. The residue was triturated with Et₂O (50 mL) to afford (5-((5-(trifluoromethyl)benzo[d]oxazol-2-yl)amino)-1,3,4-oxadiazol-2-yl)methanol as off white solid. Yield: 1 g (57%); ¹H NMR (400 MHz, DMSO-d₆): δ 7.69 (s, 1H), 7.38 (d, J=8.2 Hz, 1H), 7.25 (d, J=8.2 Hz, 1H), 4.43 (s, 2H); MS (ESI+) for CHNOS m/z 301.23 [M+H]⁺.

### N-(5-(Chloromethyl)-1,3,4-oxadiazol-2-yl)-5-(trifluoromethyl)benzo[d]oxazol-2-amine

To a stirred suspension of (5-((5-(trifluoromethyl)benzo[d]oxazol-2-yl)amino)-1,3,4-oxadiazol-2-yl)methanol (400 mg, 13.3 mmol) in DCM (20 mL) were added DMF (cat.) and SOCl₂ (2.0 mL) slowly at 0° C. The reaction mixture was stirred at rt for 4 h. The TLC showed reaction to be complete. The reaction was evaporated under N₂ atmosphere to afford N-(5-(chloromethyl)-1,3,4-oxadiazol-2-yl)-5-(trifluoromethyl)benzo[d]oxazol-2-amine as off white viscous solid. Yield: 400 mg (crude). The residue was used in next step as such.

### N-(5-(morpholinomethyl)-1,3,4-oxadiazol-2-yl)-5-(trifluoromethyl)benzo[d]oxazol-2-amine

To a mixture of N-(5-(chloromethyl)-1,3,4-oxadiazol-2-yl)-5-(trifluoromethyl)benzo[d]oxazol-2-amine (400 mg) obtained from above step in DMF (5 mL) were added K₂CO₃ (1.85 g, 13.3 mmol), KI (110 mg, 0.66 mmol) and morphiline (0.2 mL, 1.5 mmol) at rt under N₂ atmosphere. The reaction mixture was stirred at 80° C. for 8 h. The TLC showed reaction to be complete. The solvent was removed under reduced pressure. The residue was diluted with H₂O (25 mL) and extracted with 10% IPA in CHCl₃ (3×25 mL). The organics layer was washed with brine (20 mL), dried (Na₂SO₄), filtered and concentrated under reduced pressure. The crude residue was purified by prep HPLC to afford N-(5-(morpholinomethyl)-1,3,4-oxadiazol-2-yl)-5-(trifluoromethyl)benzo[d]oxazol-2-amine as off white solid. Yield: 150 mg (32%); ¹H NMR (400 MHz, DMSO-d₆): δ 7.75 (d, J=8.5 Hz, 1H), 7.71 (s, 1H), 7.64 (d, J=8.5 Hz, 1H), 4.28 (s, 2H), 3.65 (bs, 4H), 2.98 (bs, 4H); MS (ESI+) for CHNOS m/z 370.23 [M+H]⁺.

The following intermediates were prepared in a similar manner to (5-((5-(Trifluoromethyl)benzo[d]oxazol-2-yl)amino)-1,3,4-oxadiazol-2-yl) methanol.

| | | | | Spectral Data |
|---|---|---|---|---|
| Name | Int | Structure | Yield | 1H NMR & LCMS |
| (5-((6- Chlorobenzo[d] oxazol-2- yl)amino)-1,3,4- oxadiazol-2- yl)methanol | 116 | 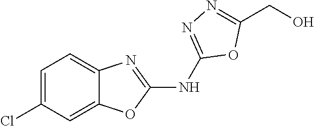 [SMILES: OCc1nnc(Nc2nc3ccc(Cl)cc3o2)o1] | 58% | MS (ESI+) for CHNOS m/z 267.19 [M + H]; ¹H NMR (400 MHz, DMSO-d₆): δ 7.44 (s, 1H), 7.28 (d, J = 7.4 Hz, 1H), 7.14 (d, J = 7.4 Hz, 1H), 4.46 (s, 2H). |
| (5-((6-Chloro-5- (trifluoromethyl) benzo[d]oxazol-2- yl)amino)-1,3,4- oxadiazol-2- yl)methanol | 117 | 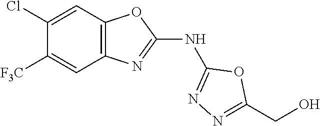 [SMILES: OCc1nnc(Nc2nc3cc(C(F)(F)F)c(Cl)cc3o2)o1] | 63% | MS (ESI+) for CHNOS m/z 335.02 [M + H]; ¹H NMR (400 MHz, DMSO-d₆): δ 7.65 (s, 1H), 7.52 (s, 1H), 5.59 (bs, 1H), 4.42 (s, 2H). |

The following intermediates were prepared in a similar manner to (N-(5-(chloromethyl)-1,3,4-oxadiazol-2-yl)-5-(trifluoromethyl)benzo[d]oxazol-2-amine.

| | | | | Spectral Data |
|---|---|---|---|---|
| Name | Int | Structure | Yield | 1H NMR & LCMS |
| 6-Chloro-N-(5- (chloromethyl)- 1,3,4-oxadiazol- 2-yl)benzo[d] oxazol-2-amine | 118 | 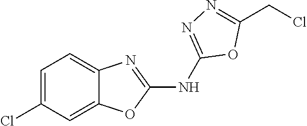 [SMILES: ClCc1nnc(Nc2nc3ccc(Cl)cc3o2)o1] | Used crude | No data recorded. |
| 6-Chloro-N-(5- (chloromethyl)- 1,3,4-oxadiazol- 2-yl)-5- (trifluoromethyl) benzo[d]oxazol- 2-amine | 119 | 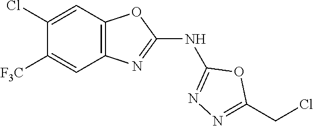 [SMILES: FC(F)(F)c1cc2nc(Nc3nnc(CCl)o3)oc2cc1Cl] | Used crude | No data recorded. |

The following examples were prepared in a similar manner to N-(5-(morpholinomethyl)-1,3,4-oxadiazol-2-yl)-5-(trifluoromethyl)benzo[d]oxazol-2-amine following synthetic route 5.

| | | | | Spectral Data |
|---|---|---|---|---|
| Name | Ex | Structure | Yield | 1H NMR & LCMS |
| N-(5- (Pyrrolidin-1- ylmethyl)-1,3,4- oxadiazol-2- yl)-5- (trifluoromethyl) benzo[d] oxazol-2-amine | 71 | 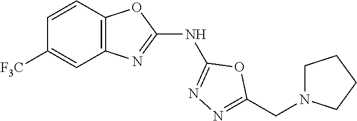 [SMILES: FC(F)(F)c1ccc2oc(Nc3nnc(CN4CCCC4)o3)nc2c1] | 2% | MS (ESI+) for CHNOS m/z 354.25 [M + H]⁺; LC purity 99.2% (Ret. Time- 4.76 min); ¹H NMR (400 MHz, DMSO-d₆): δ 7.76 (d, J = 8.6 Hz, 1H), 7.71 (s, 1H), |
| | | | | 7.65 (d, J = 8.6 Hz, |
| | | | | 1H), 4.69 (s, 2H), |
| | | | | 3.35 (bs, 4H), 1.96 |
| | | | | (bs, 4H). |
| N-(5- (Piperidin-1- ylmethyl)- 1,3,4- oxadiazol-2- yl)-5- (trifluoromethyl) benzo[d] | 72 | 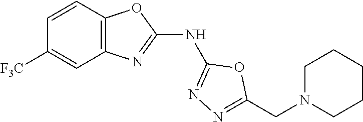 [SMILES: FC(F)(F)c1ccc2oc(Nc3nnc(CN4CCCCC4)o3)nc2c1] | 3% | MS (ESI+) for CHNOS m/z 368.0 [M + H]⁺; LC purity 98% (Ret. Time- 4.61 min); ¹H NMR (400 MHz, DMSO-d₆): δ 7.77 (d, J = 8.5 Hz, 1H), 7.76 (s, 1H), |
| oxazol-2-amine | | | | 7.65 (d, J = 8.5 Hz, |
| | | | | 1H), 4.56 (s, 2H), |
| | | | | 3.22 (bs, 4H), 1.74 |
| | | | | (bs, 4H), 1.51 (bs, 2H). |
| N-(5-((2- Methylpyrrolidin- 1-yl)methyl)- 1,3,4- oxadiazol-2- yl)-5- (trifluoromethyl) benzo[d] oxazol-2-amine | 73 | 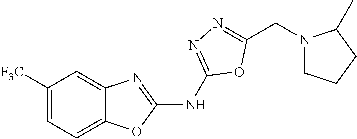 [SMILES: CC1CCCN1Cc1nnc(Nc2nc3cc(C(F)(F)F)ccc3o2)o1] | 2% | MS (ESI+) for CHNOS m/z 368.32 [M + H]⁺; LC purity 99.5% (Ret. Time- 4.61 min); ¹H NMR (400 MHz, DMSO-d₆ + D₂O): δ 7.77 (d, J = 8.5 Hz, 1H), 7.72 (s, 1H), 7.65 (d, J = 8.5 |
| | | | | Hz, 1H), 4.74 (d, J = |
| | | | | 15.0 Hz, 1H), 4.54 (d, |
| | | | | J = 15.0 Hz, 1H), 3.58 |
| | | | | (bs, 2H), 3.29 (bs, |
| | | | | 1H), 2.20 (bs, 1H), |
| | | | | 1.91-1.98 (m, 2H), |
| | | | | 1.63 (bs, 1H), 1.34 (d, |
| | | | | J = 6.5, 3H). |
| N-(5-((3,3- diFluoro- pyrrolidin-1- yl)methyl)- 1,3,4- oxadiazol-2- yl)-5- (trifluoromethyl) | 74 | 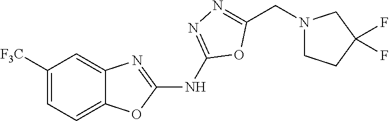 [SMILES: FC1(F)CCN(Cc2nnc(Nc3nc4cc(C(F)(F)F)ccc4o3)o2)C1] | 3% | MS (ESI+) for CHNOS m/z 390.28 [M + H]⁺; LC purity 98.9% (Ret. Time- 4.74 min); ¹H NMR (400 MHz, DMSO-d₆): δ 7.69-7.76 (m, 2H), 7.61 (d, J = 8.5 Hz, |
| benzo[d] | | | | 1H), 3.94 (s, 2H), |
| oxazol-2-amine | | | | 3.05-3.13 (m, 2H), |
| | | | | 2.86-2.91 (m, 2H), |
| | | | | 2.23-2.36 (m, 2H). |
| N-(5-((3- Methoxy- pyrrolidin-1- yl)methyl)- 1,3,4- oxadiazol-2- yl)-5- (trifluoromethyl) | 75 | 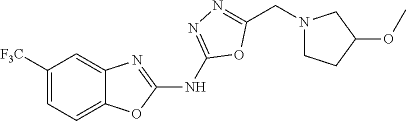 [SMILES: COC1CCN(Cc2nnc(Nc3nc4cc(C(F)(F)F)ccc4o3)o2)C1] | 3% | MS (ESI+) for CHNOS m/z 384.23 [M + H]⁺; LC purity 91.7% (Ret. Time- 4.28 min); ¹H NMR (400 MHz, DMSO-d₆ + D₂O): δ 7.70 (d, J = 8.6 Hz, 1H), 7.61 (s, |
| benzo[d] | | | | 1H), 7.65 (d, J = 8.6 |
| oxazol-2-amine | | | | Hz, 1H), 4.64 (s, 2H), |
| | | | | 4.13 (s, 1H), 3.40- |
| | | | | 3.58 (m, 4H), 3.21 (s, |
| | | | | 3H), 2.17 (bs, 1H), |
| | | | | 2.09 (bs, 1H). |
| 1-((5-((5- (triFluoromethyl) benzo[d]oxazol- 2-yl)amino)- 1,3,4- oxadiazol-2- yl)methyl) pyrrolidine-3- | 76 | 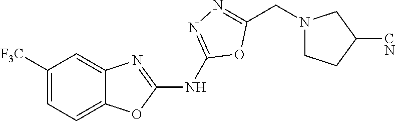 [SMILES: N#CC1CCN(Cc2nnc(Nc3nc4cc(C(F)(F)F)ccc4o3)o2)C1] | 3% | MS (ESI+) for CHNOS m/z 379.22 [M + H]⁺; LC purity 95.8% (Ret. Time- 4.39 min); ¹H NMR (400 MHz, DMSO-d₆): δ 7.74 (d, J = 8.5 Hz, 1H), 7.71 (s, 1H), |
| carbonitrile | | | | 7.62 (d, J = 8.5 Hz, |
| | | | | 1H), 4.14 (s, 2H), |
| | | | | 3.41 (bs, 1H), 2.70- |
| | | | | 3.20 (m, 4H), 2.28- |
| | | | | 2.34 (m, 1H), 2.01- |
| | | | | 2.09 (m, 1H). |
| 6-Chloro-N- (5-(pyrrolidin- 1-ylmethyl)- 1,3,4- oxadiazol-2- yl)benzo[d] oxazol-2-amine | 77 | 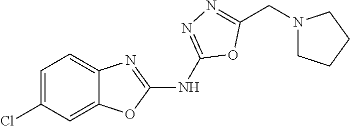 [SMILES: Clc1ccc2nc(Nc3nnc(CN4CCCC4)o3)oc2c1] | 2% | MS (ESI+) for CHNOS m/z 320.20 [M + H]⁺; LC purity 98.7% (Ret. Time- 4.18 min); ¹H NMR (400 MHz, DMSO-d₆): δ 7.67 (d, J = 1.7 Hz, 1H), 7.38 (d, J = 8.4 |
| | | | | Hz, 1H), 7.30 (dd, J = |
| | | | | 8.5, 1.8 Hz, 1H), 3.84 |
| | | | | (s, 2H), 2.61 (bs, 4H), |
| | | | | 1.73 (bs, 4H). |
| N-(5-((3- methylpyrrolidin- 1-yl)methyl)- 1,3,4- oxadiazol-2- yl)-5- (trifluoromethyl) benzo[d] | 78 | 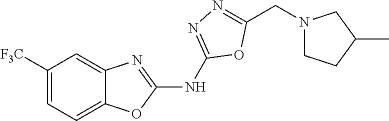 [SMILES: CC1CCN(Cc2nnc(Nc3nc4cc(C(F)(F)F)ccc4o3)o2)C1] | 2% | MS (ESI+) for CHNOS m/z 368.0 [M + H]⁺; LC purity 97.3% (Ret. Time-5.90 min); ¹H NMR (400 MHz, CD₃OD): δ 7.60-7.71 (m, 3H), 4.71 (s, 2H), |
| oxazol-2-amine | | | | 3.50-3.80 (m, 3H), |
| | | | | 3.06 (bs, 1H), 2.54 |
| | | | | (bs, 1H), 2.30 (bs, |
| | | | | 1H), 1.74 (bs, 1H), |
| | | | | 1.16 (s, 3H). |
| N-(5-((3- Fluoropyrrolidin- 1-yl)methyl)- 1,3,4- oxadiazol-2- yl)-5- (trifluoromethyl) benzo[d] | 79 | 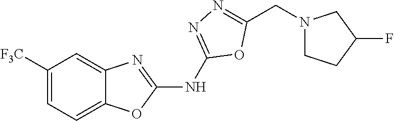 [SMILES: FC1CCN(Cc2nnc(Nc3nc4cc(C(F)(F)F)ccc4o3)o2)C1] | 4% | MS (ESI+) for CHNOS m/z 372.36 [M + H]⁺; LC purity 99.1% (Ret. Time- 4.45 min); ¹H NMR (400 MHz, DMSO-d₆₊ dTFA): δ 7.72 (d, J = 8.8 Hz, 1H), 7.71 (s, |
| oxazol-2-amine | | | | 1H), 7.61 (d, J = |
| | | | | 8.8 Hz, 1H), 5.54 (s, |
| | | | | 0.5H), 5.42 (s, 0.5H), |
| | | | | 4.83 (s, 2H), 3.48- |
| | | | | 4.01 (m, 4H), 2.26 |
| | | | | (bs, 2H). |
| 6-Chloro-N- (5-(pyrrolidin- 1-ylmethyl)- 1,3,4- oxadiazol-2- yl)-5- (trifluoromethyl) benzo[d] oxazol-2-amine | 80 | 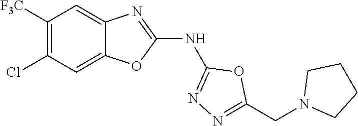 [SMILES: FC(F)(F)c1cc2nc(Nc3nnc(CN4CCCC4)o3)oc2cc1Cl] | 21% | MS (ESI+) for CHNOS m/z 388.2 [M + H]⁺; LC purity 97.7% (Ret. Time- 2.70); ¹H NMR (400 MHz, DMSO-d₆): δ 10.99 (bs, 1H), 8.07 (s, 1H), 7.79 (s, 1H), 4.72 (s, 2H), 3.38 |
| | | | | (bs, 4H), 1.97 (bs, 4H). |
| 6-Chloro-N- (5-((dimethyl- amino)methyl)- 1,3,4- oxadiazol-2- yl)benzo[d] oxazol-2-amine | 81 | 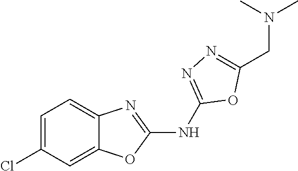 [SMILES: CN(C)Cc1nnc(Nc2nc3ccc(Cl)cc3o2)o1] | 2% | MS (ESI+) for CHNOS m/z 294.25 [M + H]⁺; LC purity 99.8% (Ret. Time- 4.06); ¹H NMR (400 MHz, DMSO-d₆) δ 7.77 (d, J = 1.8 Hz, 1H), 7.44 (d, J = 8.4 Hz, 1H), 7.38 (dd, J = 1.8, 8.4 Hz, 1H), 4.52 (s, 2H), 2.81 (s, 6H). |
| N-(5- ((Dimethyl- amino)methyl)- 1,3,4- oxadiazol-2- yl)-5- (trifluoromethyl) benzo[d] oxazol-2-amine | 82 | 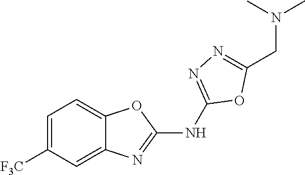 [SMILES: CN(C)Cc1nnc(Nc2nc3cc(C(F)(F)F)ccc3o2)o1] | 2% | MS (ESI+) for CHNOS m/z 328.14 [M + H]⁺; LC purity 99.2% (Ret. Time- 4.62); ¹H NMR (400 MHz, DMSO-d₆) δ 7.76 (d, J = 8 4 Hz, 1H), 7.71 (s, 1H), 7.65 (d, J = 8.4 Hz, 1H), 4.56 (s, 2H), 2.84 (s, 6H). |

### Synthetic Route 6

### 5-Methyl-N-(6-(trifluoromethyl)-1H-benzo[d]imidazol-2-yl)-1,3,4-oxadiazol-2-amine (Example 83)

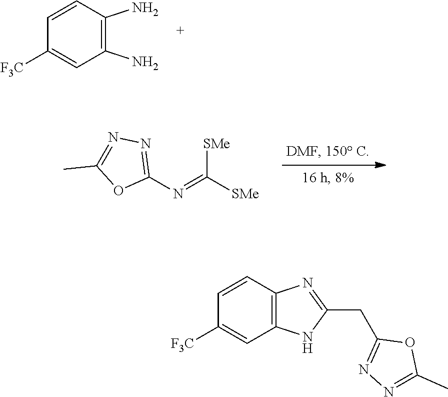 [SMILES: Nc1ccc(C(F)(F)F)cc1N ; CSC(=Nc1nnc(C)o1)SC ; Cc1nnc(Cc2nc3ccc(C(F)(F)F)cc3[nH]2)o1]

A reaction mixture of 4-(trifluoromethyl)benzene-1,2-diamine (500 mg, 2.84 mmol) and dimethyl (5-methyl-1,3,4-oxadiazol-2-yl)carbonimidodithioate (576 mg, 2.84 mmol) in DMF (5 mL) was stirred at 150° C. for 16 h. The TLC showed reaction to be complete. The reaction mixture was cooled to rt and poured into ice-water (50 mL). The solid precipitated was filtered, washed with H₂O (100 mL), triturated with Et2O (25 mL) and dried under reduced pressure to obtain 5-methyl-N-(6-(trifluoromethyl)-1H-benzo[d]imidazol-2-yl)-1,3,4-oxadiazol-2-amine as an off white solid. Yield: 62 mg (8.0%); ¹H NMR (400 MHz, DMSO-d₆): δ 12.1 (bs, 2H), 7.64 (s, 1H), 7.46 (bs, 2H), 2.38 (s, 3H); MS (ESI+) for CHNOS m/z 284.11 [M+H]⁺.

The following examples were prepared in a similar manner to 5-methyl-N-(6-(trifluoromethyl)-1H-benzo[d]imidazol-2-yl)-1,3,4-oxadiazol-2-amine following synthetic route 6.

| | | | | Spectral Data |
|---|---|---|---|---|
| Name | Ex | Structure | Yield | 1H NMR & LCMS |
| Methyl 2-((5- methyl-1,3,4- oxadiazol-2- yl)amino)-1H- benzo[d] imidazole-5- carboxylate | 84 | 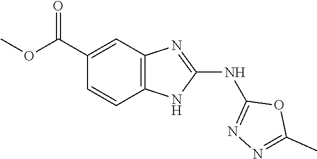 | 24% | MS (ESI+) for CHNOS m/z 274.10 [M + H]⁺; LC purity 99.2% (Ret. Time-4.29 min); ¹H NMR (400 MHz, DMSO- d₆): δ 12.5 (bs, 2H), 7.93 (s, 1H), 7.78 (d, J = 8.0 Hz, 1H), 7.39 (d, J = 8.0 Hz, 1H), 3.86 (s, 3H), 2.39 (s, 3H). |
| N-(5-Chloro- 1H-benzo[d] imidazol-2-yl)- 5-methyl-1,3,4- oxadiazol-2- amine | 85 | 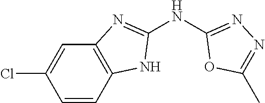 [SMILES: Cc1nnc(Nc2nc3cc(Cl)ccc3[nH]2)o1] | 26% | MS (ESI+) for CHNOS m/z 250.02 [M + H]⁺; LC purity 97.4% (Ret. Time-4.58 min); ¹H NMR (400 MHz, DMSO- d₆): δ 11.90 (bs, 2H), 7.36 (s, 1H), 7.29 (d, J = 8.3 Hz, 1H), 7.14 (d, J = 8.0 Hz, |
| | | | | 1H), 2.37 (s, 3H). |
| 5-Methyl-N- (5-(trifluoro- methyl)-1H- benzo[d] imidazol-2-yl)- 1,3,4- thiadiazol-2- | 86 | 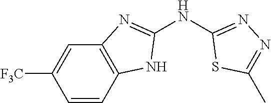 [SMILES: Cc1nnc(Nc2nc3cc(C(F)(F)F)ccc3[nH]2)s1] | 18% | MS (ESI+) for CHNOS m/z 300.10 [M + H]⁺; LC purity 99.2% (Ret. Time-5.10 min); ¹H NMR (400 MHz, DMSO-d₆): δ 12.05 (bs, 2H), 7.63 (s, 1H), 7.48 (d, J = 7.6 Hz, 1H), 7.38 (d, J = |
| amine | | | | 7.6 Hz, 1H), 2.50 (s, 3H). |
| N-(4-Fluoro- 1H- benzo[d] imidazol-2-yl)- 5-methyl-1,3,4- oxadiazol-2- amine | 87 | 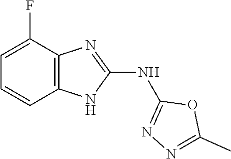 [SMILES: Cc1nnc(Nc2nc3c(F)cccc3[nH]2)o1] | 10% | MS (ESI+) for CHNOS m/z 234.11 [M + H]⁺; LC purity 99.7% (Ret. Time-4.09 min); ¹H NMR (400 MHz, DMSO-d₆): δ 12.50 (bs, 2H), 7.31 (d, J = 7.6 Hz, 1H), 7.96-7.12 (m, 2H), 2.37 (s, 3H). |
| 5-Methyl-N- (1-methyl-5- (trifluoro- methyl)-1H- benzo[d] imidazol-2-yl)- 1,3,4- | 88 | 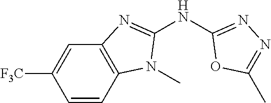 [SMILES: Cc1nnc(Nc2nc3cc(C(F)(F)F)ccc3n2C)o1] | 16% | MS (ESI+) for CHNOS m/z 298.08 [M + H]⁺; LC purity 97.8% (Ret. Time-5.46 min); ¹H NMR (400 MHz, DMSO-d₆): δ 12.01 (bs, 1H), 7.8 (s, 1H), 7.55 (s, 2H), 3.58 (s, 3H), 2.39 (s, 3H). |
| oxadiazol-2- | | | | |
| amine | | | | |
| 5-Methyl-N- (1-methyl-6- (trifluoro- methyl)-1H- benzo[d] imidazol-2-yl)- 1,3,4- oxadiazol-2- | 89 | 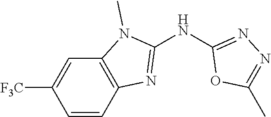 [SMILES: Cc1nnc(Nc2nc3ccc(C(F)(F)F)cc3n2C)o1] | 4% | MS (ESI+) for CHNOS m/z 298.12 [M + H]⁺; LC purity 98% (Ret. Time-5.45 min); ¹H NMR (400 MHz, DMSO- d₆): δ 7.79 (s, 1H), 7.66 (d, J = 8.7 Hz, 1H), 7.50 (d, J = 8.7 Hz, 1H), 3.59 (s, 3H), 2.39 (s, 3H) Hz, 1H). |
| amine | | | | |
| N-(1,4- Dimethyl-1H- benzo[d] imidazol-2-yl)- 5-methyl-1,3,4- oxadiazol-2- amine | 90 | 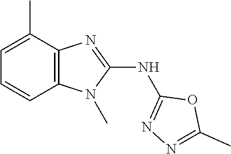 [SMILES: Cc1nnc(Nc2nc3c(C)cccc3n2C)o1] | 5% | MS (ESI+) for CHNOS m/z 244.15 [M + H]⁺; LC purity 99.2% (Ret. Time-4.86 min); ¹H NMR (400 MHz, DMSO- d₆): δ 7.25 (d, J = 7.9 Hz, 1H), 7.13-7.19 (m, 1H), 7.02 (d, J = 7.6 Hz, 1H), 3.55 (s, 3H), 2.47 (s, 3H), 2.39 (s, 3H). |

### Synthetic Route 7

### N-(6-Chloro-1H-benzo[d]imidazol-2-yl)-1,3,4-oxadiazol-2-amine (Example 91)

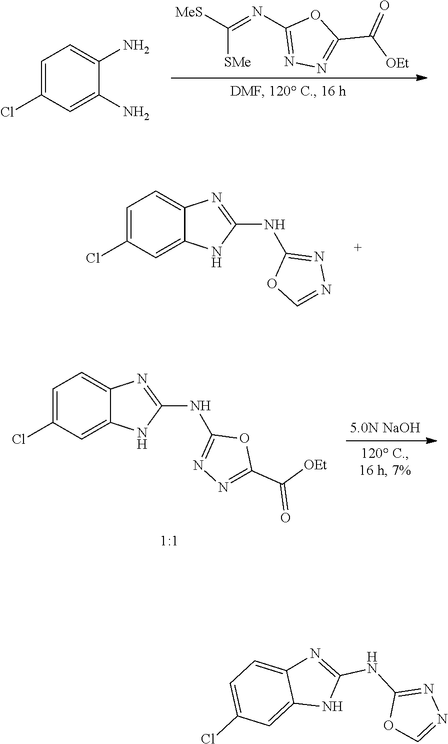 [SMILES: Nc1ccc(Cl)cc1N ; CCOC(=O)c1nnc(N=C(SC)SC)o1 ; Clc1ccc2nc(Nc3nnco3)[nH]c2c1 ; CCOC(=O)c1nnc(Nc2nc3ccc(Cl)cc3[nH]2)o1 ; Clc1ccc2nc(Nc3nnco3)[nH]c2c1]

A reaction mixture of 4-(trifluoromethyl)benzene-1,2-diamine (500 mg, 2.84 mmol) and dimethyl (5-methyl-1,3,4-oxadiazol-2-yl)carbonimidodithioate (576 mg, 2.84 mmol) in DMF (5 mL) was stirred at 120° C. for 16 h. The TLC showed complete consumption of SMs. The reaction mixture was cooled to rt and poured into ice-water (50 mL). The solid precipitated was filtered, washed with H₂O (30 mL) and dried to afford mixture of N-(6-chloro-1H-benzo[d]imidazol-2-yl)-1,3,4-oxadiazol-2-amine and ethyl 5-((6-chloro-1H-benzo[d]imidazol-2-yl)amino)-1,3,4-oxadiazole-2-carboxylate in 1:1 ratio as a brown solid. To this residue in DMF (5 mL) was added 5 N NaOH solution (5 mL). The resulted reaction mixture was stirred at 120° C. for 16 h. The TLC showed reaction to be complete. The reaction mixture was diluted with H₂O (50 mL) and extracted with EtOAC (3×20 mL). The aqueous layer was acidified to pH 1 with 1N HCl solution and extracted with EtOAC (3×20 mL). The organic layer was washed with brine (50 mL), dried (Na₂SO₄), filtered and concentrated under reduced pressure. The residue was purified by column chromatography using silica gel (100-200 mesh), eluting with 5% MeOH in DCM to afford N-(6-chloro-1H-benzo[d]imidazol-2-yl)-1,3,4-oxadiazol-2-amine as a brown solid. Yield: 60 mg (7.%); ¹H NMR (400 MHz; DMSO-d₆): δ 12.70 (bs, 1H), 12.42 (bs, 1H), 8.61 (s, 1H), 7.61 (bs, 1H), 7.58 (d, J=8.6 Hz, 1H), 7.23 (d, J=8.6 Hz, 1H); MS (ESI+) for CHNOS m/z 231.66 [M+H]⁺.

The following example was prepared in a similar manner to N-(6-chloro-1H-benzo[d]imidazol-2-yl)-1,3,4-oxadiazol-2-amine following synthetic route 7.

| | | | | Spectral Data |
|---|---|---|---|---|
| Name | Ex | Structure | Yield | 1H NMR & LCMS |
| N-(6-Chloro-1- methyl-1H- benzo[d] imidazol-2-yl)-1,3,4- oxadiazol-2- amine | 92 | 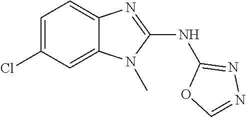 [SMILES: Cn1c(Nc2nnco2)nc2ccc(Cl)cc21] | 7% | MS (ESI+) for CHNOS m/z 250.03 [M + H]⁺; LC purity 98.5% (Ret. Time-4.29 min); ¹H NMR (400 MHz, DMSO-d₆): δ 12.26 (bs, 1H), 8.29 (s, 1H), 7.87 (d, J = 1.6 Hz, 1H), 7.64 (d, J = 8.6 Hz, 1H), 7.33 (dd, J = |
| | | | | 1.6, 8.6 Hz, 1H), 3.81 (s, 3H). |

### Synthetic Route 8

### N-(5-((Methylamino)methyl)-1,3,4-oxadiazol-2-yl)-5-(trifluoromethyl)benzo[d]oxazol-2-amine (Example 93)

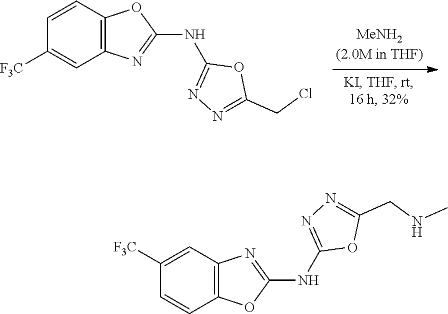 [SMILES: FC(F)(F)c1ccc2oc(Nc3nnc(CCl)o3)nc2c1 ; CNCc1nnc(Nc2nc3cc(C(F)(F)F)ccc3o2)o1]

To a solution of N-(5-(chloromethyl)-1,3,4-oxadiazol-2-yl)-5-(trifluoromethyl)benzo[d]oxazol-2-amine (250 mg, 0.786 mmol) in methylamine (2M in THF, 25 mL) was added KI (261 mg, 1.57 mmol) at rt for 16 h. The TLC showed reaction to be complete. The solvent was removed under reduced pressure. The crude residue was purified by prep to afford N-(5-((methylamino)methyl)-1,3,4-oxadiazol-2-yl)-5-(trifluoromethyl)benzo[d]oxazol-2-amine as an off white solid. Yield: 95 mg (32%); MS (ESI+) for CHNOS m/z 314.21 [M+H]⁺; LC purity 99.6% (Ret. Time—4.03); ¹H NMR (400 MHz, DMSO-d₆): δ 7.74 (d, J=8.4 Hz, 1H), 7.70 (bs, 1H), 7.63 (d, J=8.4 Hz, 1H), 4.48 (s, 2H), 2.69 (s, 3H).

### Synthetic Route 9

### N-Methyl-N-((5-((5-(trifluoromethyl)benzo[d]oxazol-2-yl)amino)-1,3,4-oxadiazol-2-yl)methyl)acetamide (Example 94)

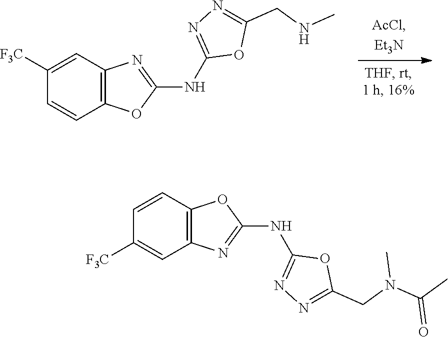 [SMILES: CNCc1nnc(Nc2nc3cc(C(F)(F)F)ccc3o2)o1 ; CC(=O)N(C)Cc1nnc(Nc2nc3cc(C(F)(F)F)ccc3o2)o1]

To a solution of N-(5-((methylamino)methyl)-1,3,4-oxadiazol-2-yl)-5-(trifluoromethyl)benzo[d]oxazol-2-amine (75 mg, 0.239 mmol) in THF (5 mL) at 0° C. were added Et₃N (0.1 mL, 0.718 mmol), followed by acetyl chloride in THF (21 mg, 0.264 mmol) slowly. The reaction mixture was stirred at rt for 1 h. The TLC showed reaction to be complete. The solvent was removed under reduced pressure. The residue was diluted with H₂O (20 mL) and extracted with EtOAc (3×20 mL). The organics were dried (Na₂SO₄), filtered and concentrated under reduced pressure to give the crude residue. The crude residue was purified by prep HPLC to afford N-methyl-N-((5-((5-(trifluoromethyl)benzo[d]oxazol-2-yl)amino)-1,3,4-oxadiazol-2-yl)methyl)acetamide as an off white solid. Yield: 14 mg (16%); MS (ESI+) for CHNOS m/z 356.23 [M+H]⁺; LC purity 94.3% (Ret. Time—5.29); ¹H NMR at 373 K (400 MHz, DMSO-d₆): δ 7.71 (s, 1H), 7.68 (d, J=8.4 Hz, 1H), 7.57 (d, J=8.4 Hz, 1H), 4.69 (s, 2H), 3.05 (bs, 3H), 2.09 (s, 3H).

### Synthetic Route 10

### Piperazin-1-yl(2-((5-(trifluoromethyl)benzo[d]oxazol-2-yl)amino)thiazol-4-yl)methanone hydrochloride (Example 95)

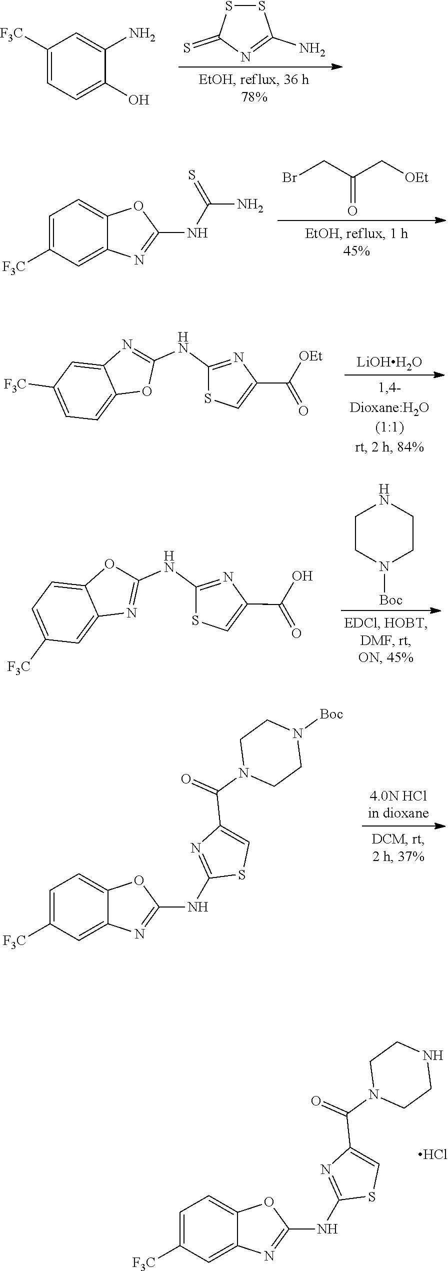

### 1-(5-(Trifluoromethyl)benzo[d]oxazol-2-yl)thiourea

To a stirred solution of 2-amino-4-(trifluoromethyl)phenol (2.3 g, 12.0 mmol) in EtOH (20 mL) was added xanthate hydride (2.33 g, 15.0 mmol). The reaction mixture was stirred at 100° C. for 36 h. The TLC showed reaction to be complete. The solvent was reduced to half volume under reduced pressure. The solid was filtered, triturated with diethyl ether (50 mL) and dried under reduced pressure to afford 1-(5-(trifluoromethyl)benzo[d]oxazol-2-yl)thiourea as an off white solid. Yield: 3.0 g (88%); ¹H NMR (400 MHz, DMSO-d₆): δ 12.44 (s, 1H), 9.66 (s, 1H), 9.57 (s, 1H), 7.96 (1H), 7.84 (d, J=8.4 Hz, 1H), 7.64 (d, J=8.4 Hz, 1H); MS (ESI+) for CHNOS m/z 260.15 [M−H]⁺.

### Ethyl 2-((5-(trifluoromethyl)benzo[d]oxazol-2-yl)amino)thiazole-4-carboxylate

To a stirred solution of 1-(5-(trifluoromethyl)benzo[d]oxazol-2-yl)thiourea (1.1 g, 4.2 mmol) at 100° C. was added ethyl bromo pyruvate (0.82 mL, 5.5 mmol) and the reaction mixture was stirred at 100° C. for 0.5 h. The TLC showed reaction to be complete. The reaction mixture was cooled to rt and stirred for 15 min. The solid precipitated was filtered and washed with diethyl ether (20 mL) to afford ethyl 2-((5-(trifluoromethyl)benzo[d]oxazol-2-yl)amino)thiazole-4-carboxylate as off white solid. Yield: 3.0 g (88%); ¹H NMR (400 MHz, DMSO-d₆): δ 13.57 (bs, 1H), 8.07 (s, 1H), 7.89 (s, 1H), 7.78 (d, J=8.0 Hz, 1H), 7.57 (d, J=8.0 Hz, 1H), 4.29 (q, J=6.8 Hz, 2H), 1.31 (t, J=6.8 Hz, 3H); MS (ESI+) for CHNOS m/z 358.13 [M+H]⁺.

### 2-((5-(Trifluoromethyl)benzo[d]oxazol-2-yl)amino)thiazole-4-carboxylic acid

To the solution of ethyl 2-((5-(trifluoromethyl)benzo[d]oxazol-2-yl)amino)thiazole-4-carboxylate (400 mg, 1.12 mmol) in 1,4-dioxane (10 mL) was added a solution of LiOH (328 mg, 7.82 mmol) in H₂O (10 mL) at rt. The reaction was stirred further for 2 h. The reaction mixture was poured into ice-water (20 mL) and extracted with EtOAc (3×20 mL). The aqueous layer was acidified to pH 1 using 1N HCl solution. The precipitated solid was filtered, washed with water (25 mL) and dried under vacuum to give 2-((5-(trifluoromethyl)benzo[d]oxazol-2-yl)amino)thiazole-4-carboxylic acid as off white solid. Yield: 310 mg (84%); ¹H NMR (400 MHz, DMSO) δ 13.37 (bs, 1H), 7.93 (s, 1H), 7.87 (s, 1H), 7.75 (d, J=8.2 Hz, 1H), 7.55 (d, J=8.2 Hz, 1H); MS (ESI+) for CHNOS m/z 278.10 [M+H]⁺.

### tert-Butyl 4-(2-((5-(trifluoromethyl)benzo[d]oxazol-2-yl)amino)thiazole-4-carbonyl)piperazine-1-carboxylate

To the stirred solution of 2-((5-(trifluoromethyl)benzo[d]oxazol-2-yl)amino)thiazole-4-carboxylic acid (310 mg, 0.94 mmol) in DMF (5 mL) were added EDCl (269 mg, 1.41 mmol), HOBt (190 mg, 1.41 mmol), DiPEA (0.5 mL, 2.83 mmol) and 1-Boc-piperazine (262 mg, 1.41 mmol). The reaction mixture was stirred at rt for 12 h. The TLC showed the reaction to be complete. The reaction mixture was diluted with DCM (20 mL) and subsequently washed with 1N HCl (20 mL), aq. NaHCO₃ (20 mL) and brine solution (20 mL). The organics were dried (Na₂SO₄), filtered and concentrated under reduced pressure to give the crude residue. The crude material was triturated with Et₂O (20 mL), filtered and dried under vacuum to tert-butyl 4-(2-((5-(trifluoromethyl)benzo[d]oxazol-2-yl)amino)thiazole-4-carbonyl)piperazine-1-carboxylate as an off white solid. Yield: 200 mg (45%); ¹H NMR (400 MHz, DMSO-d₆): δ 13.28 (bs, 1H), 7.87 (s, 1H), 7.75 (d, J=8.6 Hz, 1H), 7.52-7.56 (m, 2H), 3.63 (bs, 4H), 3.39 (bs, 4H), 1.42 (s, 9H); MS (ESI+) for CHNOS m/z 498.16 [M+H]⁺.

### Piperazin-1-yl(2-((5-(trifluoromethyl)benzo[d]oxazol-2-yl)amino)thiazol-4-yl)methanone hydrochloride

To a solution of tert-butyl 4-(2-((5-(trifluoromethyl)benzo[d]oxazol-2-yl)amino)thiazole-4-carbonyl)piperazine-1-carboxylate (200 mg, 0.40 mmol) in CH₂Cl₂ (10.0 mL) was added 4 N HCl in 1,4-dioxane (10 mL) and stirred at rt for 1 h. The TLC showed reaction to be complete. The reaction mixture was concentrated under reduced pressure. The residue was triturated with Et₂O (10 mL), filtered and dried under vacuum to afford piperazin-1-yl(2-((5-(trifluoromethyl)benzo[d]oxazol-2-yl)amino)thiazol-4-yl)methanone hydrochloride as an off white solid. Yield: 60 mg (37%); ¹H NMR (400 MHz, DMSO-d₆): δ9.37 (bs, 2H), 7.89 (s, 1H), 7.78 (d, J=8.4 Hz, 1H), 7.69 (s, 1H), 7.57 98 (d, J=8.4 Hz, 1H), 3.77 (bs, 4H), 3.17 (bs, 4H); MS (ESI+) for CHNOS m/z 398.34 [M+H]⁺.

The following intermediate was prepared in a similar manner to 1-(5-(trifluoromethyl)benzo[d]oxazol-2-yl)thiourea.

| | | | | Spectral Data |
|---|---|---|---|---|
| Name | Int | Structure | Yield | 1H NMR & LCMS |
| 1-(5- Chlorobenzo[d] oxazol-2- yl)thiourea | 120 | 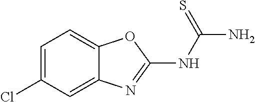 [SMILES: NC(=S)Nc1nc2cc(Cl)ccc2o1] | 60% | MS (ESI+) for CHNOS m/z 226.0 [M − H]; ¹H NMR (400 MHz, DMSO-d₆): δ 12.34 (s, 1H), 9.54 (s, 2H), 7.61-7.66 (m, 2H), 7.30 (d, J = 8.4 Hz, 1H). |

The following intermediate was prepared in a similar manner to ethyl 2-((5-(trifluoromethyl)benzo[d]oxazol-2-yl)amino)thiazole-4-carboxylate.

| | | | | Spectral Data |
|---|---|---|---|---|
| Name | Int | Structure | Yield | 1H NMR & LCMS |
| Ethyl 2-((5- chlorobenzo[d] oxazol-2- yl)amino)thiazole- 4-carboxylate | 121 | 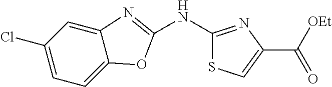 [SMILES: CCOC(=O)c1csc(Nc2nc3cc(Cl)ccc3o2)n1] | 60% | MS (ESI+) for CHNOS m/z 324.14 [M + H]⁺; ¹H NMR (400 MHz, DMSO- d₆): δ 13.45 (bs, 1H), 8.05 (s, 1H), 7.57-7.61 (m, 2H), 7.23 (d, J = 8.3 Hz, |
| | | | | 1H), 4.28 (q, J = 7.0 Hz, |
| | | | | 2H), 1.30 (t, J = 7.0 Hz, 3H). |

The following example was prepared in a similar manner to 2-((5-(trifluoromethyl)benzo[d]oxazol-2-yl)amino)thiazole-4-carboxylic acid following synthetic route 10.

| | | | | Spectral Data |
|---|---|---|---|---|
| Name | Ex | Structure | Yield | 1H NMR & LCMS |
| 2-((5- Chlorobenzo[d] oxazol-2- yl)amino) thiazole-4- carboxylic acid | 96 | 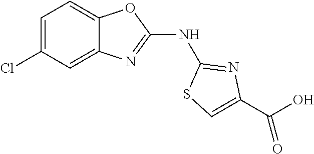 [SMILES: O=C(O)c1csc(Nc2nc3cc(Cl)ccc3o2)n1] | 48% | MS (ESI+) for CHNOS m/z 296.09 [M + H]+; 1H NMR (400 MHz, DMSO-d6): δ 12.82 (bs, 1H), 7.81 (bs, 1H), 7.55 (bs, 2H), 6.96 (bs, 1H). |

The following examples were prepared in a similar manner to tert-butyl 4-(2-((5-(trifluoromethyl)benzo[d]oxazol-2-yl)amino) thiazole-4-carbonyl) piperazine-1-carboxylate following synthetic route 10.

| | | | | Spectral Data |
|---|---|---|---|---|
| Name | Ex | Structure | Yield | 1H NMR & LCMS |
| 2-((5- Chlorobenzo[d] oxazol-2- yl)amino)-N-(2- (dimethylamino) ethyl)thiazole-4- carboxamide | 97 | 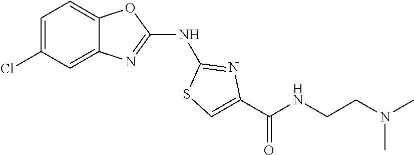 [SMILES: CN(C)CCNC(=O)c1csc(Nc2nc3cc(Cl)ccc3o2)n1] | 3% | MS (ESI+) for CHNOS m/z 366.16 [M + H]⁺; LC purity 98.5% (Ret. Time-4.80 min); ¹H NMR (400 MHz, DMSO-d₆): δ 9.40 (bs, 1H), 7.86 (bs, 1H), 7.32 (bs, 1H), 7.12 (bs, 1H), 6.85 (bs, 1H), 3.23 (bs, 2H), 2.40 (bs, 2H), 2.19 (s, 6H). |

### Synthetic Route 11

### N-(2-fluoropyridin-4-yl)-2-((5-(trifluoromethyl)benzo[d]oxazol-2-yl)amino)thiazole-4-carboxamide (Example 98)

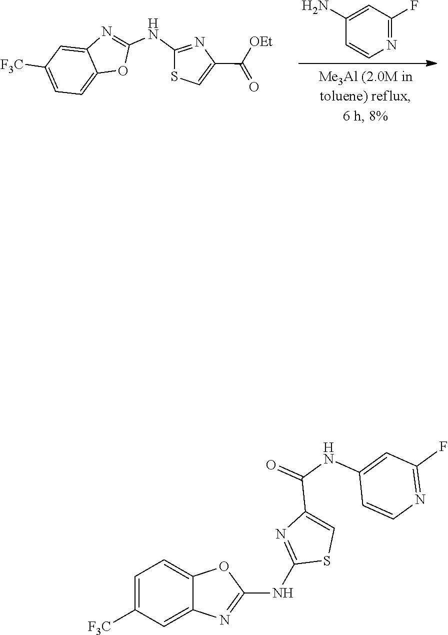 [SMILES: CCOC(=O)c1csc(Nc2nc3cc(C(F)(F)F)ccc3o2)n1 ; Nc1ccnc(F)c1 ; O=C(Nc1ccnc(F)c1)c1csc(Nc2nc3cc(C(F)(F)F)ccc3o2)n1]

To a solution of ethyl 2-((5-(trifluoromethyl)benzo[d]oxazol-2-yl)amino)thiazole-4-carboxylate (400 mg, 1.1 mmol) and 2-fluoropyridin-4-amine (125 mg, 1.1 mmol) was added Me₃Al (2M in toluene, 2.8 mL, 5.6 mmol) dropwise at rt. The reaction mixture was refluxed for 6 h. The TLC showed reaction to be complete. Reaction mixture was allowed to come to rt, poured in water (50 mL) and extracted with EtOAc (3×50 mL). The organic layer was washed with water (100 mL) and brine (50 mL). The organics were dried (Na₂SO₄), filtered and concentrated under reduced pressure to afford N-(2-fluoropyridin-4-yl)-2-((5-(trifluoromethyl)benzo[d]oxazol-2-yl)amino)thiazole-4-carboxamide as a brown solid. Yield: 40 mg (8.0%); ¹H NMR (400 MHz, DMSO-d₆): δ13.35 (bs, 1H), 10.72 (s, 1H), 8.16 (d, J=5.6 Hz, 1H), 8.12 (s, 1H), 7.83 (s, 1H), 7.75 (d, J=8.0 Hz, 1H), 7.66-7.72 (m, 1H), 7.62 (s, 1H), 7.56 (d, J=8.0 Hz, 1H); MS (ESI+) for CHNOS m/z 424.29 [M+H]⁺.

The following example was prepared in a similar manner to N-(2-fluoropyridin-4-yl)-2-((5-(trifluoromethyl)benzo[d]oxazol-2-yl)amino) thiazole-4-carboxamide following synthetic route 11.

| | | | | Spectral Data |
|---|---|---|---|---|
| Name | Ex | Structure | Yield | 1H NMR & LCMS |
| 2-((5-Chloro- benzo[d] oxazol-2- yl)amino) thiazole-4- carboxamide | 99 | 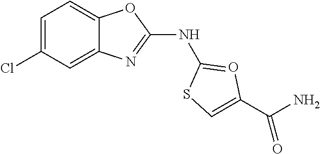 | 2% | MS (ESI+) for CHNOS m/z 295.18 [M + H]⁺; LC purity 99.6% (Ret. Time-5.29 min); ¹H NMR (400 MHz, DMSO-d₆): δ 8.94 (bs, 1H), 8.47 (bs, 1H), 6.75-7.40 (m, 5H). |

### Synthetic Route 12

### N-(4-Chlorothiazol-2-yl)-5-(trifluoromethyl)benzo[d]oxazol-2-amine (Example 100)

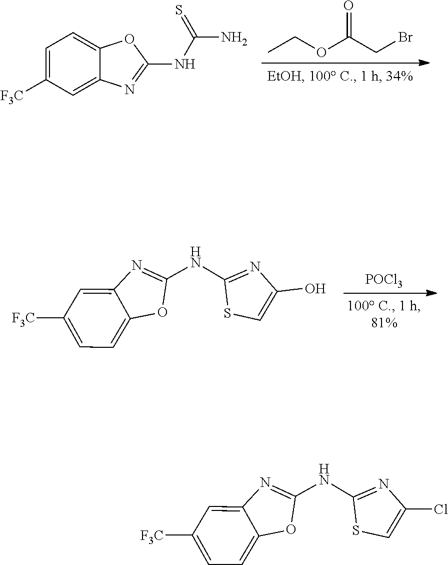

### 2-((5-(Trifluoromethyl)benzo[d]oxazol-2-yl)amino)thiazol-4-ol

To a stirred solution of 1-(5-(trifluoromethyl)benzo[d]oxazol-2-yl)thiourea (800 mg, 3.06 mmol) in EtOH (5 mL) at 100° C. was added ethyl bromopyruvate (665 mg, 3.98 mmol) and stirred at 100° C. for 30 min. The TLC showed reaction to be complete. The reaction mixture was allowed to cool to rt. The solid precipitated was filtered and washed with Et₂O to give 2-((5-(trifluoromethyl)benzo[d]oxazol-2-yl)amino)thiazol-4-ol as an off white solid. Yield: 300 mg (34%); ¹H NMR (400 MHz, DMSO-d₆): δ12.65 (bs, 1H), 8.01 (s, 1H), 7.82 (d, J=8.4 Hz, 1H), 7.65 (d, J=8.4 Hz, 1H), 4.14 (s, 2H); MS (ESI+) for CHNOS m/z 302.22 [M+H]⁺.

### N-(4-Chlorothiazol-2-yl)-5-(trifluoromethyl)benzo[d]oxazol-2-amine

A solution of 2-((5-(trifluoromethyl)benzo[d]oxazol-2-yl)amino)thiazol-4-ol (350 mg, 1.16 mmol) in POCl₃ (1.7 mL, 11.6 mmol) was heated at 100° C. for 1 h. The TLC showed reaction to be complete. The solvent was removed under reduced pressure. The residue was recrystalized with Et₂O to afford N-(4-chlorothiazol-2-yl)-5-(trifluoromethyl)benzo[d]oxazol-2-amine as brown solid. Yield: 300 mg (81%); ¹H NMR (400 MHz, DMSO-d₆): δ 13.42 (bs, 1H), 7.90 (s, 1H), 7.80 (d, J=8.3 Hz, 1H), 7.59 (d, J=8.3 Hz, 1H), 7.26 (s, 1H); MS (ESI+) for CHNOS m/z 320.18 [M+H]⁺.

### Synthetic Route 13

### 2-((6-(trifluoromethyl)benzo[d]oxazol-2-yl)amino)thiazole-4-carboxamide

### Example 101

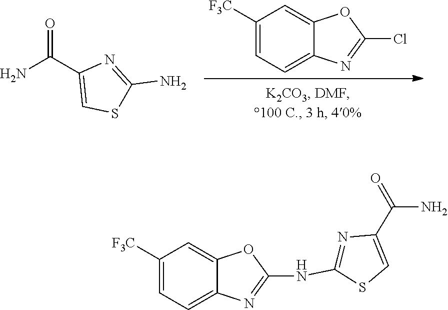 [SMILES: NC(=O)c1csc(N)n1 ; FC(F)(F)c1ccc2nc(Cl)oc2c1 ; NC(=O)c1csc(Nc2nc3ccc(C(F)(F)F)cc3o2)n1]

### 2-((6-(triFluoromethyl)benzo[d]oxazol-2-yl)amino)thiazole-4-carboxamide

To a solution of 2-chloro-6-(trifluoromethyl)benzo[d]oxazole (500 mg, 2.26 mmol) in DMF (8.0 mL) were added 2-aminothiazole-4-carboxamide (323 mg, 2.26 mmol) and K₂CO₃ (937 mg, 6.78 mmol). The resulting mixture was stirred at 100° C. for 3 h. TLC showed the reaction to be complete. The reaction mixture was poured in to ice water (50 mL). The solid precipitated was filtered and washed with water (50 mL) and dried by azeotropic distillation using toluene. Thus obtained solid was triturated with DCM (10 mL) followed by Et₂O (10 mL) and dried under vacuum. The solid was further purified by prep HPLC to afford 2-((6-(trifluoromethyl)benzo[d]oxazol-2-yl)amino)thiazole-4-carboxamide as an off white solid. Yield: 30 mg (4.0%); MS (ESI+) for CHNOS m/z 328.99 [M+H]⁺; LC purity 98.0% (Ret. Time—5.55 min); ¹H NMR (400 MHz, DMSO-d₆): δ 13.02 (s, 1H), 7.96 (s, 1H), 7.88 (bs, 1H), 7.78 (s, 1H), 7.58-7.73 (m, 3H).

### Intermediate 122

### 5-Amino-1,3,4-oxadiazole-2-carboxamide

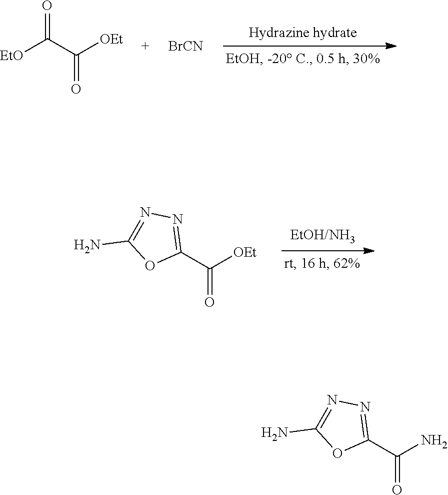

### Ethyl 5-amino-1,3,4-oxadiazole-2-carboxylate

To a solution of diethyl oxalate (30 g, 205 mmol) in EtOH (50 mL) was added hydrazine hydrate (8.1 mL) in EtOH (20 mL) drop wise at −20° C. The reaction mixture was stirred at −20° C. for 0.5 h and filtered. To filtrate was added water (15 mL) and cyanogen bromide (16.5 g, 164 mmol) at rt. The reaction mixture was stirred at rt for 1 h. The precipitated solid was filtered, washed with Et₂O (100 mL) and dried under vacuum to afford ethyl 5-amino-1,3,4-oxadiazole-2-carboxylate as a white solid. Yield: 10 g (31%); ¹H NMR (400 MHz, DMSO-d₆): δ7.78 (s, 2H), 4.32 (q, J=7.0 Hz, 2H), 1.29 (t, J=7.0 Hz, 3H); MS (ESI+) for CHNOS m/z 158.02 [M+H]⁺.

### 5-Amino-1,3,4-oxadiazole-2-carboxamide

To a solution of ethyl 5-amino-1,3,4-oxadiazole-2-carboxylate (1.5 g, 95 mmol) in EtOH (5.0 mL) at −78° C. in sealed tube was added EtOH/NH₃ (20.0 mL). The reaction mixture was stirred at rt for 16 h. The TLC showed reaction to be complete. The solid precipitated was filtered, washed with H₂O (10 mL) followed by Et₂O (10 mL) and dried under vacuum to afford 5-mino-1,3,4-oxadiazole-2-carboxamide as a white solid. Yield: 1.01 g (81%); ¹H NMR (400 MHz, DMSO-d₆): δ 8.13 (s, 1H), 7.80 (s, 1H), 7.50 (bs, 2H); MS (ESI+) for CHNOS m/z 128.92 [M+H]⁺.

The following example was prepared in a similar manner to 2-((6-(trifluoromethyl)benzo[d]oxazol-2-yl)amino)thiazole-4-carboxamide following synthetic route 13.

| | | | | Spectral Data |
|---|---|---|---|---|
| Name | Ex | Structure | Yield | 1H NMR & LCMS |
| 5-((6- (Trifluoro- methyl) benzo[d]oxazol- 2-yl)amino)- 1,3,4- oxadiazole- 2-carboxamide | 102 | 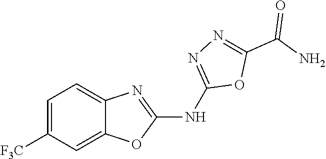 [SMILES: NC(=O)c1nnc(Nc2nc3ccc(C(F)(F)F)cc3o2)o1] | 3% | MS (ESI+) for CHNOS m/z 361.07 [M − H]⁺; LC purity 97.6% (Ret. Time- 5.43 min); ¹H NMR (400 MHz, DMSO-d₆): δ 8.03- 8.24 (m, 2H), 7.79-7.90 (m, 1H), 7.68 (bs, 1H), 7.54 (bs, 1H), 7.23-7.38 (m, 2H). |

### Intermediate 123

### tert-Butyl 4-(5-amino-1,3,4-oxadiazol-2-yl)piperidine-1-carboxylate

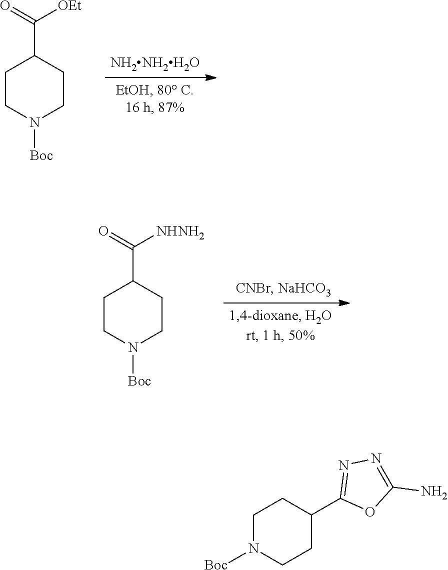 [SMILES: CCOC(=O)C1CCN(C(=O)OC(C)(C)C)CC1 ; CC(C)(C)OC(=O)N1CCC(C(=O)NN)CC1 ; CC(C)(C)OC(=O)N1CCC(c2nnc(N)o2)CC1]

### tert-Butyl 4-(hydrazinecarbonyl)piperidine-1-carboxylate

To a solution of 1-(tert-butyl) 4-ethyl piperidine-1,4-dicarboxylate (5 g, 19.4 mmol) in EtOH (50 mL) was added hydrazine hydrate (9.7 g, 19.4 mmol) dropwise. The mixture was refluxed for 16 h. The TLC showed reaction to be complete. The solvent was removed under reduced pressure. The residue was triturated with Et₂O (100 ml) to afford tert-butyl 4-(hydrazinecarbonyl)piperidine-1-carboxylate as off white solid. Yield: 4.1 g (87%); ¹H NMR (400 MHz, DMSO-d₆): δ8.99 (s, 1H), 3.91 (bs, 6H), 2.67 (bs, 2H), 2.17-2.25 (m, 1H), 1.56-1.61 (m, 2H), 1.44 (s, 9H); MS (ESI+) for CHNOS m/z 244.31 [M+H]⁺.

### tert-Butyl 4-(5-amino-1,3,4-oxadiazol-2-yl)piperidine-1-carboxylate

To a solution of tert-butyl 4-(hydrazinecarbonyl)piperidine-1-carboxylate (2 g, 80.0 mmol) in 1,4 dioxane (5 mL) was added NaHCO₃ (800 mg, 84.0 mmol), H₂O (1.0 mL) and BrCN (937 mg, 84.0 mmol) at rt. The reaction mixture was stirred at rt for 1 h. The TLC showed reaction to be complete. The reaction mixture poured into aq. sat. NaHCO₃ solution (50 mL) and extracted with EtOAc (3×50 mL) to afford tert-butyl 4-(hydrazinecarbonyl)piperidine-1-carboxylate as an off white solid. Yield: 1.1 g (50%); ¹H NMR (400 MHz, DMSO-d₆): δ 6.89 (s, 2H), 3.84-3.89 (m, 2H), 2.90-2.98 (m, 3H), 1.85-1.91 (m, 2H), 1.47-1.56 (m, 2H), 1.44 (s, 9H); MS (ESI+) for CHNOS m/z 268.29 [M+H]⁺.

### Intermediate 124

### 2-Bromo-5-(trifluoromethyl)benzo[d]oxazole

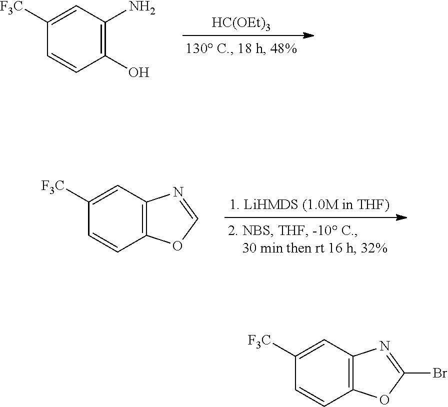

### 5-(triFluoromethyl)benzo[d]oxazole

A solution of 2-amino-4-(trifluoromethyl)phenol (5 g, 28.2 mmol) in triethoxymethane (30 g, 283 mmol) was heated at 130° C. for 5 h. The TLC showed reaction to be complete. The solvent was removed under reduced pressure. The residue was purified by column chromatography using silica gel (100-200 mesh), eluting with 4% EtOAc in hexane to afford 5-(trifluoromethyl)benzo[d]oxazole as a yellow solid. Yield: 2.5 g (48%); ¹H NMR (400 MHz, DMSO-d₆): δ8.20 (s, 1H), 8.10 (s, 1H), 7.63-7.74 (m, 2H).

### 2-Bromo-5-(trifluoromethyl)benzo[d]oxazole

To a solution of 5-(trifluoromethyl)benzo[d]oxazole (2 g, 10.98 mmol) in dry THF (20 mL) was added LiHMDS (6 mL, 1 M in THF, 32.96 mmol) at −10° C. slowly. The reaction mixture was stirred at −10° C. for 30 min and added NBS (2.8 g, 16.48 mmol). The reaction mixture was allowed to come to rt and stirred for 16 h. The TLC showed reaction to be complete. The reaction mixture was quenched with aq NH₄Cl solution (50 mL) and extracted with ethyl acetate (3×50 mL). The organic layer was washed with saturated aq NaHCO₃ solution (50 mL) followed by brine (50 mL), dried (Na₂SO₄), filtered and concentrated under reduced pressure to give the residue. The residue was purified by column chromatography using silica gel (100-200 mesh), eluting with 3% EtOAc in hexane to afford 2-bromo-5-(trifluoromethyl)benzo[d]oxazole as a white solid. Yield: 900 mg (32%); ¹H NMR (400 MHz, CDCl₃): δ7.99 (s, 1H), 759-7.69 (m, 2H).

The following intermediate was prepared in a similar manner to tert-butyl 4-(hydrazinecarbonyl)piperidine-1-carboxylate.

| | | | | Spectral Data |
|---|---|---|---|---|
| Name | Int | Structure | Yield | 1H NMR & LCMS |
| tert-Butyl 4-(2- hydrazinyl-2- oxoethyl) piperazine- 1-carboxylate | 125 | 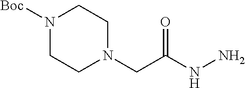 [SMILES: CC(C)(C)OC(=O)N1CCN(CC(=O)NN)CC1] | 72% | MS (ESI+) for CHNOS m/z 259.09 [M + H]⁺; ¹H NMR (400 MHz, DMSO-d₆ + D₂O): δ 3.29 (s, 4H), 2.92 (s, 2H), 2.33 (s, 4H), 1.35 (s, 9H). |

The following intermediates were prepared in a similar manner to tert-Butyl 4-(5-amino-1,3,4-oxadiazol-2-yl)piperidine-1-carboxylate.

| | | | | Spectral Data |
|---|---|---|---|---|
| Name | Int | Structure | Yield | 1H NMR & LCMS |
| tert-Butyl (R)-2- (5-amino-1,3,4- oxadiazol-2- yl)pyrrolidine-1- carboxylate | 126 | 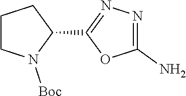 [SMILES: CC(C)(C)OC(=O)N1CCC[C@@H]1c1nnc(N)o1] | 72% | MS (ESI+) for CHNOS m/z 255.11 [M + H]⁺; ¹H NMR (400 MHz, DMSO-d₆): δ 6.93 (bs, 2H), 4-70-4.82 (m, 1H), 3.32 (s, 2H), 2.17-2.24 (m, 1H), 1.80 (bs, 3H), 1.39 (s, 4H), 1.26 (s, 5H). |
| tert-Butyl (S)-2- (5-amino-1,3,4- oxadiazol-2- yl)pyrrolidine-1- carboxylate | 127 | 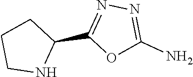 [SMILES: Nc1nnc([C@@H]2CCCN2)o1] | 82% | MS (ESI+) for CHNOS m/z 255.11 [M + H]⁺; 1H NMR (400 MHz, DMSO-d₆): δ 6.93 (bs, 2H), 4-70- 4.82 (m, 1H), 3.32 (s, 2H), 2.17- 2.24 (m, 1H), 1.80 (bs, 3H), 1.39 |
| | | | | (s, 4H), 1.26 (s, 5H). |
| tert-bBtyl 4-((5- amino-1,3,4- oxadiazol-2- yl)methyl) piperazine-1- | 128 | 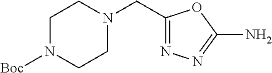 [SMILES: CC(C)(C)OC(=O)N1CCN(Cc2nnc(N)o2)CC1] | 34% | MS (ESI+) for CHNOS m/z 284.23 [M + H]⁺; 1H NMR (400 MHz, DMSO-d₆): δ 6.99 (bs, 2H), 3.69 (s, 2H), 3.29 (s, 4H), 2..37 (s, 4H), 1.38 (s, 9H). |
| carboxylate | | | | |
| tert-Butyl 4-(5- amino-1,3,4- oxadiazol-2- yl)piperazine-1- carboxylate | 129 | 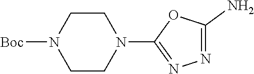 [SMILES: CC(C)(C)OC(=O)N1CCN(c2nnc(N)o2)CC1] | 51% | MS (ESI+) for CHNOS m/z 270.10 [M + H]⁺; 1H NMR (400 MHz, CDCl₃): δ 4.63 (bs, 2H), 3.50- 3.53 (m, 4H), 3.343.35 (m, 4H), 1.47 (s, 9H). |

### Intermediate 130

### 2-Chloro-6-(trifluoromethyl)oxazolo[4,5-c]pyridine

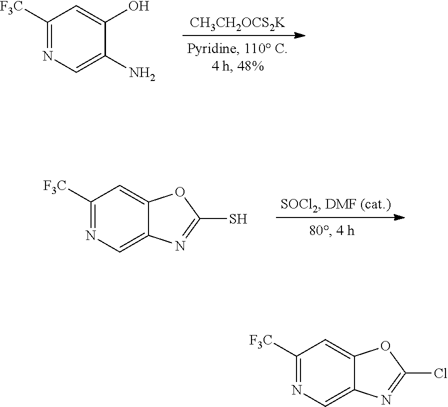

### 6-(Trifluoromethyl)oxazolo[4,5-c]pyridine-2-thiol

To a solution of 5-amino-2-(trifluoromethyl)pyridin-4-ol (2.0 g, 11.2 mmol) in pyridine (20 mL) was added potassium ethyl xanthate (2.2 g, 13.4 mmol) at rt. The reaction mixture was stirred at 110° C. for 4 h. The TLC showed reaction to be complete. Reaction mixture was cooled to rt and acidified to pH 4-5 by slow addition of 1.0N HCl. The reaction mixture was extracted with EtOAC (3×25 mL). The organics were dried (Na₂SO₄), filtered and concentrated under reduced pressure. The residue was triturated with Et₂O (25 mL) to give 6-(trifluoromethyl)oxazolo[4,5-c]pyridine-2-thiol as a brown solid. Yield: 1.1 g (50%); ¹H NMR (400 MHz, DMSO-d₆): δ 8.63 (s, 1H), 8.20 (s, 1H); MS (ESI+) for CHNOS m/z 220.93 [M+H]⁺.

### 2-Chloro-6-(trifluoromethyl)oxazolo[4,5-c]pyridine

To a solution of 6-(trifluoromethyl)oxazolo[4,5-c]pyridine-2-thiol (300 mg, 1.77 mmol) in SOCl₂ (3 mL) was added DMF (cat) at rt. The reaction mixture was stirred at 80° C. for 4 h. The TLC showed reaction to be complete. The solvent was removed under reduced pressure under N₂ to give 2-chloro-6-(trifluoromethyl)oxazolo[4,5-c]pyridine as brown liquid. Yield: 400 mg (crude). The crude was proceeded further without any purification.

### Intermediate 131

### 4-Amino-6-(trifluoromethyl)pyridin-3-ol

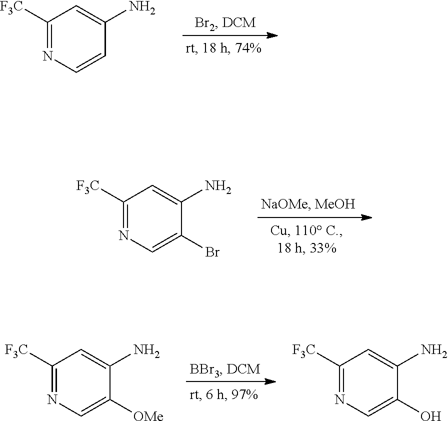

### 5-Bromo-2-(trifluoromethyl)pyridin-4-amine

To a solution of 2-(trifluoromethyl)pyridin-4-amine (10 g, 62.0 mmol) in DCM (150 mL) was added a solution of Br₂ in DCM (3.2 mL, 62.0 mmol) slowly at 0° C. The reaction mixture was further stirred at rt for 18 h. The TLC showed reaction to be complete. The reaction mixture was washed with saturated aq NaHCO₃ solution (200 mL) and H₂O (100 mL). The organics were dried (Na₂SO₄), filtered and concentrated under reduced pressure. The solid was purified by column chromatography using silica gel (100-200 mesh), eluting with DCM to afford 5-bromo-2-(trifluoromethyl)pyridin-4-amine as an off white solid. Yield: 11 g (74%); ¹H NMR (400 MHz, CDCl₃): δ8.47 (s, 1H), 6.97 (s, 1H), 4.92 (bs, 2H).

### 5-Methoxy-2-(trifluoromethyl)pyridin-4-amine

To a solution of 5-bromo-2-(trifluoromethyl)pyridin-4-amine (2.5 g, 10.4 mmol) in MeOH (10 mL) were added Cu powder (660 mg, 10.4) and freshly prepared sodium methoxide (2.5 g Na in 40 mL MeOH, 104 mmol) slowly in a sealed tube. The tube was sealed and reaction mixture was stirred at 100° C. for 18 h. The TLC showed reaction to be complete. The reaction mixture was filtered through a celite bed. The filtrate was concentrated under reduced pressure. The residue was purified by column chromatography using silica gel (100-200 mesh), eluting with DCM to afford 5-methoxy-2-(trifluoromethyl)pyridin-4-amine as a pink solid. Yield: 1.3 g (33%); ¹H NMR (400 MHz, DMSO-d₆): δ 8.02 (s, 1H), 6.93 (s, 1H), 4.38 (bs, 2H), 3.97 (s, 3H); MS (ESI+) for CHNOS m/z 193.24 [M+H]⁺.

### 4-Amino-6-(trifluoromethyl)pyridin-3-ol

To a solution of 5-methoxy-2-(trifluoromethyl)pyridin-4-amine (800 mg, 4.2 mmol) in DCM (10 mL) was added BBr₃ (1.2 mL, 12.5 mmol) slowly at 0° C. The reaction mixture was stirred at rt for 6 h. The TLC showed reaction to be complete. The solvent was removed under reduced pressure. The residue was basified to pH 8 by saturated aq. NaHCO₃ solution and extracted with EtOAc (3×25 mL). The organic layer was washed with H₂O (50 mL), dried (Na₂SO₄), filtered and concentrated under reduced pressure to afford 4-amino-6-(trifluoromethyl)pyridin-3-ol as a pink semi solid. Yield: 720 mg (97%); ¹H NMR (400 MHz, CDCl₃): δ7.94 (s, 1H), 6.91 (s, 1H), 4.71 (bs, 2H); MS (ESI+) for CHNOS m/z 179.23 [M+H]⁺.

### Intermediate 132

### N-Methyl-5-(trifluoromethyl)benzo[d]oxazol-2-amine

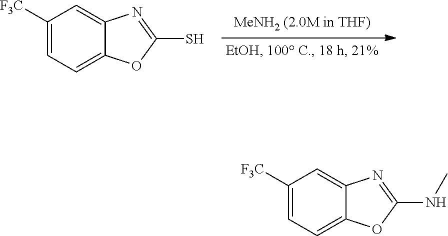

A mixture of 5-(trifluoromethyl)benzo[d]oxazole-2-thiol (1 g, 4.56 mmol) and methyl amine (2 M in THF) in EtOH (7 mL) was taken in a sealed tube. The tube was sealed and reaction mixture was stirred at 100° C. for 18 h. The TLC showed reaction to be complete. The solvent was removed under reduced pressure. Yield: 410 mg (29%); ¹H NMR (400 MHz, CDCl₃): δ 7.61 (s, 1H), 7.29 (bs, 2H), 4.95 (bs, 1H), 3.15 (d, J=4.6 Hz, 3H); MS (ESI+) for CHNOS m/z 217.0 [M+H]⁺.

The following intermediate was prepared in a similar manner to 6-(Trifluoromethyl) oxazolo[4,5-c]pyridine-2-thiol.

| | | | | Spectral Data |
|---|---|---|---|---|
| Name | Int | Structure | Yield | 1H NMR & LCMS |
| 4- Fluoro- benzo[d] oxazole- 2-thiol | 133 | 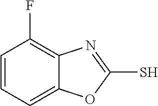 [SMILES: Fc1cccc2oc(S)nc12] | 60% | MS (ESI+) for CHNOS m/z 168.17 [M + H]⁺; ¹H NMR (400 MHz, DMSO-d₆): δ 14.49 (bs, 1H), 7.38 (d, J = 8.0 Hz, 1H), 7.09- 7.29 (m, 2H). |

The following intermediate was prepared in a similar manner to 2-chloro-6-(trifluoromethyl) oxazolo[4,5-c]pyridine.

| | | | | Spectral Data |
|---|---|---|---|---|
| Name | Int | Structure | Yield | 1H NMR & LCMS |
| 2-Chloro-4- fluoro- benzo[d] oxazole | 134 | 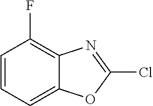 [SMILES: Fc1cccc2oc(Cl)nc12] | Used crude | Crude data showed desired product. |

### Intermediate 135

### 6-(Trifluoromethyl)oxazolo[4,5-c]pyridin-2-amine

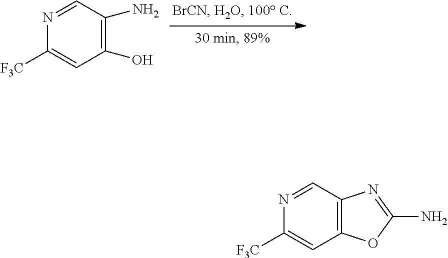 [SMILES: Nc1cnc(C(F)(F)F)cc1O ; Nc1nc2cnc(C(F)(F)F)cc2o1]

To a solution of 5-amino-2-(trifluoromethyl)pyridin-4-ol (500 mg, 2.80 mmol) in H₂O (5 mL) was added cyanogen bromide (442 mg, 4.21 mmol) at rt in portions. The resulting mixture was stirred at 100° C. for 30 min. The TLC showed reaction to be complete. The mixture was allowed to cool to room temperature, basified with aq NaHCO₃ solution and extracted with EtOAc (3×25 mL). The organic layer was dried (Na₂SO₄), filtered and concentrated under reduced pressure to afford 6-(trifluoromethyl)oxazolo[4,5-c]pyridin-2-amine as a brown solid. Yield: 510 (89%); ¹H NMR (400 MHz, DMSO-d₆): δ 8.58 (s, 1H), 8.16 (bs, 2H), 8.02 (s, 1H); MS (ESI+) for CHNOS m/z 202.23 [M−H]⁺.

### Intermediate 136

### 6-(Trifluoromethyl)oxazolo[5,4-b]pyridin-2-amine

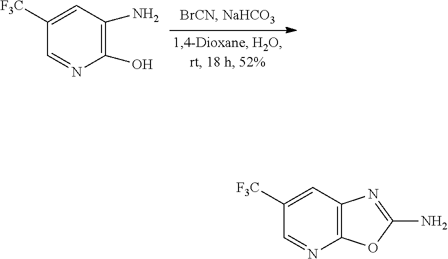 [SMILES: Nc1cc(C(F)(F)F)cnc1O ; Nc1nc2cc(C(F)(F)F)cnc2o1]

To a solution of 3-Amino-5-(trifluoromethyl)pyridin-2-ol (1.5 g, 8.4 mmol) in Dioxane:H₂O (7:3, 30 mL) were added sodium bicarbonate (3.5 g, 42 mmol) and cynaogen bromide (1.8 g, 16.8 mmol) at rt. The reaction mixture was stirred at rt for 18 h. The TLC showed reaction to be complete. The reaction mixture was diluted with aq. saturated NaHCO₃ (100 mL) and extracted with ETOAc (3×50 mL). The organic layer was dried (Na₂SO₄), filtered and concentrated under reduced pressure. The residue was triturated with Et₂O (25 mL) and dried under vacuum to gave 6-(trifluoromethyl)oxazolo[5,4-b]pyridin-2-amine as a light yellow solid. Yield: 890 mg (52%); ¹H NMR (400 MHz, DMSO-d₆): δ 8.24 (s, 1H), 8.13 (bs, 2H), 7.88 (s, 1H); MS (ESI−) for CHNOS m/z 202.06 [M−H]⁺.

### Intermediate 137

### 2-Amino-3-chlorophenol

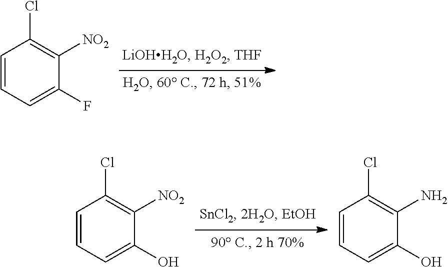 [SMILES: O=[N+]([O-])c1c(F)cccc1Cl ; O=[N+]([O-])c1c(O)cccc1Cl ; Nc1c(O)cccc1Cl]

### 3-Chloro-2-nitrophenol

To a solution of 1-chloro-3-fluoro-2-nitrobenzene (10 g, 57.1 mmol) in THF (65 mL) and H₂O (100 mL) mixture was added LiOH.H₂O (9.6 g, 22.8 mmol) at rt. The reaction mixture was sealed and stirred at 60° C. for 72 h. The reaction mixture was cooled to rt and poured in aq saturated sodium thiosulphate (100 mL) solution. The resulted mixture was acidified with 1N HCl and extracted with EtOAC (3×100 mL). The organic layer was washed with brine (100 mL), dried (Na₂SO₄), filtered and concentrated under reduced pressure. The residue was purified by column chromatography using silica gel (100-200 mesh), eluting with 10% EtOAc in hexane to afford 3-chloro-2-nitrophenol as a yellow liquid. Yield: 6.0 g (51%); ¹H NMR (400 MHz, DMSO-d₆): δ 11.50 (bs, 1H), 7.36-7.43 (m, 1H), 7.04-7.12 (m, 2H); MS (ESI−) for CHNOS m/z 172.07[M−H]⁺.

### 2-Amino-3-chlorophenol

To a solution of 3-chloro-2-nitrophenol (2.5 g, 14.5 mmol) in EtOH (30 mL) was added SnCl₂.2H₂O (13 g, 57.8 mmol) at rt. The reaction mixture was stirred at 90° C. for 2 h. The TLC showed reaction to be complete. The reaction mixture was cooled to rt and concentrated under reduced pressure. The ice-water (50 mL) was added to residue and basified to pH 7 with aq NH₃ solution. The mixture was extracted with EtOAc (3×50 mL). The organic layer was washed with brine (50 mL), dried (Na₂SO₄), filtered and concentrated under reduced pressure. The residue was triturated with hexane (25 mL) to afford 2-amino-3-chlorophenol as an off white solid. Yield: 1.8 g (80%); ¹H NMR (400 MHz, DMSO-d₆): δ9.58 (bs, 1H), 6.69 (d, J=8.0 Hz, 1H), 6.63 (d, J=8.0 Hz, 1H), 6.38-6.48 (m, 1H), 4.05 (bs, 2H); MS (ESI+) for CHNOS m/z 144.09 [M+H]⁺.

### Intermediate 138

### 5-(Pyrrolidin-1-ylmethyl)-4H-1,2,4-triazol-3-amine

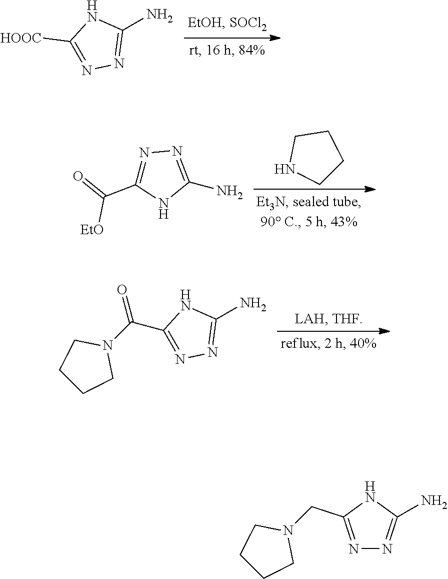 [SMILES: Nc1nnc(C(=O)O)[nH]1 ; CCOC(=O)c1nnc(N)[nH]1 ; C1CCNC1 ; Nc1nnc(C(=O)N2CCCC2)[nH]1 ; Nc1nnc(CN2CCCC2)[nH]1]

### Ethyl 5-amino-4H-1,2,4-triazole-3-carboxylate

To a solution of 5-amino-4H-1,2,4-triazole-3-carboxylic acid (3 g, 23.4 mmol) in EtOH (30 mL) was added thionyl chloride (6.8 mL, 93.6 mmol) slowly at rt. The reaction mixture was stirred at rt for 16 h. The TLC showed reaction to be complete. The reaction mixture was allowed to cool to rt and concentrated under vacuum. The residue was basified to pH 6 with saturated aqueous NaHCO₃ solution. The precipitated solid was filtered, washed with H₂O (100 mL) and dried under reduced pressure to obtain ethyl 5-amino-4H-1,2,4-triazole-3-carboxylate as an off white solid. Yield: 3.0 g (84%); ¹H NMR (400 MHz, DMSO-d₆): δ 12.63 (bs, 1H), 6.22 (bs, 2H), 4.21 (q, J=6.2 Hz, 2H), 1.25 (t, J=6.2 Hz, 3H); MS (ESI+) for CHNOS m/z 157.17 [M+H]⁺.

### (5-Amino-4H-1,2,4-triazol-3-yl)(pyrrolidin-1-yl)methanone

To a mixture of ethyl 5-amino-4H-1,2,4-triazole-3-carboxylate and pyrrolidine (2 g, 12.7 mmol) was added Et₃N (3.6 mL, 25.6 mmol) at rt. The reaction mixture was sealed and stirred at 90° C. for 5 h. The TLC showed reaction to be complete. The solvent was removed under reduced pressure. The residue was diluted with water (10 mL). The solid precipitated was filtered, washed with H₂O (10 mL) and dried under vacuum to afford 5-amino-4H-1,2,4-triazol-3-yl)(pyrrolidin-1-yl)methanone as an off white solid. Yield: 700 mg (30%); ¹H NMR (400 MHz, DMSO-d₆): δ 12.14 (bs, 1H), 6.93 (bs, 2H), 3.71 (s, 2H), 3.42 (bs, 2H), 1.79-1.85 (m, 4H); MS(ESI+) for CHNOS m/z 182.23[M+H]⁺.

### 5-(Pyrrolidin-1-ylmethyl)-4H-1,2,4-triazol-3-amine

To a solution of 5-amino-4H-1,2,4-triazol-3-yl)(pyrrolidin-1-yl)methanone (500 mg, 2.76 mmol) in dry THF (5 mL) was added LAH (2.3 mL, 2.4 M in THF, 5.5 mmol) slowly at 0° C. The reaction mixture warmed to rt and refluxed for 2 h. The TLC showed reaction to complete. The reaction mixture was cooled to rt and quenched with 10% aq NaOH slowly and filtered through small celite pad. The celite pad was washed with 10% MeOH in DCM (25 mL). The filtrate was concentrated under reduced pressure. The residue was purified by comb flash on C18 column to afford 5-(pyrrolidin-1-ylmethyl)-4H-1,2,4-triazol-3-amine as an off white solid. Yield: 700 mg (30%); ¹H NMR (400 MHz, DMSO-d₆+D₂O): 54.12 (s, 2H), 3.25 (bs, 4H), 1.90 (bs, 4H); MS (ESI+) for CHNOS m/z 168.29 [M+H]⁺.

The following intermediate was prepared in a similar manner to (5-amino-4H-1,2,4-triazol-3-yl) (pyrrolidin-1-yl) methanone.

| | | | | Spectral Data |
|---|---|---|---|---|
| Name | Int | Structure | Yield | 1H NMR & LCMS |
| (5-Amino-1,3,4- oxadiazol-2- yl)(pyrrolidin-1- yl)methanone | 139 | 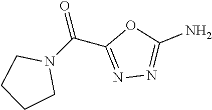 [SMILES: Nc1nnc(C(=O)N2CCCC2)o1] | 51% | MS (ESI+) for CHNOS m/z 183.17 [M + H]⁺; ¹H NMR (400 MHz, DMSO- d₆): δ 7.53 (bs, 2H), 3.80- 3.85 (m, 2H), 3.43-3.49 (m, 2H), 1.80-1.94 (m, 4H). |
| 5-Amino-N,N- dimethyl-4H- 1,2,4-triazole-3- carboxamide | 140 | 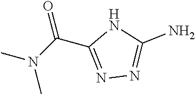 | Used crude | MS (ESI+) for CHNOS m/z 156.09 [M + H]⁺. |

The following intermediate was prepared in a similar manner to (5-(pyrrolidin-1-ylmethyl)-4H-1,2,4-triazol-3-amine.

| | | | | Spectral |
|---|---|---|---|---|
| | | | | Data |
| | | | | 1H NMR |
| Name | Int | Structure | Yield | & LCMS |
| 5-((Dimethyl- amino) methyl)-4H- 1,2,4- triazol-3- amine | 141 | 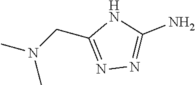 | Used crude | MS (ESI−) for CHNOS m/z 141.10 [M − H]⁺. |

### Intermediate 142

### 5-(Pyrrolidin-1-ylmethyl)-1,3,4-oxadiazol-2-amine

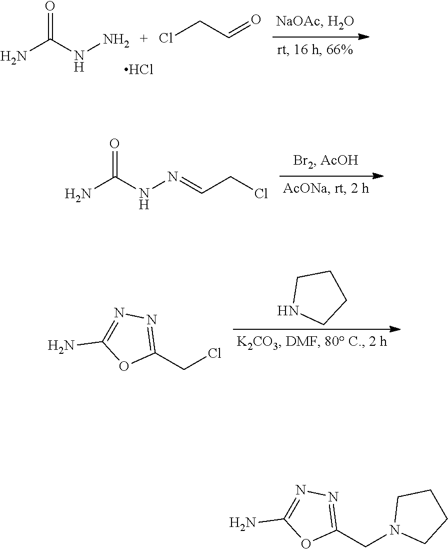

### (E)-2-(2-Chloroethylidene)hydrazine-1-carboxamide

To a solution of hydrazinecarboxamide hydrochloride (10 g, 90 mmol) in H₂O (100 mL) were added AcONa (11.1 g, 135 mmol) and 2-chloroacetaldehyde (50% in H₂O, 14.5 g, 180 mmol) slowly at rt. The reaction mixture was stirred at rt for 16 h.

The TLC showed reaction to be complete. The precipitated solid was filtered, washed with H₂O (200 mL) and dried under reduced pressure to afford (E)-2-(2-chloroethylidene)hydrazine-1-carboxamide as an off white solid. Yield: 8.0 g (66%); ¹H NMR (400 MHz, DMSO-d₆): δ 10.21 (bs, 1H), 7.18 (t, J=6.0 Hz, 1H), 6.31 (bs, 2H), 4.25 (d, J=6.0 Hz, 2H).

### 5-(Chloromethyl)-1,3,4-oxadiazol-2-amine

To a solution of (E)-2-(2-chloroethylidene)hydrazine-1-carboxamide (10 g, 74.0 mmol) and AcONa (60.7 g, 740 mmol) in glacial acetic acid (100 mL) was added Br₂ in AcOH (11.39 g, 222 mmol) slowly at rt. The reaction mixture was stirred at rt for 2 h. The TLC showed reaction to be complete. The reaction mixture was poured into ice-water (200 mL), and extracted with EtOAc (3×100 mL). The organic layer was washed with brine (100 mL), dried (Na₂SO₄), filtered and concentrated under reduced pressure to afford crude compound 5-(chloromethyl)-1,3,4-oxadiazol-2-amine. 5-(pyrrolidin-1-ylmethyl)-1,3,4-oxadiazol-2-amine. Yield: 8 g (crude); MS (ESI+) for CHNOS m/z 134.17 [M+H]⁺. The crude residue was used in next step without further purification.

### 5-(Pyrrolidin-1-yl methyl)-1,3,4-oxadiazol-2-amine

To a solution of 5-(chloromethyl)-1,3,4-oxadiazol-2-amine (3 g, 22.5 mmol) in DMF (50 mL) were added pyrrolidine (3.2 g, 45.1 mmol) and K₂CO₃ (9.3 g, 67.6 mmol) at rt. The reaction mixture was stirred at 80° C. for 2 h. The TLC showed reaction to be complete. The reaction mixture was diluted with H₂O (100 mL) and extracted with EtOAC (3×100 mL). The organic layer was washed with brine (100 mL), dried (Na₂SO₄), filtered and concentrated under reduced pressure. The crude residue was triturated with Et₂O (100 mL) to afford 5-(pyrrolidin-1-ylmethyl)-1,3,4-oxadiazol-2-amine as a brown solid. Which was further purified by prep HPLC. Yield: 380 mg (10%); MS (ESI+) for CHNOS m/z 169.26 [M+H]⁺; 6.93 (bs, 2H), 3.62 (s, 2H), 2.45-2.51 (m, 4H), 1.65-1.70 (m, 4H).

### Intermediate 143

### 4-(2-Methoxyethyl)-5-methyl-4H-1,2,4-triazol-3-amine hydrochloride

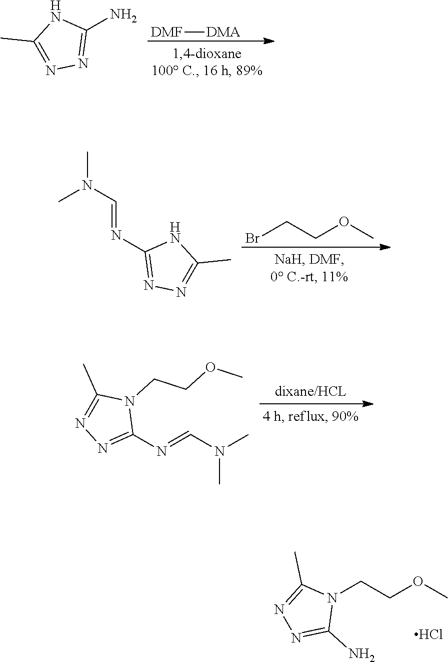

### (E)-N,N-dimethyl-N-(5-methyl-4H-1,2,4-triazol-3-yl)formimidamide

To a stirred solution of of 5-methyl-4H-1,2,4-triazol-3-amine (5 g, 51.1 mmol) in 1,4-dioxane (50 mL) was added DMF-DMA (12.1 g, 102 mmol) at rt. The reaction mixture was stirred at 100° C. for 16 h. The TLC showed reaction to be completed. The precipitated solid was filtered, washed with Et₂O (25 mL) and dried under vacuum to afford (E)-N,N-dimethyl-N′-(5-methyl-4H-1,2,4-triazol-3-yl)formimidamide as a white solid. Yield: 6.99 g (89%); (MS (ESI+) for CHNOS m/z 154.15 [M+H]⁺. ¹H NMR (400 MHz, DMSO-d₆): δ 12.38 (bs, 1H), 8.37 (s, 1H), 3.06 (s, 3H), 2.93 (s, 3H), 2.08 (s, 3H).

### (E)-N-(4-(2-Methoxyethyl)-5-methyl-4H-1,2,4-triazol-3-yl)-N,N-dimethylformimidamide

To a suspension of (E)-N,N-dimethyl-N′-(5-methyl-4H-1,2,4-triazol-3-yl)formimidamide (5.4 g, 35.3 mmol) in DMF (100 mL) was added NaH (60% in mineral oil, 4.3 g, 106 mmol) portion wise at 0° C. The reaction mixture was stirred for 1 h and added 1-bromo-2-methoxyethane (5 mL, 52.9 mmol). The reaction mixture slowly warmed to rt and stirred for 16 h. The TLC showed reaction to be completed.

The reaction mixture was diluted with H₂O (100 mL) and extracted with 10% MeOH in DCM (3×100 mL). The organic layer was dried over Na₂SO₄, filtered and concentrated under reduced pressure to afford (E)-N′-(4-(2-methoxyethyl)-5-methyl-4H-1,2,4-triazol-3-yl)-N,N-dimethylformimidamide as a light yellow solid. Yield 800 mg (11%). (MS (ESI+) for CHNOS m/z 212.14 [M+H]⁺. ¹H NMR (400 MHz, DMSO-d₆): δ 8.37 (s, 1H), 3.90 (t, J=5.7 Hz, 2H), 3.63 (t, J=5.7 Hz, 2H), 3.21 (s, 3H), 2.89 (s, 3H), 2.98 (s, 3H), 2.26 (s, 3H).

### 4-(2-Methoxyethyl)-5-methyl-4H-1,2,4-triazol-3-amine hydrochloride

A mixture of (E)-N′-(4-(2-methoxyethyl)-5-methyl-4H-1,2,4-triazol-3-yl)-N,N-dimethylformimidamide (400 mg, 1.89 mmol) in 4M Dioxane/HCl (4 mL) was stirred at 100° C. for 4 h. The TLC showed reaction to be complete. The solvent was evaporated under reduced pressure, triturated with Et2O (10 mL) and dried to afford 4-(2-methoxyethyl)-5-methyl-4H-1,2,4-triazol-3-amine hydrochloride as a waxy solid. Yield: 275 mg (90%); (MS (ESI+) for CHNOS m/z 157.1[M+H]⁺.

The following intermediates were prepared in a similar manner to 4-(2-methoxyethyl)-5-methyl-4H-1,2,4-triazol-3-amine hydrochloride.

| | | | | Spectral Data |
|---|---|---|---|---|
| Name | Int | Structure | Yield | 1H NMR & LCMS |
| 4,5-Dimethyl-4H- 1,2,4-triazol-3- amine hydrochloride | 144 | 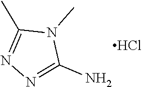 | 95% | MS (ESI+) for CHNOS m/z 113.14 [M + H]⁺; ¹H NMR (400 MHz, DMSO-d₆): δ 13.75 (bs, 1H), 8.92 (bs, 2H), 3.52 (s, 3H), 2.23 (s, 3H). |

The following examples were prepared in a similar manner to 2-((6-(trifluoromethyl)benzo[d]oxazol-2-yl)amino)thiazole-4-carboxamide following synthetic route 13.

| | | | | Spectral Data |
|---|---|---|---|---|
| Name | Ex | Structure | Yield | 1H NMR & LCMS |
| N-(5- Cyclopropyl- 1,3,4- oxadiazol-2- yl)-5-(trifluoro- methyl)benzo[d] oxazol-2-amine | 103 | 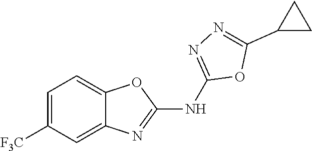 [SMILES: FC(F)(F)c1ccc2oc(Nc3nnc(C4CC4)o3)nc2c1] | 21% | MS (ESI+) for CHNOS m/z 311.08 [M + H]⁺; LC purity 94.2% (Ret. Time- 5.05 min); ¹H NMR (400 MHz, DMSO- d₆): δ 7.47 (s, 1H), 7.26 (d, J = 7.6 Hz, 1H), 7.12 (d, J = 8.9 Hz, 1H), 2.03- 2.07 (m, 1H), 0.93-0.97 (m, 2H), 0.85 (bs, 2H). |
| N-(5-Methyl- 1,3,4- oxadiazol-2- yl)-6-(trifluoro- methyl)benzo[d] oxazol-2-amine | 104 | 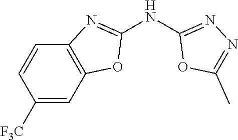 [SMILES: Cc1nnc(Nc2nc3ccc(C(F)(F)F)cc3o2)o1] | 5% | MS (ESI+) for CHNOS m/z 285.04 [M + H]⁺; LC purity 97.5% (Ret. Time- 5.73 min); ¹H NMR (400 MHz, DMSO-d₆): δ 13.0 (bs, 1H), 7.81 (s, 1H), 7.37-7.51 (m, 2H), 2.33 (S, 3H). |
| N-(5- Cyclopropyl- 1,3,4- oxadiazol-2- yl)-6-(trifluoro- methyl)benzo[d] oxazol-2-amine | 105 | 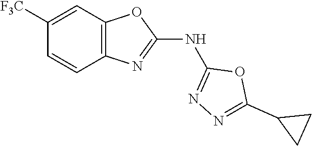 [SMILES: FC(F)(F)c1ccc2nc(Nc3nnc(C4CC4)o3)oc2c1] | 4% | MS (ESI+) for CHNOS m/z 311.08 [M + H]⁺; LC purity 98.9% (Ret. Time- 6.16 min); ¹H NMR (400 MHz, DMSO- d₆): δ 7.89 (s, 1H), 7.38- 7.51 (m, 2H), 1.97 (bs, 1H), 0.95 (bs, 2H), 0.78 (bs, 2H). |
| N-(5-Methyl- 1,3,4- oxadiazol-2- yl)oxazolo[4,5- c]pyridin-2- amine | 106 | 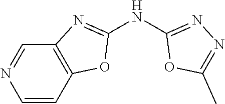 [SMILES: Cc1nnc(Nc2nc3cnccc3o2)o1] | 10% | MS (ESI+) for CHNOS m/z 218.04 [M + H]⁺; LC purity 81.2% (Ret. Time- 5.46 min); ¹H NMR (400 MHz, DMSO- d₆): δ 8.35 (s, 1H), 8.01 (d, J = 5.2 Hz, 1H), 7.18 (d, J = 5.2 Hz, 1H), 2.88 (s, 3H). |
| tert-Butyl 4-(5- ((5-(trifluoro- methyl)benzo[d] oxazol-2- yl)amino)- 1,3,4- oxadiazol-2- yl)piperidine-1- carboxylate | 107 | 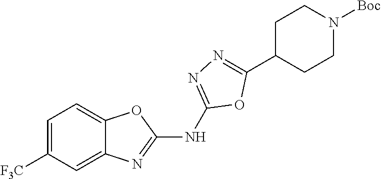 [SMILES: CC(C)(C)OC(=O)N1CCC(c2nnc(Nc3nc4cc(C(F)(F)F)ccc4o3)o2)CC1] | 40% | ¹H NMR (400 MHz, DMSO-d₆): δ 7.50 (d, J = 8.6 Hz, 1H), 7.22-7.47 (m, 2H), 7.13-7.17 (m, 1H), 3.89 (bs, 2H), 3.01 (bs, 3H), 1.99 (bs, 2H), 1.51 (bs, 2H), 1.41 (s, 9H). |
| N-(5-Isopropyl- 1,3,4- oxadiazol-2- yl)-5-(trifluoro- methyl)benzo[d] oxazol-2-amine | 108 | 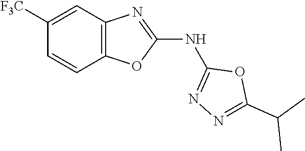 [SMILES: CC(C)c1nnc(Nc2nc3cc(C(F)(F)F)ccc3o2)o1] | 3% | MS (ESI+) for CHNOS m/z 313.3 [M + H]⁺; LC purity 98.3% (Ret. Time- 6.36 min); ¹H NMR (400 MHz, DMSO- d₆): δ 7.39-7.75 (m, 3H), 2.90 (bs, 1H), 1.08 (bs, 6H). |
| 6-Chloro-N-(5- methyl-1,3,4- oxadiazol-2- yl)benzo[d] oxazol-2-amine | 109 | 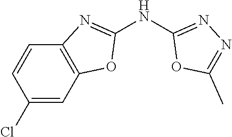 [SMILES: Cc1nnc(Nc2nc3ccc(Cl)cc3o2)o1] | 4% | MS (ESI+) for CHNOS m/z 251.1 [M + H]⁺; LC purity 97.0% (Ret. Time- 5.07 min); ¹H NMR (400 MHz, DMSO-d₆): δ 7.71 (s, 1H), 7.41 (d, J = 8.4 Hz, 1H), 7.28-7.34 (m, 1H), 2.42 (s, 3H). |
| 5-(Trifluoro- methyl)-N-(5- (trifluoro- methyl)-1,3,4- oxadiazol-2- yl)benzo[d] oxazol-2-amine | 110 | 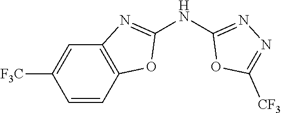 [SMILES: FC(F)(F)c1ccc2oc(Nc3nnc(C(F)(F)F)o3)nc2c1] | 32% | MS (ESI+) for CHNOS m/z 339.05 [M + H]⁺; LC purity 99.7% (Ret. Time- 4.86 min); ¹H NMR (400 MHz, DMSO- d₆): δ 7.92 (s, 1H), 7.85 (d, J = 8.5 Hz, 1H), 7.54 (d, J = 8.5 Hz, 1H). |
| N-(5-Methyl- 1,3,4- thiadiazol-2- yl)-5-(trifluoro- methyl)benzo[d] oxazol-2-amine | 111 | 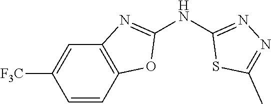 [SMILES: Cc1nnc(Nc2nc3cc(C(F)(F)F)ccc3o2)s1] | 12% | MS (ESI+) for CHNOS m/z 301.04 [M + H]⁺; LC purity 98.8% (Ret. Time- 6.24 min); ¹H NMR (400 MHz, DMSO-d₆): δ 7.24- 7.36 (m, 2H), 7.14 (d, J = 8.5 Hz, 1H), 2.49 (s, 3H). |
| N-(5- Methyloxazol- 2-yl)-5-(trifluoro- methyl)benzo[d] oxazol-2-amine | 112 | 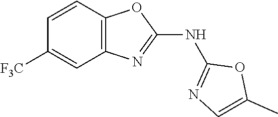 [SMILES: Cc1cnc(Nc2nc3cc(C(F)(F)F)ccc3o2)o1] | 2% | MS (ESI+) for CHNOS m/z 284.08 [M + H]⁺; LC purity 98.6% (Ret. Time- 3.83 min); ¹H NMR (400 MHz, DMSO-d₆): δ 7.62- 7.66 (m, 2H), 7.49 (d, J = 8.5 Hz, 1H), 6.48 (d, J = 1.3 Hz, 1H), 2.23 (s, 3H). |
| N-(4,5- Dimethyloxazol- 2-yl)-5-(trifluoro- methyl)benzo[d] oxazol-2-amine | 113 | 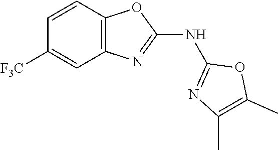 [SMILES: Cc1nc(Nc2nc3cc(C(F)(F)F)ccc3o2)oc1C] | 6% | MS (ESI+) for CHNOS m/z 298.10 [M + H]⁺; LC purity 99.1% (Ret. Time- 6.04 min); ¹H NMR (400 MHz, DMSO-d₆): δ 11.76 (bs, 1H), 7.67 (s, 1H), 7.63 (d, J = 8.4 Hz, 1H), 7.47 (d, J = 8.4 Hz, 1H), 2.17 (s, 3H), 2.09 (s, 3H). |
| N-(1,3,4- Oxadiazol-2- yl)-6-(trifluoro- methyl)benzo[d] oxazol-2-amine | 114 | 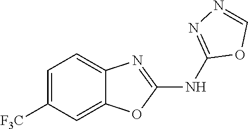 [SMILES: FC(F)(F)c1ccc2nc(Nc3nnco3)oc2c1] | 7% | MS (ESI+) for CHNOS m/z 271.04 [M + H]⁺; LC purity 95.5% (Ret. Time- 4.25 min); ¹H NMR (400 MHz, DMSO-d₆): δ 8.84 (s, 1H), 7.94 (s, 1H), 7.64 (d, J = 8.1 Hz, 1H), 7.55 (d, J = 8.1 Hz, 1H). |
| 6-Chloro-N- (1,3,4- oxadiazol-2- yl)benzo[d] oxazol-2-amine | 115 | 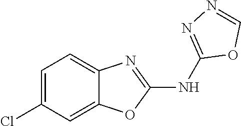 [SMILES: Clc1ccc2nc(Nc3nnco3)oc2c1] | 10% | MS (ESI+) for CHNOS m/z 237.01 [M + H]⁺; LC purity 96.3% (Ret. Time- 4.15 min); ¹H NMR (400 MHz, DMSO-d₆): δ 8.82 (s, 1H), 7.71 (s, 1H), 7.40 (d, J = 8.1 Hz, 1H), 7.33 (d, J = 8.1 Hz, 1H |
| N-(4H-1,2,4- Triazol-3-yl)-5- (trifluoro- methyl)benzo[d] oxazol-2-amine | 116 | 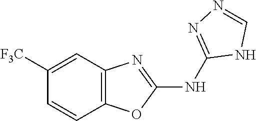 [SMILES: FC(F)(F)c1ccc2oc(Nc3nnc[nH]3)nc2c1] | 6% | MS (ESI+) for CHNOS m/z 270.11 [M + H]⁺; LC purity 98.1% (Ret. Time- 4.60 min); 1H NMR (400 MHz, DMSO-d6): δ 8.25 (s, 1H), 7.63-7.68 (m, 2H), 7.48 (d, J = 8.8 Hz, 1H). |
| N-(5-Methyl- 4H-1,2,4-triazol- 3-yl)-5-(trifluoro- methyl)benzo[d] oxazol-2-amine | 117 | 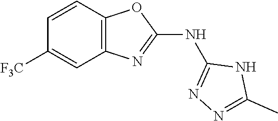 [SMILES: Cc1nnc(Nc2nc3cc(C(F)(F)F)ccc3o2)[nH]1] | 6% | MS (ESI+) for CHNOS m/z 284.14 [M + H]⁺; LC purity 99% (Ret. Time- 4.73 min); 1H NMR (400 MHz, DMSO- d6 + d-TFA): δ 7.71 (d, J = 8.5 Hz, 1H), 7.67 (s, 1H), 7.61 (d, J = 8.5 Hz, 1H), 2.46 (s, 3H). |
| N-(5-Methyl- 1,3,4-oxadiazol- 2-yl)-4-(trifluoro- methyl)benzo[d] oxazol-2-amine | 118 | 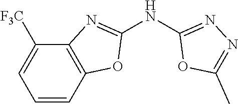 [SMILES: Cc1nnc(Nc2nc3c(C(F)(F)F)cccc3o2)o1] | 18% | MS (ESI+) for CHNOS m/z 285.18 [M + H]⁺; LC purity 99.4% (Ret. Time- 4.22 min); 1H NMR (400 MHz, DMSO- d6): δ 7.31 (d, J = 7.7 Hz, 1H), 7.17 (d, J = 7.7 Hz, 1H), 6.81- 6.88 (m, 1H), 2.28 (s, 3H). |
| 4-Chloro-N-(5- methyl-1,3,4- oxadiazol-2- yl)benzo[d] oxazol-2-amine | 119 | 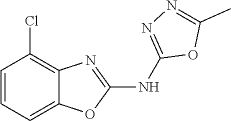 [SMILES: Cc1nnc(Nc2nc3c(Cl)cccc3o2)o1] | 15% | MS (ESI+) for CHNOS m/z 251.14 [M + H]⁺; LC purity 99.4% (Ret. Time- 3.91 min); 1H NMR (400 MHz, DMSO- d6): δ 7.05 (d, J = 8.2 Hz, 1H), 6.96 (d, J = 8.2 Hz, 1H), 6.71-6.77 (m, 1H), 2.27 (S, 3H). |
| 4-Chloro-N- (1,3,4- oxadiazol-2- yl)benzo[d] oxazol-2-amine | 120 | 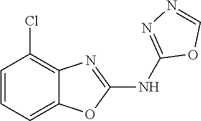 [SMILES: Clc1cccc2oc(Nc3nnco3)nc12] | 32% | MS (ESI+) for CHNOS m/z 237.11 [M + H]⁺; LC purity 97.4% (Ret. Time- 3.42 min); 1H NMR (400 MHz, DMSO-d6): δ 8.36 (s, 1H), 7.09 (d, J = 8.0 Hz, 1H), 6.99 (d, J = 8.0 Hz), 6.74 -6.80 (m, 1H). |
| 7-Chloro-N-(5- methyl-4H- 1,2,4-triazol-3- yl)benzo[d] oxazol-2-amine | 121 | 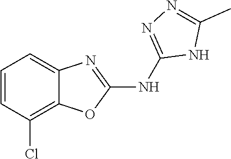 [SMILES: Cc1nnc(Nc2nc3cccc(Cl)c3o2)[nH]1] | 2% | MS (ESI+) for CHNOS m/z 250.11 [M + H]⁺; LC purity 96.4% (Ret. Time- 4.38 min); 1H NMR (400 MHz, DMSO-d6 + dTFA): 7.29-7.35 (m, 1H), 7.24-7.28 (m, 2H), 2.47 (s, 3H). |
| 6-Chloro-N-(5- methyl-4H- 1,2,4-triazol-3- yl)benzo[d] oxazol-2-amine | 122 | 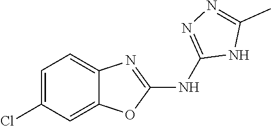 [SMILES: Cc1nnc(Nc2nc3ccc(Cl)cc3o2)[nH]1] | 2% | MS (ESI+) for CHNOS m/z 250.15 [M + H]⁺; LC purity 99.3% (Ret. Time- 4.94 min); 1H NMR (400 MHz, DMSO- d6 + dTFA): 7.97 (d, J = 1.8 Hz), 7.71 (dd, J = 1.8, 8.6 Hz, 1H), 7.46 (dd, J = 1.8, 8.6 Hz, 1H), 2.30 (s, 3H). |
| 4,6-Dichloro- N-(5-methyl- 4H-1,2,4- triazol-3- yl)benzo[d] oxazol-2-amine | 123 | 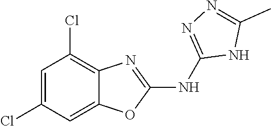 [SMILES: Cc1nnc(Nc2nc3c(Cl)cc(Cl)cc3o2)[nH]1] | 3% | MS (ESI+) for CHNOS m/z 284.15 [M + H]⁺; LC purity 99.4% (Ret. Time- 5.01 min); 1H NMR (400 MHz, DMSO-d6 + d- TFA): δ 7.62 (d, J = 1.8 Hz, 1H), 7.50 (d, J = 1.8 Hz, 1H), 2.40 (s, 3H). |
| 4-Fluoro-N-(5- methyl-4H- 1,2,4-triazol-3- yl)benzo[d] oxazol-2-amine | 124 | 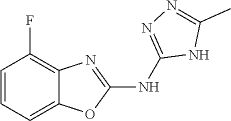 [SMILES: Cc1nnc(Nc2nc3c(F)cccc3o2)[nH]1] | 2% | MS (ESI+) for CHNOS m/z 234.20 [M + H]⁺; LC purity 99% (Ret. Time- 4.02 min); 1H NMR (400 MHz, DMSO- d6 + D₂O): δ 7.19-7.33 (m, 1H), 6.92-7.18 (m, 2H), 2.32 (s, 3H). |
| 6-chloro-N-(5- methyl-4H- 1,2,4-triazol-3- yl)-5-(trifluoro- methyl)benzo[d] oxazol-2-amine | 125 | 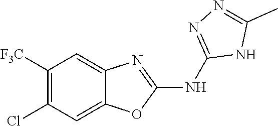 [SMILES: Cc1nnc(Nc2nc3cc(C(F)(F)F)c(Cl)cc3o2)[nH]1] | 7% | MS (ESI+) for CHNOS m/z 318.20 [M + H]⁺; LC purity 99.8% (Ret. Time- 5.15 min); 1H NMR (400 MHz, DMSO-d6 + d- TFA): δ 7.84 (s, 1H), 7.71 (s, 1H), 2.46 (s, 3H). |
| (S)-N-(5-(1- Methylpyrrolidin- 2-yl)-1,3,4- oxadiazol-2- yl)-5-(trifluoro- methyl)benzo[d] oxazol-2-amine | 126 | 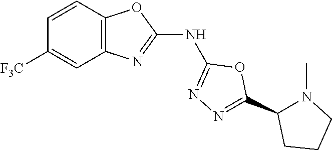 [SMILES: CN1CCC[C@H]1c1nnc(Nc2nc3cc(C(F)(F)F)ccc3o2)o1] | 2% | MS (ESI+) for CHNOS m/z 354.33 [M + H]⁺; LC purity 98.2% (Ret. Time- 4.73 min); 1H NMR (400 MHz, DMSO- d6): δ 7.60-7.66 (m, 2H), 7.51 (d, J = 8.4 Hz, 1H), 3.56 (t, J = 7.6 Hz, 1H), 3.01-3.08 (m, 1H), 2.36-2.42 (m, 1H), 2.32 (s, 3H), 2.15-2.20 (m, 1H), 2.01-2.08 (m, 1H), 1.80-1.92 |
| | | | | (m, 2H). |
| (R)-N-(5-(1- methylpyrrolidin- 2-yl)-1,3,4- oxadiazol-2- yl)-5-(trifluoro- methyl)benzo[d] oxazol-2-amine | 127 | 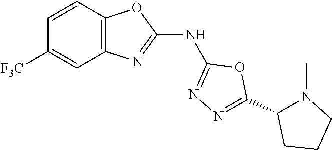 [SMILES: CN1CCC[C@@H]1c1nnc(Nc2nc3cc(C(F)(F)F)ccc3o2)o1] | 1% | MS (ESI+) for CHNOS m/z 354.0 [M + H]⁺; LC purity 96% (Ret. Time- 4.93 min); 1H NMR (400 MHz, DMSO- d6): δ 7.64-7.72 (m, 2H), 7.56 (d, J = 8.4 Hz, 1H), 3.62 (t, J = 7.6 Hz, 1H), 3.04-3.11 (m, 1H), 2.35-2.43 (m, 1H), 2.32 (s, 3H), 2.16-2.22 (m, 1H), 2.01-2.09 (m, 1H), 1.80- |
| | | | | 1.93 (m, 2H). |
| N-(5- (Pyrrolidin-1- ylmethyl)-4H- 1,2,4-triazol-3- yl)-5-(trifluoro- methyl)benzo[d] oxazol-2-amine | 128 | 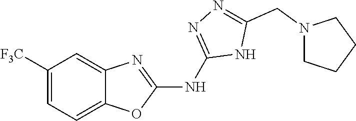 [SMILES: FC(F)(F)c1ccc2oc(Nc3nnc(CN4CCCC4)[nH]3)nc2c1] | 2% | MS (ESI+) for CHNOS m/z 353.32 [M + H]⁺; LC purity 99% (Ret. Time- 4.72 min); 1H NMR (400 MHz, DMSO- d6): δ 7.49-7.69 (m, 2H), 7.39 (d, J = 7.3 Hz, 1H), 3.83 (s, 2H), 2.75 (bs, 4H), 1.71 (bs, 4H). |
| 4,6-di Chloro- N-( 5- (pyrrolidin-1- ylmethyl)-4H- 1,2,4-triazol-3- yl)benzo[d] oxazol-2-amine | 129 | 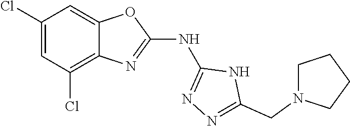 [SMILES: Clc1cc(Cl)c2nc(Nc3nnc(CN4CCCC4)[nH]3)oc2c1] | 5% | MS (ESI+) for CHNOS m/z 353.33 [M + H]⁺; LC purity 96.7% (Ret. Time- 4.82 min); 1H NMR (400 MHz, CD₃OD): δ 7.29 (d, J = 1.7 Hz, 1H), 7.19 (d, J = 1.7 Hz, 1H), 4.15 (s, 2H), 3.19-3.24 (m, 4H), 1.99-2.04 (m, 4H). |
| 4-Chloro-N-(5- (pyrrolidin-1- ylmethyl)-4H- 1,2,4-triazol-3- yl)benzo[d] oxazol-2-amine | 130 | 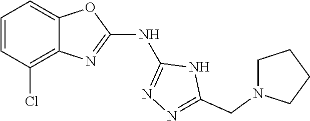 [SMILES: Clc1cccc2oc(Nc3nnc(CN4CCCC4)[nH]3)nc12] | 2% | MS (ESI+) for CHNOS m/z 319.36 [M + H]⁺; LC purity 99.8% (Ret. Time- 4.33 min); 1H NMR (400 MHz, DMSO- d6): δ 7.37 (bs, 1H), 7.20 (bs, 1H), 7.02 (bs, 1H), 3.82 (s, 2H), 2.67 (bs, 4H), 1.77 (bs, 4H). |
| 7-Chloro-N-(5- (pyrrolidin-1- ylmethyl)-4H- 1,2,4-triazol-3- yl)benzo[d] oxazol-2-amine | 131 | 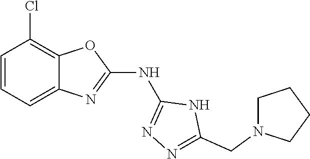 [SMILES: Clc1cccc2nc(Nc3nnc(CN4CCCC4)[nH]3)oc12] | 3% | MS (ESI+) for CHNOS m/z 319.35 [M + H]⁺; LC purity 99% (Ret. Time- 4.48 min); 1H NMR (400 MHz, CD₃OD): δ 7.28 (dd, J = 0.9, 7.6 Hz, 1H), 7.14-7.20 (m, 1H), 7.10 (dd, J = 0.9, 7.6 Hz, 1H), 3.92 (s, 2H), 2.90-2.95 (m, 4H), 1.89-1.98 (m, 4H). |
| 6-Chloro-N-(5- (pyrrolidin-1- ylmethyl)-4H- 1,2,4-triazol-3- yl)benzo[d] oxazol-2-amine | 132 | 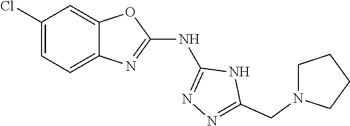 [SMILES: Clc1ccc2nc(Nc3nnc(CN4CCCC4)[nH]3)oc2c1] | 3% | MS (ESI+) for CHNOS m/z 319.35 [M + H]⁺; LC purity 99.5% (Ret. Time- 4.48 min); 1H NMR (400 MHz, DMSO- d6): δ 7.55 (s, 1H), 7.32 (d, J = 8.3 Hz, 1H), 7.20 (dd, J = 1.6, 8.3 Hz, 1H), 3.73 (s, 2H), 2.63 (bs, 4H), 1.70-1.75 (m, 4H). |
| (5-((4,6-Dichloro- benzo[d]oxazol- 2-yl)amino)- 1,3,4- oxadiazol-2- yl)(pyrrolidin-1- yl)methanone | 133 | 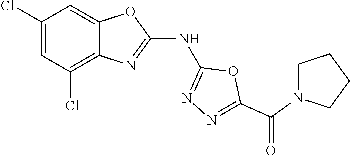 [SMILES: O=C(c1nnc(Nc2nc3c(Cl)cc(Cl)cc3o2)o1)N1CCCC1] | 14% | MS (ESI+) for CHNOS m/z 368.35 [M + H]⁺; LC purity 93.9% (Ret. Time- 5.61); ¹H NMR (400 MHz, DMSO- d₆): δ 7.30 (d, J = 2.0 Hz, 1H), 7.13 (d, J = 2.0 Hz, 1H), 3.88 (t, J = 6.6 Hz, 2H), 3.48 (t, J = 6.6 Hz, 2H), 1.88-1.96 (m, 2H), 1.79-1.87 (m, 2H). |
| (5-((4-Chloro- benzo[d]oxazol- 2-yl)amino)- 1,3,4- oxadiazol-2- yl)(pyrrolidin-1- yl)methanone | 134 | 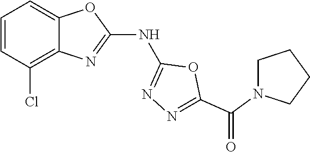 [SMILES: O=C(c1nnc(Nc2nc3c(Cl)cccc3o2)o1)N1CCCC1] | 11% | MS (ESI+) for CHNOS m/z 334.37 [M + H]⁺; LC purity 97.4% (Ret. Time- 3.98); ¹H NMR (400 MHz, DMSO-d₆): δ 7.14 (d, J = 7.4 Hz, 1H), 7.03 (d, J = 7.4 Hz, 1H), 6.77-6.90 (m, 1H), 3.89 (t, J = 6.8 Hz, 2H), 3.48 (t, J = 6.8 Hz, 2H), 1.88-1.97 (m, 2H), 1.79-1.88 (m, 2H). |
| 5-Chloro-N- (1,3,4- oxadiazol-2- yl)benzo[d] oxazol-2-amine | 135 | 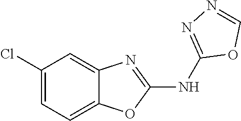 [SMILES: Clc1ccc2oc(Nc3nnco3)nc2c1] | 6% | MS (ESI+) for CHNOS m/z 236.92 [M + H]⁺; LC purity 98.8% (Ret. Time- 4.11); ¹H NMR (400 MHz, DMSO- d₆) δ 12.70 (bs, 1H), 8.85 (s, 1H), 7.54 (d, J = 8.6 Hz, 1H), 7.43 (d, J = 2.0 Hz, 1H), 7.26 (dd, J = 2.0, 8.6 Hz, 1H). |
| N-(5-((Dimethyl- amino)methyl)- 4H-1,2,4-triazol- 3-yl)-5-(trifluoro- methyl)benzo[d] oxazol-2-amine | 136 | 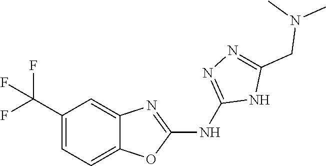 [SMILES: CN(C)Cc1nnc(Nc2nc3cc(C(F)(F)F)ccc3o2)[nH]1] | 2% | MS (ESI+) for CHNOS m/z 327.35 [M + H]⁺; LC purity 99.5% (Ret. Time- 4.24); ¹H NMR (400 MHz, DMSO-d₆): δ 12.24 (bs, 1H), 7.61 (bs, 1H), 7.57 (d, J = 8.3 Hz, 1H), 7.40 (d, J = 8.3 Hz, 1H), 3.59 (s, 2H), 2.29 (s, 6H). |
| 6-Chloro-N-(5- (pyrrolidin-1- ylmethyl)-4H- 1,2,4-triazol-3- yl)-5-(trifluoro- methyl)benzo[d] oxazol-2-amine | 137 | 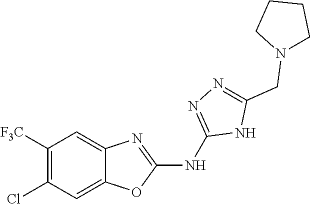 [SMILES: FC(F)(F)c1cc2nc(Nc3nnc(CN4CCCC4)[nH]3)oc2cc1Cl] | 5% | MS (ESI+) for CHNOS m/z 387.33 [M + H]⁺; LC purity 97.1% (Ret. Time- 5.07); ¹H NMR (400 MHz, MeOD) δ 7.64 (s, 1H), 7,52 (s, 1H), 4.10 (s, 2H), 3.16 (bs, 4H), 2.01 (bs, 4H). |
| 6-Chloro-N- (1,3,4- oxadiazol-2- yl)-5-(trifluoro- methyl)benzo[d] oxazol-2-amine | 138 | 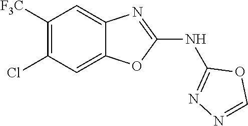 [SMILES: FC(F)(F)c1cc2nc(Nc3nnco3)oc2cc1Cl] | 13% | MS (ESI+) for CHNOS m/z 305.02 [M + H]⁺; LC purity 99.0% (Ret. Time- 4.49); ¹H NMR (400 MHz, DMSO-d₆ + d-TFA): δ 8.85 (s, 1H), 7.98 (s, 1H), 7.78 (s, 1H). |
| 6-Chloro-N-(5- isopropyl-4H- 1,2,4-triazol-3- yl)-5-(trifluoro- methyl)benzo[d] oxazol-2-amine | 139 | 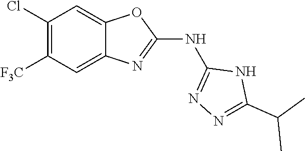 [SMILES: CC(C)c1nnc(Nc2nc3cc(C(F)(F)F)c(Cl)cc3o2)[nH]1] | 25% | MS (ESI+) for CHNOS m/z 346.28 [M + H]⁺; LC purity 90.5% (Ret. Time- 3.09 ; ¹H NMR (400 MHz, DMSO-d₆): δ 11.72 (bs, 1H), 7.42 (s, 1H), 7.35 (s, 1H), 2.71- 2.80 (m, 1H), 1.18 (d, J = 6.9 Hz, 6H). |
| 6-Chloro-N-(1- methyl-1H- imidazol-4-yl)- 5-(trifluoro- methyl)benzo[d] oxazol-2-amine | 140 | 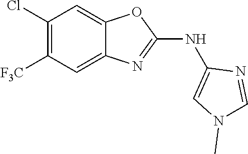 [SMILES: Cn1cnc(Nc2nc3cc(C(F)(F)F)c(Cl)cc3o2)c1] | 20% | MS (ESI+) for CHNOS m/z 317.34 [M + H]⁺; LC purity 99.9% (Ret. Time- 5.13; ¹H NMR (400 MHz, DMSO-d₆): δ; 11.21 (bs, 1H), 7.93 (s, 1H), 7.77 (s, 1H), 7.47 (s, 1H), 7.27 (s, 1H), 3.67 (s, 3H). |
| 6-Chloro-4- methyl-N-(5- methyl-4H- 1,2,4-triazol-3- yl)benzo[d] oxazol-2-amine | 141 | 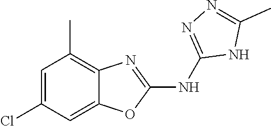 [SMILES: Cc1nnc(Nc2nc3c(C)cc(Cl)cc3o2)[nH]1] | 10% | MS (ESI+) for CHNOS m/z 264.27 [M + H]⁺; LC purity 99.90% (Ret. Time- 4.83; ¹H NMR at 295.5 K (400 MHz, DMSO-d₆): δ 7.32 (s, 1H), 7.05 (s, 1H), 2.46 (s, 3H), 2.31 (s, 3H). |
| 6-Chloro-N-(4- (2-methoxyethyl)- 5-methyl-4H- 1,2,4-triazol-3- yl)-5-(trifluoro- methyl)benzo[d] oxazol-2-amine | 142 | 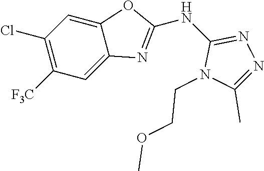 [SMILES: COCCn1c(C)nnc1Nc1nc2cc(C(F)(F)F)c(Cl)cc2o1] | 2% | MS (ESI+) for CHNOS m/z 376.13 [M + H]⁺; LC purity 97.2% (Ret. Time- 5.52); ¹H NMR (400 MHz, DMSO-d₆): δ 11.42 (bs, 1H), 7.98 (s, 1H), 7.86 (s, 1H), 4.22 (t, J = 5.0 Hz, 2H) 3.65 (t, J = 5.0 Hz, 2H), 3.23 (s, 3H), 2.38 (s, 3H). |
| N-(4,5- dimethyl-4H- 1,2,4-triazol-3- yl)-5-(trifluoro- methyl)benzo[d] oxazol-2-amine | 143 | 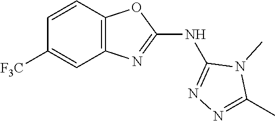 [SMILES: Cc1nnc(Nc2nc3cc(C(F)(F)F)ccc3o2)n1C] | 10% | MS (ESI+) for CHNOS m/z 298.22 [M + H]⁺; LC purity 99.8% (Ret. Time- 5.60 min); 1H NMR (400 MHz, DMSO- d₆): δ 12.45 (s, 1H), 7.58 (d, J = 8.2 Hz, 1H), 7.56 (s, 1H), 7.42 (d, J = 8.2 Hz, 1H), 3.58 (s, 3H), 2.33 (s, 3H). |
| 6-Chloro-1- methyl-N-(5- methyl-4H-1,2,4- triazol-3-yl)-5- (trifluoromethyl)- 1H-benzo[d] imidazol-2-amine | 144 | 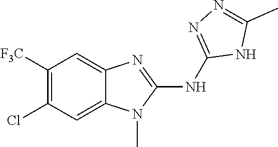 [SMILES: Cc1nnc(Nc2nc3cc(C(F)(F)F)c(Cl)cc3n2C)[nH]1] | 63% | MS (ESI+) for CHNOS m/z 331.06 [M + H]⁺; LC purity 99.5% (Ret. Time- 5.90 min); 1H NMR (400 MHz, DMSO-d₆): δ 8.08 (s, 2H), 7.52 (bs, 2H), 4.08 (s, 3H), 2.19 (s, 3H). |

### Synthetic Route 14

### N-(5-(Piperidin-4-yl)-1,3,4-oxadiazol-2-yl)-5-(trifluoromethyl)benzo[d]oxazol-2-amine hydrochloride (Example 145)

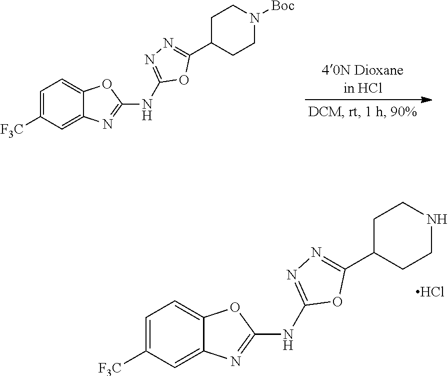 [SMILES: CC(C)(C)OC(=O)N1CCC(c2nnc(Nc3nc4cc(C(F)(F)F)ccc4o3)o2)CC1 ; FC(F)(F)c1ccc2oc(Nc3nnc(C4CCNCC4)o3)nc2c1 ; Cl]

To a solution of tert-butyl 4-(5-((5-(trifluoromethyl)benzo[d]oxazol-2-yl)amino)-1,3,4-oxadiazol-2-yl)piperidine-1-carboxylate (250 mg, 53 mmol) in CH₂Cl₂ (4 mL) was added 4 N HCl in 1,4-dioxane (8 mL) and stirred at rt for 1 h. The TLC showed reaction to be complete. The reaction mixture was concentrated under reduced pressure. The residue was triturated with DCM (10 mL), filtered and dried under vacuum to afford N-(5-piperidin-4-yl)-1,3,4-oxadiazol-2-yl)-5-(trifluoromethyl)benzo[d]oxazol-2-amine hydrochloride an off white solid. Yield: 185 mg (90%); ¹H NMR (400 MHz, DMSO-d₆): δ 8.96 (bs, 1H), 8.78 (bs, 1H), 7.69-7.75 (m, 2H), 7.61 (d, J=8.6 Hz, 1H), 3.23-3.34 (m, 3H), 2.99-3.07 (m, 2H), 2.14-2.19 (m, 2H), 1.87-1.99 (m, 2H); MS (ESI+) for CHNOS m/z 354.10 [M+H]⁺.

The following examples were prepared following synthetic route 13 & 14.

| | | | | Spectral Data |
|---|---|---|---|---|
| Name | Ex | Structure | Yield | 1H NMR & LCMS |
| (S)-N-(5- (pyrrolidin-2- yl)-1,3,4- oxadiazol-2- yl)-5-(trifluoro- methyl)benzo[d] oxazol-2-amine hydrochloride | 146 | 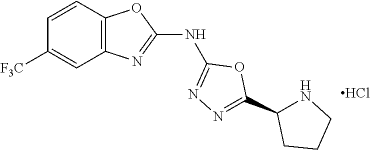 [SMILES: FC(F)(F)c1ccc2oc(Nc3nnc([C@@H]4CCCN4)o3)nc2c1 ; Cl] | 38% | MS (ESI+) for CHNOS m/z 340.24 [M + H]⁺; LC purity 99.8% (Ret. Time- 4.19 min); ¹H NMR (400 MHz, DMSO-d₆): δ 10.25 (bs, 2H), 7.76 (d, J = 8.5 Hz, 1H), 7.72 (s, 1H), 7.64 (d, J = 8.5 Hz, 1H), 4.93 (t, J = 7.8 Hz, 1H), 3.31 (t, J = 7.4 Hz, 2H), 2.35-2.49 (m, 1H), 2.23- 2.34 (m, 1H), 1.97-2.18 (m, 2H). |
| N-(5- (Piperazin-1- ylmethyl)-1,3,4- oxadiazol-2- yl)-5-(trifluoro- methyl)benzo[d] oxazol-2-amine hydrochloride | 147 | 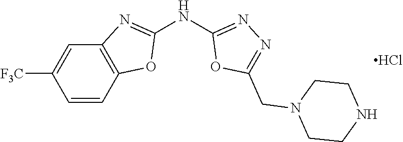 [SMILES: FC(F)(F)c1ccc2oc(Nc3nnc(CN4CCNCC4)o3)nc2c1 ; Cl] | 55% | MS (ESI+) for CHNOS m/z 369.15 [M + H]⁺; LC purity 97.8% (Ret. Time- 4.60 min); ¹H NMR (400 MHz, DMSO-d₆): δ 9.20 (bs, 2H), 7.75 (d, J = 8.4 Hz, 1H), 7.71 (s, 1H), 7.63 (d, J = 8.4 Hz, 1H), 4.05 (s, 2H), 3.35 (s, 4H), 2.93 (s, 4H). |
| tert-butyl 4-(5-((5- (trifluoromethyl) benzo[d]oxazol-2- yl)amino)-1,3,4- oxadiazol-2- yl)piperazine- | 148 | 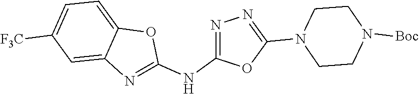 [SMILES: CC(C)(C)OC(=O)N1CCN(c2nnc(Nc3nc4cc(C(F)(F)F)ccc4o3)o2)CC1] | | MS (ESI+) for CHNOS m/z 455.08 [M + H]⁺; LC purity 95.0% (Ret. Time- 3.11 min); 1H NMR (400 MHz, DMSO-d₆): δ 10.86 (bs, 1H), 7.90-8.14 (m, 2H) 7.65-7.82 (m, 1H), 3.37-3.46 (m, 8H), 1.42 |
| 1-carboxylate | | | | (s, 9H). |
| 5-Chloro-N- (1,3,4-oxadiazol- 2-yl)-6-(trifluoro- methyl)benzo[d] oxazol-2-amine | 149 | 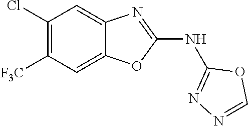 [SMILES: FC(F)(F)c1cc2oc(Nc3nnco3)nc2cc1Cl] | 12% | MS (ESI+) for CHNOS m/z 305.03 [M + H]⁺; LC purity 99.8% (Ret. Time- 3.30 min); 1H NMR (400 MHz, DMSO-d₆): δ 8.79 (s, 1H), 7.98 (s, 1H), 7.54 (s, 1H) |
| 6-Chloro-N-(4H- 1,2,4-triazol-3- yl)benzo[d] oxazol-2-amine | 150 | 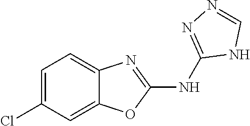 [SMILES: Clc1ccc2nc(Nc3nnc[nH]3)oc2c1] | 3% | MS (ESI+) for CHNOS m/z 236.06 [M + H]⁺; LC purity 99.5% (Ret. Time- 3.87 min); 1H NMR (400 MHz, DMSO-d₆): δ 13.40 (bs, 1H), 8.20 (bs, 1H), 7.62 (bs, 1H), 7.36 (d, J = 8.4 Hz, 1H), 7.25 (dd, J = 1.7, 8.4 Hz, 1H), |
| 6-Chloro-N- (1,3,4- thiadiazol-2- yl)benzo[d] oxazol-2-amine | 151 | 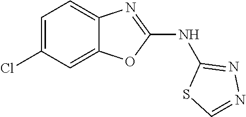 [SMILES: Clc1ccc2nc(Nc3nncs3)oc2c1] | 15% | MS (ESI+) for CHNOS m/z 253.01 [M + H]⁺; LC purity 98.2% (Ret. Time- 5.78 min); 1H NMR (400 MHz, DMSO-d₆): δ 14.39 (bs, 1H), 8.91 (bs, 1H), 7.69 (s, 1H), 7.50 d, J = 8.4 Hz, 1H), 7.28 (dd, J = 1.8 , 8.4 Hz, 1H). |
| 5,6-Dichloro- N-(1,3,4- oxadiazol-2- yl)benzo[d] oxazol-2-amine | 152 | 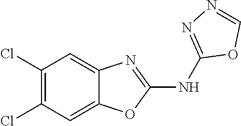 [SMILES: Clc1cc2nc(Nc3nnco3)oc2cc1Cl] | 19% | MS (ESI+) for CHNOS m/z 271.00 [M + H]⁺; LC purity 98.2% (Ret. Time- 3.11 min); 1H NMR (400 MHz, DMSO-d₆): δ 12.67 (bs, 1H), 8.87 (s, 1H), 7.96 (s, 1H), 7.58 (s, 1H). |

### Synthetic Route 15

### 6-Chloro-N-(5-methyl-4H-1,2,4-triazol-3-yl)-5-(trifluoromethyl)-1H-benzo[d]imidazol-2-amine (Example 153)

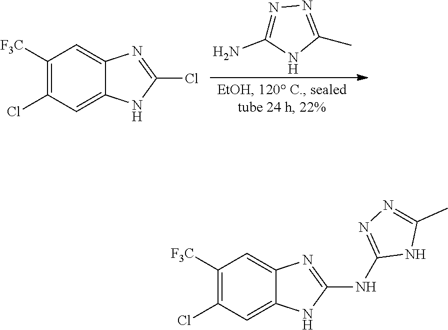

A mixture of 2,6-dichloro-5-(trifluoromethyl)-1H-benzo[d]imidazole (150 mg, 590 mmol) and 5-methyl-4H-1,2,4-triazol-3-amine (63 mg, 649 mmol) in EtOH (10 mL) was stirred at 120° C. in a sealed tube for 24 h. The TLC showed reaction to be complete. The reaction mixture was concentrated under reduced pressure. The residue was purified by prep HPLC purification to afford 6-chloro-N-(5-methyl-4H-1,2,4-triazol-3-yl)-5-(trifluoromethyl)-1H-benzo[d]imidazol-2-amine as a white solid. Yield: 41 mg (22%); ¹H NMR (400 MHz, DMSO-d₆+D₂O): δ 7.58-8.20 (m, 2H), 2.19 (s, 3H). (MS (ESI+) for CHNOS m/z 316.99 [M+H]⁺.

### Synthetic Route 16

### 4-Methyl-N-(5-methyl-1,3,4-oxadiazol-2-yl)oxazolo[4,5-c]pyridin-2-amine

### Example 154

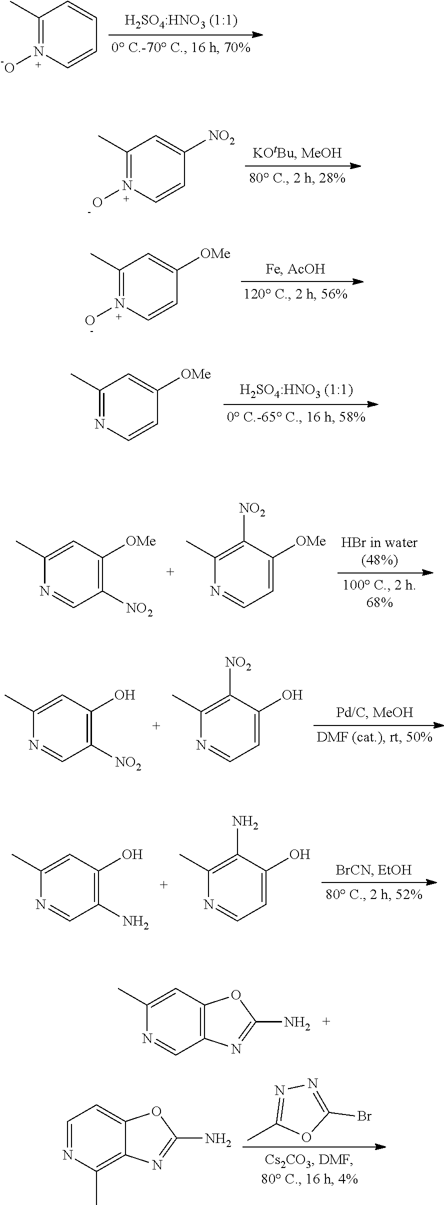

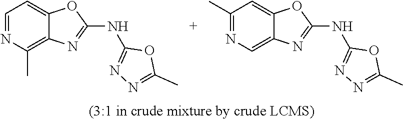

### 2-Methyl-4-nitropyridine 1-oxide

To a solution of 2-methylpyridine 1-oxide (4 g, 36.0 mmol) in conc. H₂SO₄ (10 mL) was added fuming HNO₃ (10 mL) slowly at 0° C. in a sealed tube. The reaction mixture was stirred at 70° C. for 16 h. The TLC showed reaction to be complete. Reaction was cooled to rt, quenched with ice-cold water (100 mL) and extracted with EtOAc (3×50 mL). The organics were dried (Na₂SO₄), filtered and concentrated under reduced pressure. The residue was triturated with 10% EtOAc in hexane (50 mL) to afford 2-methyl-4-nitropyridine 1-oxide as yellow solid. Yield: 4 g (70%); ¹H NMR (400 MHz, DMSO-d₆): δ 8.41-8.45 (m, 2H), 8.06-8.10 (m, 1H), 2.42 (s 3H).

### 4-Methoxy-2-methylpyridine 1-oxide

To a solution of 2-methyl-4-nitropyridine 1-oxide (2 g, 12.9 mmol) in MeOH (15 mL) was added ^{t}BuOk (4.4 g, 38.9 mmol) at rt. The reaction mixture was stirred at 80° C. for 2 h. The TLC showed reaction to be complete. The reaction mixture was concentrated under reduced pressure. The residue was diluted with H₂O (20 mL), acidified to pH 6 with 1N HCl and extracted with 10% MeOH in DCM (3×50 mL). The organic layer was washed with H₂O (50 mL), brine (50 mL), dried (Na₂SO₄), filtered and concentrated under reduced pressure to afford 4-methoxy-2-methylpyridine 1-oxide as a brown oil. Yield: 500 mg (28%); ¹H NMR (400 MHz, DMSO-d₆): δ 8.11 (d, J=7.1 Hz, 1H), 7.12 (d, J=3.3 Hz, 1H), 6.87-6.91 (m, 1H), 3.80 (s, 3H), 2.32 (s, 3H).

### 4-Methoxy-2-methylpyridine

To a solution of 4-methoxy-2-methylpyridine 1-oxide (500 g, 3.59 mmol) in acetic acid (10 mL) was added Fe (602 g, 10.79 mmol) at rt. The reaction mixture was stirred at 120° C. for 2 h. The TLC showed reaction to be complete. The reaction mixture was cooled to rt and filtered through a celite pad. The filtrate was diluted with H₂O (50 mL) and extracted with EtOAc (3×50 mL). The organic layer was washed with H₂O (100 mL), brine (100 mL), dried (Na₂SO₄), filtered and concentrated under reduced pressure to afford 4-methoxy-2-methylpyridine as a brown oil. Yield: 250 mg (56%); ¹H NMR (400 MHz, DMSO-d₆): δ8.23 (d, J=5.6 Hz, 1H), 6.82 (s, 1H), 6.76 (d, J=2.2 Hz, 1H), 3.79 (s, 3H), 2.39 (s, 3H); MS (ESI+) for CHNOS m/z 124.23 [M+H]⁺.

### 4-Methoxy-2-methyl-5-nitropyridine & 4-methoxy-2-methyl-3-nitropyridine

To a cooled solution of 4-methoxy-2-methylpyridine (700 mg, 5.69 mmol) in concentrated H₂SO₄ (10 mL) was added a mixture of H₂SO₄:HNO₃ (1:1, 2 mL) drop wise in a sealed tube. The reaction mixture was stirred at 65° C. for 16 h. The TLC showed reaction to be complete. The reaction mixture was cooled to rt, quenched with ice-cold water (100 mL) and extracted with EtOAc (3×100 mL). The organics were dried (Na₂SO₄), filtered and concentrated under reduced pressure. The residue was purified by column chromatography using silica gel (100-200 mesh), eluting with hexane to 70% EtOAc in hexane to afford a mixture of regioisomers, 4-methoxy-2-methyl-5-nitropyridine and 4-methoxy-2-methyl-3-nitropyridine in 85:15 ratio (by ¹H NMR) as a yellow solid. Yield: 550 g (58%); ¹H NMR (400 MHz, DMSO-d₆): δ 8.80 (s. 0.15H), 8.52 (d, J=5.8 Hz, 0.85H), 7.35 (s, 0.15H), 7.30 (d, J=5.8 Hz, 0.85H), 4.04 (s, 0.45H), 3.96 (s, 2.55H), 2.53 (s, 0.45H), 2.42 (s, 2.55H).

### 2-Methyl-5-nitropyridin-4-ol & 2-methyl-3-nitropyridin-4-ol

A solution of mixture of regioisomers 4-methoxy-2-methyl-5-nitropyridine and 4-methoxy-2-methyl-3-nitropyridine (400 mg, 2.38 mmol) in 33% HBr/AcOH (10 mL) was stirred at 100° C. for 2 h. The TLC showed reaction to be complete. The reaction mixture was cooled to rt, basified to pH 8 with saturated aqueous NaHCO₃ solution and extracted with EtOAc (3×20 mL). The organics were dried (Na₂SO₄), filtered and concentrated under reduced to afford a regioisomeric mixture of 2-methyl-5-nitropyridin-4-ol and 2-methyl-3-nitropyridin-4-ol as off white solid. Yield: 250 mg (68%); MS (ESI+) for CHNOS m/z 154.98 [M+H]⁺.

### 5-Amino-2-methylpyridin-4-ol & 3-amino-2-methylpyridin-4-ol

To a solution of mixture of regioisomers, 2-methyl-5-nitropyridin-4-ol and 2-methyl-3-nitropyridin-4-ol (300 g, 1.94 mmol) in MeOH (10 mL) were added 10% Pd/C (300 mg) DMF (0.1 mL). The reaction mixture was stirred at rt under H₂ balloon atmosphere for 2 h. The TLC showed reaction to be complete. The reaction mixture was passed through a pad of celite. The celite was washed with MeOH (20 mL). The filtrate was concentrated under reduced pressure. The residue was triturated with Et₂O (20 mL), dried under vacuum to afford to afford a regioisomeric mixture of 5-amino-2-methylpyridin-4-ol and 3-amino-2-methylpyridin-4-ol as off white solid. Yield: 120 mg (50%); MS (ESI+) for CHNOS m/z 125.03 [M+H]⁺.

### 6-Methyloxazolo[4,5-c]pyridin-2-amine & 4-methyloxazolo[4,5-c]pyridin-2-amine

To a solution of mixture of 5-amino-2-methylpyridin-4-ol & 3-amino-2-methylpyridin-4-ol (1.6 g, 12.9 mmol) in EtOH (20 mL) was added BrCN (2 g, 19.35 mmol) at rt. The reaction mixture was stirred at 80° C. for 2 h. The TLC showed reaction to be complete. The solvent was evaporated under reduced pressure. The residue was quenched with saturated aqueous solution of NaHCO₃ (25 mL) and extracted with EtOAc (3×25 mL). The organic layer was dried over Na₂SO₄, filtered and concentrated under reduced pressure. The residue was triturated with Et₂O (20 mL), dried under vacuum to afford a regioisomeric mixture of 6-methyloxazolo[4,5-c]pyridin-2-amine & 4-methyloxazolo[4,5-c]pyridin-2-amine as a brown solid. Yield: 1 g (52%); MS (ESI+) for CHNOS m/z 150.01 [M+H]⁺.

### 4-Methyl-N-(5-methyl-1,3,4-oxadiazol-2-yl)oxazolo[4,5-c]pyridin-2-amine

To a regioisomeric mixture of 6-methyloxazolo[4,5-c]pyridin-2-amine & 4-methyloxazolo[4,5-c]pyridin-2-amine (500 mg, 3.35 mmol) in DMF (10 mL) were added 2-bromo-5-methyl-1,3,4-oxadiazole (597 mg, 3.62 mmol) and Cs₂CO₃ (3.27 g, 10.1 mmol) at rt. The rection mixture was stirred at 100° C. for 2 h The TLC showed reaction to be complete. The reaction mixture was diluted with H₂O (50 mL) and extracted with EtOAc (3×50 mL). The organics were dried (Na₂SO₄), filtered and concentrated under reduced pressure to give the crude residue (3:1 by crude LCMS). The crude residue was purified by prep HPLC to afford the major regioisomer 4-methyl-N-(5-methyl-1,3,4-oxadiazol-2-yl)oxazolo[4,5-c]pyridin-2-amine as an off white solid. Yield: 30 mg (4%); MS (ESI+) for CHNOS m/z 232.09 [M+H]⁺; LC purity 99.8% (Ret. Time—3.12); NMR (400 MHz, DMSO-d₆): δ 8.51 (d, J=6.2 Hz, 1H), 7.91 (d, J=6.2 Hz, 1H), 2.85 (s, 3H), 2.46 (s, 3H). The minor regioisomer (6-methyl-N-(5-methyl-1,3,4-oxadiazol-2-yl)oxazolo[4,5-c]pyridin-2-amine) could not be isolated by prep HPLC.

### Synthetic Route 17

### 5-Methyl-N-(5-(trifluoromethyl)benzo[d]oxazol-2-yl)-4,5,6,7-tetrahydrooxazolo[5,4-c]pyridin-2-amine (Example 155)

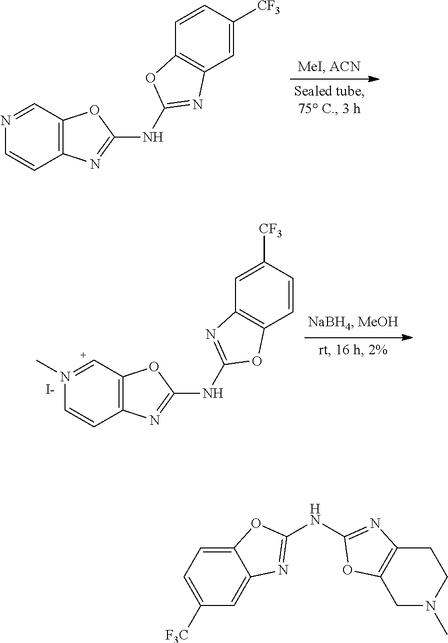

### 5-Methyl-2-((5-(trifluoromethyl)benzo[d]oxazol-2-yl)amino)oxazolo[5,4-c]pyridin-5-ium iodide

A mixture of N-(5-(trifluoromethyl)benzo[d]oxazol-2-yl)oxazolo[5,4-c]pyridin-2-amine (300 mg, 0.93 mmol) and CH₃I (200 mg, 1.4 mmol) in CH₃CN (6 mL) was stirred at 70° C. in a sealed tube for 3 h. The TLC showed reaction to be complete. The reaction mixture was evaporated under reduced pressure to afford 5-methyl-2-((5-(trifluoromethyl)benzo[d]oxazol-2-yl)amino)oxazolo[5,4-c]pyridin-5-ium iodide as a white solid and used for next step without further purification. Yield: 180 mg (crude, 74% by LCMS). MS (ESI+) for CHNOS m/z 335.13 [M+H]⁺.

### 5-Methyl-N-(5-(trifluoromethyl)benzo[d]oxazol-2-yl)-4,5,6,7-tetrahydrooxazolo[5,4-c]pyridin-2-amine

To a stirred solution of 5-methyl-2-((5-(trifluoromethyl)benzo[d]oxazol-2-yl)amino)oxazolo[5,4-c]pyridin-5-ium iodide (300 mg, 0.89 mmol) in MeOH (10 mL) was added NaBH₄ (102 mg, 2.68 mmol) at 0° C. The mixture was stirred at rt for 16 h. The TLC showed reaction to be complete. The solvent was evaporated under reduced pressure. The residue was diluted with H₂O (25 mL) and extracted with EtOAc (3×25 mL). The organic layer was dried over Na₂SO₄, filtered and concentrated under reduced pressure. The crude residue was purified by prep HPLC to afford 5-methyl-N-(5-(trifluoromethyl)benzo[d]oxazol-2-yl)-4,5,6,7-tetrahydrooxazolo[5,4-c]pyridin-2-amine as light yellow solid. Yield: 6 mg (2%); ¹H NMR (400 MHz, DMSO-d₆): δ 7.40 (bs, 1H), 7.20 (d, J=8.0 Hz, 1H), 7.07 (d, J=8.0 Hz, 1H), 2.60 (t, J=5.6 Hz, 2H), 2.56 (bs, 2H), 2.44 (bs, 2H), 2.35 (s, 3H); MS (ESI+) for CHNOS m/z 339.32 [M+H]⁺.

The following intermediates were prepared in a similar manner to 5-Methyl-2-((5-(trifluoromethyl)benzo[d]oxazol-2-yl)amino) oxazolo[5,4-c]pyridin-5-ium iodide.

| | | | | Spectral Data |
|---|---|---|---|---|
| Name | Int | Structure | Yield | 1H NMR & LCMS |
| 5-Methyl-2-((5- (trifluoromethyl) benzo[d]oxazol-2- yl)amino)oxazolo [4,5-c]pyridin-5- iumiodide | 145 | 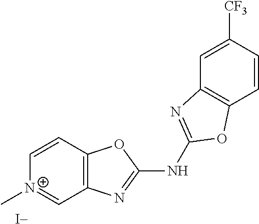 [SMILES: C[n+]1ccc2oc(Nc3nc4cc(C(F)(F)F)ccc4o3)nc2c1 ; [I-]] | 63% | MS (ESI+) for CHNOS m/z 335.0 [M]⁺. |
| 5-Methyl-2-((5- methyl-1,3,4- oxadiazol-2- yl)amino)oxazolo [4,5-c]pyridin-5- iumiodide | 146 | 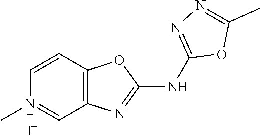 | 55% | MS (ES+) for CHNOS m/z 232.26 [M]⁺. |

The following example was prepared in a similar manner to 5-Methyl-N-(5-(trifluoromethyl)benzo[d]oxazol-2-yl)-4,5,6,7-tetrahydrooxazolo[5,4-c]pyridin-2-amine following synthetic route 17.

| | | | | Spectral Data |
|---|---|---|---|---|
| Name | Ex | Structure | Yield | 1H NMR & LCMS |
| 5-Methyl-N-(5- (trifluoromethyl) benzo[d]oxazol- 2-yl)-4,5,6,7- tetrahydro- oxazolo[4,5-c] pyridin-2-amine | 156 | 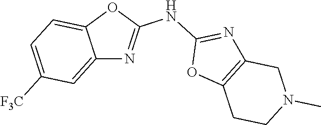 [SMILES: CN1CCc2oc(Nc3nc4cc(C(F)(F)F)ccc4o3)nc2C1] | 3% | MS (ESI+) for CHNOS m/z 339.35 [M + H]⁺; LC purity 90.05% (Ret. Time- 4.80); ¹H NMR (400 MHz, MeOD): δ 7.70 (s, 1H), 7.54 (d, J = 8.4 Hz, 1H), 7.49 (d, J = 8.4 Hz, 1H), 3.56 (s, 2H), 2.95 (t, J = 5.7 Hz, 2H), 2.78 (bs, 2H), 2.57 (s, 3H). |

### Synthetic Route 18

### N-(5-Methyl-1H-imidazol-2-yl)-5-(trifluoromethyl)benzo[d]oxazol-2-amine

### Example 157

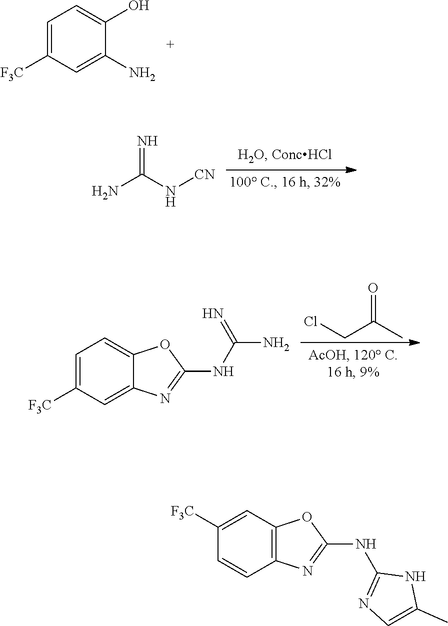

To a stirred solution of 2-amino-4-(trifluoromethyl)phenol (1 g, 5.6 mmol) in H₂O (10 mL) were added cyano-guanidin (470 mg, 5.6 mmol) and Conc. HCl (0.4 mL, 11.3 mmol) at rt. The mixture was stirred at 100° C. for 16 h. The TLC showed reaction to be complete. The reaction mixture was cooled to rt. The precipitated solid was filtered. The solid was purified by column chromatography using silica gel (100-200 mesh), eluting with 40% EtOAc in hexane to afford 1-(5-(trifluoromethyl)benzo[d]oxazol-2-yl)guanidine as a white solid. Yield: 420 mg (32%); MS (ESI+) for CHNOS m/z 245. 20 [M+H]⁺; LC purity 99.6% (Ret. Time—4.78; ¹H NMR (400 MHz, DMSO-d₆+D₂O): δ 7.57 (s, 1H), 7.45 (d, J=8.3 Hz, 1H), 7.37 (d, J=8.3 Hz, 1H).

### N-(5-Methyl-1H-imidazol-2-yl)-5-(trifluoromethyl)benzo[d]oxazol-2-amine

To a stirred solution of 1-(5-(trifluoromethyl)benzo[d]oxazol-2-yl)guanidine (100 mg, 0.40 mmol) in chloroacetone (0.5 mL) was added AcOH (0.2 mL) at rt. The mixture was stirred at 120° C. for 16 h. The TLC showed reaction to be complete. The reaction mixture was allowed to come to rt, diluted with ice-cold water (25 mL) and extracted with EtOAc (3×25 mL). The organic layer was dried over Na₂SO₄, filtered and concentrated under reduced pressure. The residue was purified by column chromatography using silica gel (100-200 mesh), eluting with hexane to 40% EtOAc in hexane to afford N-(5-methyl-1H-imidazol-2-yl)-5-(trifluoromethyl)benzo[d]oxazol-2-amine as a off white solid. Yield: 10 mg (9%); MS (ESI+) for CHNOS m/z 283.21 [M+H]⁺; LC purity 98.7% (Ret. Time—4.99; ¹H NMR (400 MHz, DMSO-d₆): δ 8.07 (s, 1H), 7.93 (d, J=8.5 Hz, 1H), 7.71 (d, J=8.5 Hz, 1H), 6.99 (s, 1H), 6.88 (bs, 2H), 2.02 (s, 3H).

### Intermediate 147

### 2-Amino-5-chloro-4-(trifluoromethyl)phenol

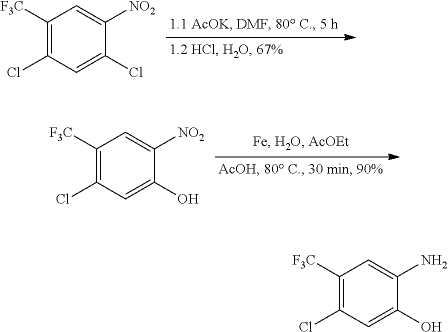 [SMILES: O=[N+]([O-])c1cc(C(F)(F)F)c(Cl)cc1Cl ; O=[N+]([O-])c1cc(C(F)(F)F)c(Cl)cc1O ; Nc1cc(C(F)(F)F)c(Cl)cc1O]

### 5-Chloro-2-nitro-4-(trifluoromethyl)phenol

To a solution of 1,5-dichloro-2-nitro-4-(trifluoromethyl)benzene (4 g, 15.4 mmol) in DMF (20 mL) was added potassium acetate (1.7 g, 16.9 mmol) portion wise. The reaction was stirred at 60° C. for 1 h and at 80° C. for 3 h. Potassium acetate (1.7 g, 16.9 mmol) was added and it was stirred at 80° C. for 1 h. The reaction mixture was cooled to rt, 1N HCl (100 mL) was added and extracted with EtOAc (3×100 mL). The organic layer was washed with water (100 mL), brine (100 mL), dried (Na₂SO₄), filtered and concentrated in vacuo. The crude residue was purified by column chromatography to afford 5-chloro-2-nitro-4-(trifluoromethyl)phenol as a yellow solid. Yield: 2.5 g (67%); ¹H NMR (400 MHz, CDCl₃): δ10.81 (s, 1H), 8.49 (s, 1H), 7.31 (s, 1H); MS (ESI+) for CHNOS m/z 240.11 [M−H]⁺.

### 2-Amino-5-chloro-4-(trifluoromethyl)phenol

To a suspension of Fe (2.9 g, 51.8 mmol) in AcOH (10 mL) and H₂O (15 mL) at 80° C. was added 5-chloro-2-nitro-4-(trifluoromethyl)phenol (2.5 g, 10.3 mmol) in EtOAc (5 mL) dropwise. The reaction mixture was heated at 80° C. for 30 min. The reaction mixture was cooled to rt, H₂O (50 mL) was added and extracted with EtOAc (3×50 mL). The organic layer was washed with water (100 mL), brine (100 mL), dried (Na₂SO₄), filtered and concentrated in vacuo to afford 2-amino-5-chloro-4-(trifluoromethyl)phenol as a white solid. Yield: 2.0 g (90%); MS (ESI+) for CHNOS m/z 210.12 [M−H]⁺.

The following intermediates were prepared in a similar manner to 5-(trifluoromethyl)benzo[d]oxazole-2-thiol.

| | | | | Spectral Data |
|---|---|---|---|---|
| Name | Int | Structure | Yield | 1H NMR & LCMS |
| 5- Chlorobenzo[d] oxazole-2-thiol | 148 | 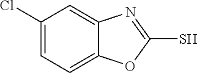 [SMILES: Sc1nc2cc(Cl)ccc2o1] | 76% | MS (ESI−) for CHNOS m/z 184.09 [M − H]⁺; ¹H NMR (400 MHz, DMSO-d₆): δ 14.05 (bs, 1H), 7.53 (d, J = 8.4 Hz, 1H), 7.29-7.33 (m, 2H). |
| 5- Fluorobenzo[d] oxazole-2-thiol | 149 | 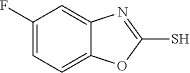 [SMILES: Fc1ccc2oc(S)nc2c1] | 84% | ¹H NMR (400 MHz, DMSO- d₆): δ 9.74 (bs, 1H), 7.29 (s, 1H), 6.91-7.01 (m, 2H). |
| 6- Chlorobenzo[d] oxazole-2-thiol | 150 | 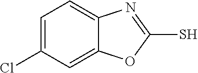 [SMILES: Sc1nc2ccc(Cl)cc2o1] | 89% | MS (ESI+) for CHNOS m/z 185.97 [M + H]⁺; ¹H NMR (400 MHz, DMSO-d₆): δ 14.02 (bs, 1H), 7.73 (s, 1H), 7.34 (d, J = 8.4 Hz, 1H), |
| | | | | 7.23 (d, J = 8.4 Hz, 1H). |
| 6- (Trifluoromethyl) benzo[d]oxazole- 2-thiol | 151 | 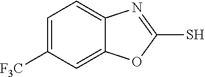 [SMILES: FC(F)(F)c1ccc2nc(S)oc2c1] | 80% | MS (ESI+) for CHNOS m/z 218.11 [M − H]⁺; ¹H NMR (400 MHz, DMSO-d₆): δ 14.12 (bs, 1H), 7.97 (s, 1H), 7.64 (d, J = 7.2 Hz, 1H), |
| | | | | 7.40 (d, J = 8.0 Hz, 1H). |
| 6-Chloro-5- (trifluoromethyl) benzo[d]oxazole-2- thiol | 152 | 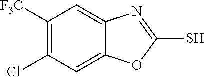 [SMILES: FC(F)(F)c1cc2nc(S)oc2cc1Cl] | 91% | MS (ESI+) for CHNOS m/z 254.03 [M − H]⁺; ¹H NMR (400 MHz, CDCl₃): δ 10.67 (bs, 1H), 7.56 (s, 2H). |

The following intermediates were prepared in a similar manner to 2-Chloro-5-(trifluoromethyl)benzo[d]oxazole.

| | | | | Spectral |
|---|---|---|---|---|
| | | | | Data |
| | | | | 1H NMR & |
| Name | Int | Structure | Yield | LCMS |
| 2,5- Dichloro- benzo[d] oxazole | 153 | 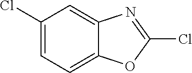 [SMILES: Clc1ccc2oc(Cl)nc2c1] | 50% | Proceeded further without purification. |
| 2-Chloro-5- fluoro- benzo[d] oxazole | 154 | 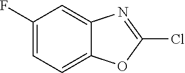 [SMILES: Fc1ccc2oc(Cl)nc2c1] | 62% | Proceeded further without purification. |
| 2,6- Dichloro- benzo[d] oxazole | 155 | 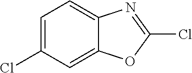 [SMILES: Clc1ccc2nc(Cl)oc2c1] | 60% | Proceeded further without purification. |
| 2-Chloro-6- (trifluoro- methyl) benzo[d] oxazole | 156 | 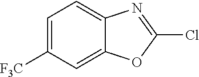 [SMILES: FC(F)(F)c1ccc2nc(Cl)oc2c1] | 26% | Proceeded further without purification. |
| 2,6- Dichloro-5- (trifluoro- methyl) benzo[d] oxazole | 157 | 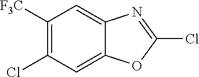 [SMILES: FC(F)(F)c1cc2nc(Cl)oc2cc1Cl] | 60% | Proceeded further without purification. |

The following examples were prepared in a similar manner to N-Cyclopropyl-2-((5-(trifluoromethyl)benzo[d]oxazol-2-yl)amino)thiazole-4-carboxamide following synthetic route 1.

| | | | | Spectral Data |
|---|---|---|---|---|
| Name | Ex | Structure | Yield | 1H NMR & LCMS |
| N-(Benzo[d]oxazol- 2-yl)-5- (trifluoromethyl) benzo[d]oxazol-2- amine | 158 | 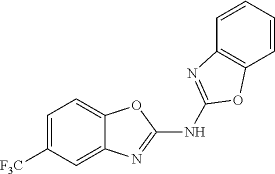 [SMILES: FC(F)(F)c1ccc2oc(Nc3nc4ccccc4o3)nc2c1] | 14% | MS (ESI+) for CHNOS m/z 320.29 [M + H]⁺; LC purity 97.6% (Ret. Time- 6.19 min); ¹H NMR (400 MHz, DMSO-d₆): δ 12.61 (bs, 1H), 7.80 (s, 1H), 7.76 (d, J = 8.6 Hz, 1H), 7.52-7.62 (m, 3H), 7.25-7.38 (m, 2H). |
| N-(6- Chlorobenzo[d] oxazol-2- yl)oxazolo[4,5- c]pyridin-2-amine | 159 | 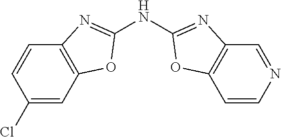 [SMILES: Clc1ccc2nc(Nc3nc4cnccc4o3)oc2c1] | 7% | MS (ESI+) for CHNOS m/z 287.06 [M + H]⁺; LC purity 95.5% (Ret. Time- 4.17 min); ¹H NMR (400 MHz, DMSO-d₆): δ 8.48 (d, J = 7.7 Hz, 1H), 8.06- 8.10 (m, 2H), 7.79 (d, J = 8.5 Hz, 1H), 7.50 (d, |
| | | | | J = 8.5 Hz, 1H), 6.48 |
| | | | | (d, J = 7.7 Hz, 1H). |
| N-(5- Fluorobenzo[d] oxazol-2- yl)oxazolo[4,5- c]pyridin-2-amine | 160 | 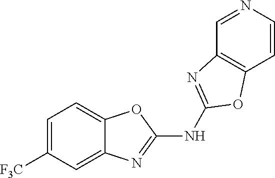 [SMILES: FC(F)(F)c1ccc2oc(Nc3nc4cnccc4o3)nc2c1] | 5% | MS (ESI+) for CHNOS m/z 271.08 [M + H]⁺; LC purity 98.4% (Ret. Time- 3.85 min); ¹H NMR (400 MHz, DMSO-d₆): δ 8.16-8.18 (m, 1H), 7.76-7.82 (m, 2H), 7.59-7.61 (m, 1H), 7.16-7.24 (m, 1H), 6.16 (d, J = 7.6 Hz, 1H). |

### Synthetic Route 19

### N-(Oxazolo[4,5-c]pyridin-2-yl)-6-(trifluoromethyl)oxazolo[4,5-c]pyridin-2-amine

### Example 161

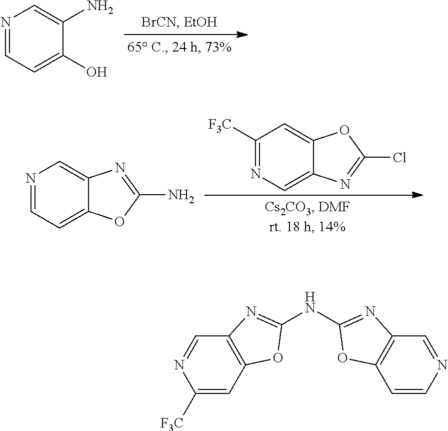 [SMILES: Nc1cnccc1O ; Nc1nc2cnccc2o1 ; FC(F)(F)c1cc2oc(Cl)nc2cn1 ; FC(F)(F)c1cc2oc(Nc3nc4cnccc4o3)nc2cn1]

### Oxazolo[4,5-c]pyridin-2-amine

To a solution of 3-aminopyridin-4-ol (3 g, 27.2 mmol) in EtOH (40 mL) was added cyanogen bromide (3.5 g, 32.7 mmol) at rt portion wise. The reaction mixture was stirred at 65° C. for 24 h The TLC showed reaction to be complete. The solvent was removed under reduced pressure. The residue was basified with saturated aq. NaHCO₃ solution (200 mL) and extracted with EtOAc (5×100 mL). The organic layer was washed with brine (100 mL), dried (Na₂SO₄), filtered and concentrated under reduce pressure. The residue was triturated with Et₂O (100 mL) and dried under vacuum to oxazolo[4,5-c]pyridin-2-amine. Yield: 2.7 g (73%); ¹H NMR (400 MHz, DMSO-d₆): δ 8.46 (s, 1H), 8.19 (d, J=5.2 Hz, 1H), 7.74 (bs, 2H), 7.43 (d, J=5.2 Hz, 1H); MS (ESI+) for CHNOS m/z 135.95 [M+H]⁺.

### N-(Oxazolo[4,5-c]pyridin-2-yl)-6-(trifluoromethyl)oxazolo[4,5-c]pyridin-2-amine

To a solution of 2-chloro-6-(trifluoromethyl)oxazolo[4,5-c]pyridine (500 mg, 2.25 mmol) in DMF (10 mL) were added oxazolo[4,5-c]pyridin-2-amine (334 mg, 2.47 mmol) and Cs₂CO₃ (7.4 g, 225 mmol). The resulting mixture was stirred at rt for 24 h. The TLC showed the reaction to be complete. The reaction mixture was poured in to ice water (50 mL) and extracted with 10% MeOH/DCM mixture (3×50 mL). The organics were dried (Na₂SO₄), filtered and concentrated under reduced pressure to give the crude residue. The residue was purified by prep HPLC to afford N-(oxazolo[4,5-c]pyridin-2-yl)-6-(trifluoromethyl)oxazolo[4,5-c]pyridin-2-amine as an off solid. Yield: 104 mg (14%); ¹H NMR (400 MHz, DMSO-d₆): δ 8.87 (s, 1H), 8.81 (s, 1H), 8.55 (d, J=6.0 Hz, 1H), 8.15 (s, 1H), 7.93 (d, J=6.0 Hz, 1H); MS (ESI+) for CHNOS m/z 322.02 [M+H]⁺.

### Intermediate 158

### 2-Chloro-6-(trifluoromethyl)oxazolo[4,5-c]pyridine

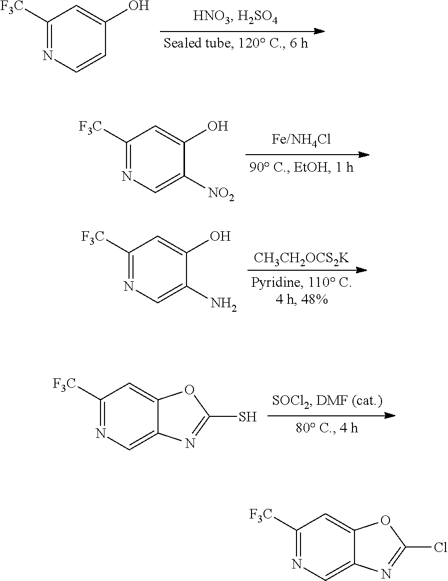

### 5-Nitro-2-(trifluoromethyl)pyridin-4-ol

To a cooled solution of 2-(trifluoromethyl)pyridin-4-ol (1.95 g, 11.9 mmol) in concentrated H₂SO₄ (4.8 mL) in sealed tube was added fuming HNO₃ (12 mL) dropwise. The reaction mixture was stirred at 120° C. for 6 h. The TLC showed the reaction to be complete. The reaction was cooled to room temperature, quenched with ice-cold water and extracted with EtOAc (3×100 mL). The organics were dried (Na₂SO₄), filtered and concentrated under reduced pressure to afford 5-nitro-2-(trifluoromethyl)pyridin-4-ol as brown solid. Yield: 2.2 g (crude); MS (ESI+) for CHNOS m/z 209.20 [M+H]⁺.

### 5-Amino-2-(trifluoromethyl)pyridin-4-ol

To a solution of 5-nitro-2-(trifluoromethyl)pyridin-4-ol (2.2 g, 10.5 mmol) were added ammonium chloride (2.9 g, 52.8 mmol), Fe powder (2.9 g, 52.8 mmol) and water (3.0 mL). The reaction mixture was stirred at 90° C. for 1 h. The TLC showed the reaction to be complete. Reaction mixture was cooled to room temperature and filtered through a celite bed. The filtrate was concentrated, diluted with water (25 mL) and extracted with EtOAc (3×50 mL). The organics were dried (Na₂SO₄), filtered and concentrated under reduced pressure to afford 5-amino-2-(trifluoromethyl)pyridin-4-ol as a brown liquid. Yield: 890 mg (crude); MS (ESI+) for CHNOS m/z 179.01 [M+H]⁺.

### 6-(Trifluoromethyl)oxazolo[4,5-c]pyridine-2-thiol

To a solution of 5-amino-2-(trifluoromethyl)pyridin-4-ol (2.0 g, 11.2 mmol) in pyridine (20 mL) was added potassium ethyl xanthate (2.2 g, 13.4 mmol) at rt. The reaction mixture was stirred at 110° C. for 4 h. The TLC showed the reaction to be complete. The reaction mixture was cooled to rt and acidified to pH 4-5 by slow addition of 1.0N HCl. The reaction mixture was extracted with EtOAc (3×25 mL). The organics were dried (Na₂SO₄), filtered and concentrated under reduced pressure. The residue was triturated with Et₂O (25 mL) to give 6-(trifluoromethyl)oxazolo[4,5-c]pyridine-2-thiol as a brown solid. Yield: 1.1 g (50%); ¹H NMR (400 MHz, DMSO-d₆): δ 8.63 (s, 1H), 8.20 (s, 1H); MS (ESI+) for CHNOS m/z 220.93 [M+H]⁺.

### 2-Chloro-6-(trifluoromethyl)oxazolo[4,5-c]pyridine

To a solution of 6-(trifluoromethyl)oxazolo[4,5-c]pyridine-2-thiol (300 mg, 1.77 mmol) in SOCl₂ (3 mL) was added DMF (cat) at rt. The reaction mixture was stirred at 80° C. for 4 h. The TLC showed the reaction to be complete. The solvent was removed under reduced pressure under N₂ to give 2-chloro-6-(trifluoromethyl)oxazolo[4,5-c]pyridine as brown liquid. Yield: 400 mg (crude). The crude was proceeded further without any purification.

### Intermediate 159

### 5-Amino-2-(trifluoromethyl)pyridin-4-ol

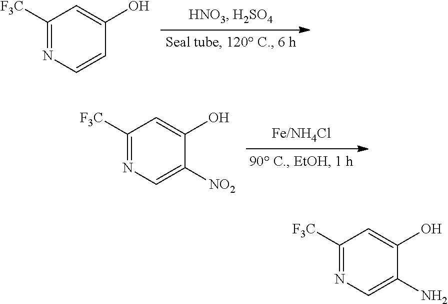 [SMILES: Oc1ccnc(C(F)(F)F)c1 ; O=[N+]([O-])c1cnc(C(F)(F)F)cc1O ; Nc1cnc(C(F)(F)F)cc1O]

### 5-Nitro-2-(trifluoromethyl)pyridin-4-ol

To a cooled solution of 2-(trifluoromethyl)pyridin-4-ol (1.95 g, 11.9 mmol) in concentrated H2SO4 (4.8 mL) in a sealed tube was added fuming HNO₃ (12 mL) dropwise. The reaction mixture was stirred at 120° C. for 6 h. The reaction was cooled to room temperature and quenched with ice-cold water. The mixture was extracted with EtOAc (3×100 mL), the organics were dried (Na₂SO₄), filtered and concentrated in vacuo to afford 5-nitro-2-(trifluoromethyl)pyridin-4-ol as brown solid. Yield: 2.2 g (crude); MS (ESI+) for CHNOS m/z 209.20 [M+H]⁺.

### 5-Amino-2-(trifluoromethyl)pyridin-4-ol

To a solution of 5-nitro-2-(trifluoromethyl)pyridin-4-ol (2.2 g, 10.5 mmol) in ethanol (20 mL) was added ammonium chloride (2.9 g, 52.8 mmol), Fe powder (2.9 g, 52.8 mmol) and water (3.0 mL). The reaction mixture was stirred at 90° C. for 1 h. The reaction mixture was cooled to room temperature and filtered through a celite bed. The filtrate was concentrated in vacuo, diluted with water (25 mL) and extracted with EtOAc (3×50 mL). The organics were dried (Na₂SO₄), filtered and concentrated in vacuo to afford 5-amino-2-(trifluoromethyl)pyridin-4-ol as a brown liquid. Yield: 890 mg (crude); MS (ESI+) for CHNOS m/z 179.01 [M+H]⁺.

### Intermediate 160

### Oxazolo[4,5-c]pyridin-2-amine

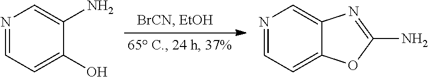 [SMILES: Nc1cnccc1O ; Nc1nc2cnccc2o1]

### Oxazolo[4,5-c]pyridin-2-amine

To a solution of 3-aminopyridin-4-ol (3 g, 27.2 mmol) in EtOH (40 mL) was added cyanogen bromide (3.5 g, 32.7 mmol) at rt portion wise. The reaction mixture was stirred at 65° C. for 24 h. The TLC showed reaction to be complete. The solvent was removed under reduced pressure. The residue was basified with saturated aq. NaHCO₃ solution (200 mL) and extracted with EtOAc (5×100 mL). The organic layer was washed with brine (100 mL), dried (Na₂SO₄), filtered and concentrated under reduce pressure. The residue was triturated with Et₂O (100 mL) and and dried under vacuum to oxazolo[4,5-c]pyridin-2-amine. Yield: 2.7 g (73%); ¹H NMR (400 MHz, DMSO-d₆): δ 8.46 (s, 1H), 8.19 (d, J=5.2 Hz, 1H), 7.74 (bs, 2H), 7.43 (d, J=5.2 Hz, 1H); MS (ESI+) for CHNOS m/z 135.95 [M+H]⁺.

### Intermediate 161

### 1-Tosyl-1H-benzo[d]imidazol-2-amine

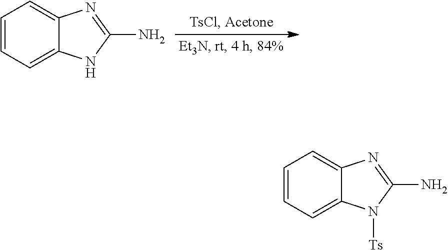 [SMILES: Nc1nc2ccccc2[nH]1 ; Cc1ccc(S(=O)(=O)n2c(N)nc3ccccc32)cc1]

To a solution of 1H-benzo[d]imidazol-2-amine (5 g, 37.5 mmol) in acetone (50 mL) were added triethylamine (15.8 mmol, 112.7 mmol) and Tscl (8.5 g, 45.1 mmol) in acetone (25 mL) slowly. The reaction mixture was stirred at rt for 4 h. The TLC showed reaction to be completed. The solvent was removed under reduced pressure. The residue was added to H₂O (50 mL) and extracted with EtOAc (3×50 mL). The organics layer was washed with brine (100 mL), dried (Na₂SO₄), filtered and concentrated under reduced pressure. The residue was triturated with DCM (100 mL), dried under vacuum to afford 1-tosyl-1H-benzo[d]imidazol-2-amine as a brown solid. Yield: 9 g (84%); ¹H NMR (400 MHz, DMSO-d₆): δ 10.14 (bs, 1H), 7.93 (d, J=8.3 Hz, 2H), 7.66 (d, J=8.0 Hz, 1H), 7.45 (d, J=8.3 Hz, 2H), 7.30 (bs, 2H), 7.09-7.15 (m, 2H), 6.99-7.06 (m, 1H), 2.35 (s, 3H); MS (ESI+) for CHNOS m/z 288.09 [M+H]⁺.

The following intermediate was prepared in a similar manner to 1-tosyl-1H-benzo[d]imidazol-2-amine.

| | | | | Spectral Data |
|---|---|---|---|---|
| Name | Int | Structure | Yield | 1H NMR & LCMS |
| 5-Chloro-1-tosyl- 1H- benzo[d] imidazol-2-amine | 162 | 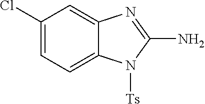 [SMILES: Cc1ccc(S(=O)(=O)n2c(N)nc3cc(Cl)ccc32)cc1] | 61% | MS (ESI+) for CHNOS m/z 322.29 [M + H]⁺; ¹H NMR (400 MHz, DMSO-d₆): δ 7.89-7.97 (m, 2H) 7.63 (d, J = 8.8 Hz, 1H), 7.42-7.49 (m, 2H), 7.34 (bs, 1H), 7.21 (bs, 1H), 7.12-7.27 (m, 1H), |
| | | | | 7.01-7.05 (m, 1H), 2.36 (s, |
| | | | | 3H). |

The following intermediates were prepared in a similar manner to oxazolo[4,5-c]pyridin-2-amine.

| | | | | Spectral Data |
|---|---|---|---|---|
| Name | Int | Structure | Yield | 1H NMR & LCMS |
| 5- Chlorobenzo[d] oxazol-2-amine | 163 | 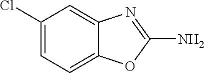 [SMILES: Nc1nc2cc(Cl)ccc2o1] | 78% | MS (ESI+) for CHNOS m/z 167.18 [M − H]⁺; ¹H NMR (400 MHz, DMSO-d₆): δ 7.55 (s, 2H), 7.49 (s, 1H), 7.10-7.19 (m, 2H). |

The following intermediates were prepared in a similar manner to dimethyl benzo[d]oxazol-2-ylcarbonimidodithioate

| | | | | Spectral Data |
|---|---|---|---|---|
| Name | Int | Structure | Yield | 1H NMR & LCMS |
| Dimethyl (5- chlorobenzo[d] oxazol-2- yl)carbon- imidodithioate | 164 | 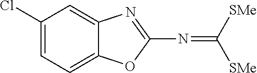 [SMILES: CSC(=Nc1nc2cc(Cl)ccc2o1)SC] | 50% | MS (ESI+) for CHNOS m/z 273.13 [M + H]⁺; ¹H NMR (400 MHz, DMSO- d₆): δ 7.75 (s, 1H), 7.65 (d, J = 8.6 Hz, 1H), 7.34-7.38 (m, 1H), 2.67 (s, 6H). |
| Dimethyl (5- (trifluoromethyl) benzo[d] oxazol- 2-yl)carbon- imidodithioate | 165 | 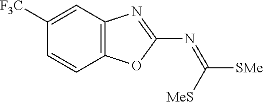 [SMILES: CSC(=Nc1nc2cc(C(F)(F)F)ccc2o1)SC] | 13% | MS (ESI+) for CHNOS m/z 306.91 [M + H]⁺. |
| Dimethyl (1- tosyl-1H- benzo[d]imidazol- 2- yl)carbon- imidodithioate | 166 | 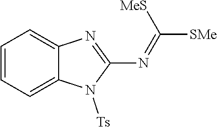 [SMILES: CSC(=Nc1nc2ccccc2n1S(=O)(=O)c1ccc(C)cc1)SC] | 35%. | MS (ESI+) for CHNOS m/z 280.97 [M + H]⁺; ¹H NMR (400 MHz, DMSO- d₆): δ 7.96 (d, J = 8.2 Hz, 2H), 7.89- 7.93 (m, 1H), 7.55-7.69 (m, 1H), 7.46 (d, J = 8.2 Hz, 2H), 7.29-7.34 (m, 2H), 2.67 (s, 6H), 2.35 (s, 3H). |
| Dimethyl (5- chloro-1-tosyl- 1H- benzo[d]imidazol- 2-yl)carbon- imidodithioate | 167 | 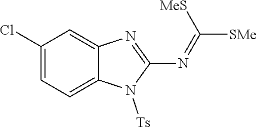 [SMILES: CSC(=Nc1nc2cc(Cl)ccc2n1S(=O)(=O)c1ccc(C)cc1)SC] | 10%. | MS (ESI+) for CHNOS m/z 426.12 [M + H]⁺; ¹H NMR (400 MHz, DMSO- d₆): δ 8.01 (d, J = 8.8 Hz, 1H), 7.82 (d, J = 8.4 Hz, 2H), 7.66 (d, J = 2.0 Hz, 1H), 7.36-7.47 (m, 3H), 2.62 (s, 6H), 2.36 (s, 3H). |

The following compounds were prepared in a similar manner to 5-Chloro-N-(5-methyl-1,3,4-oxadiazol-2-yl)benzo[d]oxazol-2-amine following synthetic route 3.

| | | | | Spectral Data |
|---|---|---|---|---|
| Name | Ex | Structure | Yield | 1H NMR & LCMS |
| N- (Benzo[d] oxazol-2-yl)-5- chlorobenzo[d] oxazol-2- amine | 162 | 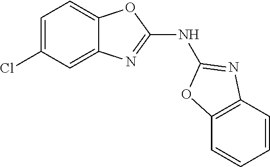 [SMILES: Clc1ccc2oc(Nc3nc4ccccc4o3)nc2c1] | 6% | MS (ESI+) for CHNOS m/z 286.06 [M + H]⁺; LC purity 98.0% (Ret. Time- 4.39 min); ¹H NMR (400 MHz, DMSO-d₆): δ 12.70 (bs, 1H), 7.48-7.54 (m, 4H), 7.18-7.31 (m, 3H). |
| N- (Benzo[d] oxazol-2-yl)-6- (trifluoromethyl) oxazolo[4,5- c]pyridin-2- amine | 163 | 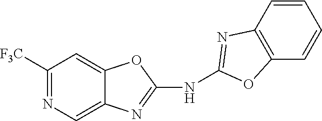 [SMILES: FC(F)(F)c1cc2oc(Nc3nc4ccccc4o3)nc2cn1] | 12% | MS (ESI+) for CHNOS m/z 320.71 [M + H]⁺; LC purity 99.9% (Ret. Time- 5.03 min); ¹H NMR (400 MHz, DMSO-d₆): δ 8.58 (s, 1H), 7.88 (s, 1H), 7.32-7.38 (m, |
| | | | | 2H), 7.10-7.16 (m, |
| | | | | 1H), 6.99-7.06 (m, |
| | | | | 1H). |
| N-(5- Chlorobenzo[d] oxazol-2- yl)oxazolo[4,5- c]pyridin-2- amine | 164 | 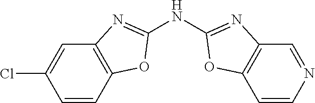 [SMILES: Clc1ccc2oc(Nc3nc4cnccc4o3)nc2c1] | 5% | MS (ESI+) for CHNOS m/z 287.02 [M + H]⁺; LC purity 98.7% (Ret. Time- 4.51 min); ¹H NMR (400 MHz, DMSO-d₆): δ 12.34 (bs, 1H), 8.73 |
| | | | | (s, 1H), 8.38 (d, J = |
| | | | | 5.2 Hz, 1H), 7.40-7.70 |
| | | | | (m, 3H), 7.23 (d, J = |
| | | | | 8.2 Hz, 1H). |
| N-(5- Chlorobenzo[d] oxazol-2-yl)-6- (trifluoromethyl) oxazolo[4,5- c]pyridin-2- amine | 165 | 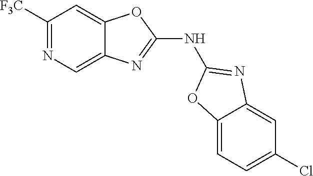 [SMILES: FC(F)(F)c1cc2oc(Nc3nc4cc(Cl)ccc4o3)nc2cn1] | 15% | MS (ESI+) for CHNOS m/z 355.03 [M + H]⁺; LC purity 99.8% (Ret. Time- 6.71 min); ¹H NMR (400 MHz, DMSO-d₆): δ 8.64 (s, 1H), 7.91 (s, 1H), 7.36 (d, J = 8.8 Hz, 2H), 7.02-7.06 (m, 1H). |
| N-(1H- Benzo[d]imidazol- 2-yl)-6- (trifluoromethyl) oxazolo[4,5- c]pyridin-2- amine | 166 | 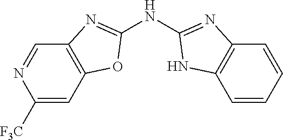 [SMILES: FC(F)(F)c1cc2oc(Nc3nc4ccccc4[nH]3)nc2cn1] | 27% | MS (ESI+) for CHNOS m/z 320.09 [M + H]⁺; LC purity 98.7% (Ret. Time- 5.82 min); ¹H NMR (400 MHz, DMSO-d₆): δ 12.45 (bs, 2H), 8.76 (s, 1H), 8.06 (s, 1H), 7.44-7.48 (m, 2H), |
| | | | | 7.19-7.24 (m, 2H). |
| N-(1H- Benzo[d]imidazol- 2-yl)-5- (trifluoromethyl) benzo[d]oxazol- 2-amine | 167 | 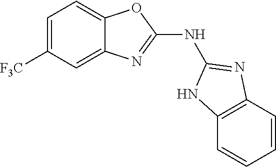 [SMILES: FC(F)(F)c1ccc2oc(Nc3nc4ccccc4[nH]3)nc2c1] | 30% | MS (ESI+) for CHNOS m/z 319.10 [M + H]⁺; LC purity 99.8% (Ret. Time- 4.62 min); ¹H NMR (400 MHz, DMSO-d₆): δ 12.33 (bs, 2H), 7.59-7.66 (m, 2H), 7.41-7.49 (m, 3H), 7.16-7.25 (m, 2H). |
| N-(5-Chloro- 1H- benzo[d]imidazol- 2-yl)oxazolo [4,5-c]pyridin-2- amine | 168 | 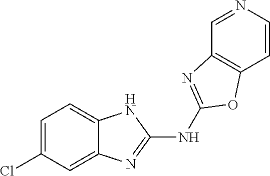 [SMILES: Clc1ccc2[nH]c(Nc3nc4cnccc4o3)nc2c1] | 28% | MS (ESI+) for CHNOS m/z 286.04 [M + H]⁺; LC purity 97.5% (Ret. Time- 4.62 min); ¹H NMR (400 MHz, DMSO-d₆ + d-TFA): δ 8.87 (s, 1H), 8.62 d, J = 5.9 Hz, 1H), 8.05 (d, J = 5.9 Hz, 1H), 7.50 (s, 1H), 7.45 d, J = 8.4 |
| | | | | Hz, 1H), 7.24 d, J = |
| | | | | 8.0 Hz, 1H). |

### Synthetic Route 20

### N-(benzo[d]oxazol-2-yl)-N-methyl-6-(trifluoromethyl)oxazolo[4,5-c]pyridin-2-amine (Example 169)

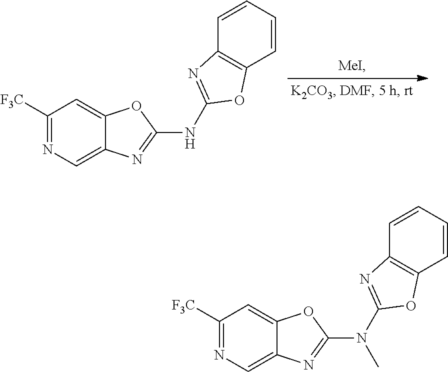 [SMILES: FC(F)(F)c1cc2oc(Nc3nc4ccccc4o3)nc2cn1 ; CN(c1nc2ccccc2o1)c1nc2cnc(C(F)(F)F)cc2o1]

To a solution of N-(benzo[d]oxazol-2-yl)-6-(trifluoromethyl)oxazolo[4,5-c]pyridin-2-amine (300 mg, 0.93 mmol) in DMF (5 mL) were added K₂CO₃ (388 mg, 2.81 mmol) and methyl iodide (0.2 mL, 2.81 mmol) at rt. The reaction mixture was stirred at rt for 5 h. The TLC showed the reaction to be complete. The solvent was removed in vacuo and water (5.0 mL) was added to residue and extracted with EtOAc (3×25 mL). The organics layer was washed with brine (20 mL), dried (Na₂SO₄), filtered and concentrated in vacuo. The residue was purified by column chromatography to give N-(benzo[d]oxazol-2-yl)-N-methyl-6-(trifluoromethyl)oxazolo[4,5-c]pyridin-2-amine as an off white solid. Yield: 160 mg (51%); ¹H NMR (400 MHz, DMSO-d₆): δ 9.11 (s, 1H), 7.91 (s, 1H), 7.72 (d, J=7.8 Hz, 1H), 7.61 (d, J=6.8 Hz, 1H), 7.32-7.41 (m, 2H), 4.11 (s, 3H); MS (ESI+) for CHNOS m/z 335.09 [M+H]⁺.

### Synthetic Route 21

### N-(6-(Trifluoromethyl)-1H-imidazo[4,5-c]pyridin-2-yl)benzo[d]oxazol-2-amine

### Example 170

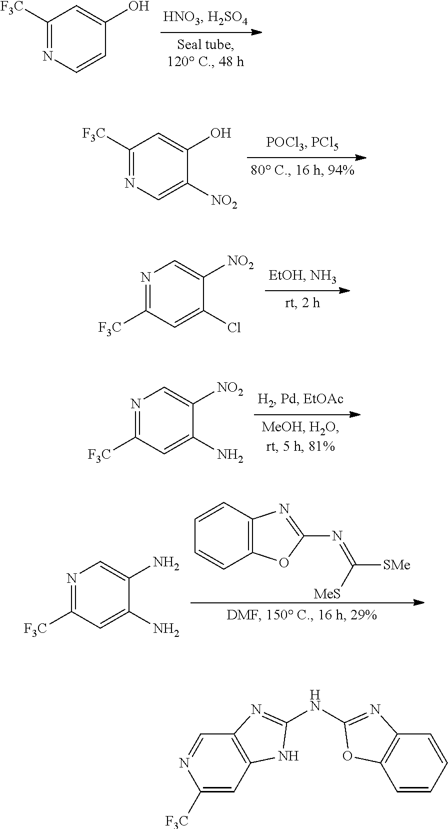

### 5-Nitro-2-(trifluoromethyl)pyridin-4-ol

To a cooled solution of 2-(trifluoromethyl)pyridin-4-ol (10 g, 11.9 mmol) in concentrated H₂SO₄ (4.8 mL) was added fuming HNO₃ (12 mL) dropwise. The reaction mixture was stirred at 120° C. for 48 h in a sealed tube. The TLC showed the reaction to be complete. The reaction was cooled to room temperature, quenched with ice-cold water and extracted with EtOAc (3×100 mL). The organics were dried (Na₂SO₄), filtered and concentrated under reduced pressure to afford 5-nitro-2-(trifluoromethyl)pyridin-4-ol as brown solid. Yield: 3.0 g (23%); ¹H NMR (400 MHz, DMSO-d₆): δ 9.08 (s, 1H), 7.43 (s, 1H) MS (ESI+) for CHNOS m/z 208.98 [M+H]⁺.

### 4-Chloro-5-nitro-2-(trifluoromethyl)pyridine

To a stirred solution of 5-nitro-2-(trifluoromethyl)pyridin-4-ol (3.9 g, 0.014 mol) in POCl₃ (2 mL, 0.021 mol) was added PCl₅ (4.5 g, 0.021 mol) at room temperature. The reaction was stirred at 80° C. for 16 h. The TLC showed the reaction to be complete. The reaction mixture was cooled to room temperature, diluted with DCM (100 mL) and washed with water (100 mL), sat. NaHCO₃ solution (100 mL) and brine (100 mL). The organics were dried (Na₂SO₄), filtered and concentrated under reduced pressure to afford 4-chloro-5-nitro-2-(trifluoromethyl)pyridine as yellow oil. Yield: 3.0 g (94%); ¹H NMR (400 MHz; DMSO-d₆): δ 9.42 (s, 1H), 8.56 (s, 1H); MS (ESI+) for CHNOS m/z 227.34 [M+H]⁺.

### 5-Nitro-2-(trifluoromethyl)pyridin-4-amine

To a stirred solution of 4-chloro-5-nitro-2-(trifluoromethyl)pyridine (1.0 g, 4.42 mmol) in EtOH (20 mL) NH₃ gas was purged at −78° C. for 15 min. The reaction mixture was stirred at room temperature for 2 h in a sealed tube. The TLC showed the reaction to be complete. The reaction mixture was evaporated under reduced pressure to afford 5-nitro-2-(trifluoromethyl)pyridin-4-amine as a yellow solid. Yield: 1.0 g (crude); ¹H NMR (400 MHz; DMSO-d₆): 59.02 (s, 1H), 7.39 (s, 1H); MS (ESI+) for CHNOS m/z 208.20 [M+H]⁺.

### 6-(Trifluoromethyl)pyridine-3,4-diamine

To a stirred solution of 5-nitro-2-(trifluoromethyl)pyridin-4-amine (1 g, 4.83 mmol) in MeOH/EtOAc (1.5:1) 50% Pd/C (1 g) was added at room temperature. The reaction mixture was stirred at room temperature for 5 h under H₂ atmosphere (1 atm). The TLC showed the reaction to be complete. The mixture was filtered through a celite bed and washed with MeOH (50 mL). The filtrate was evaporated under vacuum to afford 6-(trifluoromethyl)pyridine-3,4-diamine as a viscous liquid. Yield: 700 mg (81%); ¹H NMR (400 MHz; DMSO-d₆): δ 7.69 (s, 1H), 6.82 (s, 1H), 5.73 (bs, 2H), 5.08 (bs, 2H); MS (ESI+) for CHNOS m/z 178.03 [M+H]⁺.

### N-(6-(Trifluoromethyl)-1H-imidazo[4,5-c]pyridin-2-yl)benzo[d]oxazol-2-amine

A mixture of 6-(trifluoromethyl)pyridine-3,4-diamine (400 mg, 2.25 mmol) and dimethyl benzo[d]oxazol-2-ylcarbonimidodithioate (537 mg, 2.25 mmol) in DMF (10 mL) was stirred at 150° C. for 16 h. The TLC showed reaction to be complete. The reaction mixture was cooled to room temperature and poured into ice-water (50 mL). The solid precipitated was filtered, washed with H₂O (100 mL), triturated with Et₂O (25 mL) and dried under reduced pressure to give N-(6-(trifluoromethyl)-1H-imidazo[4,5-c]pyridin-2-yl)benzo[d]oxazol-2-amine as an off white solid. Yield: 210 mg (29%); ¹H NMR (400 MHz, DMSO-d₆): δ 8.74 (s, 1H), 7.82 (s, 1H), 7.49-7.53 (m, 2H), 7.22-7.28 (m, 1H), 7.14-7.20 (m, 1H); MS (ESI+) for CHNOS m/z 320.08 [M+H]⁺.

### Synthetic Route 22

### N-(Benzo[d]oxazol-2-yl)-4-methyloxazolo[4,5-c]pyridin-2-amine (Example 171)

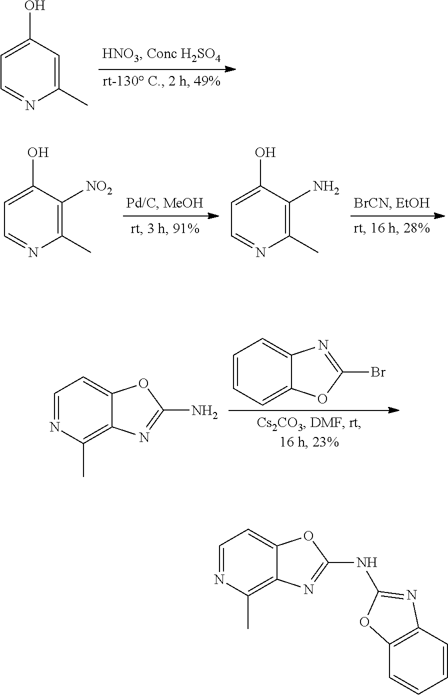

### 2-Methyl-3-nitropyridin-4-ol

To a solution of fuming nitric acid (6.6 mL, 158.8 mmol) and conc. sulphuric acid (6.6 mL, 123.8 mmol) was added 2-methylpyridin-4-ol (3 g, 27.5 mmol) portionwise at rt. The reaction mixture was heated at 130° C. for 2 h. The TLC showed reaction to be complete. The reaction mixture was cooled to rt, poured over ice and neutralized to pH ˜7 by using Na₂CO₃. The yellow precipitated solid was filtered and dried under vacuum at 60° C. The solid was taken in MeOH (50 mL) and stirred for 2 h at rt. The suspension was filtered and solid was discarded. The filtrate was concentrated under reduced pressure to afford the 2-methyl-3-nitropyridin-4-ol as a yellow solid.

Yield: 2.0 g (49%); ¹H NMR (400 MHz, DMSO-d₆): δ 8.53 (d, J=3.2 Hz, 1H), 7.47 (d, J=6.1 Hz, 1H), 5.95-6.05 (m, 1H), 2.08 (s, 3H); MS (ESI+) for CHNOS m/z 155.24 [M+H]⁺.

### 3-Amino-2-methyl pyridin-4-ol

To a solution of 2-methyl-3-nitropyridin-4-ol (1.5 g, 9.74 mmol) in MeOH (60 mL) was added 10% Pd/C (1.5 g). The reaction mixture was stirred at rt under H₂ balloon atmosphere for 3 h. The TLC showed reaction to be complete. The reaction mixture was passed through a pad of celite. The celite was washed with MeOH (100 mL). The filtrate was concentrated under reduced pressure to afford 3-amino-2-methylpyridin-4-ol as a brown semi solid. Yield: 1.1 g (91%); ¹H NMR (400 MHz, DMSO-d₆): δ7.17 (d, J=5.8 Hz, 1H), 5.84 (d, J=5.8 Hz, 1H), 3.75 (bs, 2H), 2.06 (s, 3H). MS (ESI+) for CHNOS m/z 125.14 [M+H]⁺.

### 4-Methyloxazolo[4,5-c]pyridin-2-amine

To a solution of 3-amino-2-methylpyridin-4-ol (500 mg, 4.03 mmol) in EtOH (10 mL) was added cynaogen bromide (1.3 g, 12.1 mmol) at rt. The reaction mixture was heated at rt for 16 h. The TLC showed reaction to be complete. The reaction mixture was poured in sat NaHCO₃ solution (50 mL) and extracted with 10% MeOH in DCM (3×30 mL). The organics layer was washed with brine (50 mL), dried (Na₂SO₄), filtered and concentrated under reduced pressure. The residue was purified by column chromatography using silica gel (100-200 mesh), eluting with 10% MeOH in EtOAc to afford 4-methyloxazolo[4,5-c]pyridin-2-amine as a light yellow solid. Yield: 170 mg (28%); ¹H NMR (400 MHz, DMSO-d₆): δ 8.05 (d, J=5.2 Hz, 1H), 7.62 (bs, 2H), 7.26 (d, J=5.2 Hz, 1H), 2.46 (s, 3H); MS (ESI+) for CHNOS m/z 150.19 [M+H]⁺.

### N-(Benzo[d]oxazol-2-yl)-4-methyloxazolo[4,5-c]pyridin-2-amine

To a solution of 4-methyloxazolo[4,5-c]pyridin-2-amine (170 mg, 1.14 mmol) in DMF (5 mL) were added 2-bromobenzo[d]oxazole (337 mg, 1.71 mmol) and Cs₂CO₃ (1.1 g, 3.42 mmol). The resulting mixture was stirred at rt for 16 h The TLC showed reaction to be complete. The solvent was removed under reduced pressure. The residue was triturated with 10% IPA in CHCl₃ (5×20 mL). The filtrate was concentrated under reduced pressure. The residue was purified by prep HPLC to afford N-(benzo[d]oxazol-2-yl)-4-methyloxazolo[4,5-c]pyridin-2-amine as an off white solid. Yield: 73 mg (23%); ¹H NMR (400 MHz, DMSO-d₆): δ 8.55 (d, J=6.2 Hz, 1H), 7.94 (d, J=6.2 Hz, 1H), 7.65 (d, J=7.6 Hz, 1H), 7.57 (d, J=7.6 Hz, 1H), 7.37-7.43 (m, 1H), 7.31-7.36 (m, 1H), 2.90 (s, 3H); MS (ESI+) for CHNOS m/z 267.22 [M+H]⁺.

### Synthetic Route 23

### 6-Chloro-N-(5-methylisoxazol-3-yl)benzo[d]oxazol-2-amine (Example 172)

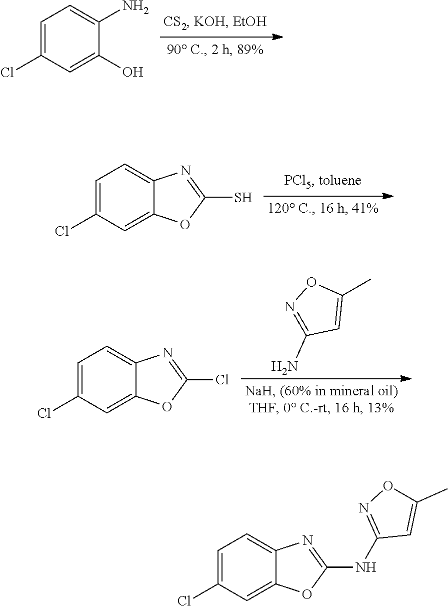

### 6-Chlorobenzo[d]oxazole-2-thiol

To a solution of KOH (4.7 g, 83.8 mmol) in EtOH (100 mL) were added 2-amino-5-chlorophenol (4.0 g, 27.8 mmol) and CS₂ (5.10 mL, 83.8 mmol) at rt. The reaction mixture was refluxed for 2 h. TLC showed the reaction to be complete. The solvent was removed under reduced pressure to give the crude residue. The residue was acidified with 1.0N HCl (100 mL) and extracted with EtOAc (3×100 mL). The organic layer was washed with brine (100 mL), dried (Na₂SO₄), filtered and concentrated under reduced pressure to afford 6-chlorobenzo[d]oxazole-2-thiol as an off-white solid. Yield: 4.6 g (89%); 1H NMR (400 MHz, DMSO-d6): δ 14.02 (bs, 1H), 7.73 (s, 1H), 7.34 (d, J=8.4 Hz, 1H), 7.23 (d, J=8.4 Hz, 1H); MS (ESI+) for CHNOS m/z 185.97 [M+H]⁺

### 2,6-Dichlorobenzo[d]oxazole

To a solution of 6-chlorobenzo[d]oxazole-2-thiol (5.0 g, 27.1 mmol) in toluene (150 mL) was added PCl₅ (28.2 g, 136 mmol) portion wise at rt. The reaction mixture was heated at 120° C. for 16 h. TLC showed the reaction to be complete. The reaction mixture was concentrated under reduced pressure to dryness. The residue was dissolved in Et₂O (100 mL). The insoluble solid was filtered and the filtrate was concentrated under reduced pressure. The crude residue was purified by column chromatography using silica gel (100-200 mesh), eluting with hexane to 3% EtOAc in hexane to afford 2,6-dichlorobenzo[d]oxazole as an orange solid. Yield: 2.1 g (41%); 1H NMR (400 MHz, DMSO-d6): δ 8.01 (d, J=1.6 Hz, 1H), 7.78 (d, J=8.6 Hz, 1H), 7.49 (dd, J=1.6, 8.6 Hz, 1H).

### 6-Chloro-N-(5-methylisoxazol-3-yl)benzo[d]oxazol-2-amine

To a solution of 5-methylisoxazol-3-amine (300 mg, 3.06 mmol) in dry THF (10 mL) was added sodium hydride (60% in mineral oil, 366 mg, 9.17 mmol) at 0° C. The resulting mixture was stirred at 0° C. for 15 min and 2,6-Dichlorobenzo[d]oxazole (575 mg, 3.06 mmol) was added. The reaction mixture was further stirred at rt for 16 h. TLC showed the reaction to be complete. The reaction mixture was quenched with sat. aq. NH₄Cl solution (20 mL) and extracted with EtOAc (3×20 mL). The organic layer was washed with brine (20 mL), dried (Na₂SO₄), filtered and concentrated under reduce pressure. The residue was triturated with Et₂O (25 mL) and dried under vacuum to give N-cyclopropyl-2-((5-(trifluoromethyl)benzo[d]oxazol-2-yl)amino)thiazole-4-carboxamide as a yellow solid. Yield: 99 mg (13%); MS (ESI+) for CHNOS m/z 249.99 [M+H]⁺; LC purity 99.8% (Ret. Time=5.96 min); ¹H NMR (400 MHz, DMSO-d₆+D₂O): 67.60 (s, 1H), 7.39 (d, J=7.5 Hz, 1H), 7.25 (d, J=7.5 Hz, 1H), 6.62 (s, 1H), 2.35 (s, 3H).

### Intermediate 168

### 2-Nitro-5-(Pyrrolidin-1-yl)phenol

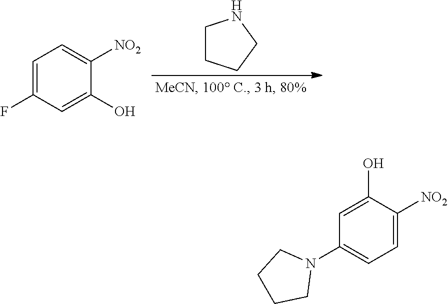 [SMILES: O=[N+]([O-])c1ccc(F)cc1O ; C1CCNC1 ; O=[N+]([O-])c1ccc(N2CCCC2)cc1O]

A mixture of 5-fluoro-2-nitrophenol (5.0 g, 31.8 mmol) and pyrrolidine (6.8 g, 95.5 mmol) in CH₃CN (30 mL) was stirred in a sealed tube at 100° C. for 3 h. TLC showed the reaction to be complete. The reaction mixture was cooled to rt and concentrated under reduced pressure. The residue was triturated with hexane (25 mL) and dried under reduced pressure to afford 2-nitro-5-(pyrrolidin-1-yl)phenol. Yield: 5.3 g (80%); MS (ESI+) for CHNOS m/z 209.30 [M+H]⁺.

The following intermediates were prepared in a similar manner to 2-nitro-5-(pyrrolidin-1-yl)phenol.

| | | | | Spectral Data |
|---|---|---|---|---|
| Name | Int | Structure | Yield | 1H NMR & LCMS |
| 2-Nitro-5- (piperidin-1- yl)phenol | 169 | 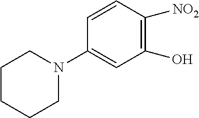 [SMILES: O=[N+]([O-])c1ccc(N2CCCCC2)cc1O] | 58% | MS (ESI+) for CHNOS m/z 222.97 [M + H]⁺; ¹H NMR (400 MHz, DMSO-d₆): δ 10.57 (bs, 1H), 7.82 (d, J = 9.5 Hz, 1H), 6.59 ((d, J = 9.5 Hz, 1H), 6.35 (s, 1H), 3.47 (bs, 4H), 1.53-1.62 (m, 6H) |
| 5-Morpholino- 2-nitrophenol | 170 | 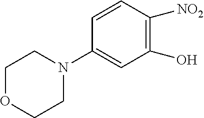 [SMILES: O=[N+]([O-])c1ccc(N2CCOCC2)cc1O] | 95% | MS (ESI+) for CHNOS m/z 224.95 [M + H]⁺; ¹H NMR (400 MHz, DMSO-d₆): δ 10.90 (bs, 1H), 7.87 (d, J = 9.5 Hz, 1H), 6.64 (dd, J = 2.4, 9.5 Hz, 1H), 6.43 (d, J = 2.4 Hz, 1H), 3.67-3.72 (m, 4H), 3.39-3.43 (m, 4H) |

### Intermediate 171

### 4-Fluoro-5-methoxy-2-nitrophenol

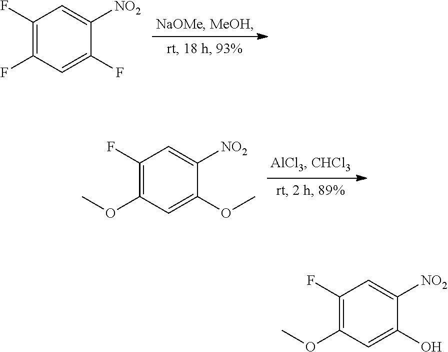

### 1-Fluoro-2,4-di methoxy-5-nitrobenzene

To a solution of 1,2,4-trifluoro-5-nitrobenzene (10 g, 56.5 mmol) in MeOH (80 mL) was added sodium methoxide (25% in MeOH, 27.0 mL, 124 mmol) slowly at 0° C. The reaction mixture was stirred at rt for 18 h. TLC showed the reaction to be complete. The reaction mixture was evaporated under reduced pressure, diluted with EtOAc (200 mL), washed with aqueous 1.0M citric acid (200 mL) and brine (100 mL). The organics were dried (Na₂SO₄), filtered and concentrated under reduced pressure to give 1-fluoro-2,4-dimethoxy-5-nitrobenzene as a yellow solid. Yield: 10.6 g (93%); ¹H NMR (400 MHz, DMSO-d₆): δ 7.95-7.99 (m, 1H), 7.01-7.03 (m, 1H), 4.01 (bs, 6H); MS (ESI+) for CHNOS m/z 202.09 [M+H⁺.

### 4-Fluoro-5-methoxy-2-nitrophenol

To a solution of 1-fluoro-2,4-dimethoxy-5-nitrobenzene (6.0 g, 29.8 mmol) in CHCl₃ (50 mL) was added AlCl₃ (6.0 g, 44.8 mmol) portion wise at 0° C. The reaction mixture was stirred at 70° C. for 1 h. TLC showed the reaction to be complete. The reaction mixture was poured into ice-water (100 mL), acidified to pH 2 with 1.0 N HCl and extracted with EtOAc (3×100 mL). The organics were dried (Na₂SO₄), filtered and concentrated under reduced pressure. The crude residue was purified by column chromatography using silica gel (100-200 mesh), eluting with 40% EtOAc in hexane to give 4-fluoro-5-methoxy-2-nitrophenol as a yellow solid. Yield: 5.0 g (89%); ¹H NMR (400 MHz, DMSO-d₆): δ 10.98 (bs, 1H), 7.72-7.98 (m, 1H), 6.70-6.95 (m, 1H), 3.92 (s, 3H); MS (ESI−) for CHNOS m/z 186.06 [M−H]⁻.

The following intermediates were prepared in a similar manner to 1-fluoro-2,4-dimethoxy-5-nitrobenzene.

| | | | | Spectral Data |
|---|---|---|---|---|
| | | | | 1H NMR & |
| Name | Int | Structure | Yield | LCMS |
| 1-Chloro-2,4- dimethoxy-5- nitrobenzene | 172 | 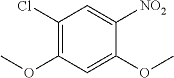 | 76% | MS (ESI+) for CHNOS m/z 218.16 [M + H]⁺; ¹H NMR |
| | | | | (400 MHz, |
| | | | | DMSO-d₆): δ |
| | | | | 8.09 (s, 1H), |
| | | | | 6.97 (s, 1H), |
| | | | | 4.03 (s, 3H), |
| | | | | 4.00 (s, 1H) |
| 1,5- Dimethoxy-2- methyl-4- nitrobenzene | 173 | 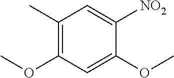 | 69% | MS (ESI+) for CHNOS m/z 198.11 [M + H]⁺; ¹H NMR |
| | | | | (400 MHz, |
| | | | | CDCl₃): δ 7.84 |
| | | | | (s, 1H), 6.45 |
| | | | | (s, 1H), 3.98 |
| | | | | (s, 3H), 3.92 |
| | | | | (s, 3H), 2.16 |
| | | | | (s, 3H) |

The following intermediates were prepared in a similar manner to 4-fluoro-5-methoxy-2-nitrophenol (step-2).

| | | | | Spectral Data |
|---|---|---|---|---|
| Name | Int | Structure | Yield | 1H NMR & LCMS |
| 4-Chloro-5- methoxy-2- nitrophenol | 174 | 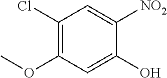 | 87% | MS (ESI−) for CHNOS m/z 202.06 [M − H]⁻; ¹H NMR (400 MHz, DMSO-d₆): δ 11.21 (bs, 1H), 8.07 (s, 1H), 6.81 (s, 1H), 3.93 (s, 3H) |
| 5-Methoxy-4- methyl-2- nitrophenol | 175 | 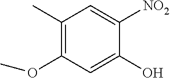 | 94% | MS (ESI−) for CHNOS m/z 182.13 [M − H]⁻; ¹H NMR (400 MHz, CDCl₃): δ 11.03 (bs, 1H), 7.85 (s, 1H), 6.47 (s, 1H), 3.90 (s, 3H), 2.15 (s, 3H) |

### Intermediate 176

### 5-Chloro-2-nitro-4-(trifluoromethoxy)phenol

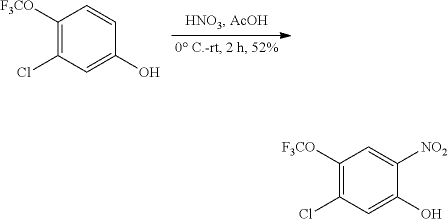 [SMILES: Oc1ccc(OC(F)(F)F)c(Cl)c1 ; O=[N+]([O-])c1cc(OC(F)(F)F)c(Cl)cc1O]

To a solution of 3-chloro-4-(trifluoromethoxy)phenol (5.0 g, 28.3 mmol) in acetic acid (20 mL) was added a solution of nitric acid (1.4 mL, 33.96 mmol) in acetic acid (4.0 mL) at 0° C. slowly. The reaction mixture was stirred at rt for 2 h. TLC showed the reaction to be complete. The reaction mixture was poured in to ice-water (200 mL), extracted with EtOAC (3×100 mL) and washed with brine (200 mL). The organics were dried (Na₂SO₄), filtered and concentrated under reduced pressure. The crude residue was purified by column chromatography using silica gel (100-200 mesh), eluting with 5% EtOAc in hexane to give 5-chloro-2-nitro-4-(trifluoromethoxy)phenol as a yellow solid. Yield: 3.2 g (52%). ¹H NMR (400 MHz, DMSO-d₆): 11.98 (bs, 1H), 8.15 (s, 1H), 7.36 (s, 1H). MS (ESI−) for CHNOS m/z 256.07 [M−H]⁻.

The following intermediates were prepared in a similar manner to 5-chloro-2-nitro-4-(trifluoromethoxy) phenol.

| | | | | Spectral Data |
|---|---|---|---|---|
| Name | Int | Structure | Yield | 1H NMR & LCMS |
| 5-Fluoro-2- nitro-4- (trifluoromethoxy) phenol | 177 | 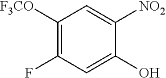 [SMILES: O=[N+]([O-])c1cc(OC(F)(F)F)c(F)cc1O] | 52% | MS (ESI−) for CHNOS m/z 240.16 [M − H]⁻; ¹H NMR (400 MHz, DMSO-d₆): δ 11.98 (bs, 1H), 8.20-8.26 (m, 1H), 7.15-7.20 (m, 1 H). |
| | | | | ¹H NMR (400 MHz, DMSO- |
| | | | | d₆, Fluorine decoupled): δ |
| | | | | 11.99 (bs, 1H), 8.24 (s, |
| | | | | 1H), 7.18 (s, 1H) |
| 5-Chloro-4- methyl-2- nitrophenol | 178 | 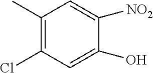 | 30% | MS (ESI−) for CHNOS m/z 186.16 [M − H]⁻; ¹H NMR (400 MHz, CDCl₃): δ 10.44 (bs, 1H), 7.98 (s, 1H), 7.20 (s, 1H), 2.36 (s, 3H). |
| 4-Chloro-2- nitro-5- (trifluoromethoxy) phenol | 179 | 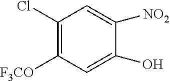 [SMILES: O=[N+]([O-])c1cc(Cl)c(OC(F)(F)F)cc1O] | 73% | MS (ESI−) for CHNOS m/z 256.10 [M − H]⁻; ¹H NMR (400 MHz, DMSO-d₆): δ 11.98 (bs, 1H), 8.28 (s, 1H), 7.25 (s, 1H) |
| 5-Fluoro-4- methoxy-2- nitrophenol | 180 | 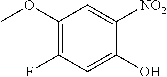 | 16% | MS (ESI−) for CHNOS m/z 186.15 [M − H]⁻; ¹H NMR (400 MHz, DMSO-d₆): δ 10.88 (bs, 1H), 7.62-7.71 (m, 1H), 6.99-7.06 (m, 1H), |
| | | | | 3.85 (s, 3H) |
| 4-Fluoro-2- nitro-5- (trifluoromethoxy) phenol | 181 | 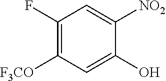 [SMILES: O=[N+]([O-])c1cc(F)c(OC(F)(F)F)cc1O] | 57% | MS (ESI−) for CHNSO m/z 240.05 [M − H]⁻; ¹H NMR (400 MHz, DMSO-d₆): δ 11.61 (bs, 1H), 8.15- 8.23 (m, 1H), 7.24-7.27 (m, 1H) |
| 5-Isopropyl-2- nitrophenol | 182 | 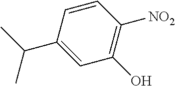 | 15% | MS (ESI−) for CHNOS m/z 180.28 [M − H]⁻; ¹H NMR (400 MHz, DMSO-d₆): δ 10.74 (bs, 1H), 7.28-7.38 (m, 1H), 6.86-6.94 (m, 2H), 2.65-2.74 (m, 1H), 1.18 (d, |
| | | | | J = 6.9 Hz, 6H) |

### Intermediate 183

### 4-Fluoro-5-methyl-2-nitrophenol

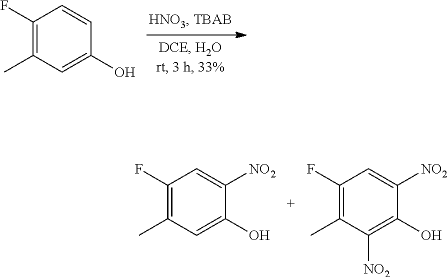

To a solution of 4-fluoro-3-methylphenol (10.0 g, 79.3 mmol), in DCE:H₂O (1:2, 150 mL) were added TBAB (2.6 g, 7.93 mmol) and HNO₃ (6.6 mL, 15.9 mmol) at rt. The reaction mixture was stirred at rt for 3 h. TLC showed the reaction to be complete. The reaction mixture was poured in to ice-water (100 mL) and extracted with DCM (3×100 mL). The organic layer was washed with brine (100 mL), dried (Na₂SO₄), filtered and concentrated under reduced pressure. The crude residue was purified by column chromatography using silica gel (100-200 mesh), eluting with 5% EtOAC in hexane to give 4-fluoro-5-methyl-2-nitrophenol as a yellow solid. Yield: 4.5 g (33%); ¹H NMR (400 MHz, DMSO-d₆): δ 10.81 (bs, 1H), 7.73-7.77 (m, 1H), 7.01-7.05 (m, 1H), 2.32 (s, 3H); MS (ESI−) for CHNOS m/z 170.05 [M−H]⁻. The formation of exact regioisomer was further confirmed by fluorine decoupled NMR.

The following intermediate was prepared in a similar manner to 4-fluoro-5-methyl-2-nitrophenol.

| | | | | Spectral Data |
|---|---|---|---|---|
| | | | | 1H NMR & |
| Name | Int | Structure | Yield | LCMS |
| 5-Fluoro-4- methyl-2- nitrophenol | 184 | 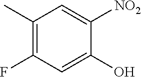 | 42% | MS (ESI−) for CHNOS m/z 170.10 [M − H]⁻; ¹H NMR (400 MHz, DMSO- |
| | | | | d₆): δ 11.14 |
| | | | | (bs, 1H), 7.90-7.94 |
| | | | | (m, 1H), 6.86-6.91 |
| | | | | (m, 1H), 2.50 (s, |
| | | | | 3H) |
| 4-chloro-5- methyl-2- nitrophenol | 185 | 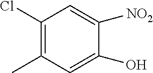 | 20% | MS (ESI−) for CHNOS m/z 186.15 [M − H]⁻ |

### Intermediate 186

### 2-Nitro-5-(trifluoromethoxy)phenol

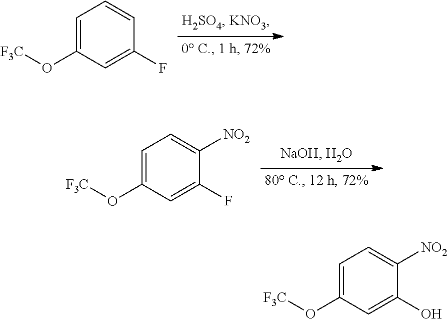 [SMILES: Fc1cccc(OC(F)(F)F)c1 ; O=[N+]([O-])c1ccc(OC(F)(F)F)cc1F ; O=[N+]([O-])c1ccc(OC(F)(F)F)cc1O]

### 2-Fluoro-1-nitro-4-(trifluoromethoxy)benzene

To a solution of 1-fluoro-3-(trifluoromethoxy)benzene (2.0 g, 11.1 mmol) in H₂SO₄ (5.0 mL) was added KNO₃ (1.34 g, 13.3 mmol) at 0° C. The reaction mixture was stirred at 0° C. for 1 h. TLC showed the reaction to be complete. The reaction mixture was diluted with H₂O (50 mL) and extracted with EtOAc (3×50 ml). The organic layer was dried over Na₂SO₄ and concentrated under reduced pressure. The residue was triturated with 10% EtOAc in hexane to afford 2-fluoro-1-nitro-4-(trifluoromethoxy)benzene as a yellow liquid. Yield: 1.8 g (crude). The crude data showed product and it was used in the next step without further purification.

### 2-Nitro-5-(trifluoromethoxy)phenol

To a solution of 2-fluoro-1-nitro-4-(trifluoromethoxy)benzene (1.8 g, 7.90 mmol) in H₂O (10 mL) was added NaOH (950 mg, 23.8 mmol) at rt. Then reaction mixture was stirred at 80° C. for 12 h. TLC showed the reaction to be complete. The reaction mixture was concentrated under reduced pressure. The residue was triturated with diethyl ether (20 mL), followed by acetone (20 mL) and dried under reduced pressure to afford 2-nitro-5-(trifluoromethoxy)phenol as a yellow solid. Yield: 1.3 g (72%); MS (ESI+) for CHNOS m/z 222.02 [M+H]⁻. LC purity 89-3% (Ret. Time-1.99 min).

### Intermediate 187

### 2-Amino-5-isopropylphenol

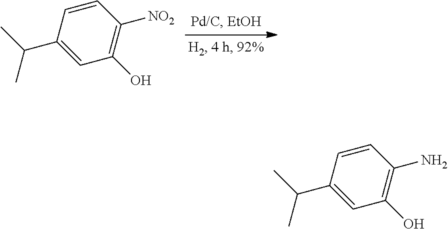

To a solution of 5-isopropyl-2-nitrophenol (2.09 g, 11 mmol) in EtOH (50 mL) was added 10% Pd/C (1.0 g). The reaction mixture was stirred at rt under H₂ balloon atmosphere for 4 h. TLC showed the reaction to be complete. The reaction mixture was passed through a pad of celite and the celite was washed with EtOH (100 mL). The filtrate was concentrated under reduced pressure to afford 2-amino-5-isopropylphenol as a yellowish solid. Yield: 1.2 g (70%); MS (ESI+) for CHNOS m/z 152.11 [M+H]⁺.

The following intermediates was prepared in a similar manner to 2-amino-5-isopropylphenol.

| | | | | Spectral Data |
|---|---|---|---|---|
| Name | Int | Structure | Yield | 1H NMR & LCMS |
| 2-Amino-5- (pyrrolidin-1- yl)phenol | 188 | 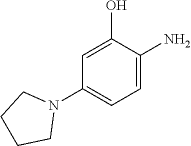 [SMILES: Nc1ccc(N2CCCC2)cc1O] | 51% | Crude data showed product. Used in next step without further purification. |
| 2-Amino-5- (piperidin-1- yl)phenol | 189 | 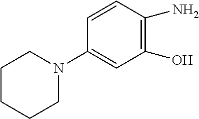 [SMILES: Nc1ccc(N2CCCCC2)cc1O] | 46% | Crude data showed product. Used in next step without further purification. |
| 2-Amino-5- morpholino- phenol | 190 | 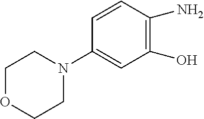 [SMILES: Nc1ccc(N2CCOCC2)cc1O] | 42% | Crude data showed product. Used in next step without further purification |
| 2-Amino-4- fluoro-5- methylphenol | 191 | 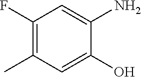 | 92% | MS (ESI−) for CHNOS m/z 140.0 [M − H]⁻; ¹H NMR (400 MHz, DMSO-d₆): δ 8.79 (bs, 1H), 6.40-6.48 (m, 1H), 6.29-6.35 (m, 1H), 4.56 (bs, |
| | | | | 2H), 1.99 (s, 3H) |
| 2-Amino-5- fluoro-4- methylphenol | 192 | 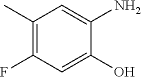 | 89% | MS (ESI+) for CHNOS m/z 142.06 [M + H]⁺; ¹H NMR (400 MHz, DMSO-d₆): δ 6.37-6.45 (m, 2H), 4.36 (bs, 2H), 1.99 (s, 3H) |
| 2-amino-4- fluoro-5- methoxyphenol | 193 | 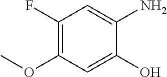 | 99% | MS (ESI−) for CHNOS m/z 156.16 [M + H]⁻ |
| 2-Amino-5- fluoro-4- (trifluoromethoxy) phenol | 194 | 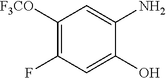 [SMILES: Nc1cc(OC(F)(F)F)c(F)cc1O] | 98% | MS (ESI−) for CHNOS m/z 210.16 [M − H]⁻; ¹H NMR (400 MHz, DMSO-d₆): δ 10.05 (bs, 1H), 6.60-6.65 (m, 2H), 4.74 (bs, 2H) |
| 2-Amino-5- methoxy-4- methylphenol | 195 | 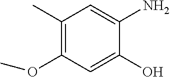 | 55% | The crude data showed product. It was used in next step without further purification |
| 2-Amino-5- fluoro-4- methoxypheno | 196 | 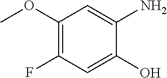 | 61% | The crude data showed product. It was used in next step without further purification |
| 2-Amino-5- (trifluoromethoxy) phenol | 197 | 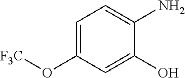 [SMILES: Nc1ccc(OC(F)(F)F)cc1O] | 86% | MS (ESI+) for CHNOS m/z 192.03 [M + H]⁺; LC purity 60% (Ret. Time- 1.88 min). |

### Intermediate 198

### 5-Chloro-4-methoxy-2-nitrophenol

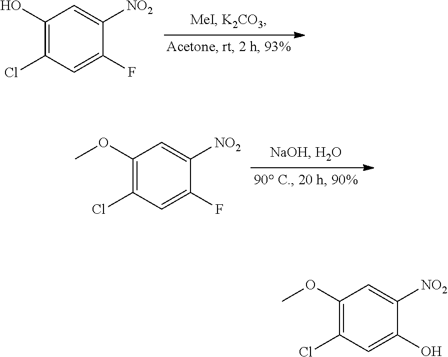

### 1-Chloro-5-fluoro-2-methoxy-4-nitrobenzene

To a solution of 2-chloro-4-fluoro-5-nitrophenol (5.0 g, 26.1 mmol) in acetone (100 mL) were added K₂CO₃ (18 g, 131 mmol) and methyl iodide (8.0 mL, 131 mmol) at rt. The reaction mixture was stirred at rt for 2 h. TLC showed the reaction to be complete. The reaction mixture was evaporated under reduced pressure, diluted with H₂O (100 mL) and extracted with EtOAc (3×100 mL). The organic layer was washed with H₂O (100 mL), brine (100 mL), dried (Na₂SO₄), filtered and concentrated under reduced pressure to give 1-chloro-5-fluoro-2-methoxy-4-nitrobenzene as a yellow solid. Yield: 5.0 g (93%); ¹H NMR (400 MHz, DMSO-d₆): δ 7.94-7.97 (m, 1H), 7.81-7.83 (m, 1H) 3.95 (s, 3H).

### 5-Chloro-4-methoxy-2-nitrophenol

To a solution of 1-chloro-5-fluoro-2-methoxy-4-nitrobenzene (4.0 g, 19.5 mmol) in H₂O (50 mL) was added NaOH (8.0 g, 195 mmol) at rt. The reaction mixture was stirred at 90° C. for 20 h. TLC showed the reaction to be complete. The reaction mixture was poured into ice-water (100 mL), acidified to pH 2 with 1.0 N HCl and extracted with EtOAc (3×50 mL). The organic layer was washed with brine (100 mL), dried (Na₂SO₄), filtered and concentrated under reduced pressure to give give 5-chloro-4-methoxy-2-nitrophenol as a yellow solid. Yield: 3.6 g (90%); ¹H NMR (400 MHz, DMSO-d₆): δ10.85 (bs, 1H), 7.60 (s, 1H), 7.24 (s, 1H), 3.85 (s, 3H); MS (ESI−) for CHNOS m/z 202.11 [M−H]⁻.

### Intermediate 199

### 2-Amino-5-cyclopropyl phenol

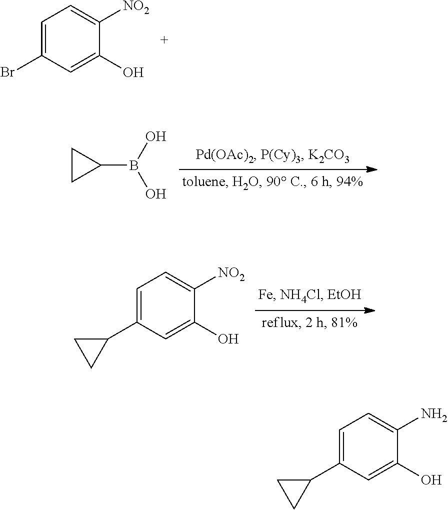 [SMILES: O=[N+]([O-])c1ccc(Br)cc1O ; "+" ; OB(O)C1CC1 ; O=[N+]([O-])c1ccc(C2CC2)cc1O ; Nc1ccc(C2CC2)cc1O]

### Cyclopropyl-2-nitrophenol

A mixture of 5-bromo-2-nitrophenol (5.0 g, 22.9 mmol), cyclopropylboronic acid (2.6 g, 29.9 mmol) and K₂CO₃ (10 g, 68.8 mmol) in toluene (70 mL) and H₂O (7.0 mL) was purged with N₂ gas at rt for 1 h. After N₂ purging, palladium acetate (260 mg, 1.15 mmol) and tricyclohexylphosphine (650 mg, 2.29 mmol) were added to this reaction mixture at rt. The reaction mixture was again purged with N₂ gas for 15 minutes at rt and stirred further at 90° C. for 6 h. TLC showed the reaction to be complete. The reaction mixture was cooled to rt, diluted with H₂O (100 mL) and extracted with EtOAc (3×50 mL). The organic layer was washed with brine (100 mL), dried (Na₂SO₄), filtered and concentrated under reduced pressure to obtain 5-cyclopropyl-2-nitrophenol as a brown viscous oil. Yield: 3.90 g (95%); ¹H NMR (400 MHz, DMSO): δ 10.68 (bs, 1H), 7.81 (d, J=8.7 Hz, 1H), 6.82 (s, 1H), 6.66 (d, J=8.7 Hz, 1H), 1.92-1.99 (m, 1H), 1.02-1.08 (m, 2H), 0.73-0.79 (m, 2H); MS (ESI−) for CHNOS m/z 177.97 [M−H]⁺.

### 2-Amino-5-cyclopropyl phenol

To a mixture of 5-cyclopropyl-2-nitrophenol (500 mg, 2.79 mmol) in EtOH (5.0 mL) and H₂O (5.0 mL) were added Fe powder (781 mg, 13.95 mmol) and ammonium chloride (740 mg 13.95 mmol) at rt. The reaction mixture was stirred at 90° C. for 2 h. TLC showed the reaction to be complete. The mixture was cooled to rt and filtered through celite pad. The filtrate was concentrated. The residue was diluted with H₂O (20 mL) and extracted with EtOAc (3×25 mL). The organics were dried (Na₂SO₄), filtered and concentrated under reduced pressure to afford 2-amino-5-cyclopropylphenol as a yellow solid. Yield: 337 mg (81%); MS (ESI+) for CHNOS m/z 149.92 [M+H]⁺.

The following intermediates were prepared in a similar manner to 2-amino-5-cyclopropylphenol (Step-2).

| | | | | Spectral Data |
|---|---|---|---|---|
| Name | Int | Structure | Yield | 1H NMR & LCMS |
| 2-Amino-4- chloro-5- methylphenol | 200 | 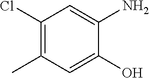 | 57% | MS (ESI−) for CHNOS m/z 156.04 [M − H]⁻; ¹H NMR (400 MHz, DMSO-d₆): δ 9.13 (bs, 1H), 6.53-6.59 (m, 2H), 4.58 (bs, 2H), 2.08 (s, |
| | | | | 3H) |
| 2-Amino-5- chloro-4- methoxyphenol | 201 | 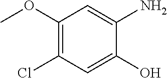 | 93% | MS (ESI+) for CHNOS m/z 174.14 [M + H]⁺; ¹H NMR (400 MHz, DMSO-d₆): δ 8.88 (bs, 1H), 6.60 (s, 1H), 6.40 (s, 1H), 4.70 (bs, 2H), |
| | | | | 3.66 (s, 3H) |
| 2-Amino-5- chloro-4- (trifluoromethoxy) phenol | 202 | 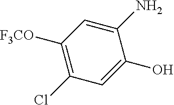 [SMILES: Nc1cc(OC(F)(F)F)c(Cl)cc1O] | 92% | MS (ESI+) for CHNOS m/z 228.17 [M + H]⁺; ¹H NMR (400 MHz, DMSO-d₆): δ .983 (bs, 1H), 6.72 (s, 1H), 6.67 (s, 1H), 5.06 (bs, 2H) |
| 2-Amino-5- chloro-4- methylphenol | 203 | 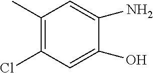 | 48% | MS (ESI+) for CHNOS m/z 157.99 [M + H]⁺; ¹H NMR (400 MHz, DMSO-d₆): δ 9.20 (bs, 1H), 6.61 (s, 1H), 6.49 (s, 1H), 4.56 (bs, 2H), |
| | | | | 2.09 (s, 3H) |
| 2-Amino-5- chloro-5- (trifluoromethoxy) phenol | 204 | 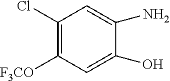 [SMILES: Nc1cc(Cl)c(OC(F)(F)F)cc1O] | 73% | MS (ESI−) for CHNOS m/z 226.03 [M − H]⁻; ¹H NMR (400 MHz, DMSO-d₆): δ 9.86 (bs, 1H), 6.72 (s, 1H), 6.69 (s, 1H), 5.03 (bs, 2H) |
| 2-Amino-4- fluoro-5- (trifluoromethoxy) phenol | 205 | 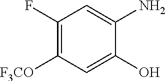 [SMILES: Nc1cc(F)c(OC(F)(F)F)cc1O] | 55% | MS (ESI−) for CHNOS m/z 210.12 [M − H]⁻; ¹H NMR (400 MHz, DMSO-d₆): δ 9.50 (bs, 1H), 6.62-6.66 (m, 1H), 6.48-6.55 (m, 1H), |
| | | | | 5.05, (bs, 2H) |
| 2-Chlorobenzo [d]oxazol-6- amine | 206 | 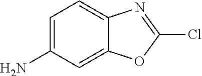 [SMILES: Nc1ccc2nc(Cl)oc2c1] | 63% | Crude data showed product. Proceeded further without purification |
| 2-Amino-5- (hydroxymethyl) phenol | 207 | 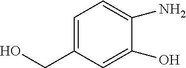 | 39% | MS (ESI+) for CHNOS m/z 139.93 [M + H]⁺; |
| 2-Amino-4- chloro-5- methoxyphenol | 208 | 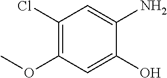 | 71% | MS (ESI+) for CHNOS m/z 174.04 [M + H]⁺; ¹H NMR (400 MHz, DMSO-d₆): δ 9.50 (bs, 1H), 6.61 (s, 1H), 6.49 (s, 1H), 4.38 (bs, 2H), |
| | | | | 3.32 (s, 3H) |

### Intermediate 209

### 2-Amino-5-chloro-3-methyl phenol

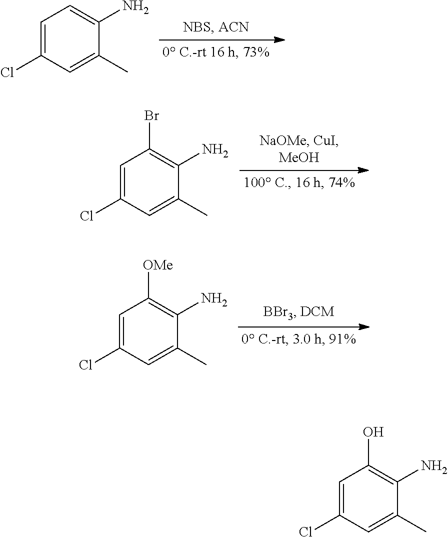

### 2-Bromo-4-chloro-6-methylaniline

To a solution of 4-chloro-2-methylaniline (15.0 g, 106.38 mmol) in ACN (150 mL) was added NBS (20.8 g, 110 mmol) at 0° C. slowly. The reaction mixture was stirred at rt for 16 h. TLC showed the reaction to be complete. The reaction mixture was diluted with H₂O (200 mL) and extracted with ethyl acetate (3×200 mL). The organic layer was washed with saturated aq NaHCO₃ solution (200 mL). The organic layer was washed with brine (200 mL), dried (Na₂SO₄), filtered and concentrated under reduced pressure. The residue was purified by column chromatography using silica gel (100-200 mesh), eluting with 5% EtOAc in hexane to afford 2-bromo-4-chloro-6-methylaniline as a light brown solid. Yield: 17.1 g (73%); ¹H NMR (400 MHz, CDCl₃): δ7.29 (d, J=1.9 Hz, 1H), 7.26 (s, 1H), 6.99 (bs, 1H), 3.90 (bs, 2H), 2.19 (s, 3H).

### 4-Chloro-2-methoxy-6-methylaniline

To a solution of 2-bromo-4-chloro-6-methylaniline (5.0 g, 22.8 mmol) and CuI (4.78 g, 25 mmol) in MeOH (50 mL) was added sodium methoxide solution (25% in MeOH, 25 mL) slowly at rt. The mixture was stirred at 100° C. for 16 h. TLC showed the reaction to be complete. The solvent was evaporated under reduced pressure. The residue was diluted with aq. saturated NH₄Cl solution (100 mL) and extracted with EtOAc (2×100 mL). The organic layer was washed with brine (100 mL), dried (Na₂SO₄), filtered and concentrated under reduced pressure. The residue was purified by column chromatography using silica gel (100-200 mesh), eluting with 5% EtOAc in hexane to afford 4-chloro-2-methoxy-6-methylaniline as dark brown liquid. Yield: 2.9 g (74%); (MS (ESI+) for CHNOS m/z 172.07 [M+H]⁺. ¹H NMR (400 MHz, DMSO-d₆): δ 6.72 (d, J=1.4 Hz, 1H), 6.65 (s, 1H), 4.53 (bs, 2H), 3.77 (s, 3H), 2.06 (s, 3H).

### 2-Amino-5-chloro-3-methyl phenol

To a solution of 4-chloro-2-methoxy-6-methylaniline (2.7 g, 15.7 mmol) in DCM (50 mL) was added BBr₃ (19.7 g, 78 mmol) at 0° C. slowly. The reaction mixture was stirred at rt for 3 h. TLC showed the reaction to be complete. The reaction mixture was neutralized with aq. NaHCO₃ solution (50 mL) at 0° C. and extracted with DCM (3×100 mL). The organic layer was washed with brine (50 mL), dried (Na₂SO₄), filtered and concentrated under reduced pressure to afford 2-amino-5-chloro-3-methylphenol as a brown solid. Yield: 2.27 g (91%); MS (ESI+) for CHNOS m/z 156.15 [M+H]⁺; ¹H NMR (400 MHz, DMSO-d₆): δ 9.46 (bs, 1H), 6.54 (s, 1H), 6.50 (s, 1H), 4.32 (bs, 2H), 2.03 (s, 3H).

The following intermediates were prepared in a similar manner to 6-Chlorobenzo[d]oxazole-2-thiol following Synthetic Route 23 (step 1).

| | | | | Spectral Data |
|---|---|---|---|---|
| Name | Int | Structure | Yield | 1H NMR & LCMS |
| 6- Isopropylbenzo [d]oxazole-2- thiol | 210 | 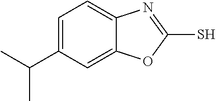 | 83% | MS (ESI−) for CHNOS m/z 192.02 [M − H]^{+; 1}HNMR (400 MHz, DMSO-d₆): δ 13.91 (bs, 1H), 7.32 (dd, J = 1.7, 6.8 Hz, 1H), 7.19- 7.24 (m, 2H), 3.19-3.32 (m, 1H), 1.22 (d, J = 6.8 Hz, 6H) |
| 6- nitrobenzo[d] oxazole-2-thiol | 211 | 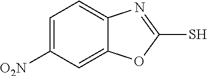 [SMILES: O=[N+]([O-])c1ccc2nc(S)oc2c1] | 81% | MS (ESI−) for CHNOS m/z 195.20 [M − H]^{+; 1}HNMR (400 MHz, DMSO-d₆): δ 14.39 (bs, 1H), 8.42 (bs, 1H), 8.22 (dd, J = 2.0, 8.7 Hz, 1H), |
| | | | | 7.40 (d, J = 8.7 Hz, 1H), |
| 5-Fluoro-6- methylbenzo[d] oxazole-2- thiol | 212 | 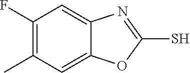 | 92% | MS (ESI+) for CHNOS m/z 184.05 [M + H]^{+; 1}HNMR (400 MHz, DMSO-d₆): δ 13.91 (bs, 1H), 7.40-7.57 (m, 1H), 7.04-7.18 (m, |
| | | | | 1H), 2.26 (s, 3H) |
| 6-Fluoro-5- methylbenzo[d] oxazole-2- thiol | 213 | 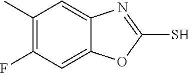 | 64% | MS (ESI+) for CHNOS m/z 184.0 [M + H]^{+; 1}HNMR (400 MHz, DMSO-d₆): δ 13.90 (bs, 1H), 7.47-7.54 (m, 1H), 7.12-7.19 (m, 1H), 2.27 (s, 3H) |
| 6-Chloro-5- methoxybenzo [d]oxazole-2- thiol | 214 | 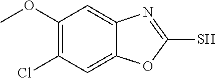 | 32% | MS (ESI−) for CHNOS m/z 214.11 [M − H]^{-; 1}HNMR (400 MHz, CDCl₃): δ 10.17 (s, 1H), 7.40 (s, 1H), 6.75 (s, 1H), 3.92 (s, 3H) |
| 6-Chloro-5- (trifluoromethoxy) benzo[d]oxazole- 2-thiol | 215 | 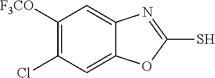 [SMILES: FC(F)(F)Oc1cc2nc(S)oc2cc1Cl] | 84% | MS (ESI−) for CHNOS m/z 268.08 [M − H]^{-; 1}HNMR (400 MHz, DMSO-d₆): δ 14.32 (bs, 1H), 8.01 (s, 1H), 7.41 (s, 1H) |
| 5-Fluoro-6- methoxybenzo [d]oxazole-2- thiol | 216 | 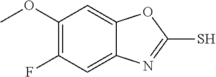 | 35% | MS (ESI−) for CHNOS m/z 198.15 [M − H]^{-; 1}HNMR (400 MHz, DMSO-d₆): δ 13.86 (bs, 1H), 7.47-7.55 (m, 1H), 7.15-7.26 (m, 1H), 3.85 (s, 3H) |
| 6-Fluoro-5- (trifluoromethoxy) benzo[d] oxazole-2-thiol | 217 | 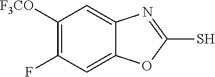 [SMILES: Fc1cc2oc(S)nc2cc1OC(F)(F)F] | 88% | MS (ESI−) for CHNOS m/z 252.19 [M − H]^{-; 1}HNMR (400 MHz, DMSO-d₆): δ 14.10 (bs, 1H), 7.81-7.92 (m, 1H), 7.39-7.52 (m, 1H), |
| 6-Methoxy-5- methylbenzo[d] oxazole-2- thiol | 218 | 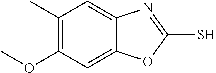 | 74% | MS (ESI+) for CHNOS m/z 196.09 [M + H]^{+; 1}HNMR (400 MHz, DMSO-d₆): δ 13.60 (bs, 1H), 7.23 (s, 1H), 7.05 (s, 1H), 3.73 (s, 3H), 2.18 (s, 3H) |
| 6-Fluoro-5- methoxybenzo [d]oxazole-2- thiol | 219 | 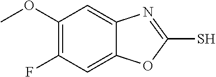 | 70% | MS (ESI−) for CHNOS m/z 198.0 [M − H]^{-; 1}HNMR (400 MHz, DMSO-d₆): δ 14.01 (bs, 1H), 7.59-7.72 (m, 1H), 6.90- 7.13 (m, 1H), 3.87 (s, 3H) |
| 6- (triFluoromethoxy) benzo[d] oxazole-2-thiol | 220 | 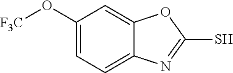 [SMILES: FC(F)(F)Oc1ccc2nc(S)oc2c1] | 54% | MS (ESI+) for CHNOS m/z 233.9 [M + H]⁺; LC purity 97.2% (Ret. Time- 1.70 min.) ¹H NMR (400 MHz; DMSO-d₆): δ 14.08 (bs, 1H), 7.73 (s, 1H), 7.31 (d, |
| | | | | J = 7.4 Hz, 2H). |

The following intermediates were prepared in a similar manner to 2,6-dichlorobenzo[d]oxazole following synthetic route 23 (step 2).

| | | | | Spectral Data |
|---|---|---|---|---|
| Name | Int | Structure | Yield | 1H NMR & LCMS |
| 2-chloro-6- nitrobenzo[d]ox- azole | 221 | 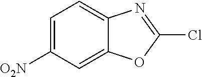 [SMILES: O=[N+]([O-])c1ccc2nc(Cl)oc2c1] | 62% | Crude data showed product. Proceeded further without purification |
| 2-Chloro-6- (trifluoromethoxy) benzo[d]oxazole | 222 | 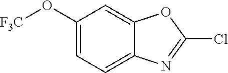 [SMILES: FC(F)(F)Oc1ccc2nc(Cl)oc2c1] | 48% | Crude data showed product. Proceeded further without purification |

### Intermediate 223

### 2,6-Dichloro-4-methylbenzo[d]oxazole

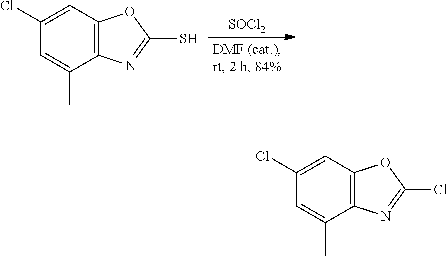

To a solution of 6-chloro-4-methylbenzo[d]oxazole-2-thiol (1.3 g, 6.5 mmol) in DCM (50 mL) were added DMF (0.5 mL) and SOCl₂ (12 mL) slowly at 0° C. The mixture was stirred at rt for 2 h. TLC showed the reaction to be complete. The solvent was evaporated under reduced pressure and the residue was diluted ice-water (20 mL) and extracted with EtOAc (3×25 mL). The organic layer was washed with brine (50 mL), dried (Na₂SO₄), filtered and concentrated under reduced pressure to afford 2,6-dichloro-4-methylbenzo[d]oxazole as a light brown solid. Yield: 1.1 g (85%); ¹H NMR (400 MHz, DMSO-d₆): δ7.80 (s, 1H), 7.35 (s, 1H), 2.48 (s, 3H).

The following intermediates were prepared in a similar manner to 2,6-Dichloro-4-methylbenzo[d]oxazole.

| | | | | Spectral Data |
|---|---|---|---|---|
| Name | Int | Structure | Yield | 1H NMR & LCMS |
| 2-Chloro-6- isopropylbenzo[d] oxazole | 224 | 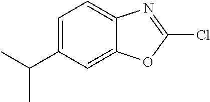 | 68% | ¹HNMR (400 MHz, CDCl₃): δ 7.25-7.34 (m, 2H), 7.19- 7.22 (m, 1H), 3.47-3.56 (m, 1H), 1.37 (d, J = 6.7 Hz, 6H) |
| 2,6-dichloro-5- methoxybenzo[d] oxazole | 225 | 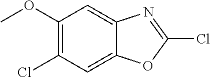 | 99% | ¹NMR (400 MHz, DMSO- d₆): δ 8.03 (s, 1H), 7.54 (s, 1H), 3.90 (s, 3H |
| 2,6-diChloro-5- (trifluoromethoxy) benzo[d]oxazole | 226 | 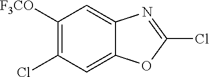 [SMILES: FC(F)(F)Oc1cc2nc(Cl)oc2cc1Cl] | 97% | ¹H NMR (400 MHz, DMSO-d₆): δ 8.34 (s, 1H), 8.12 (s, 1H). |
| 2-Chloro-5- fluoro-6- methoxybenzo[d] oxazole | 227 | 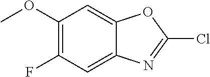 | 95% | ¹H NMR (400 MHz, DMSO-d₆): 7.61-7.79 (m, 2H), 3.90 (s, 3H). |
| 2-Chloro-6- fluoro-5- (trifluoromethoxy) benzo[d]oxazole | 228 | 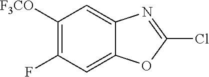 [SMILES: Fc1cc2oc(Cl)nc2cc1OC(F)(F)F] | 97% | ¹H NMR (400 MHz, DMSO-d₆): δ 8.13-8.29 (m, 2H) |
| 2-Chloro-5- fluoro-6- methylbenzo[d] oxazole | 229 | 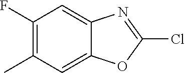 | 52% | ¹H NMR (400 MHz, DMSO- d₆): δ 7.71-7.77 (m, 1H), 7.56-7.65 (m, 1H), 2.34 (s, 3H) |
| 2-Chloro-6- fluoro-5- methylbenzo[d] oxazole | 230 | 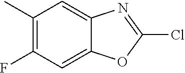 | 48% | ¹H NMR (400 MHz, DMSO- d₆): δ 7.57-7.87 (m, 2H), 2.32 (s, 3H) |
| 2-Chloro-6- methoxy-5- methylbenzo[d] oxazole | 231 | 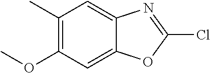 | 45% | ¹H NMR (400 MHz, DMSO- d₆): δ 7.51 (s, 1H), 7.40 (s, 1H), 3.85 (s, 3H), 2.22 (s, 3H) |
| 2-Chloro-6- fluoro-5- methoxybenzo[d] oxazole | 232 | 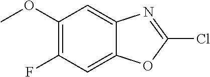 | 39% | ¹H NMR (400 MHz, DMSO- d₆): δ 7.85-7.93 (m, 1H), 7.53-7.63 (m, 1H), 3.89 (s, 3H). |

### Synthetic Route 24

### 5-Chloro-N-(thiazol-4-yl)benzo[d]oxazol-2-amine (Example 173)

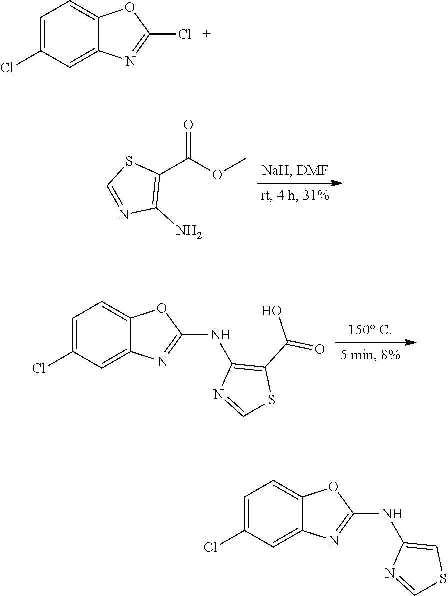

### 4-((5-Chlorobenzo[d]oxazol-2-yl)amino)thiazole-5-carboxylic acid

To a solution of methyl 4-aminothiazole-5-carboxylate (250 g, 1.58 mmol) in DMF (10 mL) was added NaH (60%, 190 g, 4.81 mmol) at 0° C. The suspension was stirred at 0° C. for 30 min and added 2,5-dichlorobenzo[d]oxazole (300 mg, 1.60 mmol). The reaction mixture was stirred at rt for 4 h. TLC showed the reaction to be complete. The reaction mixture was concentrated to dryness, diluted with H₂O (25 mL) and extracted with EtOAc (2×25 mL). The aqueous layer was acidified to pH 1-2 with 1.0N HCl. The solid precipitated was filtered and dried under vaccum to afford 4-((5-chlorobenzo[d]oxazol-2-yl)amino)thiazole-5-carboxylic acid as a brown solid. Yield: 150 mg (31%); ¹H NMR (400 MHz; DMSO-d₆): δ 11.79 (s, 1H), 9.25 (s, 1H), 7.95 (s, 1H), 7.08-7.72 (m, 3H); MS (ESI−) for CHNOS m/z 293.98 [M−H]⁻; LC purity 48.7% (Ret. Time-1.37 min).

### 5-Chloro-N-(thiazol-4-yl)benzo[d]oxazol-2-amine

4-((5-chlorobenzo[d]oxazol-2-yl)amino)thiazole-5-carboxylic acid (150 mg, 0.50 mmol) was heated at 150° C. for 5 min. TLC showed the reaction to be complete. The crude reaction mixture was purified by prep HPLC to afford 5-chloro-N-(thiazol-4-yl)benzo[d]oxazol-2-amine as an off white solid. Yield: 10 mg (8.0%); ¹H NMR (400 MHz; DMSO-d₆): δ 11.82 (bs, 1H), 9.03 (s, 1H), 7.66-7.71 (m, 2H), 7.45 (d, J=8.3 Hz, 1H), 7.27 (dd, J=1.9, 8.3 Hz, 1H); MS (ESI+) for CHNOS m/z 251.99 [M+H]; LC purity 97.2% (Ret. Time-5.78 min).

### Synthetic Route 25

### 5-Fluoro-N-(1,3,4-oxadiazol-2-yl)-6-(trifluoromethoxy)benzo[d]oxazol-2-amine

### Example 174

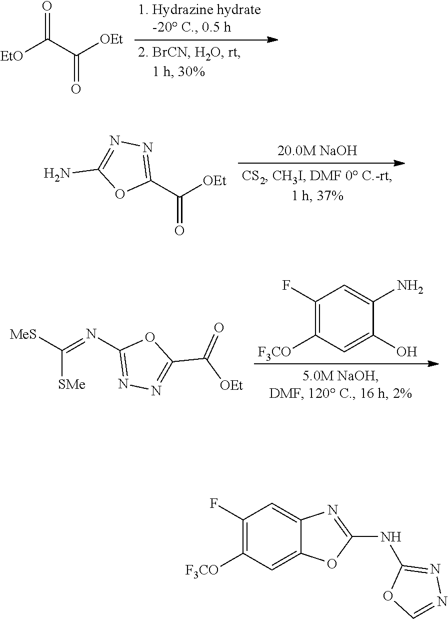 [SMILES: CCOC(=O)C(=O)OCC ; CCOC(=O)c1nnc(N)o1 ; CCOC(=O)c1nnc(N=C(SC)SC)o1 ; Nc1cc(F)c(OC(F)(F)F)cc1O ; Fc1cc2nc(Nc3nnco3)oc2cc1OC(F)(F)F]

### Ethyl 5-amino-1,3,4-oxadiazole-2-carboxylate

To a solution of diethyl oxalate (30.0 g, 205 mmol) in EtOH (50 mL) was added hydrazine hydrate (8.1 mL) in EtOH (20 mL) drop wise at −20° C. The reaction mixture was stirred at −20° C. for 0.5 h and filtered. To filtrate was added water (15 mL) and cyanogen bromide (16.5 g, 164 mmol) at r tans the reaction mixture was stirred at rt for 1 h. The precipitated solid was filtered, washed with Et₂O (100 mL) and dried under vacuum to afford ethyl 5-amino-1,3,4-oxadiazole-2-carboxylate as a white solid. Yield: 10 g (31%); ¹H NMR (400 MHz, DMSO-d₆): δ7.78 (s, 2H), 4.32 (q, J=7.0 Hz, 2H), 1.29 (t, J=7.0 Hz, 3H); MS (ESI+) for CHNOS m/z 158.02 [M+H]⁺.

### Ethyl 5-((bis(methylthio)methylene)amino)-1,3,4-oxadiazole-2-carboxylate

To a suspension of ethyl 5-amino-1,3,4-oxadiazole-2-carboxylate (20 g, 127 mmol) in DMF (200 mL) was added 20.0 M NaOH (6.35 mL, 127 mmol) at rt. The reaction mixture was stirred for 10 min and CS₂ (21.6 mL, 318.4 mmol) was added dropwise and the reaction mixture was further stirred for 10 min. An additional portion of 20.0 M NaOH (6.35 mL, 127 mmol) was added and reaction mixture was again stirred for 10 min. Finally, CH₃I (20 mL, 318.4 mmol) was added dropwise at rt. The reaction mixture was stirred at rt for 30 min. TLC showed the reaction to be complete. The mixture was poured into ice-water (400 mL) and the precipitated solid was filterd, washed with water (100 mL) followed by hexane (50 mL) and dried under reduced pressure to obtain ethyl 5-((bis(methylthio)methylene)amino)-1,3,4-oxadiazole-2-carboxylate as an off white solid solid. Yield: 12.5 g (37%); MS (ESI+) for CHNOS m/z 262.21 [M+H]⁺; ¹H NMR (400 MHz, DMSO-d₆): δ 4.40 (q, J=7.1 Hz, 2H), 2.68 (s, 6H), 1.31 (t, J=7.1 Hz, 3H).

### 5-Fluoro-N-(1,3,4-oxadiazol-2-yl)-6-(trifluoromethoxy)benzo[d]oxazol-2-amine

To a solution of 2-amino-4-fluoro-5-(trifluoromethoxy)phenol (700 mg, 3.3 mmol) in DMF (20 mL) was added 5.0 N NaOH solution (1.3 mL, 6.6 mmol) at rt. The reaction mixture was stirred at rt for 20 min and ethyl 5-((bis(methylthio)methylene)amino)-1,3,4-oxadiazole-2-carboxylate (865 mg, 3.3 mmol) was added at rt. The reaction mixture was stirred at 120° C. for 16 h. TLC showed the reaction to be complete. The reaction mixture was allowed to cool to rt, poured into ice-water (50 mL), acidified to pH 4-5 with 1.0N HCl and extracted with EtOAc (3×50 mL). The organics were washed with ice-cold water (2×50 mL), brine (100 mL), dried (Na₂SO₄), filtered and concentrated under reduced pressure and triturated with Et₂O (10 mL). The crude residue was further purified by prep HPLC to afford 5-fluoro-N-(1,3,4-oxadiazol-2-yl)-6-(trifluoromethoxy)benzo[d]oxazol-2-amine as off white solid Yield: 25 mg (2.0%); MS (ESI+) for CHNOS m/z 305.00 [M+H]⁺; LC purity 98.4% (Ret. Time-4.98 min); ¹H NMR (400 MHz, DMSO-d₆): δ 12.91 (bs, 1H), 8.86 (s, 1H), 7.94-8.01 (m, 1H), 7.44-7.56 (m, 1H).

### Intermediate 233

### 5-(Pyrrolidin-1-yl)-1,3,4-oxadiazol-2-amine

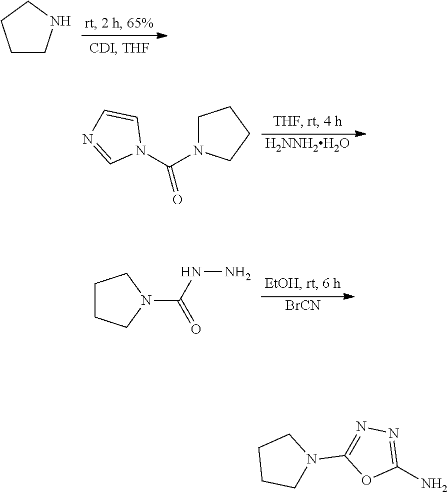

### (1H-imidazol-1-yl)(pyrrolidin-1-yl)methanone

To a solution of pyrrolidine (1.0 g, 14.0 mmol) in THF (20 mL) was added 1,1′-carbonyldiimidazole (6.8 g, 42.2 mmol) portion wise at rt. The reaction mixture was stirred at rt for 2 h. TLC showed the reaction to be complete. The mixture was diluted with H₂O (20 mL) and extracted with 10% MeOH in DCM (3×40 mL). The organics were washed with ice-cold water (3×20 mL), dried (Na₂SO₄), filtered and concentrated under reduced pressure to afford (1H-imidazol-1-yl)(pyrrolidin-1-yl)methanone as an off white solid. Yield: 1.51 g (65%); ¹H NMR (400 MHz, DMSO-d₆): δ 8.13 (s, 1H), 7.57 (s, 1H), 7.01 (s, 1H), 3.52 (bs, 4H), 1.85-1.89 (m, 4H); MS (ESI+) for CHNOS m/z 166.13 [M+H]⁺.

### Pyrrolidine-1-carbohydrazide

To a solution of (1H-imidazol-1-yl)(pyrrolidin-1-yl)methanone (7.0 g, 42.4 mmol) in THF (100 mL) was added hydrazine hydrate (22.0 mL, 424 mmol) at rt. The reaction mixture was stirred at rt for 4 h. TLC showed the reaction to be complete. The reaction mixture was concentrated under reduced pressure. The residue was triturated with Et₂O (50 mL), dried under vacuum to afford pyrrolidine-1-carbohydrazide as off colourless waxy solid. Yield: 7.5 g (Crude). MS (ESI+) for CHNOS m/z 129.92 [M+H]⁺.

### 5-(Pyrrolidin-1-yl)-1,3,4-oxadiazol-2-amine

To a solution of pyrrolidine-1-carbohydrazide (7.0 g, 54.2 mmol) in EtOH (100 mL) was added cynaogen bromide (11.3 g, 108.5 mmol) at rt. The reaction mixture was stirred at rt for 6 h. TLC showed the reaction to be complete. The reaction mixture was concentrated under reduced pressure and the residue was triturated with EtOH (50 mL), dried under vacuum to afford 5-(pyrrolidin-1-yl)-1,3,4-oxadiazol-2-amine as off white solid. Yield: 1.1 g (crude); MS (ESI+) for CHNOS m/z 155.16 [M+H]⁺; ¹H NMR (400 MHz, DMSO-d₆): δ 6.31 (bs, 2H), 3.21-3.33 (m, 4H), 1.86-1.90 (m, 4H).

The following intermediate was prepared in a similar manner to pyrrolidine-1-carbohydrazide.

| | | | | Spectral Data |
|---|---|---|---|---|
| Name | Int | Structure | Yield | 1H NMR & LCMS |
| Tetrahydrofuran- 3- carbohydrazide | 234 | 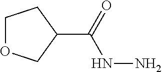 | 48% | MS (ESI+) for CHNOS m/z 131.10 [M + H]⁺ Crude data showed product. Proceeded further without purification |

The following intermediates were prepared in a similar manner to 5-(pyrrolidin-1-yl)-1,3,4-oxadiazol-2-amine.

| | | | | Spectral Data |
|---|---|---|---|---|
| Name | Int | Structure | Yield | 1H NMR & LCMS |
| 5-Cyclopropyl- 1,3,4-oxadiazol- 2-amine | 235 | 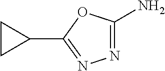 | 37% | MS (ESI+) for CHNOS m/z 126.21 [M + H]⁺; ¹H NMR (400 MHz, DMSO-d₆): δ 6.80 (bs, 2H), 1.90-2.05 (m, 1H), 0.90-1.08 (m, 2H), 0.75-0.90 (m, 2H) |
| 5-(Tetrahydrofuran- 3-yl)-1,3,4- oxadiazol-2- amine | 236 | 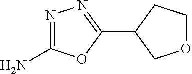 [SMILES: Nc1nnc(C2CCOC2)o1] | 42% | MS (ESI+) for CHNOS m/z 156.11 [M + H]⁺. Crude data showed product. Proceeded further without purification. |
| 5-Iospropyl- 1,3,4-oxadiazol- 2-amine | 237 | 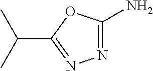 | 20% | ¹H NMR (400 MHz, DMSO- d₆): δ 6.83 (s, 2H), 2.91- 3.01 (m, 1H), 1.21 (d, J = 6.9 Hz, 6H) |

### Intermediate 238

### Ethyl 4-aminooxazole-2-carboxylate

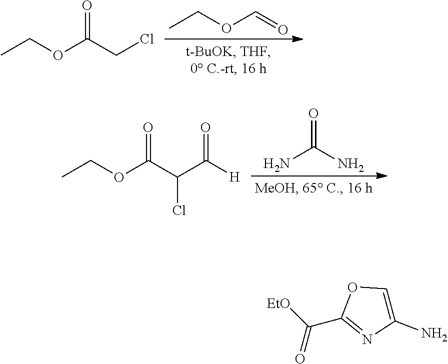

### Ethyl 2-chloro-3-oxopropanoate

To a suspension of potassium tert-butaoxide (16.4 g, 146 mmol) in Et₂O (300 mL) at 0° C. was added a mixture of ethyl 2-chloroacetate (15 g, 122 mmol) and ethyl formate (9 g, 122 mmol) in Et₂O (50 mL) slowly. The reaction was further stirred at rt for 16 h. TLC showed the reaction to be complete. The precipitated solid was filtered and washed with Et₂O (100 mL). The solid was added to ice-cold H₂O (200 mL), acidified to pH 5-6 with 1.0 N HCl and extracted with Et₂O (3×200 mL). The organics layer was washed with brine (100 mL), dried (Na₂SO₄), filtered and concentrated under reduced pressure to afford ethyl 2-chloro-3-oxopropanoate as a yellow oil, which was used in next step without further purification.

### Ethyl 4-aminooxazole-2-carboxylate

A mixture of ethyl 2-chloro-3-oxopropanoate (17 g, 113 mmol) and urea (33 g, 565 mmol) in MeOH (200 mL) was stirred at reflux for 18 h. TLC showed the reaction to be complete. The reaction mixture was cooled to rt and solvent was removed under reduced pressure. The residue was diluted with H₂O (100 mL) and extracted with 10% MeOH in DCM (3×100 mL). The organics layer was washed with brine (100 mL), dried (Na₂SO₄), filtered and concentrated under reduced pressure to afford ethyl 4-aminooxazole-2-carboxylate as off white solid (4.5 g crude). MS (ESI+) for CHNOS m/z 156.97 [M+H]⁺. The crude material was used in next step without further purification.

The following intermediates were prepared in a similar manner to ethyl 5-((bis(methylthio)methylene)amino)-1,3,4-oxadiazole-2-carboxylate.

| | | | | Spectral Data |
|---|---|---|---|---|
| Name | Int | Structure | Yield | 1H NMR & LCMS |
| Dimethyl (5- cyclopropyl- 1,3,4-oxadiazol- 2- yl)carbonimido- dithioate | 239 | 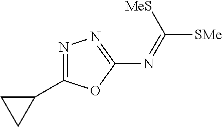 [SMILES: CSC(=Nc1nnc(C2CC2)o1)SC] | 60% | MS (ESI+) for CHNOS m/z 230.19 [M + H]⁺; ¹H NMR (400 MHz, DMSO-d₆): δ 2.56-2.80 (bs, 6H), 2.12- 2.20 (m, 1H), 1.09-1.18 (m, 2H), 0.90-1.07 (m, 2H) |
| Ethyl 4- ((bis(methylthio) methylene)amino) oxazole-2- carboxylate | 240 | 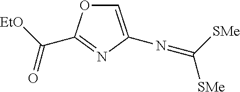 [SMILES: CCOC(=O)c1nc(N=C(SC)SC)co1] | 27% | MS (ESI+): for CHNOS m/z 260.90 [M + H]⁺; ¹H NMR (400 MHz, DMSO-d₆): δ 7.99 (s, 1H), 4.31 (q, J = 7.0 Hz, 2H), 2.63 (s, 6H), 1.29 (t, J = 7.0 Hz, 3H), |

### Intermediate 241

### 1-(2-Chlorobenzo[d]oxazol-6-yl)pyrrolidin-2-one

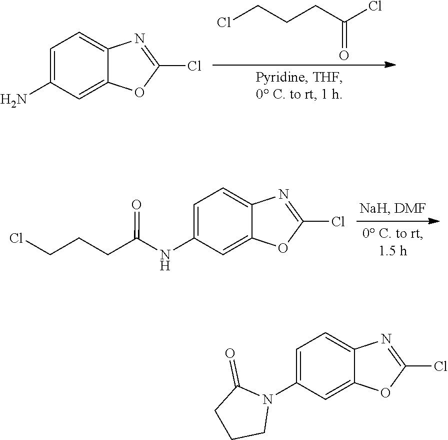 [SMILES: Nc1ccc2nc(Cl)oc2c1 ; O=C(Cl)CCCCl ; O=C(CCCCl)Nc1ccc2nc(Cl)oc2c1 ; O=C1CCCN1c1ccc2nc(Cl)oc2c1]

### 4-Chloro-N-(2-chlorobenzo[d]oxazol-6-yl)butanamide

To a solution of 2-chlorobenzo[d]oxazol-6-amine (1.0 g, 5.9 mmol) in THF (20 mL) were added pyridine (932 mg, 11.8 mmol) and 4-chlorobutanoyl chloride (1.0 g, 7.1 mmol) at 0° C. The reaction mixture was warmed to rt and stirred for 1 h. TLC showed the reaction to be complete. The reaction mixture was poured in to ice water (25 mL) and extracted with EtOAc (3×25 mL). The organics were washed with saturated NaHCO₃ solution, dried over (Na₂SO₄), filtered and concentrated under reduced pressure to give the crude residue. The residue was triturated with Et₂O (20 mL), filtered and dried under vacuum to afford 4-chloro-N-(2-chlorobenzo[d]oxazol-6-yl)butanamide as off brown solid. Yield: 1.4 g (crude). The crude data showed product and it was used for next step.

### 1-(2-Chlorobenzo[d]oxazol-6-yl)pyrrolidin-2-one

To a solution of 4-chloro-N-(2-chlorobenzo[d]oxazol-6-yl)butanamide (900 mg, 3.3 mmol) in DMF (10 mL) was added NaH (60%) (330 mg, 8.2 mmol) at 0° C. The reaction mixture was stirred at rt for 1.5 h. TLC showed the reaction to be complete. The reaction mixture was quenched with ice-cold water (50 mL) and extracted with EtOAc (3×50 mL). The organics were dried (Na₂SO₄), filtered and concentrated under reduced pressure. The residue was triturated with Et₂O (10 mL), filtered and dried under vacuum to afford 1-(2-chlorobenzo[d]oxazol-6-yl)pyrrolidin-2-one as an off white solid. Yield: 700 mg (crude). The crude data showed product and it was used in the next step without further purification

### Intermediate 242

### 4-Fluorobenzo[d]oxazol-2 (3H)-one

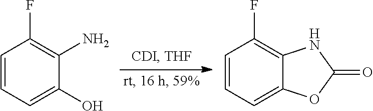 [SMILES: Nc1c(O)cccc1F ; O=c1[nH]c2c(F)cccc2o1]

To a solution of 2-amino-3-fluorophenol (2.5 g, 19.6 mmol) in THF (50 mL) was added CDI (15.9 g, 98.4 mmol) at rt. The reaction mixture was stirred at rt for 16 h. TLC showed the reaction to be complete. The reaction mixture was diluted with H₂O (100 mL) and extracted with EtOAc (3×100 mL). The organics were dried (Na₂SO₄), filtered and concentrated under reduced pressure. The residue was purified by column chromatography using silica gel (100-200 mesh), eluting with 10% EtOAc in hexane to afford 4-fluorobenzo[d]oxazol-2 (3H)-one as a pale brown solid. Yield: 1.8 g (59%); ¹H NMR (400 MHz, DMSO-d₆): δ 12.25 (bs, 1H), 7.12-7.19 (m, 1H), 7.06-7.12 (m, 2H); MS (ESI−) for CHNOS m/z 151.90 [M−H]⁺.

### Intermediate 243

### 2-Amino-5-chloropyridin-3-ol

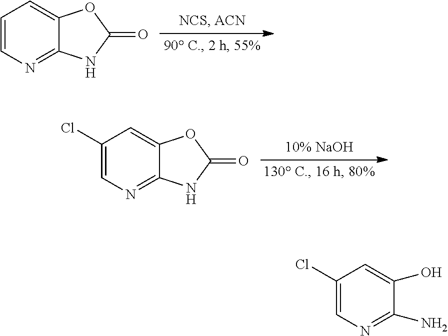 [SMILES: O=c1[nH]c2ncccc2o1 ; O=c1[nH]c2ncc(Cl)cc2o1 ; Nc1ncc(Cl)cc1O]

### 6-Chlorooxazolo[4,5-b]pyridin-2 (3H)-one

To a solution of oxazolo[4,5-b]pyridin-2 (3H)-one (5.0 g, 36.7 mmol) was added N-chlorosuccinimide (5.0 g, 45.8 mmol) at rt. The resulting reaction mixture was stirred at 90° C. for 2 h. TLC showed the reaction to be complete. The solvent was removed under reduced pressure and the residue was diluted with H₂O (100 mL) and extracted with EtOAc (3×100 mL). The organic layer was washed with H₂O (100 mL), dried (Na₂SO₄), filtered and concentrated under reduced pressure to 6-chlorooxazolo[4,5-b]pyridin-2 (3H)-one as a brown solid. Yield: 5.0 g (55%); ¹H NMR (400 MHz, DMSO-d₆): δ12.88 (bs, 1H), 8.08 (d, J=1.8 Hz, 1H), 7.91 (d, J=1.8 Hz, 1H); MS (ESI+) for CHNOS m/z 170.98 [M+H]⁺.

### 2-Amino-5-chloropyridin-3-ol

A suspension of 6-chlorooxazolo[4,5-b]pyridin-2 (3H)-one (6.0 g, 35.3 mmol) in 10% NaOH solution (200 mL) was stirred at 130° C. for 16 h. TLC showed the reaction to be complete. The reaction mixture was cooled to rt, neutralized with 6.0 N HCl and extracted with EtOAc (3×200 mL). The organic layer was washed with brine (200 mL), dried (Na₂SO₄), filtered and concentrated under reduced pressure to afford 2-amino-5-chloropyridin-3-ol as a brown solid. Yield: 4.0 g (80%); ¹H NMR (400 MHz, DMSO-d₆): δ10.03 (bs, 1H), 7.41 (d, J=2.1 Hz, 1H), 6.82 (d, J=2.1 Hz, 1H), 5.68 (bs, 2H); MS (ESI+) for CHNOS m/z 145.13 [M+H]⁺.

The following intermediate was prepared in a similar manner to 6-chlorooxazolo[4,5-b]pyridin-2 (3H)-one.

| | | | | Spectral Data |
|---|---|---|---|---|
| Name | Int | Structure | Yield | 1H NMR & LCMS |
| 6-chloro-4- fluorobenzo[d]ox- azol-2(3H)-one | 244 | 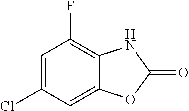 [SMILES: O=c1[nH]c2c(F)cc(Cl)cc2o1] | 52% | MS (ESI−) for CHNOS m/z 185.94 [M − H]⁺; ¹H NMR (400 MHz, DMSO-d₆): δ 12.45 (bs, 1H), 7.41 (s, 1H), 7.30-7.35 (m, 1H) |

The following intermediate was prepared in a similar manner to 2-amino-5-chloropyridin-3-ol.

| | | | | Spectral Data |
|---|---|---|---|---|
| Name | Int | Structure | Yield | 1H NMR & LCMS |
| 2-Amino-5- chloro-3- fluorophenol | 245 | 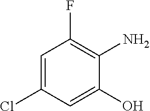 | 81% | MS (ESI−) for CHNOS m/z 159.95 [M − H]⁺; ¹H NMR (400 MHz, DMSO-d₆): δ 9.78 (bs, 1H), 6.60-6.79 (m, 1H), 6.54 (bs, 1H), 4.56 (bs, 2H) |

The following compounds were prepared in a similar manner to 5-Fluoro-N-(1,3,4-oxadiazol-2-yl)-6-(trifluoromethoxy)benzo[d]oxazol-2-amine.

| | | | | Spectral Data |
|---|---|---|---|---|
| Name | Ex | Structure | Yield | 1H NMR & LCMS |
| 6-Chloro-N- (5- cyclopropyl- 1,3,4- oxadiazol-2- yl)benzo[d]ox- azol-2-amine | 175 | 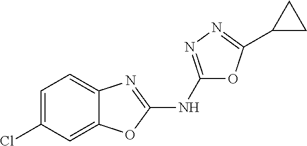 [SMILES: Clc1ccc2nc(Nc3nnc(C4CC4)o3)oc2c1] | 5% | MS (ESI+) for CHNOS m/z 277.06 [M + H]⁺; LC purity 98.2% (Ret. Time- 5.98); ¹H NMR (400 MHz, DMSO- d₆ + d-TFA): δ 7.65 (s, 1H), 7.41 (d, J = 8.4 Hz, 1H), 7.29 (d, J = 8.4 Hz, 1H), 2.06-2.20 (m, 1H), 0.97- 1.17 (m, 4H) |
| 6-Chloro-4- fluoro-N- (1,3,4- oxadiazol-2- yl)benzo[d]ox- azol-2-amine | 176 | 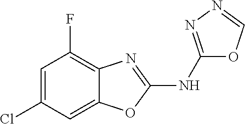 [SMILES: Fc1cc(Cl)cc2oc(Nc3nnco3)nc12] | 9% | MS (ESI+) for CHNOS m/z 254.96 [M + H]⁺; LC purity 94.5% (Ret. Time- 3.97); ¹H NMR (400 MHz, DMSO- d₆): δ 8.79 (s, 1H), 7.55 (s, 1H), 7.26-7.34 (m, 1H) |
| 2-((1,3,4- Oxadiazol-2- yl)amino)ben- zo[d]oxazol- 6-ol | 177 | 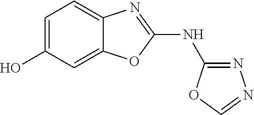 [SMILES: Oc1ccc2nc(Nc3nnco3)oc2c1] | 11% | MS (ESI+) for CHNOS m/z 219.22 [M + H]⁺; LC purity 96.7% (Ret. Time- 5.96 min); ¹H NMR (400 MHz, DMSO-d₆): δ 12.14 (bs, 1H), 9.67 (s, 1H), 8.79 (s, 1H), 7.23 (d, J = 8.5 Hz, 1H), 6.90 (d, J = 1.6 Hz, |
| | | | | 1H), 6.70 (d, d J = 1.6, 8.5 |
| | | | | Hz, 1H) |
| Methyl 2- ((1,3,4- oxadiazol-2- yl)amino)ben- zo[d]oxazole- 6-carboxylate | 178 | 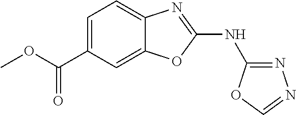 [SMILES: COC(=O)c1ccc2nc(Nc3nnco3)oc2c1] | 21% | MS (ESI+) for CHNOS m/z 260.85 [M + H]⁺; LC purity 99.8% (Ret. Time- 4.79 min); ¹H NMR (400 MHz, DMSO-d₆): δ 12.10 (bs, 1H), 8.88 (s, 1H), 7.99 (s, 1H), 7.94 (d, J = 8.2 Hz, 1H), 7.52 (d, J = 8.2 Hz, 1H), 3.87 (s, 3H) |
| 6-Bromo-N- (1,3,4- oxadiazol-2- yl)benzo[d]ox- azol-2-amine | 179 | 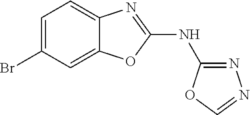 [SMILES: Brc1ccc2nc(Nc3nnco3)oc2c1] | 26% | MS (ESI−) for CHNOS m/z 278.96 [M − H]⁺; LC purity 96.4% (Ret. Time- 6.44 min); ¹H NMR (400 MHz, DMSO-d₆): δ 8.83 (s, 1H), 7.83 (s, 1H), 7.45 (dd, J = 1.3, 8.2 Hz,, 1H), 7.35 (d, J = 8.2 Hz, 1H) |
| 6-Cyclopropyl- N-(1,3,4- oxadiazol-2- yl)benzo[d]ox- azol-2-amine | 180 | 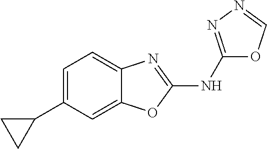 [SMILES: c1nnc(Nc2nc3ccc(C4CC4)cc3o2)o1] | 12% | MS (ESI+) for CHNOS m/z 243.04 [M + H]⁺; LC purity 99.8% (Ret. Time- 4.98 min); ¹H NMR (400 MHz, DMSO-d₆): δ 12.50 (bs, 1H), 8.79 (s, 1H), 7.28 (d, J = 8.0 Hz, 1H), 7.21 (s, 1H), 7.04 (d, J = 8.0 Hz,, 1H), 1.94-1.99 (m, 1H), 0.94- 0.97 (m, 2H), 0.67-0.70 (m, 2H) |
| (2-((1,3,4- Oxadiazol-2- yl)amino)ben- zo[d]oxazol- 6-yl)methanol | 181 | 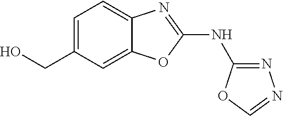 [SMILES: OCc1ccc2nc(Nc3nnco3)oc2c1] | 12% | MS (ESI+) for CHNOS m/z 233.01 [M + H]⁺; LC purity 98.6% (Ret. Time- 3.79 min); ¹H NMR (400 MHz, DMSO-d₆): δ 12.50 (bs, 1H), 8.82 (s, 1H), 7.36- 7.50 (m, 2H), 7.15 (d, J = 8.0 Hz,, 1H), 5.31 (bs, 1H), 4.53 (s, 2H) |
| 4-((6- chlorobenzo [d]oxazol-2- yl)amino)oxa- zole-2- carboxylic acid | 182 | 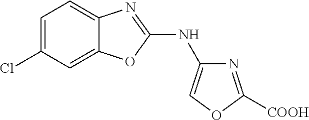 [SMILES: O=C(O)c1nc(Nc2nc3ccc(Cl)cc3o2)co1] | 9% | MS (ESI+) for CHNOS m/z 280.02 [M + H]⁺; LC purity 99.1% (Ret. Time- 3.47 min): ¹H NMR (400 MHz, DMSO-d₆): δ 10.62 (bs, 1H), 7.77 (s, 1H), 7.66 (s, 1H), 7.37 (d, J = 8.2 Hz, 1H), 7.28 (d, J = 8.2 Hz, 1H) |
| N-(1,3,4- oxadiazol-2- yl)-6- (pyrrolidin-1- yl)benzo[d]ox- azol-2-amine | 183 | 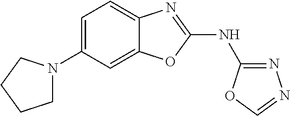 [SMILES: c1nnc(Nc2nc3ccc(N4CCCC4)cc3o2)o1] | 1% | MS (ESI+) for CHNOS m/z 272.07 [M + H]⁺; LC purity 98.5% (Ret. Time- 6.36 min); ¹H NMR (400 MHz, DMSO-d₆ + D₂O): δ 8.71 (s, 1H), 7.23 (d, J = 8.6 Hz, 1H), 6.69 (s, 1H), 6.47 (d, J = 8.6 Hz, 1H), 3.21 (bs, 4H), 1.94 (bs, 4H) |
| N-(1,3,4- Oxadiazol-2- yl)-6- (piperidin-1- yl)benzo[d]ox- azol-2-amine | 184 | 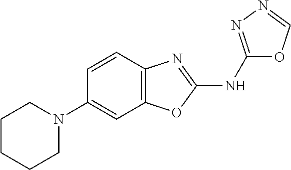 [SMILES: c1nnc(Nc2nc3ccc(N4CCCCC4)cc3o2)o1] | 4% | MS (ESI+) for CHNOS m/z 286.14 [M + H]⁺; LC purity 97.7% (Ret. Time- 3.51 min); ¹H NMR (400 MHz, DMSO-d₆): δ 12.01 (bs, 1H), 8.75 (s, 1H), 7.23 (d, J = 8.6 Hz, 1H), 7.11 (s, 1H), 6.86 (d, J = 8.6 Hz, 1H), 3.07-3.11 (m, 4H), 1.63 (bs, 4H), 1.52 (bs, 2H) |
| 6- Morpholino- N-(1,3,4- oxadiazol-2- yl)benzo[d]ox- azol-2-amine | 185 | 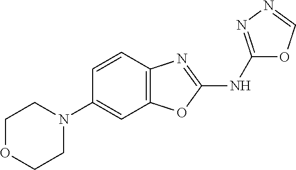 [SMILES: c1nnc(Nc2nc3ccc(N4CCOCC4)cc3o2)o1] | 2% | MS (ESI+) for CHNOS m/z 288.12 [M + H]⁺; LC purity 99.8% (Ret. Time- 4.04 min); ¹H NMR (400 MHz, DMSO-d₆): δ 12.12 (bs, 1H), 8.78 (s, 1H), 7.26 (d, J = 8.4 Hz, 1H), 7.16 (d, J = 1.7 Hz, 1H), 6.88 (dd, J = 1.7, 8.4 Hz, 1H), 3.72 (bs, 4H), 3.09 (bs, 4H) |
| 6-Nitro-N- (1,3,4- oxadiazol-2- yl)benzo[d]ox- azol-2-amine | 186 | 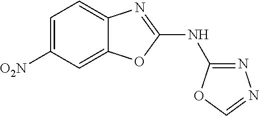 [SMILES: O=[N+]([O-])c1ccc2nc(Nc3nnco3)oc2c1] | 12% | MS (ESI+) for CHNOS m/z 248.00 [M + H]⁺; LC purity 99.1% (Ret. Time- 5.87 min); ¹H NMR (400 MHz, DMSO-d₆): δ 8.90 (s, 1H), 8.43 (d, J = 1.6 Hz, 1H), 8.24 (d, J = 1.6, 8.5 Hz, 1H), 7.57 (d, J = 8.5 Hz, 1H) |
| 5-Chloro-6- methyl-N- (1,3,4- oxadiazol-2- yl)benzo[d]ox- azol-2-amine | 187 | 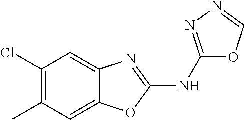 [SMILES: Cc1cc2oc(Nc3nnco3)nc2cc1Cl] | 3% | MS (ESI+) for CHNOS m/z 251.13 [M + H]⁺; LC purity 93.2% (Ret. Time- 5.75 min); ¹H NMR (400 MHz, DMSO-d₆ at 371.3K): δ 8.62 (s, 1H), 7.45 (s, 1H), 7.37 (s, 1H), 2.40 (s, 3H) |
| 6-Chloro-5- methyl-N- (1,3,4- oxadiazol-2- yl)benzo[d]ox- azol-2-amine | 188 | 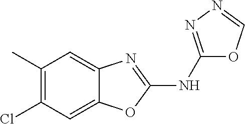 [SMILES: Cc1cc2nc(Nc3nnco3)oc2cc1Cl] | 6% | MS (ESI+) for CHNOS m/z 251.13 [M + H]⁺; LC purity 99.1% (Ret. Time- 4.65 min); ¹H NMR (400 MHz, DMSO-d₆): δ 8.82 (s, 1H), 7.69 (s, 1H), 7.35 (s, 1H), 2.36 (s, 3H) |
| 6-Chloro-4- methyl-N- (1,3,4- oxadiazol-2- yl)benzo[d]ox- azol-2-amine | 189 | 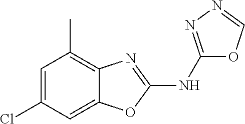 [SMILES: Cc1cc(Cl)cc2oc(Nc3nnco3)nc12] | 2% | MS (ESI+) for CHNOS m/z 251.16 [M + H]⁺; LC purity 99.7% (Ret. Time- 5.59 min); ¹H NMR (400 MHz, CD₃OD): δ 8.55 (s, 1H), 7.34 (s, 1H), 7.15 (s, 1H), 2.48 (s, 3H) |
| 5-Chloro-6- methoxy-N- (1,3,4- oxadiazol-2- yl)benzo[d]ox- azol-2-amine | 190 | 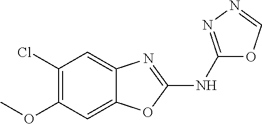 [SMILES: COc1cc2oc(Nc3nnco3)nc2cc1Cl] | 6% | MS (ESI+) for CHNOS m/z 266.96 [M + H]⁺; LC purity 95.2% (Ret. Time- 5.14 min); ¹H NMR (400 MHz, DMSO-d₆): δ 12.06 (bs, 1H), 8.82 (s, 1H), 7.52 (s, 1H), 7.44 (s, 1H), 3.88 (s, 3H). |
| 5-Chloro-N- (1,3,4- oxadiazol-2- yl)-6- (trifluoroeth- oxy)benzo[d] oxazol-2- amine | 191 | 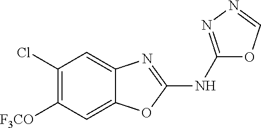 [SMILES: FC(F)(F)Oc1cc2oc(Nc3nnco3)nc2cc1Cl] | 2% | MS (ESI+) for CHNOS m/z 320.92 [M + H]⁺; LC purity 99.4% (Ret. Time- 4.54 min); ¹H NMR (400 MHz, DMSO-d₆ + d-TFA): δ 8.83 (s, 1H), 7.87 (s, 1H), 7.61 (s, 1H) |

### Synthetic Route 26

### 2-((1,3,4-oxadiazol-2-yl)amino)benzo[d]oxazole-6-carboxylic acid (Example 192)

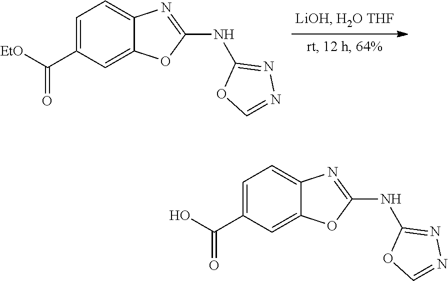 [SMILES: CCOC(=O)c1ccc2nc(Nc3nnco3)oc2c1 ; O=C(O)c1ccc2nc(Nc3nnco3)oc2c1]

To a solution of ethyl 2-((1,3,4-oxadiazol-2-yl)amino)benzo[d]oxazole-6-carboxylate (150 mg, 0.59 mmol) in THF:H₂O (2:1, 6 mL) was added lithium hydroxide (720 mg, 1.73 mmol) at rt. The reaction mixture was stirred at rt for 12 h. TLC showed the reaction to be complete. The reaction mixture was acidified to 3-4 pH with 1.0N HCl. The precipitate was filtered, washed with Et₂O (25 mL) and dried under vacuum to afford 2-((1,3,4-oxadiazol-2-yl)amino)benzo[d]oxazole-6-carboxylic acid as a white solid. Yield: 95 mg (32%); MS (ESI+) for CHNOS m/z 247.01 [M+H]⁺; LC purity 95.1% (Ret. Time—7.14); ¹H NMR (400 MHz, DMSO-d₆): δ 13.08 (bs, 1H), 8.87 (s, 1H), 7.96 (s, 1H), 7.92 (dd, J=1.2, 8.2 Hz, 1H), 7.50 (d, J=8.2 Hz, 1H).

### Synthetic Route 27

### 6-(2-methoxyethoxy)-N-(1,3,4-oxadiazol-2-yl)benzo[d]oxazol-2-amine (Example 193)

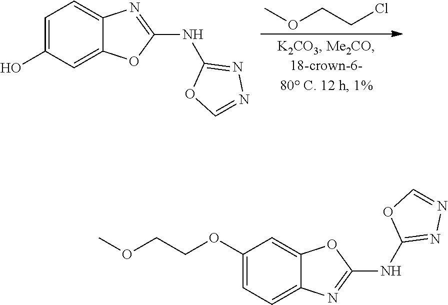

To a stirred solution of 2-((1,3,4-oxadiazol-2-yl)amino)benzo[d]oxazol-6-ol (100 mg, 0.45 mmol) in acetone (10 mL) was added 1-chloro-2-methoxyethane (52 mg, 0.55 mmol), potassium carbonate (190 mg, 1.4 mmol), and 18-Crown-6 ether. The reaction mixture was stirred at 80° C. for 16 h. TLC showed the reaction to be complete. The reaction mixture was cooled to rt and extracted with 10% IPA:CHCl₃ (3×25 mL). The organics were dried (Na₂SO₄), filtered and concentrated under reduced pressure to give 6-(2-methoxyethoxy)-N-(1,3,4-oxadiazol-2-yl)benzo[d]oxazol-2-amine after prep purification. To give as a brown solid. Yield: 1.6 mg (1.0%); ¹H NMR (400 MHz, DMSO-d₆): δ 8.78 (bs, 1H), 7.30 (d, J=9.0 Hz, 1H), 7.22 (s, 1H), 6.87 (d, J=9.0, Hz, 1H), 4.10 (bs, 2H), 3.65 (bs, 2H), 3.32 (s, 3H); MS (ESI+) for CHNOS m/z 277.35[M+H]⁺.

### Synthetic Route 28

### 6-Chloro-N-(5-ethynyl-1,3,4-oxadiazol-2-yl)benzo[d]oxazol-2-amine (Example 194)

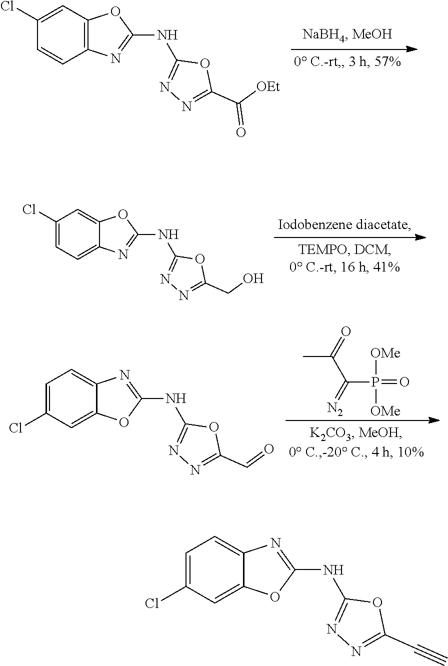

### (5-((6-chlorobenzo[d]oxazol-2-yl)amino)-1,3,4-oxadiazol-2-yl)methanol

To a stirred solution of ethyl 5-((6-chlorobenzo[d]oxazol-2-yl)amino)-1,3,4-oxadiazole-2-carboxylate (1.5 g, 4.87 mmol) in MeOH (30 mL) was added sodium borohydride (550 mg, 14.6 mmol) portionwise at 0° C. under N₂ atmosphere. The reaction was stirred at rt for 3 h. TLC showed the reaction to be complete. The solvent was removed under reduced pressure. The residue was dissolved in 5% MeOH in EtOAc (100 mL) and washed with saturated NH₄Cl solution (100 mL). The aqueous layer was extracted with 5% MeOH in EtOAC (3×50 mL). The organics layer was washed with brine (100 mL), dried (Na₂SO₄), filtered and concentrated under reduced pressure. The residue was triturated with Et₂O (20 mL) to afford 5-((6-chlorobenzo[d]oxazol-2-yl)amino)-1,3,4-oxadiazol-2-yl)methanol as off white solid. Yield: 745 mg (57%); MS (ESI+) for CHNOS m/z 267.19 [M+H]; ¹H NMR (400 MHz, DMSO-d₆): δ7.44 (s, 1H), 7.28 (d, J=7.4 Hz, 1H), 7.14 (d, J=7.4 Hz, 1H), 4.46 (s, 2H).

### 5-((6-Chlorobenzo[d]oxazol-2-yl)amino)-1,3,4-oxadiazole-2-carbaldehyde

To a solution of (5-((6-chlorobenzo[d]oxazol-2-yl)amino)-1,3,4-oxadiazol-2-yl)methanol (800 mg, 3.0 mmol) in DCM (15 mL) were added iodobezene diacetate (1.16 g, 3.60 mmol) and TEMPO (60 mg, 0.36 mmol) at 10° C. The reaction mixture was stirred at rt for 16 h. TLC showed the reaction to be complete. The mixture was diluted with ice-cold water (60 mL) and extracted with DCM (3×70 mL). The organic layer was washed with brine (60 mL), dried (Na₂SO₄) and concentrated under reduced pressure to dryness. The crude residue was purified by column chromatography using 4% MeOH in DCM to afford 5-((6-chlorobenzo[d]oxazol-2-yl)amino)-1,3,4-oxadiazole-2-carbaldehyde as an off white solid. Yield: 330 mg (41%); ¹H NMR (400 MHz, CDCl₃): δ9.86 (s, 1H), 7.48 (s, 1H), 7.35 (d, J=7.28 Hz, 1H), 7.27 (d, J=7.92 Hz, 1H). MS (ESI+) for CHNOS m/z 265.01 [M+H]⁺.

### 6-Chloro-N-(5-ethynyl-1,3,4-oxadiazol-2-yl)benzo[d]oxazol-2-amine

To a solution of 5-((6-chlorobenzo[d]oxazol-2-yl)amino)-1,3,4-oxadiazole-2-carbaldehyde (280 mg, 1.0 mmol) in dry MeOH (6.0 mL) were added K₂CO₃ (480 mg, 3.71 mmol) and Bestmann-Ohira Reagent (2.88 mL, 4.50 mmol) at 0° C. The reaction mixture was stirred at 20° C. for 4 h. TLC showed the reaction to be complete. The reaction mixture was concentrated under reduced pressure to dryness. The residue was purified by prep HPLC to afford 6-chloro-N-(5-ethynyl-1,3,4-oxadiazol-2-yl)benzo[d]oxazol-2-amine as an off white solid. Yield: 29 mg (10%); ¹H NMR (400 MHz; DMSO-d₆+d-TFA): δ 7.45 (d, J=1.7 Hz, 1H), 7.16 (d, J=8.4 Hz, 1H), 7.08 (dd, J=1.7, 8.4 Hz, 1H), 4.82 (s, 1H). MS (ESI+) for CHNOS m/z 260.99 [M+H]⁺. LCMS purity: 97.% (Ret. time: 4.52 min).

### Synthetic Route 29

### N²-(1,3,4-oxadiazol-2-yl)benzo[d]oxazole-2,6-diamine (Example 195)

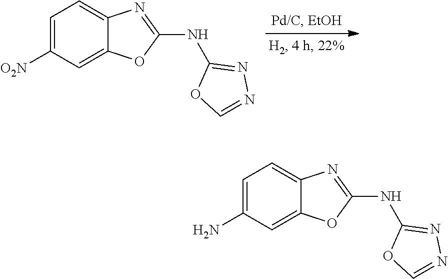 [SMILES: O=[N+]([O-])c1ccc2nc(Nc3nnco3)oc2c1 ; Nc1ccc2nc(Nc3nnco3)oc2c1]

### N²-(1,3,4-oxadiazol-2-yl)benzo[d]oxazole-2,6-diamine

To a solution of 6-nitro-N-(1,3,4-oxadiazol-2-yl)benzo[d]oxazol-2-amine (500 mg, 2.02 mmol) in EtOH (20 mL) was added 10% Pd/C (300 mg). The reaction mixture was stirred at rt under an H₂ balloon atmosphere for 4 h. TLC showed the reaction to be complete. The reaction mixture was passed through a pad of celite and washed with EtOH (50 mL). The filtrate was concentrated under reduced pressure to afford N²-(1,3,4-oxadiazol-2-yl)benzo[d]oxazole-2,6-diamine as a light brownish solid. Yield: 193 g (22%); MS (ESI−) for CHNOS m/z 218.01 [M+H]⁺; ¹H NMR (400 MHz, DMSO-d₆): δ 11.87 (bs, 1H), 8.75 (s, 1H), 7.09 (d, J=8.4 Hz, 1H), 6.66 (d, J=1.4 Hz, 1H), 6.48 (dd, J=1.4, 8.4 Hz, 1H), 5.30 (bs, 2H).

### Synthetic Route 30

### 6-Isopropoxy-N-(1,3,4-oxadiazol-2-yl)benzo[d]oxazol-2-amine (Example 196)

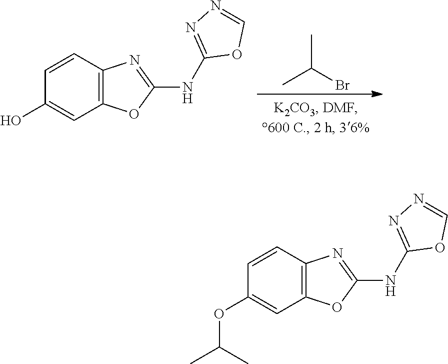

To a solution of 2-((1,3,4-oxadiazol-2-yl)amino)benzo[d]oxazol-6-ol (400 mg, 1.83 mmol) in DMF (2.0 mL) were added 2-bromopropane (180 mg, 1.47 mmol) and K₂CO₃ (506 g, 3.66 mmol). The resulting mixture was stirred at 60° C. for 2 h. TLC showed the reaction to be complete. The reaction mixture was poured in to ice-water (25 mL) and extracted with EtOAc (3×25 mL). The organic layers was washed with brine (50 mL), dried (Na₂SO₄), filtered and concentrated under reduced pressure. The crude LCMS showed 20% desired product along with dialkylated byproduct. The crude residue was purified by prep HPLC to afford 6-isopropoxy-N-(1,3,4-oxadiazol-2-yl)benzo[d]oxazol-2-amine as an off white solid. Yield: 17 mg (3.5%); MS (ESI+) for CHNOS m/z 261.10 [M+H]⁺; LC purity 97.8% (Ret. Time—4.32 min); ¹H NMR (400 MHz, DMSO-d₆): δ 9.89 (bs, 1H), 8.55 (s, 1H), 7.54 (d, J=8.6 Hz, 1H), 7.09 (d, J=2.1 Hz, 1H), 6.87 (dd, J=2.1, 8.6 Hz, 1H), 4.34-4.41 (m, 1H), 1.31 (d, J=6.7 Hz, 6H). The formation of exact regioisomer was confirmed by nOe experiment.

### Synthetic Route 31

### 5-Fluoro-6-methyl-N-(1,3,4-oxadiazol-2-yl)benzo[d]oxazol-2-amine (Example 197)

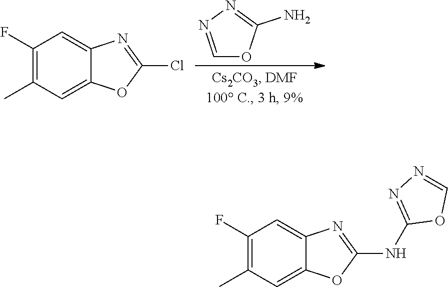

To a solution of 2-chloro-5-fluoro-6-methylbenzo[d]oxazole (710 mg, 3.82 mmol) in DMF (5.0 mL) were added 1,3,4-oxadiazol-2-amine (326 mg, 3.82 mmol) and Cs₂CO₃ (3.7 g, 11.51 mmol) at rt. The reaction mixture was stirred at 100° C. for 3 h. TLC showed the reaction to be complete. The reaction mixture was poured into ice-water (50 mL), acidified to pH 2-3 with 1.0 N HCl solution and extracted with EtOAc (3×50 mL). The organics were washed with water (3×50 mL), brine (100 mL), dried over (Na₂SO₄), filtered and concentrated under reduced pressure. The crude was triturated with diethyl ether (10 mL) and dried under reduced preesure to afford 5-fluoro-6-methyl-N-(1,3,4-oxadiazol-2-yl)benzo[d]oxazol-2-amine as an off white solid. Yield: 82 mg (9%). MS (ESI+) for CHNOS m/z 235.02 [M+H]⁺; LC purity 98.4% (Ret. Time—4.25 min); ¹H NMR (400 MHz, DMSO-d₆): δ 12.51 (bs, 1H), 8.80 (s, 1H), 7.42-7.52 (m, 1H), 7.13-7.21 (m, 1H), 2.26 (s, 3H).

### Intermediate 246

### 5-Amino-1,3,4-oxadiazole-2-carboxamide

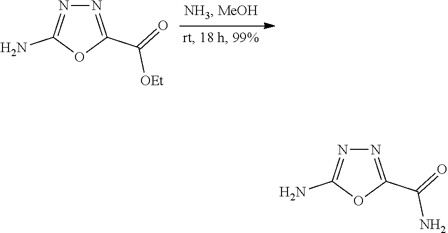 [SMILES: CCOC(=O)c1nnc(N)o1 ; NC(=O)c1nnc(N)o1]

To a solution of ethyl 5-amino-1,3,4-oxadiazole-2-carboxylate (4 g, 25.5 mmol) in MeOH (40 mL) was purged NH₃ (gas) at rt. The reaction vessel was sealed and the reaction mixture was stirred at rt for 18 h. The precipitated solid was filtered, washed with Et₂O (100 mL) and dried under vacuum to give 5-amino-1,3,4-oxadiazole-2-carboxamide as an off white solid. Yield: 3.5 g (99%); ¹H NMR (400 MHz, DMSO-d₆): δ8.12 (bs, 1H), 7.79 (bs, 1H), 7.49 (bs, 2H).

### Intermediate 247

### N-(2-chlorobenzo[d]oxazol-6-yl)-N-methylacetamide

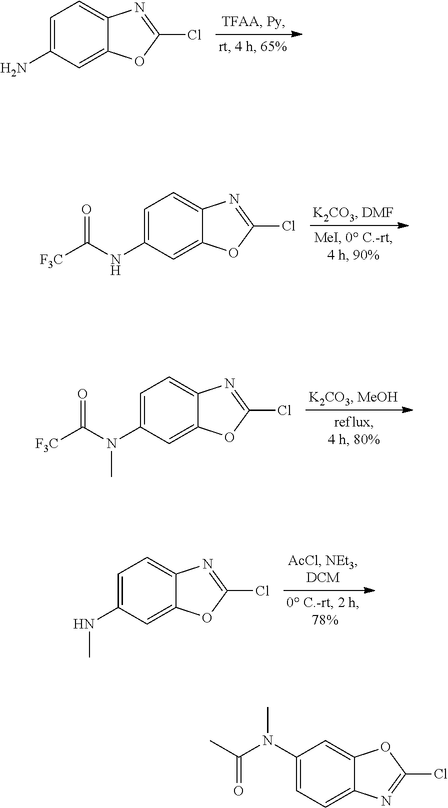 [SMILES: Nc1ccc2nc(Cl)oc2c1 ; O=C(Nc1ccc2nc(Cl)oc2c1)C(F)(F)F ; CN(C(=O)C(F)(F)F)c1ccc2nc(Cl)oc2c1 ; CNc1ccc2nc(Cl)oc2c1 ; CC(=O)N(C)c1ccc2nc(Cl)oc2c1]

### N-(2-chlorobenzo[d]oxazol-6-yl)-2,2,2-trifluoroacetamide

To a solution of 2-chlorobenzo[d]oxazol-6-amine (2.0 g, 11.9 mmol) in pyridine (8.0 mL) was added trifluoroacetic anhydride (0.9 mL, 5.9 mmol) at 0° C. The reaction mixture was stirred at rt for 3 h. TLC showed the reaction to be complete. The reaction mixture was quenched with ice-cold H₂O (50 mL) and extracted with EtOAc (3×50 mL). The organic layer was dried over Na₂SO₄, filtered and concentrated under reduced pressure to afford N-(2-chlorobenzo[d]oxazol-6-yl)-2,2,2-trifluoroacetamide as an off white solid. Yield: 2.0 g (65%). ¹H NMR (400 MHz, DMSO-d₆): δ 11.46 (bs, 1H), 8.05 (s, 1H), 7.69 (d, J=8.6 Hz, 1H), 7.50 (d, J=8.6 Hz, 1H).

### N-(2-Chlorobenzo[d]oxazol-6-yl)-2,2,2-trifluoro-N-methylacetamide

To a solution of N-(2-chlorobenzo[d]oxazol-6-yl)-2,2,2-trifluoroacetamide (1.3 g, 4.92 mmol) in DMF (5.0 mL) was added K₂CO₃ (4.92 mmol) at rt. The reaction mixture was stirred at rt for 1 h. After 1 h, the reaction mixture was cooled to 0° C. and methy iodide (0.6 mL, 9.84 mmol) was added slowly. The resulting reaction mixture was warmed to rt and stirred for 3 h. TLC showed the reaction to be complete. The reaction mixture was quenched in ice-cold H₂O (50 mL) and extracted with EtOAc (3×50 mL). The organic layer was dried over Na₂SO₄, filtered and concentrated under reduced pressure. The residue was triturated with Et₂O (50 mL) to afford N-(2-chlorobenzo[d]oxazol-6-yl)-2,2,2-trifluoro-N-methylacetamide as a waxy solid. Yield: 1.2 (90%). ¹H NMR (400 MHz, DMSO-d₆): δ 7.90 (bs, 1H), 7.74 (d, J=8.4 Hz, 1H), 7.44 (d, J=8.4 Hz, 1H), 3.56 (s, 3H).

### 2-Chloro-N-methylbenzo[d]oxazol-6-amine

To a solution of N-(2-chlorobenzo[d]oxazol-6-yl)-2,2,2-trifluoro-N-methylacetamide (1.2 g, 4.32 mmol) in MeOH (10 mL) was added K₂CO₃ (600 mg, 4.32 mmol) at rt.

The reaction mixture was refluxed for 3 h. TLC showed the reaction to be complete. The reaction mixture was cooled to rt and solvent was removed under reduced pressure. The residue was diluted with H₂O (50 mL) and extracted with EtOAc (3×50 mL). The organic layer was dried over Na₂SO₄, filtered and concentrated under reduced pressure to afford 2-chloro-N-methyl benzo[d]oxazol-6-amine as yellow waxy solid. Yield: 615 mg (80%). The crude data showed desired compound, which was used in next step without further purification.

### N-(2-chlorobenzo[d]oxazol-6-yl)-N-methylacetamide

To a solution of 2-chloro-N-methylbenzo[d]oxazol-6-amine (600 mg, 3.29 mmol) in DCM 20 mL) was added triethyl amine (1.2 mL, 9.87 mmol) at rt. The reaction mixture was cooled to 0° C. and acetyl chloride (0.45 mL, 6.58 mmmol) was added slowly. The reaction was allowed to warm to rt and stirred for 2 h at rt. TLC showed the reaction to be complete. The reaction mixture was diluted with H₂O (25 mL) and extracted with DCM (3×25 mL). The organic layer was dried over Na₂SO₄, filtered and concentrated under reduced pressure to afford N-(2-chlorobenzo[d]oxazol-6-yl)-N-methylacetamide as a yellow waxy solid. Yield: 660 mg (crude). The crude data showed desired compound, which was used in next step without further purification.

The following compounds were prepared in a similar manner to 5-fluoro-6-methyl-N-(1,3,4-oxadiazol-2-yl)benzo[d]oxazol-2-amine.

| | | | | Spectral Data |
|---|---|---|---|---|
| Name | Ex | Structure | Yield | 1H NMR & LCMS |
| 6-Chloro-N-(5- (pyrrolidin-1- yl)-1,3,4- oxadiazol-2- yl)benzo[d]oxa- zol-2-amine | 198 | 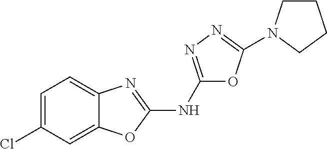 [SMILES: Clc1ccc2nc(Nc3nnc(N4CCCC4)o3)oc2c1] | 40% | MS (ESI+) for CHNOS m/z 306.0 [M + H]⁺; LC purity 98.0% (Ret. Time- 6.44 min); ¹H NMR (400 MHz, DMSO-d_{6 +} d-TFA): δ 7.32-7.72 (m, 3H), 3.56 (bs, 4H), 2.05 (bs, 4H) |
| 6-Chloro-N-(5- (tetrahydrofuran- 3-yl)-1,3,4- oxadiazol-2- yl)benzo[d]oxa- zol-2-amine | 199 | 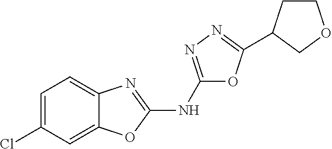 [SMILES: Clc1ccc2nc(Nc3nnc(C4CCOC4)o3)oc2c1] | 6% | MS (ESI+) for CHNOS m/z 307.07 [M + H]⁺; LC purity 96.3% (Ret. Time- 4.91 min); ¹H NMR (400 MHz, DMSO-d₆): δ 12.60 (bs, 1H), 7.71 (d, J = 1.6 Hz, 1H), 7.41 (d, J = 8.4 Hz, 1H), 7.32 (dd, J = 1.6, 8.4 Hz, 1H), 3.87-3.98 (m, 1H), 3.82-3.86 (m, 2H), |
| | | | | 3.71-3.80 (m, 1H), 3.62- |
| | | | | 3.70 (m, 1H), 2.24-2.34 |
| | | | | (m, 1H), 2.12-2.22 (m, 1H) |
| 5-((6- Chlorobenzo[d] oxazol-2- yl)amino)- 1,3,4- oxadiazole-2- carboxamide | 200 | 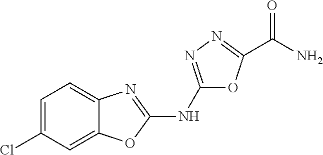 [SMILES: NC(=O)c1nnc(Nc2nc3ccc(Cl)cc3o2)o1] | 3% | MS (ESI+) for CHNOS m/z 280.10 [M + H]⁺; LC purity 99.7% (Ret. Time- 4.89 min); ¹H NMR (400 MHz, DMSO-d₆): δ 8.18 (bs, 1H), 7.81 (bs, 1H), 7.51 (s, 1H), 7.26 (d, J = 8.2 Hz, 1H), 7.18 (d, J = 8.2 Hz, 1H), 7.09 (bs, 1H) |
| 5-((6- Fluorobenzo[d] oxazol-2- yl)amino)- 1,3,4- oxadiazole-2- carboxamide | 201 | 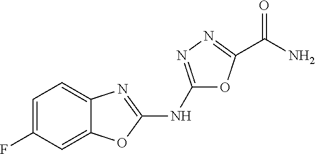 [SMILES: NC(=O)c1nnc(Nc2nc3ccc(F)cc3o2)o1] | 4% | MS (ESI+) for CHNOS m/z 264.00 [M + H]⁺; LC purity 98.8% (Ret. Time- 3.23 min); ¹H NMR (400 MHz, DMSO-d₆): δ 12.57 (bs, 1H), 8.93 (bs, 1H), 8.06 (bs, 1H), 7.60-7.70 (m, 1H), 7.41-7.50 (m, 1H), 7.12-7.23 (m, 1H) |
| 5-((5- Fluorobenzo[d] oxazol-2- yl)amino)- 1,3,4- oxadiazole-2- carboxamide | 202 | 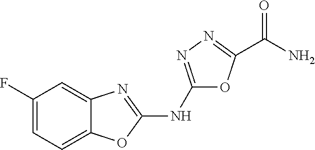 [SMILES: NC(=O)c1nnc(Nc2nc3cc(F)ccc3o2)o1] | 12% | MS (ESI+) for CHNOS m/z 264.03 [M + H]⁺; LC purity 98.9% (Ret. Time- 4.43 min); ¹H NMR (400 MHz, DMSO-d₆): δ 12.62 (bs, 1H), 8.41 (bs, 1H), 8.07 (bs, 1H), 7.53-7.67 (m, 1H), 7.23-7.34 (m, 1H), 7.01-7.18 (m, 1H) |
| 6-Fluoro-5- methyl-N- (1,3,4- oxadiazol-2- yl)benzo[d]oxa- zol-2-amine | 203 | 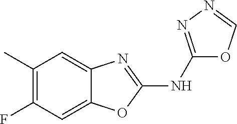 [SMILES: Cc1cc2nc(Nc3nnco3)oc2cc1F] | 4% | MS (ESI+) for CHNOS m/z 235.18 [M + H]⁺; LC purity 92.6% (Ret. Time- 4.33 min); ¹H NMR (400 MHz, DMSO-d₆): δ 12.34 (bs, 1H), 8.82 (s, 1H), 7.43-7.60 (m, 1H), 7.20- 7.38 (m, 1H), 2.32 (s, 3H) |
| 6-chloro-5- methoxy-N- (1,3,4- oxadiazol-2- yl)benzo[d]oxa- zol-2-amine | 204 | 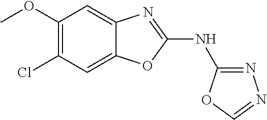 [SMILES: COc1cc2nc(Nc3nnco3)oc2cc1Cl] | 6% | MS (ESI+) for CHNOS m/z 266.99 [M + H]⁺; LC purity 97.8% (Ret. Time- 5.47 min); ¹H NMR (400 MHz, DMSO-d₆ + d-TFA: δ 8.79 (s, 1H), 7.68 (s, 1H), 7.19 (s, 1H), 3.85 (s, 3H) |
| 6-Chloro-N- (1,3,4- oxadiazol-2- yl)-5- (trifluoro- methoxy)benzo [d]ox- azol-2-amine | 205 | 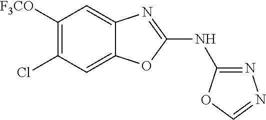 [SMILES: FC(F)(F)Oc1cc2nc(Nc3nnco3)oc2cc1Cl] | 10% | MS (ESI+) for CHNOS m/z 320.89 [M + H]⁺; LC purity 98.9% (Ret. Time- 6.08 min); ¹H NMR (400 MHz, DMSO-d₆): δ 8.66 (s, 1H), 7.99 (s, 1H), 7.53 (s, 1H) |
| 5-Fluoro-6- methoxy-N- (1,3,4- oxadiazol-2- yl)benzo[d]oxa- zol-2-amine | 206 | 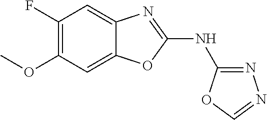 [SMILES: COc1cc2oc(Nc3nnco3)nc2cc1F] | 1% | MS (ESI−) for CHNOS m/z 249.26 [M − H]⁻; LC purity 96.3%; ¹H NMR (400 MHz, DMSO-d₆): δ 12.36 (bs, 1H), 8.82 (s, 1H), 7.49-7.62 (m, 1H), 7.21- 7.40 (m, 1H), 3.86 (s, 3H) |
| 6-Fluoro-N- (1,3,4- oxadiazol-2- yl)-5- (trifluoromethoxy) benzo[d]ox- azol-2-amine | 207 | 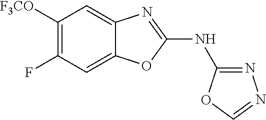 [SMILES: Fc1cc2oc(Nc3nnco3)nc2cc1OC(F)(F)F] | 11% | MS (ESI+) for CHNOS m/z 304.8 [M + H]⁺; LC purity 99.8% (Ret. Time- 6.10 min); ¹H NMR (400 MHz, DMSO-d₆): δ 12.82 (bs, 1H), 8.86 (s, 1H), 7.89-7.96 (m, 1H), 7.51- 7.60 (m, 1H) |
| 6-Methoxy-5- methyl-N- (1,3,4- oxadiazol-2- yl)benzo[d]oxa- zol-2-amine | 208 | 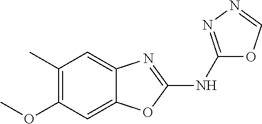 [SMILES: COc1cc2oc(Nc3nnco3)nc2cc1C] | 3% | MS (ESI+) for CHNOS m/z 247.11 [M + H]⁺; LC purity 97.7% (Ret. Time- 4.62 min); ¹H NMR (400 MHz, DMSO-d₆): δ 8.79 (s, 1H), 7.20-7.26 (m, 2H), 3.81 (s, 3H), 2.19 (s, 3H) |
| 6-Fluoro-5- methoxy-N- (1,3,4- oxadiazol-2- yl)benzo[d]oxa- zol-2-amine | 209 | 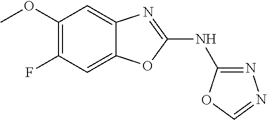 [SMILES: COc1cc2nc(Nc3nnco3)oc2cc1F] | 2% | MS (ESI+) for CHNOS m/z 250.98 [M + H]⁺; LC purity 92.9% (Ret. Time- 3.94 min); ¹H NMR (400 MHz, DMSO-d₆): δ 12.40 (bs, 1H), 8.82 (s, 1H), 7.63-7.68 (m, 1H), 7.19- 7.23 (m, 1H), 3.86 (s, 3H) |
| N-(1,3,4- Oxadiazol-2- yl)-6- (trifluoromethoxy) benzo[d]ox- azol-2-amine | 210 | 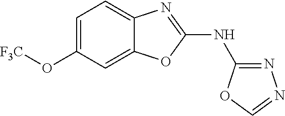 [SMILES: FC(F)(F)Oc1ccc2nc(Nc3nnco3)oc2c1] | 25% | MS (ESI+) for CHNOS m/z 287.04 [M + H]⁺; LC purity 96.3% (Ret. Time- 4.83 min); ¹H NMR (400 MHz, DMSO-d₆): δ 12.43 (bs, 1H), 8.85 (s, 1H), 7.74 (s, 1H), 7.49 (d, J = 8.5 Hz, 1H), 7.31 (d, J = |
| | | | | 8.5 Hz, 1H) |
| 6-Isopropyl-N- (1,3,4- oxadiazol-2- yl)benzo[d]oxa- zol-2-amine | 211 | 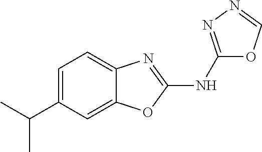 [SMILES: CC(C)c1ccc2nc(Nc3nnco3)oc2c1] | 4% | MS (ESI+) for CHNOS m/z 244.10 [M + H]+; LC purity 98.3% (Ret. Time- 5.11 min); 1H NMR (400 MHz, DMSO-d₆): δ 8.80 (s, 1H), 7.34 (d, J = 8.4 Hz, 1H), 7.01-7.22 (m, 2H), 3.33-3.40 (m, 1H), 1.26 (d, J = 6.9 Hz, 6H) |
| 1-(2-((1,3,4- oxadiazol-2- yl)amino)benz- o[d]oxazol-6- yl)pyrrolidin-2- one | 212 | 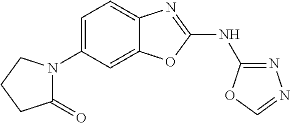 [SMILES: O=C1CCCN1c1ccc2nc(Nc3nnco3)oc2c1] | 2% | MS (ESI+) for CHNOS m/z 286.03 [M + H]+; LC purity 93.8% (Ret. Time- 5.53 min); 1H NMR (400 MHz, DMSO-d₆): δ 8.80 (s, 1H), 7.92 (s, 1H), 7.48 (d, J = 7.6 Hz, 1H), 7.39 (d, J = 7.6 Hz, 1H) 3.82- |
| | | | | 3.88 (m, 2H), 2.49 (bs, |
| | | | | 2H), 2.04-2.09 (m, 2H) |
| 6-Chloro-N-(5- (tetrahydrofuran- 2-yl)-1,3,4- oxadiazol-2- yl)benzo[d]oxa- zol-2-amine | 213 | 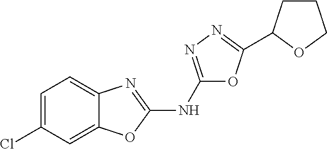 [SMILES: Clc1ccc2nc(Nc3nnc(C4CCCO4)o3)oc2c1] | 3% | MS (ESI+) for CHNOS m/z 307.04 [M + H]⁺; LC purity 99.4% (Ret. Time- 5.84 min); ¹H NMR (400 MHz, DMSO-d₆): δ 12.46 (bs, 1H), 7.34 (d, J = 1.2 Hz, 1H), 7.42 (d, J = 8.4 Hz, 1H), 7.34 (dd, J = 1.2, 8.4 Hz, 1H), 5.04 (t, J= 7.1 Hz, 1H), 3.82-3.86 (m, |
| | | | | 2H), 2.12-2.33 (m, 2H), |
| | | | | 1.90-2.10 (m, 2H) |
| 6-Chloro-N-(5- isopropyl- 1,3,4- oxadiazol-2- yl)benzo[d]oxa- zol-2-amine | 214 | 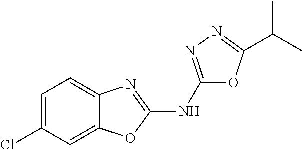 [SMILES: CC(C)c1nnc(Nc2nc3ccc(Cl)cc3o2)o1] | 7% | MS (ESI+) for CHNOS m/z 279.08 [M + H]⁺; LC purity 98.2% (Ret. Time- 6.17 min); ¹H NMR (400 MHz, DMSO-d₆): δ 7.71 (s, 1H), 7.42 (d, J = 8.3 Hz, 1H), 7.32 (d, J = 8.3 Hz, 1H), 3.06-3.15 (m, 1H), 1.29 (d, J = 6.9 Hz, 6H), |
| N-(2-((1,3,4- Oxadiazol-2- yl)amino)benz- o[d]oxazol-6- yl)-N- methylacetamide | 215 | 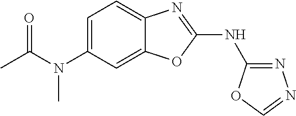 [SMILES: CC(=O)N(C)c1ccc2nc(Nc3nnco3)oc2c1] | 5% | MS (ESI+) for CHNOS m/z 274.06 [M + H]⁺; LC purity 93.8% (Ret. Time- 3.90 min); ¹H NMR (400 MHz, DMSO-d₆ + D₂O): δ 8.72 (s, 1H), 7.55 (s, 1H), 7.42 (s, 1H), 7.23 (d, J = 6.6 Hz, 1H), 3.13 (s, 3H), |
| | | | | 1.75 (s, 3H) |
| Ethyl 5-((6- chlorobenzo[d] oxazol-2- yl)amino)- 1,2,4- oxadiazole-3- carboxylate | 216 | 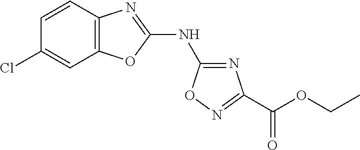 [SMILES: CCOC(=O)c1noc(Nc2nc3ccc(Cl)cc3o2)n1] | 55% | MS (ESI+) for CHNOS m/z 309.11 [M + H]⁺; LC purity 99.5% (Ret. Time- 4.34 min); ¹H NMR (400 MHz, DMSO-d₆): δ 12.88 (bs 1H), 7.84 (s, 1H), 7.47 (d, J = 8.4 Hz, 1H), 7.40 (dd, J = 1.5, 8.4 Hz, 1H), 4.39 (q, J = 7.1 Hz, 2H), 1.33 (t, J = 7.1 Hz, 3H). |

### Synthetic Route 32

### 5-((6-Chlorobenzo[d]oxazol-2-yl)amino)-1,3,4-oxadiazole-2-carbonitrile

### Example 217

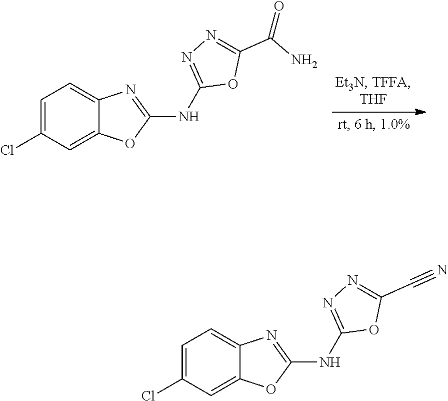 [SMILES: NC(=O)c1nnc(Nc2nc3ccc(Cl)cc3o2)o1 ; N#Cc1nnc(Nc2nc3ccc(Cl)cc3o2)o1]

To a solution of 5-((6-chlorobenzo[d]oxazol-2-yl)amino)-1,3,4-oxadiazole-2-carboxamide (120 mg, 0.43 mmol) in THF (5.0 mL) were added Et₃N (0.2 mL, 1.08 mmol), TFFA (0.2 mL, 0.860 mmol) at rt. The reaction mixture was stirred at rt for 6 h. TLC showed the reaction to be complete. The reaction poured in to EtOAc (50 mL) and washed with H₂O (3×25 mL) and brine (50 mL). The organics were dried (Na₂SO₄), filtered and concentrated under reduced pressure. The crude was purified by prep HPLC purification to give 5-((6-chlorobenzo[d]oxazol-2-yl)amino)-1,3,4-oxadiazole-2-carbonitrile as an off white solid. Yield: 4 mg (3.5%). ¹H NMR (400 MHz, DMSO-d₆): δ 7.84 (s, 1H), 7.44 (d, J=8.4 Hz, 1H), 7.40 (d, J=8.4 Hz, 1H); MS (ESI+) for CHNOS m/z 262.10 [M+H]⁺.

The following compounds were prepared in a similar manner to 5-((6-Chlorobenzo[d]oxazol-2-yl)amino)-1,3,4-oxadiazole-2-carbonitrile.

| | | | | Spectral Data |
|---|---|---|---|---|
| Name | Ex | Structure | Yield | 1H NMR & LCMS |
| 5-((6- Fluorobenzo[d] oxazol-2- yl)amino)- 1,3,4- oxadiazole-2- carbonitrile | 218 | 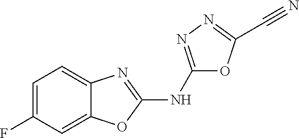 [SMILES: N#Cc1nnc(Nc2nc3ccc(F)cc3o2)o1] | 4% | MS (ESI+) for CHNOS m/z 246.06 [M + H]⁺; LC purity 98.4% (Ret. Time- 5.82; ¹H NMR (400 MHz, DMSO-d₆): δ 12.94 (bs, 1H), 7.65-7.74 (m, 1H), 7.41-7.49 (m, 1H), 7.17- 7.29 (m, 1H) |
| 5-((5- Fluorobenzo[d] oxazol-2- yl)amino)- 1,3,4- oxadiazole-2- carbonitrile | 219 | 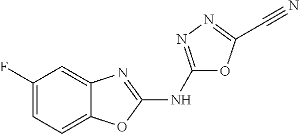 [SMILES: N#Cc1nnc(Nc2nc3cc(F)ccc3o2)o1] | 7% | MS (ESI+) for CHNOS m/z 246.06 [M + H]⁺; LC purity 99.5% (Ret. Time- 5.81; ¹H NMR (400 MHz, DMSO-d₆): δ 13.01 (bs, 1H), 7.60-7.70 (m, 1H), 7.23-7.32 (m, 1H), 7.10- 7.19 (m, 1H) |

### Synthetic Route 33

### 6-Chloro-N-(1,3,4-oxadiazol-2-yl)oxazolo[4,5-b]pyridin-2-amine (Example 220)

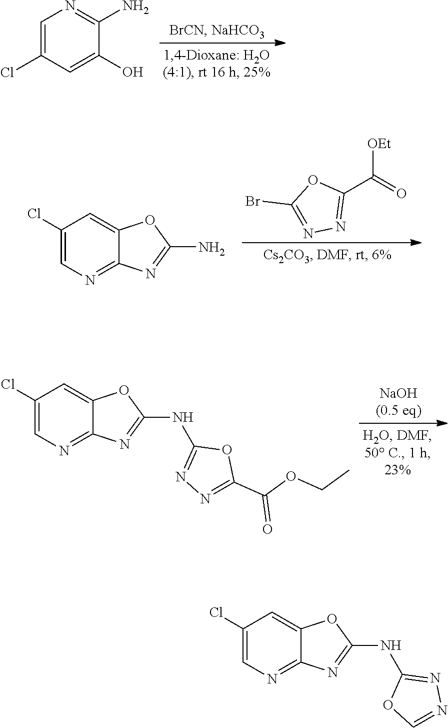 [SMILES: Nc1ncc(Cl)cc1O ; Nc1nc2ncc(Cl)cc2o1 ; CCOC(=O)c1nnc(Br)o1 ; CCOC(=O)c1nnc(Nc2nc3ncc(Cl)cc3o2)o1 ; Clc1cnc2nc(Nc3nnco3)oc2c1]

### 6-Chlorooxazolo[4,5-b]pyridin-2-amine

To a solution of 2-amino-5-chloropyridin-3-ol (1.0 g, 6.94 mmol) in dioxane:H₂O (7:3, 30 mL) were added sodium bicarbonate (2.91 g, 34.7 mmol) and cynaogen bromide (1.47 g, 13.8 mmol) at rt. The reaction mixture was stirred at rt for 16 h. TLC showed the reaction to be complete. The reaction mixture was diluted with aq. saturated NaHCO₃ (100 mL) and extracted with EtOAc (3×50 mL). The organic layer was dried (Na₂SO₄), filtered and concentrated under reduced pressure. The residue was triturated with Et₂O (25 mL) and dried under vacuum to gave 6-chlorooxazolo[4,5-b]pyridin-2-amine as a light yellow solid. Yield: 292 mg (25%); MS (ESI−) for CHNOS m/z 168.19 [M−H]⁺.

### Ethyl 5-((6-chlorooxazolo[4,5-b]pyridin-2-yl)amino)-1,3,4-oxadiazole-2-carboxylate

To a solution of 6-chlorooxazolo[4,5-b]pyridin-2-amine (500 mg, 2.95 mmol) in DMF (5.0 mL) were added ethyl 5-bromo-1,3,4-oxadiazole-2-carboxylate (980 mg, 4.43 mmol) and Cs₂CO₃ (2.88 g, 8.87 mmol) at rt. The rection mixture was stirred at rt for 16 h. TLC showed the reaction to be complete. The reaction mixture was diluted with H₂O (50 mL) and extracted with EtOAc (3×50 mL). The organics were dried (Na₂SO₄), filtered and concentrated under reduced pressure. The crude residue was purified by prep HPLC to afford 6-chlorooxazolo[4,5-b]pyridin-2-amine as an off white solid. Yield: 54 mg (6%); MS (ESI+) for CHNOS m/z 310.22 [M+H]⁺; LC purity 99.4% (Ret. Time—3.73 min); ¹H NMR (400 MHz, DMSO-d₆): δ 8.25 (s, 1H), 8.12 (s, 1H), 4.38 (q, J=7.0 Hz, 2H), 1.33 (t, J=7.0 Hz, 3H).

### 6-Chloro-N-(1,3,4-oxadiazol-2-yl)oxazolo[4,5-b]pyridin-2-amine

To a solution of ethyl 5-((6-chlorooxazolo[4,5-b]pyridin-2-yl)amino)-1,3,4-oxadiazole-2-carboxylate (250 mg, 0.80 mmol) in DMF (1.0 mL) was added 1.0N aqueous NaOH solution (0.5 mL) at rt. The reaction mixture was stirred at 50° C. for 1 h. TLC showed the reaction to be complete. The reaction mixture was diluted with H₂O (10 mL) and extracted with EtOAc (2×20 mL). The aqueous layer was acidified to pH 4-5 with 1.0N HCl and extracted with EtOAc (3×20 mL). The organics were dried (Na₂SO₄), filtered and concentrated under reduced pressure. The crude residue was triturated with Et₂O (25 mL) followed by EtOH (10 mL) to afford 6-chloro-N-(1,3,4-oxadiazol-2-yl)oxazolo[4,5-b]pyridin-2-amine as a pale yellow solid. Yield: 30 mg (4%); MS (ESI+) for CHNOS m/z 237.95 [M+H]⁺; LC purity 95.3% (Ret. Time—2.90); NMR (400 MHz, DMSO-d₆): δ 8.86 (s, 1H), 8.28 (bs, 1H), 8.13 (bs, 1H).

The following compounds were prepared in a similar manner to ethyl 5-((6-chlorooxazolo[4,5-b]pyridin-2-yl)amino)-1,3,4-oxadiazole-2-carboxylate.

| | | | | Spectral Data |
|---|---|---|---|---|
| Name | Ex | Structure | Yield | 1H NMR & LCMS |
| 6-Chloro-N- (oxazol-4- yl)benzo[d]oxa- zol-2-amine | 221 | 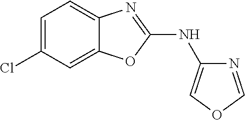 [SMILES: Clc1ccc2nc(Nc3cocn3)oc2c1] | 7% | MS (ESI+) for CHNOS m/z 236.03 [M + H]⁺; LC purity 99.0% (Ret. Time- 4.45 min); ¹H NMR (400 MHz, DMSO-d₆): δ 11.92 (bs, 1H), 7.71 (s, 1H), 7.63 (d, J = 1.2 Hz, 1H), 7.40 (d, J = 8.3 Hz, 1H), 7.22-7.30 |
| | | | | (m, 2H) |
| 6-Chloro-N- (isothiazol-3- yl)benzo[d]oxa- zol-2-amine | 222 | 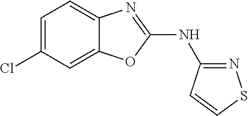 [SMILES: Clc1ccc2nc(Nc3ccsn3)oc2c1] | 3% | MS (ESI+) for CHNOS m/z 252.01 [M + H]⁺; LC purity 99.8% (Ret. Time- 6.00 min); ¹H NMR (400 MHz, DMSO-d₆): δ 11.90 (s, 1H), 9.07 (d, J = 4.7 Hz, 1H), 7.85 (d, J = 4.7 Hz, 1H), 7.73 (s, 1H), 7.47 (d, |
| | | | | J = 8.2 Hz, 1H), 7.29 d, J = |
| | | | | 8.2 Hz, 1H). |

### Synthetic Route 34

### Sodium 5-((6-fluorobenzo[d]oxazol-2-yl)amino)-1,3,4-oxadiazole-2-carboxylate

### Example 223

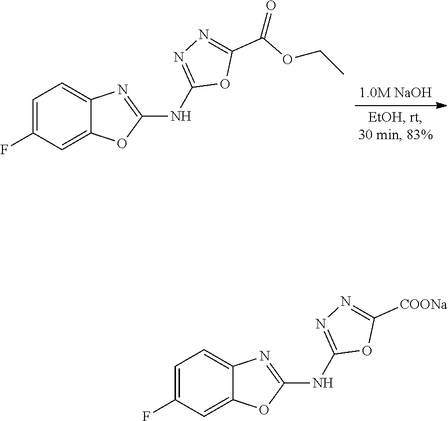

To a suspension of ethyl 5-((6-fluorobenzo[d]oxazol-2-yl)amino)-1,3,4-oxadiazole-2-carboxylate (50 mg, 0.17 mmol) in EtOH (2.0 mL) was added 1.0 M NaOH solution (0.2 mL, 0.17 mmol) at rt. The reaction mixture was stirred at rt for 30 min. TLC showed the reaction to be complete. The reaction mixture was concentrated under reduced pressure and the crude residue was triturated with Et₂O (5.0 mL), filtered and dried under vacuum to afford sodium 5-((6-fluorobenzo[d]oxazol-2-yl)amino)-1,3,4-oxadiazole-2-carboxylate as an off white solid. Yield: 40 mg (83%); ¹H NMR (400 MHz, DMSO-d₆): δ 7.08-7.21 (m, 2H), 6.79-6.88 (m, 1H). The compound was further characterised by ¹³C NMR.

The following compounds was prepared in a similar manner to sodium 5-((6-fluorobenzo[d]oxazol-2-yl)amino)-1,3,4-oxadiazole-2-carboxylate.

| | | | | Spectral Data |
|---|---|---|---|---|
| Name | Ex | Structure | Yield | 1H NMR & LCMS |
| 5 6-chloro-N- (1,2,4- oxadiazol-5- yl)benzo[d]oxa- zol-2-amine | 224 | 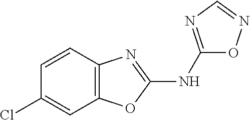 [SMILES: Clc1ccc2nc(Nc3ncno3)oc2c1] | 4% | MS (ESI+) for CHNOS m/z 237.00 [M + H]⁺; LC purity 94% (Ret. Time- 7.18; ¹H NMR (400 MHz, DMSO-d₆ at 372.6 K): δ 7.49 (s, 1H), 7.34-7.39 (m, 1H), 7.10-7.22 (m, 2H) |

### Synthetic Route 35

### 6-Chloro-N-(isoxazol-4-yl)benzo[d]oxazol-2-amine (Example 225)

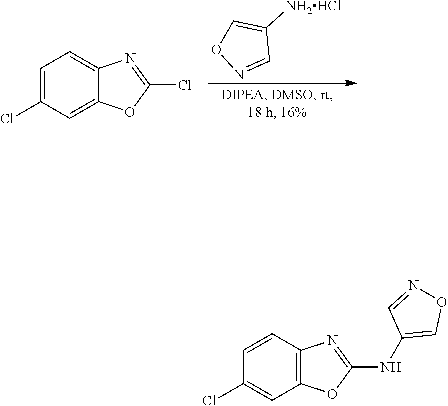

To a solution of 2,6-dichlorobenzo[d]oxazole (250 mg, 1.3 mmol) in DMSO (2.5 mL) were added isoxazol-4-amine hydrochloride (160 mg, 1.30 mmol) and DIPEA (1.0 mL, 3.30 mmol) at rt. The reaction mixture was stirred at rt for 18 h. TLC showed the reaction to be complete. The reaction was poured in to ice-water (50 mL) and extracted with EtOAc (3×25 mL). The organics were washed with water (2×50 mL), dried (Na₂SO₄), filtered and concentrated under reduced pressure. The crude was triturated with EtOH (3.0 mL) to give 6-chloro-N-(isoxazol-4-yl)benzo[d]oxazol-2-amine as an off white solid. Yield: 51 mg (16%). MS (ESI−) for CHNOS m/z 233.94 [M−H]; LC purity 96.5% (Ret. Time—5.65; (¹H NMR (400 MHz, DMSO-d₆): δ 10.84 (bs, 1H), 9.14 (s, 1H), 8.73 (s, 1H), 7.70 (s, 1H), 7.42 (d, J=8.2 Hz, 1H), 7.27 (d, J=8.2 Hz, 1H).

Biological Activity

EC₅₀ Determination:

Actively growing Neisseria gonorrhoeae on GC agar (based on; Spence et. al. (2008): Curr. Protoc. Microbiol. 8:4A.1.1-4A.1.26) plates are harvested and transferred to GC broth (based on; Spence et al (2008): Curr. Protoc. Microbiol. 8:4A.1.1-4A.1.26) to generate a liquid stock. This culture is allowed to establish and grow to mid-log phase (at 37° C./5% CO₂), finally this culture is diluted (˜10⁵ cells/ml) to generate a seed inoculum to establish plate-based broth assays. EC₅₀ values were determined by assaying growth (absorbance read at 600 nm, after 20 hours at 37° C./5% CO₂) of Neisseria gonorrhoeae across a 10-point dilution series of the test compound. The EC₅₀ value is determined from transformed data to identify the concentration of compound giving a 50% response relative to control samples (no compound).

EC₅₀ determination for Staphylococcus aureus and Enterococcus Spp. followed a similar procedure but were conducted using Isosensitest broth (Oxoid) with incubations at 37° C. in atmospheric air. Final absorbance reads were conducted during the late exponential growth phase.

### Example A: Broad Spectrum Antibacterial Activity

A list of preferred compounds of general formula (I) together with their IC₅₀ concentration against a panel of bacteria is summarized in Table 1 (below).

| | | Staphylococcus | Entercoccus | Enterococcus |
|---|---|---|---|---|
| Example | Name | aureus | faecalis | faecium |
| 60 | N-(5-(Trifluoromethyl)benzo[d]oxazol-2- | B | C | B |
| | yl)oxazolo[4,5-c]pyridin-2-amine | | | |
| 67 | N-(1,3,4-Oxadiazol-2-yl)-5- | C | B | C |
| | (trifluoromethyl)benzo[d]oxazol-2-amine | | | |
| 80 | 6-Chloro-N-(5-(pyrrolidin-1-ylmethyl)-1,3,4-oxadiazol- | C | C | B |
| | 2-yl)-5-(trifluoromethyl)benzo[d]oxazol-2-amine | | | |
| 105 | N-(5-Cyclopropyl-1,3,4-oxadiazol-2-yl)-6- | C | C | C |
| | (trifluoromethyl)benzo[d]oxazol-2-amine | | | |
| 117 | N-(5-Methyl-4H-1,2,4-triazol-3-yl)-5- | C | B | B |
| | (trifluoromethyl)benzo[d]oxazol-2-amine | | | |
| 159 | N-(6-Chlorobenzo[d]oxazol-2-yl)oxazolo[4,5- | A | A | A |
| | c]pyridin-2-amine | | | |
| 165 | N-(5-Chlorobenzo[d]oxazol-2-yl)-6- | B | B | B |
| | (trifluoromethyl)oxazolo[4,5-c]pyridin-2-amine | | | |
| 170 | N-(6-(Trifluoromethyl)-1H-imidazo[4,5-c]pyridin-2- | B | B | B |
| | yl)benzo[d]oxazol-2-amine | | | |

In the above table, the symbols used to indicate the IC50 values are:

IC₅₀≤1 μM=C

IC₅₀≤10 μM=B

IC₅₀≤100 μM=A

### Example B: Activity Against Neisseria gonorrhoeae

All exemplified examples display IC₅₀ (Inhibitory concentration) values against Neisseria gonorrhoeae equal to, or less than, 200 μM.

| | | Neisseria |
|---|---|---|
| Example | NAME | gonorrhoeae |
| 1 | N-Cyclopropyl-2-((5-(trifluoromethyl)benzo[d]oxazol-2- | D |
| | yl)amino)thiazole-4-carboxamide | |
| 2 | N-Methyl-2-((5-(trifluoromethyl)benzo[d]oxazol-2- | D |
| | yl)amino)thiazole-4-carboxamide | |
| 3 | N-Ethyl-2-((5-(trifluoromethyl)benzo[d]oxazol-2-yl)amino)thiazole- | D |
| | 4-carboxamide | |
| 4 | N-Isopropyl-2-((5-(trifluoromethyl)benzo[d]oxazol-2- | D |
| | yl)amino)thiazole-4-carboxamide | |
| 5 | N-Phenyl-2-((5-(trifluoromethyl)benzo[d]oxazol-2- | D |
| | yl)amino)thiazole-4-carboxamide | |
| 6 | N-(Thiazol-2-yl)-5-(trifluoromethyl)benzo[d]oxazol-2-amine | D |
| 7 | N-(4-Methylthiazol-2-yl)-5-(trifluoromethyl)benzo[d]oxazol-2- | D |
| | amine | |
| 8 | Ethyl 2-((5-(trifluoromethyl)benzo[d]oxazol-2-yl)amino)thiazole-4- | D |
| | carboxylate | |
| 9 | N-(3-Fluorophenyl)-2-((5-(trifluoromethyl)benzo[d]oxazol-2- | D |
| | yl)amino)thiazole-4-carboxamide | |
| 10 | N-(3-Chlorophenyl)-2-((5-(trifluoromethyl)benzo[d]oxazol-2- | D |
| | yl)amino)thiazole-4-carboxamide | |
| 11 | N-(Isoxazol-3-yl)-5-(trifluoromethyl)benzo[d]oxazol-2-amine | D |
| 12 | N-(1-Methyl-1H-1,2,3-triazol-4-yl)-5- | C |
| | (trifluoromethyl)benzo[d]oxazol-2-amine | |
| 13 | N-(4-(tert-butyl)thiazol-2-yl)-5-(trifluoromethyl)benzo[d]oxazol-2- | C |
| | amine | |
| 14 | N-(1,3,4-Thiadiazol-2-yl)-5-(trifluoromethyl)benzo[d]oxazol-2- | D |
| | amine | |
| 15 | 2-(Benzo[d]oxazol-2-ylamino)-N-cyclopropylthiazole-4- | D |
| | carboxamide | |
| 16 | N-Cyclopropyl-2-((5-methylbenzo[d]oxazol-2-yl)amino)thiazole-4- | D |
| | carboxamide | |
| 17 | 2-((5-Chlorobenzo[d]oxazol-2-yl)amino)-N-cyclopropylthiazole-4- | D |
| | carboxamide | |
| 18 | N-Cyclopropyl-2-((5-fluorobenzo[d]oxazol-2-yl)amino)thiazole-4- | D |
| | carboxamide | |
| 19 | N-Cyclopropyl-2-((6-fluorobenzo[d]oxazol-2-yl)amino)thiazole-4- | D |
| | carboxamide | |
| 20 | 2-((6-Chlorobenzo[d]oxazol-2-yl)amino)-N-cyclopropylthiazole-4- | E |
| | carboxamide | |
| 21 | 2-((5-(Trifluoromethyl)benzo[d]oxazol-2-yl)amino)thiazole-4- | D |
| | carboxamide | |
| 22 | N-(5-Methyl-1,3,4-oxadiazol-2-yl)-5- | D |
| | (trifluoromethyl)benzo[d]oxazol-2-amine | |
| 23 | N-(1,2,4-Thiadiazol-5-yl)-5-(trifluoromethyl)benzo[d]oxazol-2- | D |
| | amine | |
| 24 | N-Cyclopropyl-2-((5-(trifluoromethyl)benzo[d]oxazol-2- | D |
| | yl)amino)oxazole-4-carboxamide | |
| 25 | N-Cyclopropyl-5-methyl-2-((5-(trifluoromethyl)benzo[d]oxazol-2- | D |
| | yl)amino)thiazole-4-carboxamide | |
| 26 | N-Cyclopropyl-2-((6-(trifluoromethyl)benzo[d]oxazol-2- | E |
| | yl)amino)thiazole-4-carboxamide | |
| 27 | N-(5-Morpholinothiazol-2-yl)-6-(trifluoromethyl)benzo[d]oxazol-2- | D |
| | amine | |
| 28 | N-(5-(Piperidin-1-yl)thiazol-2-yl)-6- | D |
| | (trifluoromethyl)benzo[d]oxazol-2-amine | |
| 30 | N-Cyclopropyl-5-((6-(trifluoromethyl)benzo[d]oxazol-2-yl)amino)- | B |
| | 1,3,4-oxadiazole-2-carboxamide | |
| 31 | N-(3-Methyl-1,2,4-oxadiazol-5-yl)-5- | D |
| | (trifluoromethyl)benzo[d]oxazol-2-amine | |
| 32 | N-(5-(4-Methylpiperazin-1-yl)thiazol-2-yl)-5- | D |
| | (trifluoromethyl)benzo[d]oxazol-2-amine | |
| 33 | 2-((6-Chlorobenzo[d]oxazol-2-yl)amino)thiazole-4-carboxamide | D |
| 34 | 2-((6-Chloro-5-(trifluoromethyl)benzo[d]oxazol-2- | D |
| | yl)amino)thiazole-4-carboxamide | |
| 35 | N-(5-(4-Methylpiperazin-1-yl)thiazol-2-yl)-6- | D |
| | (trifluoromethyl)benzo[d]oxazol-2-amine | |
| 36 | 2-((7-Chlorobenzo[d]oxazol-2-yl)amino)thiazole-4-carboxamide | D |
| 37 | N-Cyclopropyl-2-((5-(trifluoromethyl)benzo[d]oxazol-2- | C |
| | yl)amino)thiazole-5-carboxamide | |
| 38 | N-(1-Methyl-1H-pyrazol-3-yl)-5-(trifluoromethyl)benzo[d]oxazol-2- | C |
| | amine | |
| 39 | 7-Chloro-N-(5-methyl-1,3,4-oxadiazol-2-yl)benzo[d]oxazol-2- | D |
| | amine | |
| 40 | 4,6-Dichloro-N-(5-methyl-1,3,4-oxadiazol-2-yl)benzo[d]oxazol-2- | E |
| | amine | |
| 41 | N-(4-methyl-4H-1,2,4-triazol-3-yl)-5- | D |
| | (trifluoromethyl)benzo[d]oxazol-2-amine | |
| 42 | 6-Chloro-N-(5-methylisoxazol-3-yl)-5- | E |
| | (trifluoromethyl)benzo[d]oxazol-2-amine | |
| 43 | 6-Chloro-N-(4H-1,2,4-triazol-3-yl)-5- | E |
| | (trifluoromethyl)benzo[d]oxazol-2-amine | |
| 44 | Methyl 2-((5-methyl-1,3,4-oxadiazol-2-yl)amino)benzo[d]oxazole- | E |
| | 5-carboxylate | |
| 45 | 4,6-Dichloro-N-(1,3,4-oxadiazol-2-yl)benzo[d]oxazol-2-amine | E |
| 46 | 6-Chloro-N-(isoxazol-3-yl)benzo[d]oxazol-2-amine | D |
| 47 | 6-Chloro-5-fluoro-N-(1,3,4-oxadiazol-2-yl)benzo[d]oxazol-2-amine | E |
| 48 | 5,6-diFluoro-N-(1,3,4-oxadiazol-2-yl)benzo[d]oxazol-2-amine | E |
| 49 | N-(5-(Piperazin-1-yl)thiazol-2-yl)-6- | D |
| | (trifluoromethyl)benzo[d]oxazol-2-amine hydrochloride | |
| 50 | 5-Chloro-N-(5-methyl-1,3,4-oxadiazol-2-yl)benzo[d]oxazol-2- | E |
| | amine | |
| 51 | N-(5-Cyclopropyl-1,3,4-oxadiazol-2-yl)-6- | D |
| | (trifluoromethyl)oxazolo[4,5-c]pyridin-2-amine | |
| 52 | Ethyl 5-((5-(trifluoromethyl)benzo[d]oxazol-2-yl)amino)-1,3,4- | D |
| | oxadiazole-2-carboxylate | |
| 53 | N-(5-Methyl-1,3,4-oxadiazol-2-yl)-6-(trifluoromethyl)oxazolo[4,5- | C |
| | c]pyridin-2-amine | |
| 54 | 6-Fluoro-N-(5-methyl-1,3,4-oxadiazol-2-yl)benzo[d]oxazol-2- | D |
| | amine | |
| 55 | Ethyl 5-((1-methyl-5-(trifluoromethyl)-1H-benzo[d]imidazol-2- | D |
| | yl)amino)-1,3,4-oxadiazole-2-carboxylate | |
| 56 | Ethyl 5-((5-(trifluoromethyl)-1H-benzo[d]imidazol-2-yl)amino)- | D |
| | 1,3,4-oxadiazole-2-carboxylate | |
| 57 | 7-Chloro-N-(1,3,4-oxadiazol-2-yl)benzo[d]oxazol-2-amine | D |
| 58 | N-(5-Methyl-1,3,4-oxadiazol-2-yl)oxazolo[4,5-c]pyridin-2-amine | C |
| 59 | N-(5-(triFluoromethyl)benzo[d]oxazol-2-yl)oxazolo[5,4-c]pyridin-2- | D |
| | amine | |
| 60 | N-(5-(triFluoromethyl)benzo[d]oxazol-2-yl)oxazolo[4,5-c]pyridin-2- | E |
| | amine | |
| 61 | Ethyl 5-((6-chlorobenzo[d]oxazol-2-yl)amino)-1,3,4-oxadiazole-2- | E |
| | carboxylate | |
| 62 | 4-Fluoro-N-(5-methyl-1,3,4-oxadiazol-2-yl)benzo[d]oxazol-2- | D |
| | amine | |
| 63 | 6-Fluoro-N-(1,3,4-oxadiazol-2-yl)benzo[d]oxazol-2-amine | D |
| 64 | 7-Chloro-N-(5-methyl-1,3,4-oxadiazol-2-yl)oxazolo[4,5-c]pyridin- | C |
| | 2-amine | |
| 65 | Ethyl 5-((6-chloro-5-(trifluoromethyl)benzo[d]oxazol-2-yl)amino)- | E |
| | 1,3,4-oxadiazole-2-carboxylate | |
| 66 | 5-Fluoro-N-(1,3,4-oxadiazol-2-yl)benzo[d]oxazol-2-amine | D |
| 67 | N-(1,3,4-Oxadiazol-2-yl)-5-(trifluoromethyl)benzo[d]oxazol-2- | E |
| | amine | |
| 68 | N-(5-(Trifluoromethyl)-1H-benzo[d]imidazol-2-yl)-1,3,4-oxadiazol- | D |
| | 2-amine | |
| 69 | N-(1-methyl-5-(trifluoromethyl)-1H-benzo[d]imidazol-2-yl)-1,3,4- | D |
| | oxadiazol-2-amine | |
| 70 | N-(5-(morpholinomethyl)-1,3,4-oxadiazol-2-yl)-5- | D |
| | (trifluoromethyl)benzo[d]oxazol-2-amine | |
| 71 | N-(5-(Pyrrolidin-1-ylmethyl)-1,3,4-oxadiazol-2-yl)-5- | D |
| | (trifluoromethyl)benzo[d]oxazol-2-amine | |
| 72 | N-(5-(Piperidin-1-ylmethyl)-1,3,4-oxadiazol-2-yl)-5- | D |
| | (trifluoromethyl)benzo[d]oxazol-2-amine | |
| 73 | N-(5-((2-Methylpyrrolidin-1-yl)methyl)-1,3,4-oxadiazol-2-yl)-5- | D |
| | (trifluoromethyl)benzo[d]oxazol-2-amine | |
| 74 | N-(5-((3,3-di Fluoropyrrolidin-1-yl)methyl)-1,3,4-oxadiazol-2-yl)-5- | D |
| | (trifluoromethyl)benzo[d]oxazol-2-amine | |
| 75 | N-(5-((3-Methoxypyrrolidin-1-yl)methyl)-1,3,4-oxadiazol-2-yl)-5- | D |
| | (trifluoromethyl)benzo[d]oxazol-2-amine | |
| 76 | 1-((5-((5-(triFluoromethyl)benzo[d]oxazol-2-yl)amino)-1,3,4- | C |
| | oxadiazol-2-yl)methyl)pyrrolidine-3-carbonitrile | |
| 77 | 6-Chloro-N-(5-(pyrrolidin-1-ylmethyl)-1,3,4-oxadiazol-2- | E |
| | yl)benzo[d]oxazol-2-amine | |
| 78 | N-(5-((3-methylpyrrolidin-1-yl)methyl)-1,3,4-oxadiazol-2-yl)-5- | D |
| | (trifluoromethyl)benzo[d]oxazol-2-amine | |
| 79 | N-(5-((3-Fluoropyrrolidin-1-yl)methyl)-1,3,4-oxadiazol-2-yl)-5- | D |
| | (trifluoromethyl)benzo[d]oxazol-2-amine | |
| 80 | 6-Chloro-N-(5-(pyrrolidin-1-ylmethyl)-1,3,4-oxadiazol-2-yl)-5- | E |
| | (trifluoromethyl)benzo[d]oxazol-2-amine | |
| 81 | 6-Chloro-N-(5-((dimethylamino)methyl)-1,3,4-oxadiazol-2- | D |
| | yl)benzo[d]oxazol-2-amine | |
| 82 | N-(5-((Dimethylamino)methyl)-1,3,4-oxadiazol-2-yl)-5- | D |
| | (trifluoromethyl)benzo[d]oxazol-2-amine | |
| 83 | 5-Methyl-N-(6-(trifluoromethyl)-1H-benzo[d]imidazol-2-yl)-1,3,4- | D |
| | oxadiazol-2-amine | |
| 84 | Methyl 2-((5-methyl-1,3,4-oxadiazol-2-yl)amino)-1H- | C |
| | benzo[d]imidazole-5-carboxylate | |
| 85 | N-(5-Chloro-1H-benzo[d]imidazol-2-yl)-5-methyl-1,3,4-oxadiazol- | D |
| | 2-amine | |
| 86 | 5-Methyl-N-(5-(trifluoromethyl)-1H-benzo[d]imidazol-2-yl)-1,3,4- | D |
| | thiadiazol-2-amine | |
| 87 | N-(4-Fluoro-1H-benzo[d]imidazol-2-yl)-5-methyl-1,3,4-oxadiazol- | D |
| | 2-amine | |
| 88 | 5-Methyl-N-(1-methyl-5-(trifluoromethyl)-1H-benzo[d]imidazol-2- | D |
| | yl)-1,3,4-oxadiazol-2-amine | |
| 89 | 5-Methyl-N-(1-methyl-6-(trifluoromethyl)-1H-benzo[d]imidazol-2- | D |
| | yl)-1,3,4-oxadiazol-2-amine | |
| 90 | N-(1,4-Dimethyl-1H-benzo[d]imidazol-2-yl)-5-methyl-1,3,4- | C |
| | oxadiazol-2-amine | |
| 91 | N-(6-Chloro-1H-benzo[d]imidazol-2-yl)-1,3,4-oxadiazol-2-amine | B |
| 92 | N-(6-Chloro-1-methyl-1H-benzo[d]imidazol-2-yl)-1,3,4-oxadiazol- | B |
| | 2-amine | |
| 93 | N-(5-((Methylamino)methyl)-1,3,4-oxadiazol-2-yl)-5- | C |
| | (trifluoromethyl)benzo[d]oxazol-2-amine | |
| 94 | N-Methyl-N-((5-((5-(trifluoromethyl)benzo[d]oxazol-2-yl)amino)- | C |
| | 1,3,4-oxadiazol-2-yl)methyl)acetamide | |
| 95 | Piperazin-1-yl(2-((5-(trifluoromethyl)benzo[d]oxazol-2- | D |
| | yl)amino)thiazol-4-yl)methanone hydrochloride | |
| 96 | 2-((5-Chlorobenzo[d]oxazol-2-yl)amino)thiazole-4-carboxylic acid | C |
| 97 | 2-((5-Chlorobenzo[d]oxazol-2-yl)amino)-N-(2- | D |
| | (dimethylamino)ethyl)thiazole-4-carboxamide | |
| 98 | N-(2-fluoropyridin-4-yl)-2-((5-(trifluoromethyl)benzo[d]oxazol-2- | E |
| | yl)amino)thiazole-4-carboxamide | |
| 99 | 2-((5-Chlorobenzo[d]oxazol-2-yl)amino)thiazole-4-carboxamide | D |
| 100 | N-(4-Chlorothiazol-2-yl)-5-(trifluoromethyl)benzo[d]oxazol-2- | D |
| | amine | |
| 101 | 2-((6-(Trifluoromethyl)benzo[d]oxazol-2-yl)amino)thiazole-4- | D |
| | carboxamide | |
| 102 | 5-((6-(Trifluoromethyl)benzo[d]oxazol-2-yl)amino)-1,3,4- | B |
| | oxadiazole-2-carboxamide | |
| 103 | N-(5-Cyclopropyl-1,3,4-oxadiazol-2-yl)-5- | E |
| | (trifluoromethyl)benzo[d]oxazol-2-amine | |
| 104 | N-(5-Methyl-1,3,4-oxadiazol-2-yl)-6- | E |
| | (trifluoromethyl)benzo[d]oxazol-2-amine | |
| 105 | N-(5-Cyclopropyl-1,3,4-oxadiazol-2-yl)-6- | E |
| | (trifluoromethyl)benzo[d]oxazol-2-amine | |
| 106 | N-(5-Methyl-1,3,4-oxadiazol-2-yl)oxazolo[4,5-c]pyridin-2-amine | C |
| 108 | N-(5-Isopropyl-1,3,4-oxadiazol-2-yl)-5- | D |
| | (trifluoromethyl)benzo[d]oxazol-2-amine | |
| 109 | 6-Chloro-N-(5-methyl-1,3,4-oxadiazol-2-yl)benzo[d]oxazol-2- | E |
| | amine | |
| 110 | 5-(Trifluoromethyl)-N-(5-(trifluoromethyl)-1,3,4-oxadiazol-2- | B |
| | yl)benzo[d]oxazol-2-amine | |
| 111 | N-(5-Methyl-1,3,4-thiadiazol-2-yl)-5- | D |
| | (trifluoromethyl)benzo[d]oxazol-2-amine | |
| 112 | N-(5-Methyloxazol-2-yl)-5-(trifluoromethyl)benzo[d]oxazol-2- | D |
| | amine | |
| 113 | N-(4,5-Dimethyloxazol-2-yl)-5-(trifluoromethyl)benzo[d]oxazol-2- | D |
| | amine | |
| 114 | N-(1,3,4-Oxadiazol-2-yl)-6-(trifluoromethyl)benzo[d]oxazol-2- | D |
| | amine | |
| 115 | 6-Chloro-N-(1,3,4-oxadiazol-2-yl)benzo[d]oxazol-2-amine | D |
| 116 | N-(4H-1,2,4-Triazol-3-yl)-5-(trifluoromethyl)benzo[d]oxazol-2- | D |
| | amine | |
| 117 | N-(5-Methyl-4H-1,2,4-triazol-3-yl)-5- | D |
| | (trifluoromethyl)benzo[d]oxazol-2-amine | |
| 118 | N-(5-Methyl-1,3,4-oxadiazol-2-yl)-4- | C |
| | (trifluoromethyl)benzo[d]oxazol-2-amine | |
| 119 | 4-Chloro-N-(5-methyl-1,3,4-oxadiazol-2-yl)benzo[d]oxazol-2- | D |
| | amine | |
| 120 | 4-Chloro-N-(1,3,4-oxadiazol-2-yl)benzo[d]oxazol-2-amine | D |
| 121 | 7-Chloro-N-(5-methyl-4H-1,2,4-triazol-3-yl)benzo[d]oxazol-2- | E |
| | amine | |
| 122 | 6-Chloro-N-(5-methyl-4H-1,2,4-triazol-3-yl)benzo[d]oxazol-2- | C |
| | amine | |
| 123 | 4,6-Dichloro-N-(5-methyl-4H-1,2,4-triazol-3-yl)benzo[d]oxazol-2- | D |
| | amine | |
| 124 | 4-Fluoro-N-(5-methyl-4H-1,2,4-triazol-3-yl)benzo[d]oxazol-2- | D |
| | amine | |
| 125 | 6-chloro-N-(5-methyl-4H-1,2,4-triazol-3-yl)-5- | E |
| | (trifluoromethyl)benzo[d]oxazol-2-amine | |
| 126 | (S)-N-(5-(1-Methylpyrrolidin-2-yl)-1,3,4-oxadiazol-2-yl)-5- | D |
| | (trifluoromethyl)benzo[d]oxazol-2-amine | |
| 127 | (R)-N-(5-(1-methylpyrrolidin-2-yl)-1,3,4-oxadiazol-2-yl)-5- | D |
| | (trifluoromethyl)benzo[d]oxazol-2-amine | |
| 128 | N-(5-(Pyrrolidin-1-ylmethyl)-4H-1,2,4-triazol-3-yl)-5- | D |
| | (trifluoromethyl)benzo[d]oxazol-2-amine | |
| 129 | 4,6-di Chloro-N-(5-(pyrrolidin-1-ylmethyl)-4H-1,2,4-triazol-3- | D |
| | yl)benzo[d]oxazol-2-amine | |
| 130 | 4-Chloro-N-(5-(pyrrolidin-1-ylmethyl)-4H-1,2,4-triazol-3- | C |
| | yl)benzo[d]oxazol-2-amine | |
| 131 | 7-Chloro-N-(5-(pyrrolidin-1-ylmethyl)-4H-1,2,4-triazol-3- | D |
| | yl)benzo[d]oxazol-2-amine | |
| 132 | 6-Chloro-N-(5-(pyrrolidin-1-ylmethyl)-4H-1,2,4-triazol-3- | D |
| | yl)benzo[d]oxazol-2-amine | |
| 133 | (5-((4,6-Dichlorobenzo[d]oxazol-2-yl)amino)-1,3,4-oxadiazol-2- | D |
| | yl)(pyrrolidin-1-yl)methanone | |
| 134 | (5-((4-Chlorobenzo[d]oxazol-2-yl)amino)-1,3,4-oxadiazol-2- | C |
| | yl)(pyrrolidin-1-yl)methanone | |
| 135 | 5-Chloro-N-(1,3,4-oxadiazol-2-yl)benzo[d]oxazol-2-amine | E |
| 136 | N-(5-((Dimethylamino)methyl)-4H-1,2,4-triazol-3-yl)-5- | D |
| | (trifluoromethyl)benzo[d]oxazol-2-amine | |
| 137 | 6-Chloro-N-(5-(pyrrolidin-1-ylmethyl)-4H-1,2,4-triazol-3-yl)-5- | E |
| | (trifluoromethyl)benzo[d]oxazol-2-amine | |
| 138 | 6-Chloro-N-(1,3,4-oxadiazol-2-yl)-5- | E |
| | (trifluoromethyl)benzo[d]oxazol-2-amine | |
| 139 | 6-Chloro-N-(5-isopropyl-4H-1,2,4-triazol-3-yl)-5- | D |
| | (trifluoromethyl)benzo[d]oxazol-2-amine | |
| 140 | 6-Chloro-N-(1-methyl-1H-imidazol-4-yl)-5- | D |
| | (trifluoromethyl)benzo[d]oxazol-2-amine | |
| 141 | 6-Chloro-4-methyl-N-(5-methyl-4H-1,2,4-triazol-3- | D |
| | yl)benzo[d]oxazol-2-amine | |
| 142 | 6-Chloro-N-(4-(2-methoxyethyl)-5-methyl-4H-1,2,4-triazol-3-yl)-5- | D |
| | (trifluoromethyl)benzo[d]oxazol-2-amine | |
| 143 | N-(4,5-dimethyl-4H-1,2,4-triazol-3-yl)-5- | D |
| | (trifluoromethyl)benzo[d]oxazol-2-amine | |
| 144 | 6-Chloro-1-methyl-N-(5-methyl-4H-1,2,4-triazol-3-yl)-5- | B |
| | (trifluoromethyl)-1H-benzo[d]imidazol-2-amine | |
| 145 | N-(5-(Piperidin-4-yl)-1,3,4-oxadiazol-2-yl)-5- | B |
| | (trifluoromethyl)benzo[d]oxazol-2-amine hydrochloride | |
| 146 | (S)-N-(5-(pyrrolidin-2-yl)-1,3,4-oxadiazol-2-yl)-5- | D |
| | (trifluoromethyl)benzo[d]oxazol-2-amine hydrochloride | |
| 147 | N-(5-(Piperazin-1-ylmethyl)-1,3,4-oxadiazol-2-yl)-5- | B |
| | (trifluoromethyl)benzo[d]oxazol-2-amine hydrochloride | |
| 148 | tert-butyl 4-(5-((5-(trifluoromethyl)benzo[d]oxazol-2-yl)amino)- | B |
| | 1,3,4-oxadiazol-2-yl)piperazine-1-carboxylate | |
| 149 | 5-Chloro-N-(1,3,4-oxadiazol-2-yl)-6- | E |
| | (trifluoromethyl)benzo[d]oxazol-2-amine | |
| 150 | 6-Chloro-N-(4H-1,2,4-triazol-3-yl)benzo[d]oxazol-2-amine | D |
| 151 | 6-Chloro-N-(1,3,4-thiadiazol-2-yl)benzo[d]oxazol-2-amine | D |
| 152 | 5,6-Dichloro-N-(1,3,4-oxadiazol-2-yl)benzo[d]oxazol-2-amine | E |
| 153 | 6-Chloro-N-(5-methyl-4H-1,2,4-triazol-3-yl)-5-(trifluoromethyl)-1H- | C |
| | benzo[d]imidazol-2-amine | |
| 154 | 4-Methyl-N-(5-methyl-1,3,4-oxadiazol-2-yl)oxazolo[4,5-c]pyridin- | C |
| | 2-amine | |
| 155 | 5-Methyl-N-(5-(trifluoromethyl)benzo[d]oxazol-2-yl)-4,5,6,7- | D |
| | tetrahydrooxazolo[5,4-c]pyridin-2-amine | |
| 156 | 5-Methyl-N-(5-(trifluoromethyl)benzo[d]oxazol-2-yl)-4,5,6,7- | D |
| | tetrahydrooxazolo[4,5-c]pyridin-2-amine | |
| 157 | N-(5-Methyl-1H-imidazol-2-yl)-5-(trifluoromethyl)benzo[d]oxazol- | B |
| | 2-amine | |
| 158 | N-(Benzo[d]oxazol-2-yl)-5-(trifluoromethyl)benzo[d]oxazol-2- | E |
| | amine | |
| 159 | N-(6-Chlorobenzo[d]oxazol-2-yl)oxazolo[4,5-c]pyridin-2-amine | B |
| 160 | N-(5-Fluorobenzo[d]oxazol-2-yl)oxazolo[4,5-c]pyridin-2-amine | B |
| 161 | N-(Oxazolo[4,5-c]pyridin-2-yl)-6-(trifluoromethyl)oxazolo[4,5- | B |
| | c]pyridin-2-amine | |
| 162 | N-(Benzo[d]oxazol-2-yl)-5-chlorobenzo[d]oxazol-2-amine | E |
| 163 | N-(Benzo[d]oxazol-2-yl)-6-(trifluoromethyl)oxazolo[4,5-c]pyridin-2- | E |
| | amine | |
| 164 | N-(5-Chlorobenzo[d]oxazol-2-yl)oxazolo[4,5-c]pyridin-2-amine | E |
| 165 | N-(5-Chlorobenzo[d]oxazol-2-yl)-6-(trifluoromethyl)oxazolo[4,5- | D |
| | c]pyridin-2-amine | |
| 166 | N-(1H-Benzo[d]imidazol-2-yl)-6-(trifluoromethyl)oxazolo[4,5- | D |
| | c]pyridin-2-amine | |
| 167 | N-(1H-Benzo[d]imidazol-2-yl)-5-(trifluoromethyl)benzo[d]oxazol-2- | D |
| | amine | |
| 168 | N-(5-Chloro-1H-benzo[d]imidazol-2-yl)oxazolo[4,5-c]pyridin-2- | D |
| | amine | |
| 169 | N-(benzo[d]oxazol-2-yl)-N-methyl-6-(trifluoromethyl)oxazolo[4,5- | B |
| | c]pyridin-2-amine | |
| 170 | N-(6-(Trifluoromethyl)-1H-imidazo[4,5-c]pyridin-2- | D |
| | yl)benzo[d]oxazol-2-amine | |
| 171 | N-(Benzo[d]oxazol-2-yl)-4-methyloxazolo[4,5-c]pyridin-2-amine | D |
| 172 | 6-Chloro-N-(5-methylisoxazol-3-yl)benzo[d]oxazol-2-amine | D |
| 173 | 5-Chloro-N-(thiazol-4-yl)benzo[d]oxazol-2-amine | C |
| 174 | 5-Fluoro-N-(1,3,4-oxadiazol-2-yl)-6- | E |
| | (trifluoromethoxy)benzo[d]oxazol-2-amine | |
| 175 | 6-Chloro-N-(5-cyclopropyl-1,3,4-oxadiazol-2-yl)benzo[d]oxazol-2- | E |
| | amine | |
| 176 | 6-Chloro-4-fluoro-N-(1,3,4-oxadiazol-2-yl)benzo[d]oxazol-2-amine | D |
| 177 | 2-((1,3,4-Oxadiazol-2-yl)amino)benzo[d]oxazol-6-ol | C |
| 178 | Methyl 2-((1,3,4-oxadiazol-2-yl)amino)benzo[d]oxazole-6- | D |
| | carboxylate | |
| 179 | 6-Bromo-N-(1,3,4-oxadiazol-2-yl)benzo[d]oxazol-2-amine | E |
| 180 | 6-Cyclopropyl-N-(1,3,4-oxadiazol-2-yl)benzo[d]oxazol-2-amine | D |
| 181 | (2-((1,3,4-Oxadiazol-2-yl)amino)benzo[d]oxazol-6-yl)methanol | C |
| 182 | 4-((6-chlorobenzo[d]oxazol-2-yl)amino)oxazole-2-carboxylic acid | B |
| 183 | N-(1,3,4-oxadiazol-2-yl)-6-(pyrrolidin-1-yl)benzo[d]oxazol-2-amine | D |
| 184 | N-(1,3,4-Oxadiazol-2-yl)-6-(piperidin-1-yl)benzo[d]oxazol-2-amine | D |
| 185 | 6-Morpholino-N-(1,3,4-oxadiazol-2-yl)benzo[d]oxazol-2-amine | C |
| 186 | 6-Nitro-N-(1,3,4-oxadiazol-2-yl)benzo[d]oxazol-2-amine | C |
| 187 | 5-Chloro-6-methyl-N-(1,3,4-oxadiazol-2-yl)benzo[d]oxazol-2- | E |
| | amine | |
| 188 | 6-Chloro-5-methyl-N-(1,3,4-oxadiazol-2-yl)benzo[d]oxazol-2- | E |
| | amine | |
| 189 | 6-Chloro-4-methyl-N-(1,3,4-oxadiazol-2-yl)benzo[d]oxazol-2- | E |
| | amine | |
| 190 | 5-Chloro-6-methoxy-N-(1,3,4-oxadiazol-2-yl)benzo[d]oxazol-2- | E |
| | amine | |
| 191 | 5-Chloro-N-(1,3,4-oxadiazol-2-yl)-6- | E |
| | (trifluoromethoxy)benzo[d]oxazol-2-amine | |
| 192 | 2-((1,3,4-oxadiazol-2-yl)amino)benzo[d]oxazole-6-carboxylic acid | C |
| 193 | 6-(2-methoxyethoxy)-N-(1,3,4-oxadiazol-2-yl)benzo[d]oxazol-2- | D |
| | amine | |
| 194 | 6-Chloro-N-(5-ethynyl-1,3,4-oxadiazol-2-yl)benzo[d]oxazol-2- | E |
| | amine | |
| 195 | N²-(1,3,4-oxadiazol-2-yl)benzo[d]oxazole-2,6-diamine | C |
| 196 | 6-Isopropoxy-N-(1,3,4-oxadiazol-2-yl)benzo[d]oxazol-2-amine | B |
| 197 | 5-Fluoro-6-methyl-N-(1,3,4-oxadiazol-2-yl)benzo[d]oxazol-2- | E |
| | amine | |
| 198 | 6-Chloro-N-(5-(pyrrolidin-1-yl)-1,3,4-oxadiazol-2- | C |
| | yl)benzo[d]oxazol-2-amine | |
| 199 | 6-Chloro-N-(5-(tetrahydrofuran-3-yl)-1,3,4-oxadiazol-2- | D |
| | yl)benzo[d]oxazol-2-amine | |
| 200 | 5-((6-Chlorobenzo[d]oxazol-2-yl)amino)-1,3,4-oxadiazole-2- | B |
| | carboxamide | |
| 201 | 5-((6-Fluorobenzo[d]oxazol-2-yl)amino)-1,3,4-oxadiazole-2- | B |
| | carboxamide | |
| 202 | 5-((5-Fluorobenzo[d]oxazol-2-yl)amino)-1,3,4-oxadiazole-2- | A |
| | carboxamide | |
| 203 | 6-Fluoro-5-methyl-N-(1,3,4-oxadiazol-2-yl)benzo[d]oxazol-2- | E |
| | amine | |
| 204 | 6-chloro-5-methoxy-N-(1,3,4-oxadiazol-2-yl)benzo[d]oxazol-2- | D |
| | amine | |
| 205 | 6-Chloro-N-(1,3,4-oxadiazol-2-yl)-5- | D |
| | (trifluoromethoxy)benzo[d]oxazol-2-amine | |
| 206 | 5-Fluoro-6-methoxy-N-(1,3,4-oxadiazol-2-yl)benzo[d]oxazol-2- | D |
| | amine | |
| 207 | 6-Fluoro-N-(1,3,4-oxadiazol-2-yl)-5- | E |
| | (trifluoromethoxy)benzo[d]oxazol-2-amine | |
| 208 | 6-Methoxy-5-methyl-N-(1,3,4-oxadiazol-2-yl)benzo[d]oxazol-2- | D |
| | amine | |
| 209 | 6-Fluoro-5-methoxy-N-(1,3,4-oxadiazol-2-yl)benzo[d]oxazol-2- | D |
| | amine | |
| 210 | N-(1,3,4-Oxadiazol-2-yl)-6-(trifluoromethoxy)benzo[d]oxazol-2- | E |
| | amine | |
| 211 | 6-Isopropyl-N-(1,3,4-oxadiazol-2-yl)benzo[d]oxazol-2-amine | D |
| 212 | 1-(2-((1,3,4-oxadiazol-2-yl)amino)benzo[d]oxazol-6-yl)pyrrolidin- | B |
| | 2-one | |
| 213 | 6-Chloro-N-(5-(tetrahydrofuran-2-yl)-1,3,4-oxadiazol-2- | E |
| | yl)benzo[d]oxazol-2-amine | |
| 214 | 6-Chloro-N-(5-isopropyl-1,3,4-oxadiazol-2-yl)benzo[d]oxazol-2- | E |
| | amine | |
| 215 | N-(2-((1,3,4-Oxadiazol-2-yl)amino)benzo[d]oxazol-6-yl)-N- | B |
| | methylacetamide | |
| 216 | Ethyl 5-((6-chlorobenzo[d]oxazol-2-yl)amino)-1,2,4-oxadiazole-3- | D |
| | carboxylate | |
| 217 | 5-((6-Chlorobenzo[d]oxazol-2-yl)amino)-1,3,4-oxadiazole-2- | E |
| | carbonitrile | |
| 218 | 5-((6-Fluorobenzo[d]oxazol-2-yl)amino)-1,3,4-oxadiazole-2- | D |
| | carbonitrile | |
| 219 | 5-((5-Fluorobenzo[d]oxazol-2-yl)amino)-1,3,4-oxadiazole-2- | D |
| | carbonitrile | |
| 220 | 6-Chloro-N-(1,3,4-oxadiazol-2-yl)oxazolo[4,5-b]pyridin-2-amine | C |
| 221 | 6-Chloro-N-(oxazol-4-yl)benzo[d]oxazol-2-amine | D |
| 222 | 6-Chloro-N-(isothiazol-3-yl)benzo[d]oxazol-2-amine | D |
| 223 | Sodium 5-((6-fluorobenzo[d]oxazol-2-yl)amino)-1,3,4-oxadiazole- | B |
| | 2-carboxylate | |
| 224 | 5 6-chloro-N-(1,2,4-oxadiazol-5-yl)benzo[d]oxazol-2-amine | B |
| 225 | 6-Chloro-N-(isoxazol-4-yl)benzo[d]oxazol-2-amine | B |

In the above table, the symbols used to indicate the IC₅₀ values are:

IC₅₀≤200 μM=A

IC₅₀≤100 μM=B

IC₅₀≤10 μM=C

IC₅₀≤1 μM=D

IC₅₀≤0.1 μM=E

### EQUIVALENTS

The foregoing description details presently preferred embodiments of the present invention. Numerous modifications and variations in practice thereof are expected to occur to those skilled in the art upon consideration of these descriptions. Those modifications and variations are intended to be encompassed within the claims appended hereto.

1-37. (canceled)

38. A compound of formula (I), or a pharmaceutically acceptable salt, hydrate, solvate, isomer, tautomer, N-oxide, ester, isotope or protected form thereof: 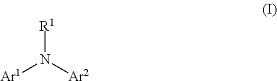
wherein R¹ is selected from hydrogen and C₁₋₄alkyl;
Ar¹ is selected from any one of the following ring systems: 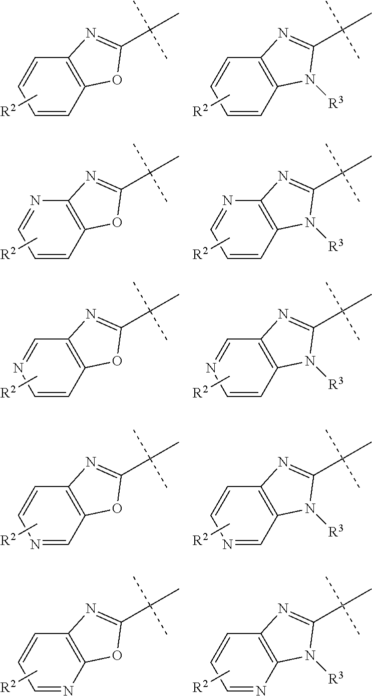
wherein R² is present, and is one or more substituents each independently selected from halogen, cyano, hydroxyl, hydroxylC₁₋₄alkyl, C₁₋₄alkyl, haloC₁₋₄alkyl, C₁₋₄alkoxy, haloC₁₋₄alkyloxy, —C₁₋₄alkylC₁₋₄alkoxy, C₁₋₄alkoxyC₁₋₄alkoxy, NR^{4A}R^{4B}, NO₂, —CONR^{4A}R^{4B}, —C₁₋₄alkylNR^{4A}R^{4B}, —C₁₋₄alkoxyNR^{4A}R^{4B}, C₃₋₇cycloalkyl, morpholinyl, C₂₋₄alkynyl and —CO₂R⁴ wherein
R³ is hydrogen or C₁₋₄alkyl,
R⁴ is hydrogen or C₁₋₄alkyl,
R^{4A} and R^{4B} are each independently selected from hydrogen, C₁₋₄alkyl, —C₁₋₄alkylC₁₋₄ alkoxy, and COR⁴, or
R^{4A} and R^{4B}, together with the nitrogen atom to which they are attached, join together to form a cyclic amino group, wherein the cyclic amino group is optionally substituted with oxo;
Ar² is a ring system selected from Groups (i), and (ii), wherein:
Group (i) is a 5-membered heteroaryl ring system selected from any one of (IIa) to (IIm): 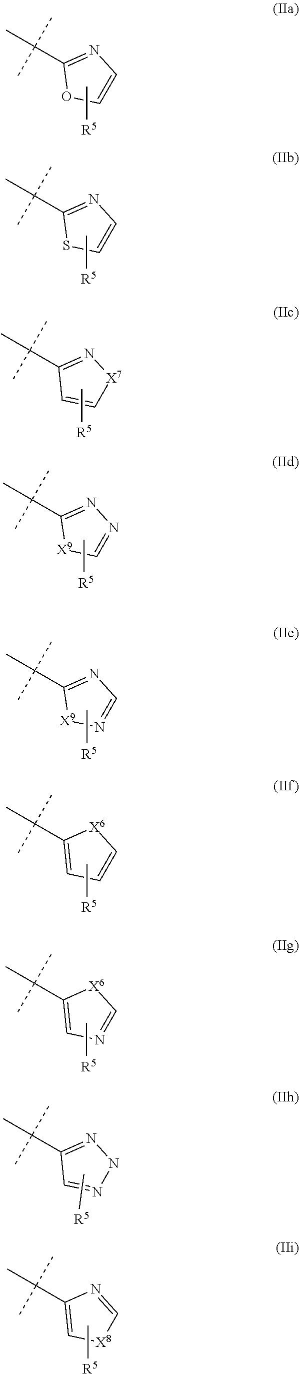 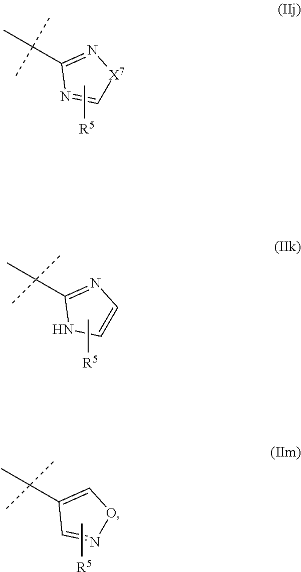
wherein X⁶, X⁷, X⁸, and X⁹ are each independently selected from O, S, and NH, and
R⁵ is one or more optional substituents each independently selected from halogen, cyano, C₁₋₄alkyl, haloC₁₋₄alkyl, C₁₋₄alkoxy, —C₁₋₄alkylC₁₋₄alkoxy, —CO₂R⁶, and -L-Q wherein:
L is a linker group selected from a direct bond, C₁₋₃alkylene and —CO—; and
Q is a group selected from NR^{5A}R^{5B}, C₃cycloalkyl and 4-7 membered heterocyclyl, wherein the 4-7 membered heterocyclyl ring is optionally substituted with one or more substituents selected from halogen, cyano, C₁₋₄alkyl, C₁₋₄alkoxy and CO₂R⁶;
R^{5A} and R^{5B} are each independently selected from hydrogen, C₁₋₄alkyl, C₃₋₇cycloalkyl, COR⁷, —C₁₋₄alkyl-NR⁸R⁹, —C₁₋₄alkylC₁₋₄alkoxy, phenyl and 5 or 6-membered heteroaryl wherein the phenyl or 5 or 6-membered heteroaryl rings are optionally substituted with one or more substituents selected from halogen and C₁₋₄alkyl; or
R^{5A} and R^{5B}, together with the nitrogen atom to which they are attached, join together to form a cyclic amino group, which cyclic amino group is optionally substituted with one or more groups selected from halogen, C₁₋₄alkyl, C₁₋₄alkoxy, cyano, and CO₂R⁶,
R⁶ is hydrogen, C₁₋₄alkyl or an alkali metal;
R⁷ is C₁₋₄alkyl
R⁸ and R⁹ are each independently selected from hydrogen and C₁₋₄alkyl;
Group (ii) is a 5,6-fused bicyclic heteroaryl ring system having the formula (III): 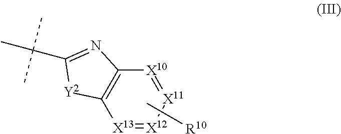
wherein Y² is selected from O and NR^{5C};
R^{5C} is hydrogen or C₁₋₄alkyl,
X¹⁰, X¹¹, X¹², and X¹³ are each independently selected from N and CH;
R¹⁰ is one or more optional substituents each independently selected from halogen, cyano, C₁₋₄alkyl, haloC₁₋₄alkyl, C₁₋₄alkoxy, and —CO₂R⁴;
PROVIDED THAT the compound of formula (I) is other than:

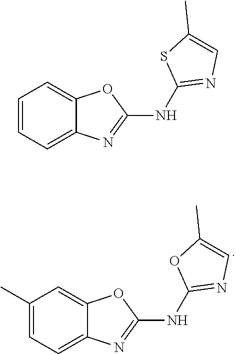 [SMILES: Cc1cnc(Nc2nc3ccccc3o2)s1 ; Cc1ccc2nc(Nc3ncc(C)o3)oc2c1]

39. A compound according to claim 38 wherein R¹ is hydrogen.

40. A compound according to claim 38 wherein Ar² is selected from Group (i).

41. A compound according to claim 38 wherein Ar¹ is selected from any one of the following ring systems:

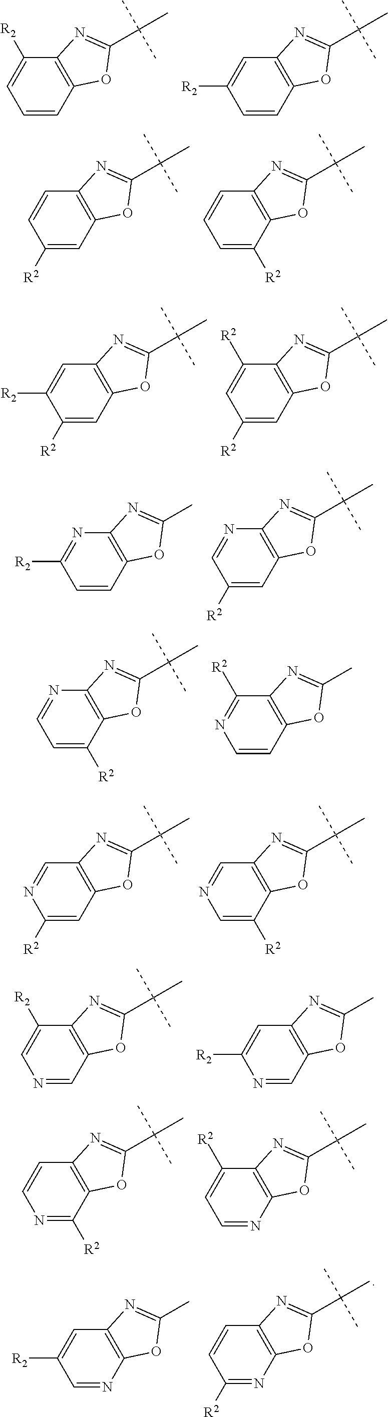

42. A compound according to claim 41, wherein R² is independently selected from any one of fluoro, chloro, methyl, ethyl, iso-propyl, cyclopropyl, methoxy, trifluoromethyl, trifluoromethyloxy (—OCF₃), —NR^{4A}R^{4B}, CO₂H, and CO₂CH₃.

43. A compound according to claim 38 wherein Ar¹ is selected from any one of the following ring systems:

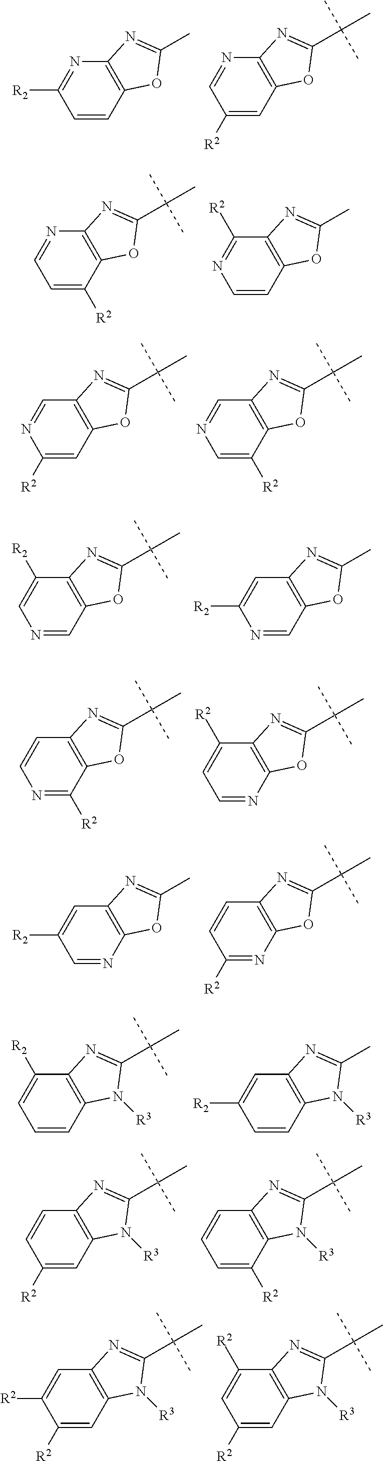

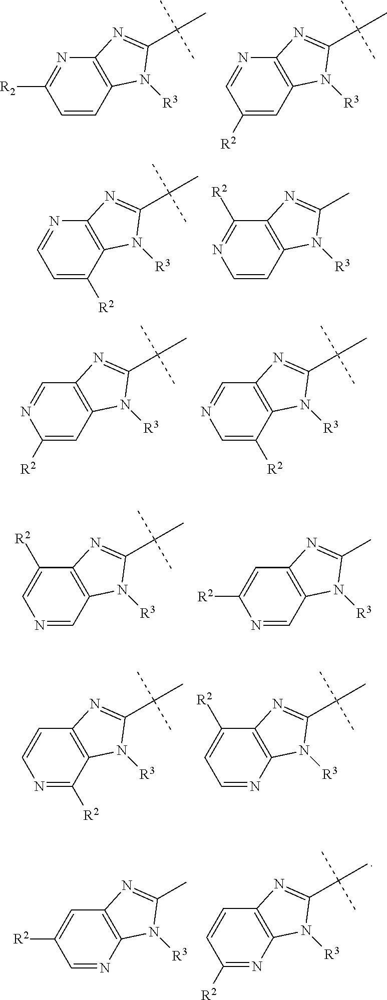

44. A compound according to claim 43, wherein R² is independently selected from any one of fluoro, chloro, methyl, ethyl, iso-propyl, cyclopropyl, methoxy, trifluoromethyl, trifluoromethyloxy (—OCF₃), —NR^{4A}R^{4B}, CO₂H, and CO₂CH₃.

45. A compound according to claim 38 wherein Ar¹ is independently selected from any one of the following ring systems:

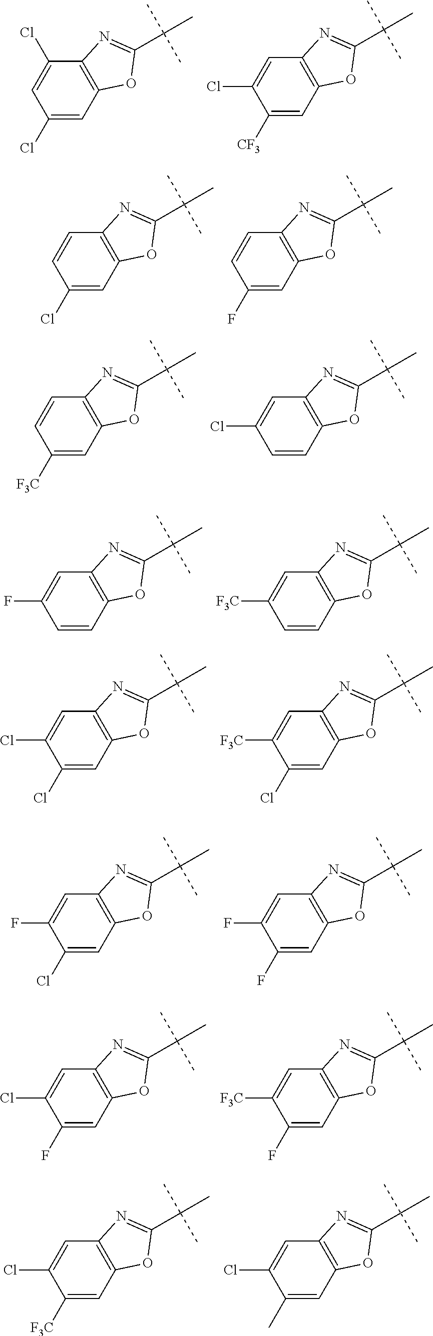

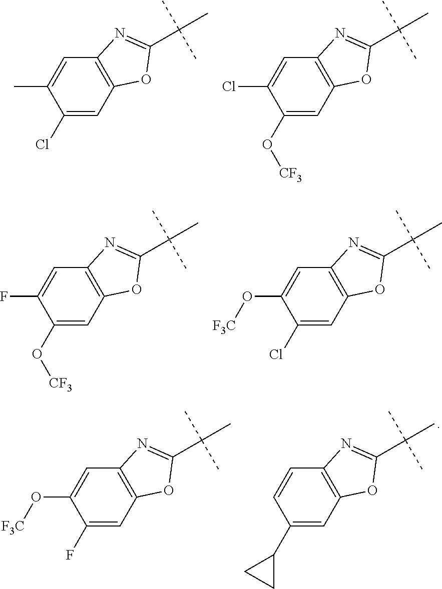

46. A compound according to claim 38 wherein Ar² is selected from any one of the following ring systems:

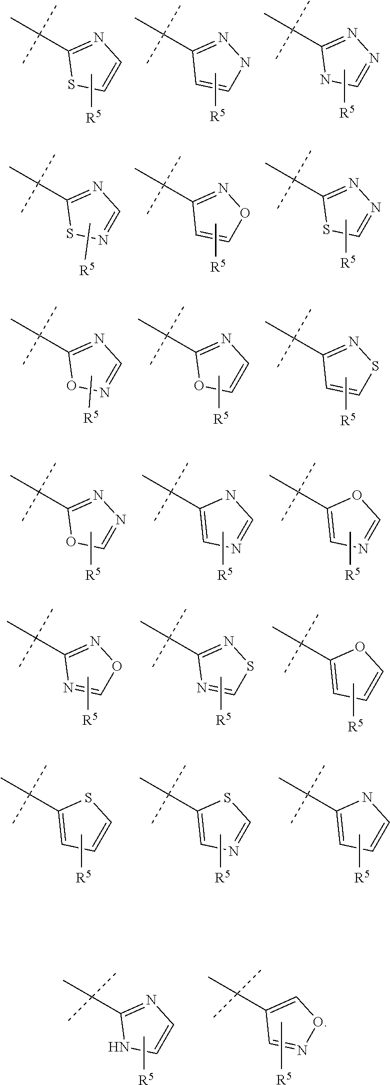

47. A compound according to claim 46, wherein Ar² is selected from any one of the following ring systems:

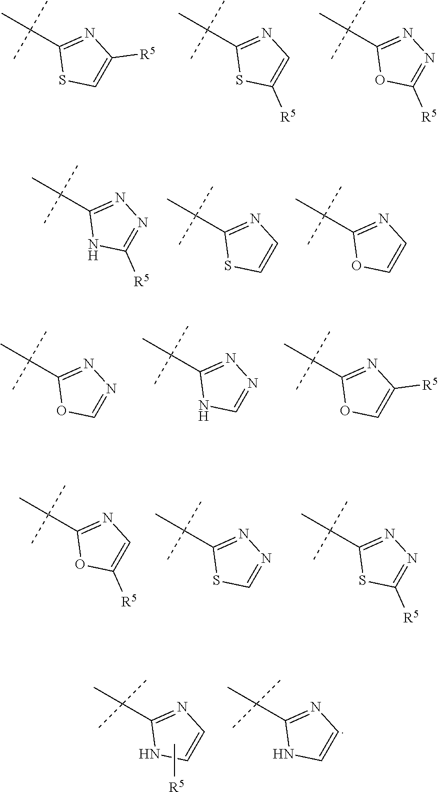

48. A compound according to claim 38 wherein R⁵ is independently selected from any one of fluoro, chloro, methyl, isopropyl, tert-butyl, trifluoromethyl, cyclopropyl, CO₂Et, —NR^{5A}R^{5B}, —CONR^{5A}R^{5B}, —CH₂NR^{5A}R^{5B}, and a ring system selected from pyrrolidinyl, morpholinyl, piperidinyl and piperazinyl, any of which rings is optionally substituted with one or more groups selected from fluoro, chloro, methyl, methoxy, cyano, and CO₂^{t}Bu;
wherein R^{5A} and R^{5B} are each independently selected from hydrogen, methyl, ethyl, isopropyl, cyclopropyl, —COCH₃, —CH₂CH₂N(CH₃)₂, —CH₂CH₂OCH₃, phenyl, and pyridyl, either of which phenyl, and pyridyl rings is optionally substituted with one or more groups selected from fluoro, chloro, and methyl; or
R^{5A} and R^{5B} which together with the nitrogen atom to which they are attached form a cyclic amino group selected from pyrrolidinyl, morpholinyl, piperidinyl, piperazinyl, any of which rings is optionally substituted with one or more groups selected from fluoro, methyl, methoxy, cyano, and CO₂^{t}Bu.

49. A compound according to claim 48, wherein R⁵ is independently selected from any one of fluoro, chloro, methyl, isopropyl, tert-butyl, trifluoromethyl, cyclopropyl, CO₂Et, —NR^{5A}R^{5B}, —CONR^{5A}R^{5B}, —CH₂NR^{5A}R^{5B}, and a ring system selected from pyrrolidinyl, morpholinyl, piperidinyl and piperazinyl, any of which rings is optionally substituted with one or more groups selected from fluoro, chloro, methyl, methoxy, cyano, and CO₂^{t}Bu.

50. A compound according to claim 49, wherein R⁵ is independently selected from any one of methyl, isopropyl, tert-butyl, cyclopropyl, —CONR^{5A}R^{5B} and —CH₂NR^{5A}R^{5B}.

51. A compound according to claim 50, and most preferably wherein R⁵ is absent.

52. A compound according to claim 38, wherein Ar² is selected from Group (i), R¹ is H and Ar¹ is selected from the following groups:

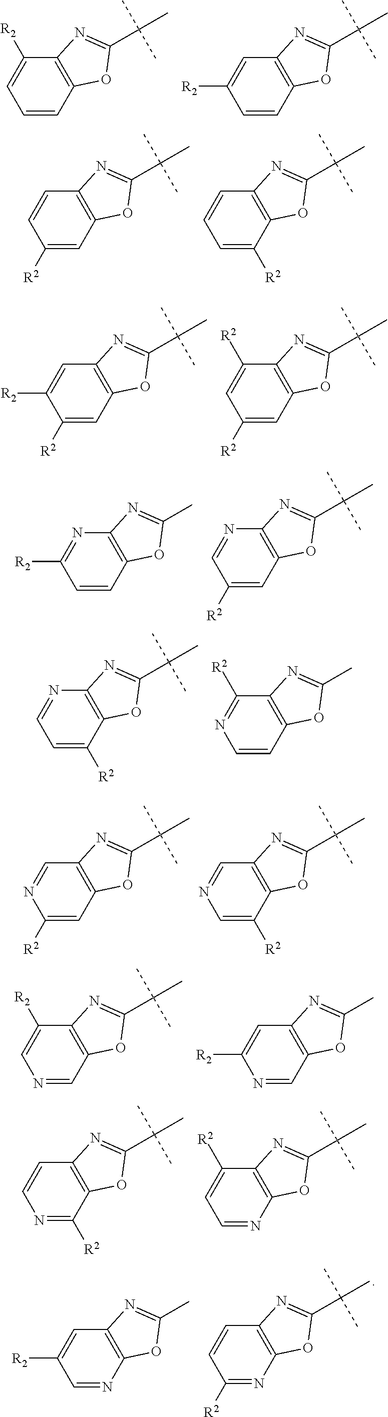

53. A compound according to claim 52, wherein Ar² is selected from Group (i), R¹ is H and Ar¹ is selected from the following groups:

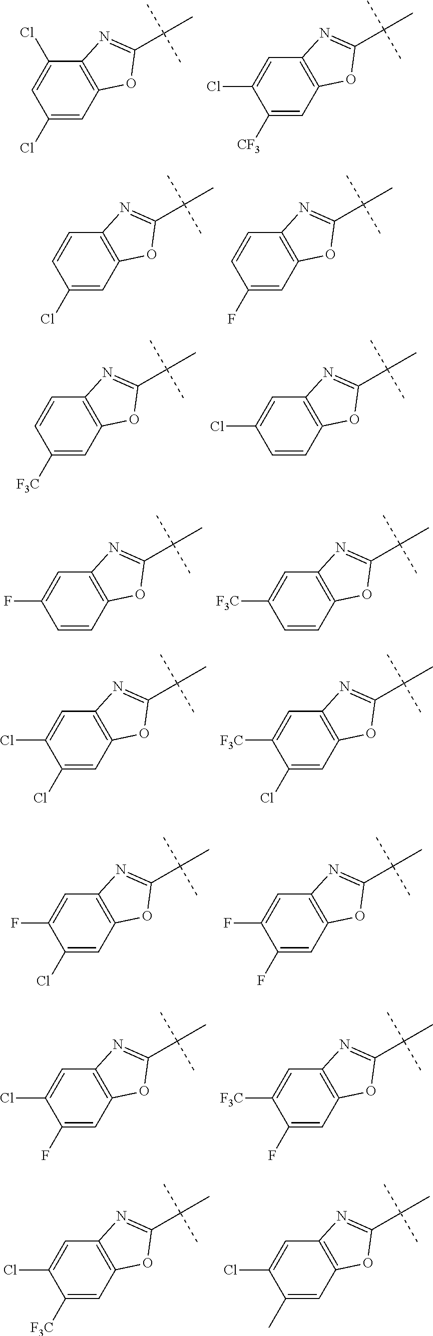

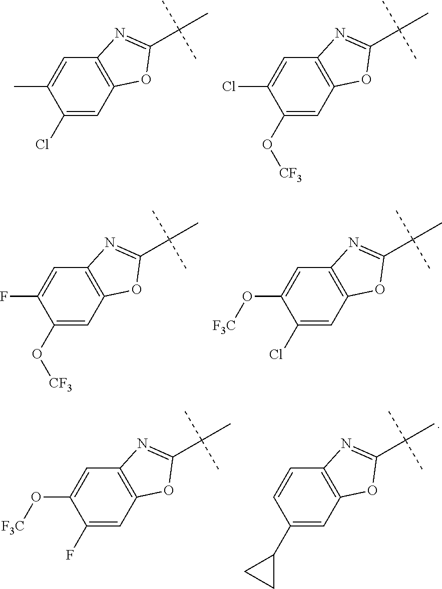

54. A compound according to claim 53, wherein Ar¹ is selected from one of the following groups: 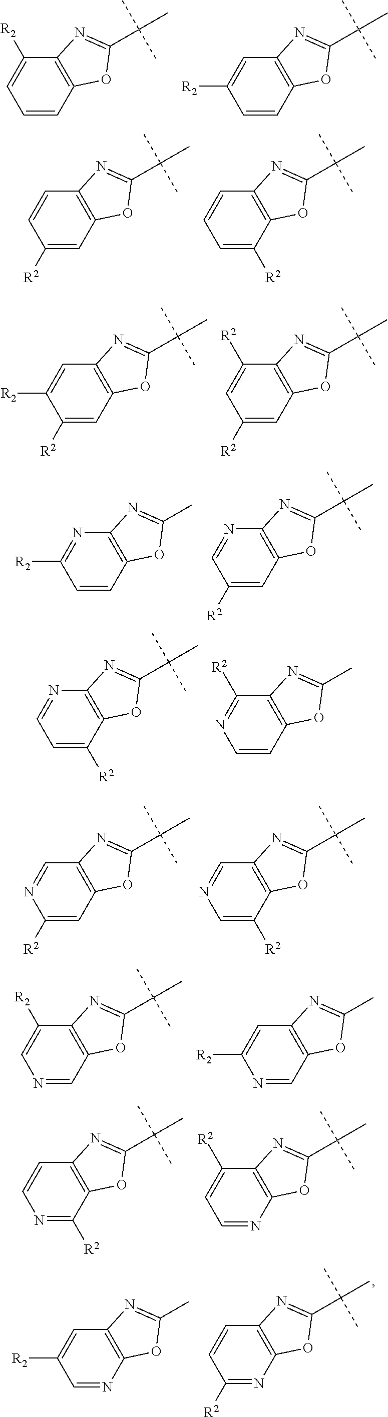
R¹ is H and Ar² is selected from any one of the following groups:

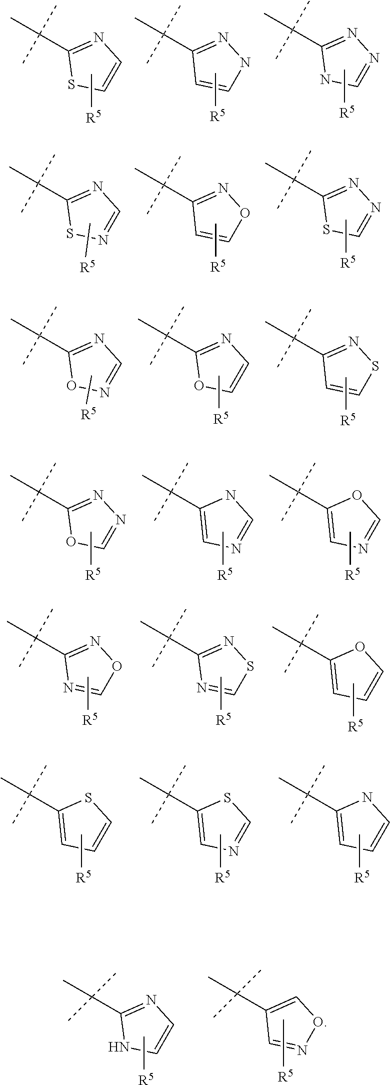

55. A compound according to claim 52, wherein Ar¹ is selected from any one of the following groups: 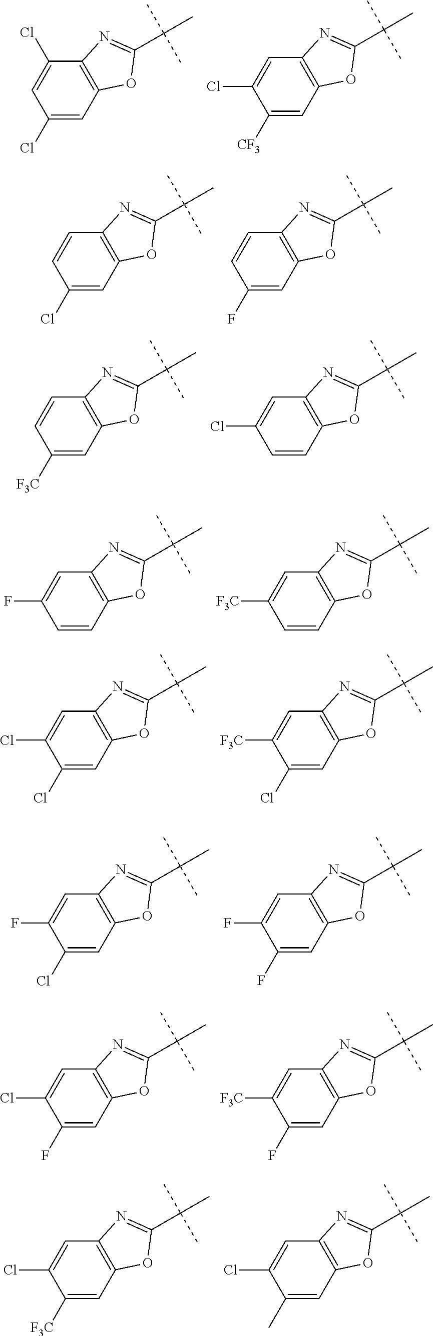 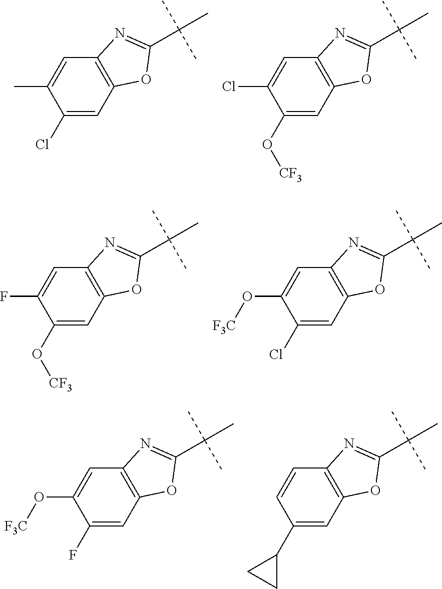
R¹ is H and Ar² is selected from any one of the following groups:

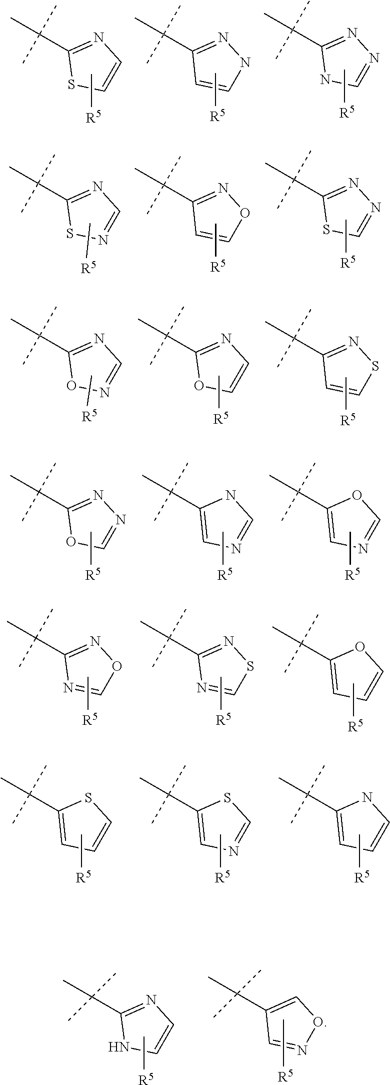

56. A compound according to claim 55, where Ar¹ is selected from any one of the following groups: 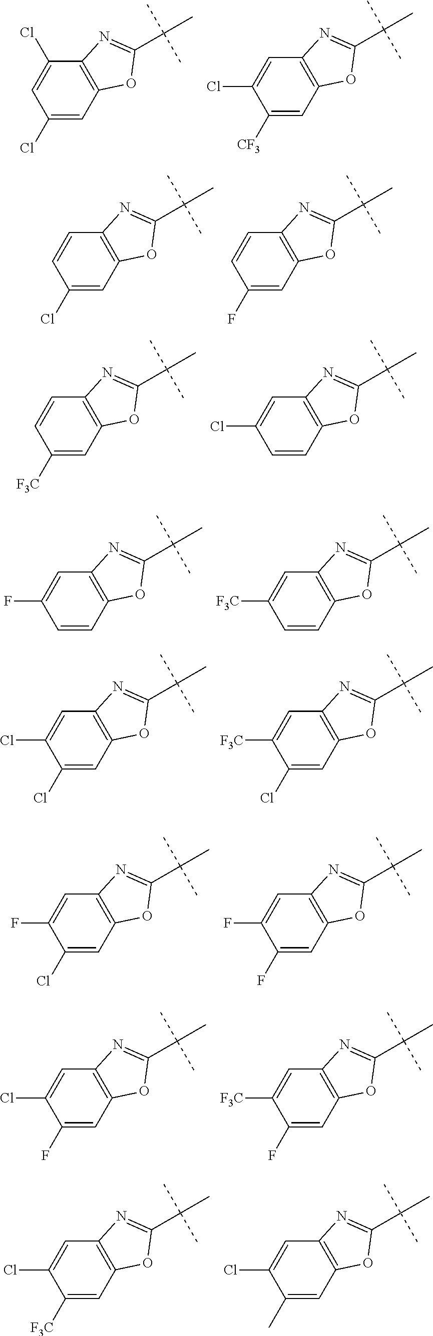 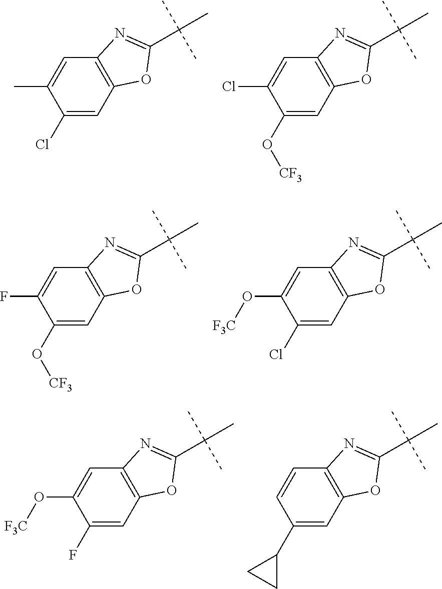
R¹ is H and Ar² is selected from any of the following groups:

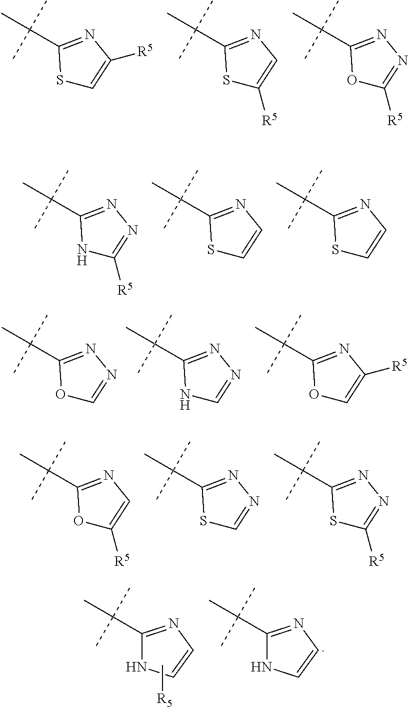

57. A compound according to claim 56, wherein Ar¹ is selected from any one of the following groups: 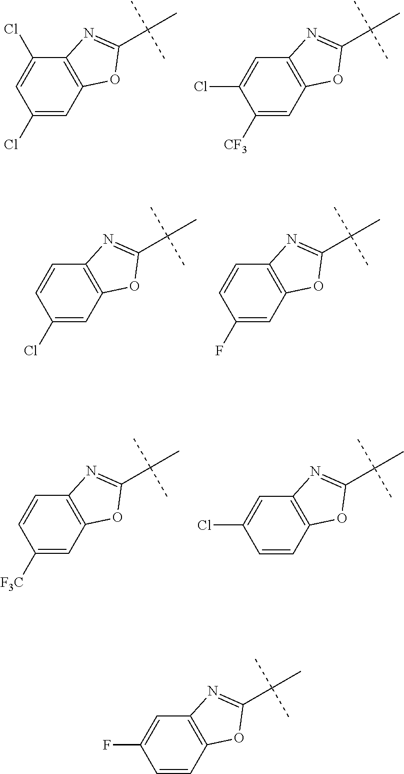 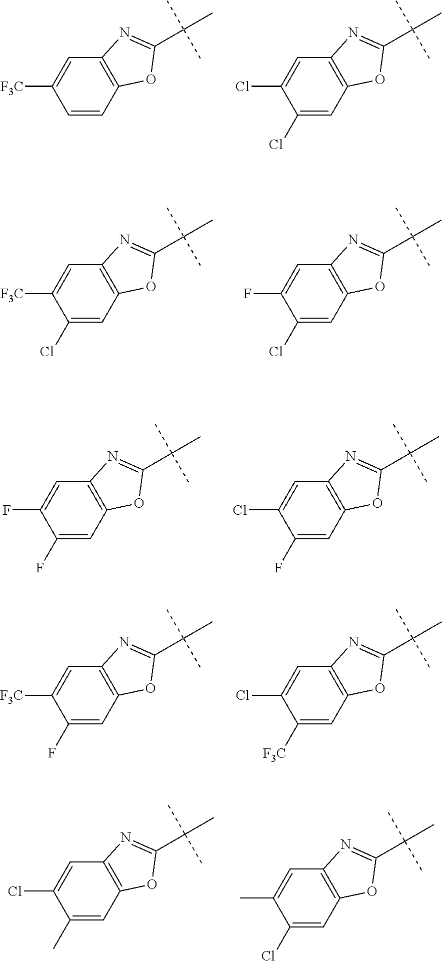 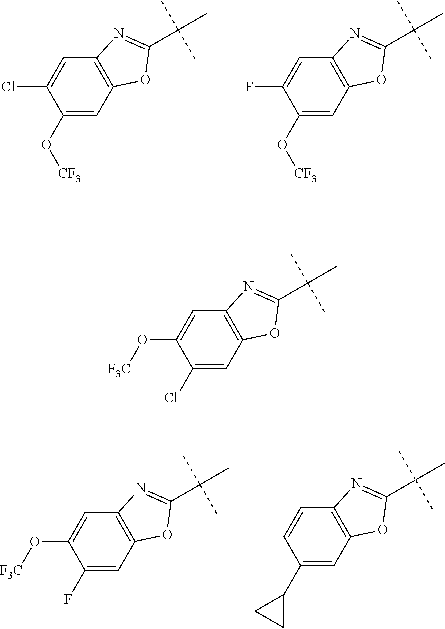
R is H and Ar² is the following group:

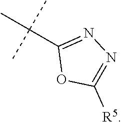

58. A compound according to claim 52, wherein Ar¹ is selected from any one of the following groups: 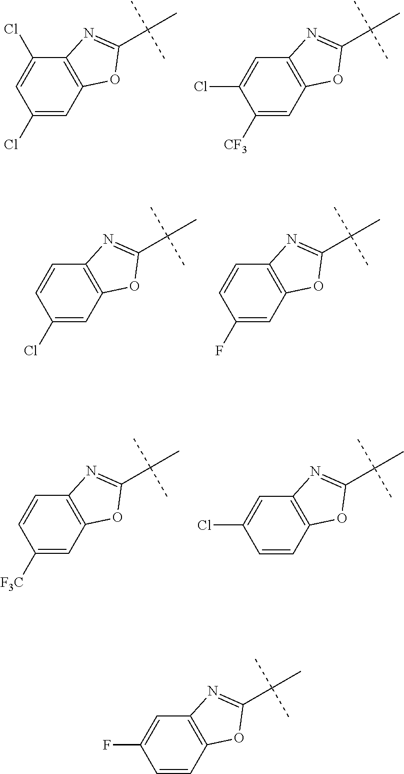 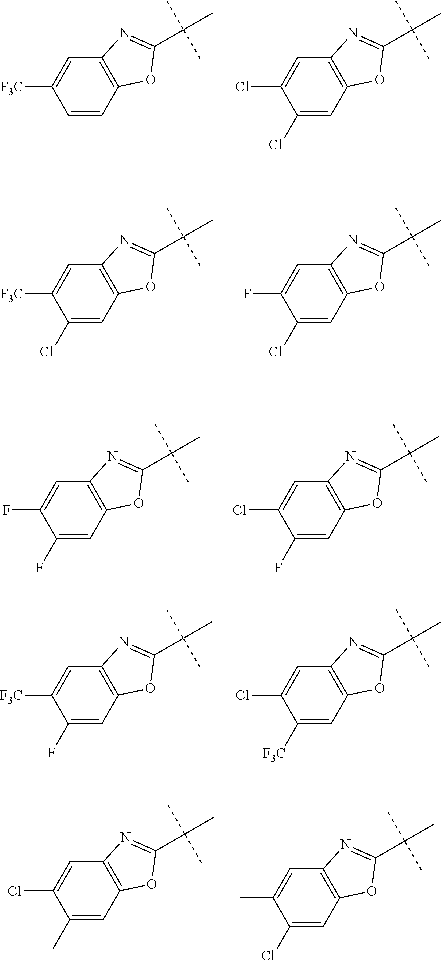 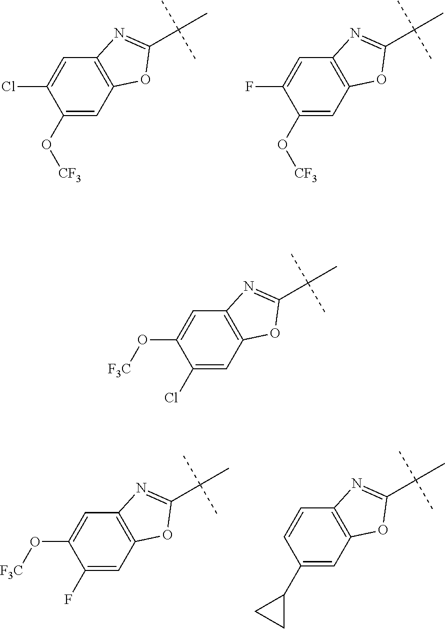
R¹ is H and Ar² is the following group: 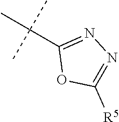
wherein R⁵ is C₁₋₄alkyl such as methyl, isopropyl, tert-butyl, cyclopropyl, —CONR^{5A}R^{5B} and —CH₂NR^{5A}R^{5B}.

59. A compound according to claim 58, wherein Ar¹ is selected from any one of the following groups: 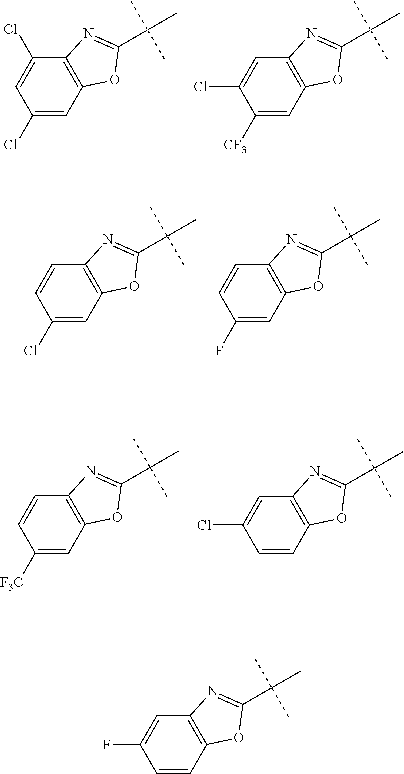 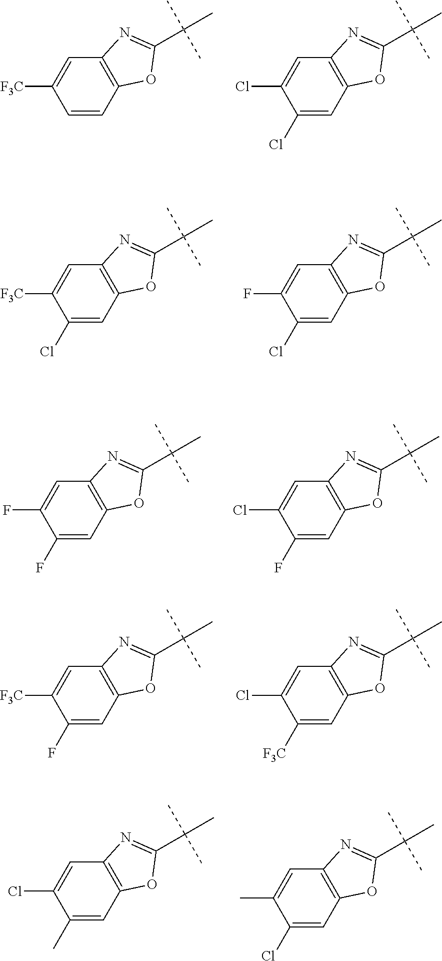 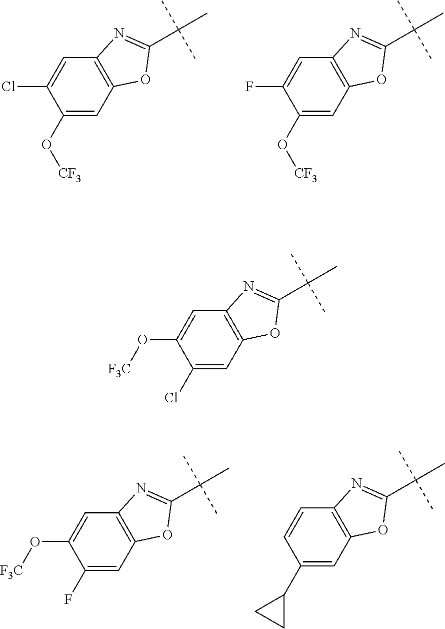
R¹ is H and Ar² is the following group:

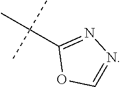

60. A compound as claimed in claim 38 which is one of the Examples and pharmaceutically acceptable salts thereof, such as:
N-Methyl-2-((5-(trifluoromethyl)benzo[d]oxazol-2-yl)amino)thiazole-4-carboxamide,
N-(Thiazol-2-yl)-5-(trifluoromethyl)benzo[d]oxazol-2-amine,
N-(Isoxazol-3-yl)-5-(trifluoromethyl)benzo[d]oxazol-2-amine,
N-(1,3,4-Thiadiazol-2-yl)-5-(trifluoromethyl)benzo[d]oxazol-2-amine,
N-(5-Methyl-1,3,4-oxadiazol-2-yl)-5-(trifluoromethyl)benzo[d]oxazol-2-amine,
N-(5-(Piperidin-1-yl)thiazol-2-yl)-6-(trifluoromethyl)benzo[d]oxazol-2-amine,
6-Chloro-N-(4H-1,2,4-triazol-3-yl)-5-(trifluoromethyl)benzo[d]oxazol-2-amine,
N-(5-Cyclopropyl-1,3,4-oxadiazol-2-yl)-6-(trifluoromethyl)oxazolo[4,5-c]pyridin-2-amine,
6-Fluoro-N-(1,3,4-oxadiazol-2-yl)benzo[d]oxazol-2-amine,
N-(5-(Trifluoromethyl)-1H-benzo[d]imidazol-2-yl)-1,3,4-oxadiazol-2-amine,
N-(5-(Pyrrolidin-1-ylmethyl)-1,3,4-oxadiazol-2-yl)-5-(trifluoromethyl)benzo[d]oxazol-2-amine,
5-Methyl-N-(5-(trifluoromethyl)-1H-benzo[d]imidazol-2-yl)-1,3,4-thiadiazol-2-amine,
2-((5-Chlorobenzo[d]oxazol-2-yl)amino)-N-(2-(dimethylamino)ethyl)thiazole-4-carboxamide,
6-Chloro-N-(1,3,4-oxadiazol-2-yl)benzo[d]oxazol-2-amine,
N-(5-(Pyrrolidin-1-ylmethyl)-4H-1,2,4-triazol-3-yl)-5-(trifluoromethyl)benzo[d]oxazol-2-amine,
6-Chloro-N-(1,3,4-oxadiazol-2-yl)-5-(trifluoromethyl)benzo[d]oxazol-2-amine,
6-Chloro-N-(4-(2-methoxyethyl)-5-methyl-4H-1,2,4-triazol-3-yl)-5-(trifluoromethyl)benzo[d]oxazol-2-amine,
N-(Benzo[d]oxazol-2-yl)-6-(trifluoromethyl)oxazolo[4,5-c]pyridin-2-amine,
6-Chloro-N-(5-methylisoxazol-3-yl)benzo[d]oxazol-2-amine,
5-Chloro-N-(thiazol-4-yl)benzo[d]oxazol-2-amine,
5-Fluoro-N-(1,3,4-oxadiazol-2-yl)-6-(trifluoromethoxy)benzo[d]oxazol-2-amine,
6-Chloro-N-(5-cyclopropyl-1,3,4-oxadiazol-2-yl)benzo[d]oxazol-2-amine,
6-Chloro-4-fluoro-N-(1,3,4-oxadiazol-2-yl)benzo[d]oxazol-2-amine,
2-((1,3,4-Oxadiazol-2-yl)amino)benzo[d]oxazol-6-ol,
Methyl 2-((1,3,4-oxadiazol-2-yl)amino)benzo[d]oxazole-6-carboxylate,
6-Bromo-N-(1,3,4-oxadiazol-2-yl)benzo[d]oxazol-2-amine,
6-Cyclopropyl-N-(1,3,4-oxadiazol-2-yl)benzo[d]oxazol-2-amine,
(2-((1,3,4-Oxadiazol-2-yl)amino)benzo[d]oxazol-6-yl)methanol,
4-((6-chlorobenzo[d]oxazol-2-yl)amino)oxazole-2-carboxylicacid,
N-(1,3,4-oxadiazol-2-yl)-6-(pyyrolidin-1-yl)benzo[d]oxazol-2-amine,
N-(1,3,4-Oxadiazol-2-yl)-6-(piperidin-1-yl)benzo[d]oxazol-2-amine,
6-Morpholino-N-(1,3,4-oxadiazol-2-yl)benzo[d]oxazol-2-amine,
6-Nitro-N-(1,3,4-oxadiazol-2-yl)benzo[d]oxazol-2-amine,
5-Chloro-6-methyl-N-(1,3,4-oxadiazol-2-yl)benzo[d]oxazol-2-amine,
6-Chloro-5-methyl-N-(1,3,4-oxadiazol-2-yl)benzo[d]oxazol-2-amine,
6-Chloro-4-methyl-N-(1,3,4-oxadiazol-2-yl)benzo[d]oxazol-2-amine,
5-Chloro-6-methoxy-N-(1,3,4-oxadiazol-2-yl)benzo[d]oxazol-2-amine,
5-Chloro-N-(1,3,4-oxadiazol-2-yl)-6-(trifluoromethoxy)benzo[d]oxazol-2-amine,
2-((1,3,4-oxadiazol-2-yl)amino)benzo[d]oxazole-6-carboxylic acid,
6-(2-methoxyethoxy)-N-(1,3,4-oxadiazol-2-yl)benzo[d]oxazol-2-amine,
6-Chloro-N-(5-ethynyl-1,3,4-oxadiazol-2-yl)benzo[d]oxazol-2-amine,
N²-(1,3,4-oxadiazol-2-yl)benzo[d]oxazole-2,6-diamine,
6-Isopropoxy-N-(1,3,4-oxadiazol-2-yl)benzo[d]oxazol-2-amine,
5-Fluoro-6-methyl-N-(1,3,4-oxadiazol-2-yl)benzo[d]oxazol-2-amine,
6-Chloro-N-(5-(pyrolidin-1-yl)-1,3,4-oxadiazol-2-yl)benzo[d]oxazol-2-amine,
6-Chloro-N-(5-(tetrahydrofuran-3-yl)-1,3,4-oxadiazol-2-yl)benzo[d]oxazol-2-amine,
5-((6-Chlorobenzo[d]oxazol-2-yl)amino)-1,3,4-oxadiazole-2-carboxamide,
5-((6-Fluorobenzo[d]oxazol-2-yl)amino)-1,3,4-oxadiazole-2-carboxamide,
5-((5-Fluorobenzo[d]oxazol-2-yl)amino)-1,3,4-oxadiazole-2-carboxamide,
6-Fluoro-5-methyl-N-(1,3,4-oxadiazol-2-yl)benzo[d]oxazol-2-amine,
6-Chloro-5-methoxy-N-(1,3,4-oxadiazol-2-yl)benzo[d]oxazol-2-amine,
6-Chloro-N-(1,3,4-oxadiazole-2-yl)-5-(trifluoromethoxy)benzo[d]oxazol-2-amine,
5-Fluoro-6-methoxy-N-(1,3,4-oxadiazol-2-yl)benzo[d]oxazol-2-amine,
6-Fluoro-N-(1,3,4-oxadiazol-2-yl)-5-(trifluoromethoxy)benzo[d]oxazol-2-amine,
6-Methoxy-5-methyl-N-(1,3,4-oxadiazol-2-yl)benzo[d]oxazol-2-amine,
6-Fluoro-5-methoxy-N-(1,3,4-oxadiazol-2-yl)benzo[d]oxazol-2-amine,
N-(1,3,4-Oxadiazol-2-yl)-6-(trifluoromethoxy)benzo[d]oxazol-2-amine,
6-Isopropyl-N-(1,3,4-oxadiazol-2-yl)benzo[d]oxazol-2-amine,
1-(2-((1,3,4-oxadiazol-2-yl)amino)benzo[d]oxazol-6-yl)pyrrolidin-2-one,
6-Chloro-N-(5-(tetrahydrofuran-2-yl)-1,3,4-oxadiazol-2-yl)benzo[d]oxazol-2-amine,
6-Chloro-N-(5-isopropyl-1,3,4-oxadiazol-2-yl)benzo[d]oxazol-2-amine,
N-(2-((1,3,4-Oxadiazol-2-yl)amino)benzo[d]oxazol-6-yl)-N-methylacetamide,
Ethyl 5-((6-chlorobenzo[d]oxazol-2-yl)amino)-1,2,4-oxadiazole-3-carboxylate,
5-((6-Chlorobenzo[d]oxazol-2-yl)amino)-1,3,4-oxadiazole-2-carbonitrile,
5-((6-Fluorobenzo[d]oxazol-2-yl)amino)-1,3,4-oxadiazole-2-carbonitrile,
5-((5-Fluorobenzo[d]oxazol-2-yl)amino)-1,3,4-oxadiazole-2-carbonitrile,
6-Chloro-N-(1,3,4-oxadiazole-2-yl)oxazolo[4,5-b]pyridine-2-amine,
6-Chloro-N-(oxazol-4-yl)benzo[d]oxazol-2-amine,
6-Chloro-N-(isothiazol-3-yl)benzo[d]oxazol-2-amine,
Sodium 5-((6-fluorobenzo[d]oxazol-2-yl)amino)-1,3,4-oxadiazole-2-carboxylate,
5,6-Chloro-N-(1,2,4-oxadiazol-5-yl)benzo[d]oxazol-2-amine,
6-Chloro-N-(isoxazol-4-yl)benzo[d]oxazol-2-amine,
and pharmaceutically acceptable salts thereof.

61. A pharmaceutical composition comprising a compound according to claim 38, together with a pharmaceutically acceptable excipient or carrier.

62. A compound according to claim 38 for use in the treatment of a bacterial infection and/or a disease caused by a bacterial infection; and preferably wherein the bacterial infection is caused by Gram-positive and/or Gram-negative bacteria.

63. A compound for use according to claim 62 wherein the bacterial infection is caused by a bacteria selected from Moraxella catarrhalis, Bacillus thuringiensis, Acinetobacter junii, Escherichia coli, Helicobacter pylori, Borelia burgdorferi, Legionella pneumophilia, Mycobacterium spp (including M. tuberculosis, M. leprae, M. avium, M. intracellulare, M. kansaii and M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus viridans, Streptococcus faecalis, Streptococcus bovis, any anaerobic species of the genus Streptococcus, Streptococcus pneumoniae, Campylobacter spp., Enterococcus spp., Haemophilus influenzae, Bacillus anthracis, Corynebacterium spp. (including C. diphtheriae), Erysipelothrix rhusiopathiae, Clostridium perfringens, Clostridium tetani, Clostridium difficile, Clostridium innocuum, Peptostreptococcus anaerobius, Bacteroides fragilis, Enterobacter aerogenes, Klebsiella spp (including K. pneumoniae), Pasteurella multocida, Bacteroides spp., Fusobacterium nucleatum, Streptobacillus monilijormis, Treponema pallidium, Treponema pertenue, Leptospira spp., Rickettsia spp. and Actinomyces spp. (including A. israelii), Acinetobater baumannii, Pseudomonas aeruginosa, and Staphylococcus epidermidis; and preferably wherein the bacterial infection is caused by a bacterium selected from: Staphylococcus aureus; Enterococcus faecalis, Enterococcus faecium and the Neisseria genus; and more preferably wherein the disease is caused by a bacterium selected from the Neisseria genus; and most preferably wherein the bacterial infection is Neisseria gonorrhoeae.

64. A compound of formula (I), or a pharmaceutically acceptable salt, hydrate, solvate, isomer, tautomer, N-oxide, ester, isotope or protected form thereof for use in the treatment of infection with, or disease caused by a bacterium selected from Moraxella catarrhalis, Acinetobacter junii, Helicobacter pylori, Borelia burgdorferi, Legionella pneumophilia, Neisseria gonorrhoeae, Neisseria meningitidis, Listeria monocytogenes, Streptococcus pyogenes (Group A Streptococcus), Streptococcus agalactiae (Group B Streptococcus), Streptococcus viridans, Streptococcus faecalis, Streptococcus bovis, any anaerobic species of the genus Streptococcus, Streptococcus pneumoniae, Campylobacter spp., Enterococcus spp., Haemophilus influenzae, Bacillus anthracis, Corynebacterium spp. (including C. diphtheriae), Erysipelothrix rhusiopathiae, Clostridium perfringens, Clostridium tetani, Clostridium difficile, Clostridium innocuum, Peptostreptococcus anaerobius, Bacteroides fragilis, Enterobacter aerogenes, Klebsiella spp (including K. pneumoniae), Pasteurella multocida, Bacteroides spp., Fusobacterium nucleatum, Streptobacillus monilijormis, Treponema pallidium, Treponema pertenue, Leptospira spp., Rickettsia spp. and Actinomyces spp. (including A. israelii), Acinetobater baumannii, and Pseudomonas aeruginosa: 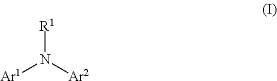
wherein R¹ is selected from hydrogen and C₁₋₄alkyl;
Ar¹ is selected from any one of the following ring systems: 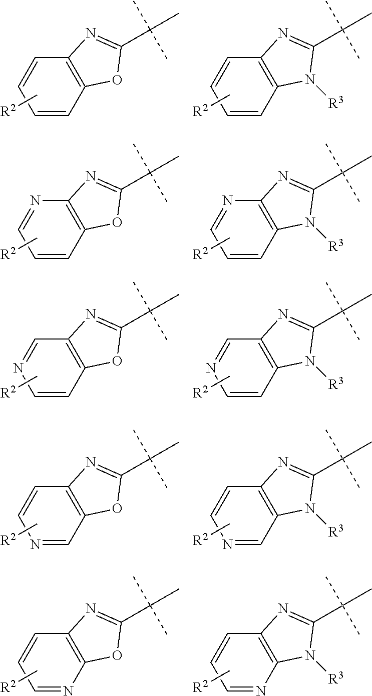
wherein R² is one or more optional substituents each independently selected from halogen, cyano, hydroxyl, hydroxylC₁₋₄alkyl, C₁₋₄alkyl, haloC₁₋₄alkyl, C₁₋₄alkoxy, haloC₁₋₄ alkyloxy, —C₁₋₄alkylC₁₋₄alkoxy, C₁₋₄alkoxyC₁₋₄alkoxy, NR^{4A}R^{4B}, NO₂, —CONR^{4A}R^{4B}, —C₁₋₄ alkylNR^{4A}R^{4B}, —C₁₋₄alkoxyNR^{4A}R^{4B}, C₃₋₇cycloalkyl, morpholinyl, C₂₋₄alkynyl and —CO₂R⁴ wherein
R³ is hydrogen or C₁₋₄alkyl,
R⁴ is hydrogen or C₁₋₄alkyl,
R^{4A} and R^{4B} are each independently selected from hydrogen, C₁₋₄alkyl, —C₁₋₄alkylC₁₋₄ alkoxy, and COR⁴, or
R^{4A} and R^{4B}, together with the nitrogen atom to which they are attached, join together to form a cyclic amino group, wherein the cyclic amino group is optionally substituted with oxo;
Ar² is a ring system selected from Groups (i), and (ii), wherein:
Group (i) is a 5-membered heteroaryl ring system selected from any one of (IIa) to (IIm): 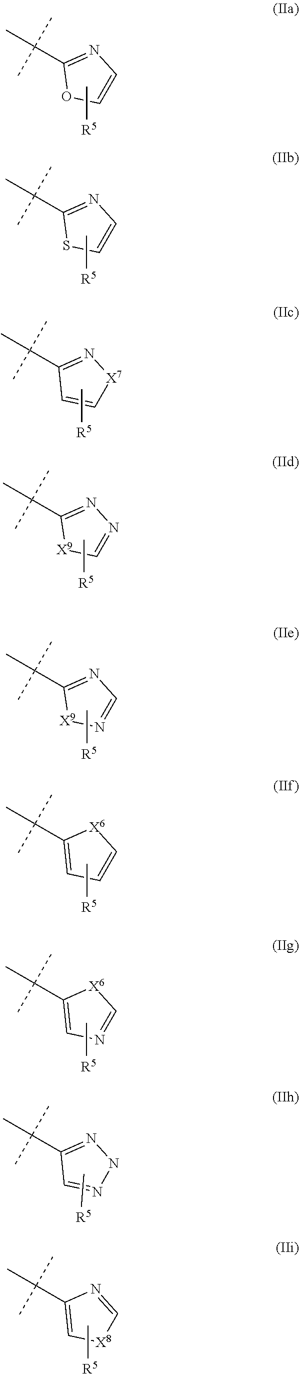 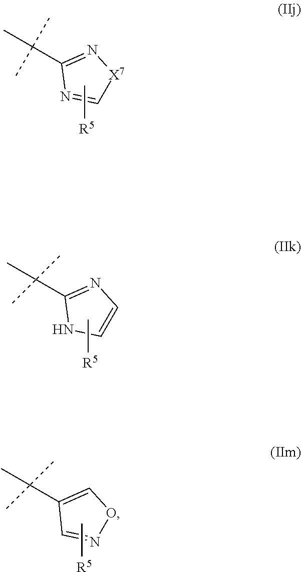
wherein X⁶, X⁷, X⁸, and X⁹ are each independently selected from O, S, and NH, and
R⁵ is one or more optional substituents each independently selected from halogen, cyano, C₁₋₄alkyl, haloC₁₋₄alkyl, C₁₋₄alkoxy, —C₁₋₄alkylC₁₋₄alkoxy, —CO₂R⁶, and -L-Q wherein:
L is a linker group selected from a direct bond, C₁₋₃alkylene and —CO—; and
Q is a group selected from NR^{5A}R^{5B}, C₃cycloalkyl and 4-7 membered heterocyclyl, wherein the 4-7 membered heterocyclyl ring is optionally substituted with one or more substituents selected from halogen, cyano, C₁₋₄alkyl, C₁₋₄alkoxy and CO₂R⁶;
R^{5A} and R^{5B} are each independently selected from hydrogen, C₁₋₄alkyl, C₃₋₇cycloalkyl, COR⁷, —C₁₋₄alkyl-NR⁸R⁹, —C₁₋₄alkylC₁₋₄alkoxy, phenyl and 5 or 6-membered heteroaryl wherein the phenyl or 5 or 6-membered heteroaryl rings are optionally substituted with one or more substituents selected from halogen and C₁₋₄alkyl; or
R^{5A} and R^{5B}, together with the nitrogen atom to which they are attached, join together to form a cyclic amino group, which cyclic amino group is optionally substituted with one or more groups selected from halogen, C₁₋₄alkyl, C₁₋₄alkoxy, cyano, and CO₂R⁶,
R⁶ is hydrogen, C₁₋₄alkyl or an alkali metal;
R⁷ is C₁₋₄alkyl
R⁸ and R⁹ are each independently selected from hydrogen and C₁₋₄alkyl;
Group (ii) is a 5,6-fused bicyclic heteroaryl ring system having the formula (III): 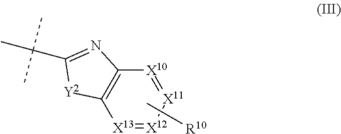
wherein Y² is selected from O and NR^{5C};
R^{5C} is hydrogen or C₁₋₄alkyl,
X¹⁰, X¹¹, X¹², and X¹³ are each independently selected from N and CH;
R¹⁰ is one or more optional substituents each independently selected from halogen, cyano, C₁₋₄alkyl, haloC₁₋₄alkyl, C₁₋₄alkoxy, and —CO₂R⁴;
PROVIDED THAT the compound of formula (I) is other than: 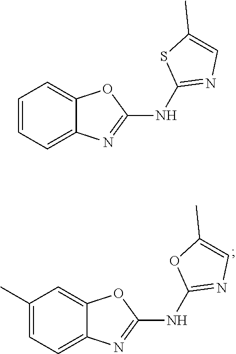 [SMILES: Cc1cnc(Nc2nc3ccccc3o2)s1 ; Cc1ccc2nc(Nc3ncc(C)o3)oc2c1]
and preferably wherein the compound of formula (I) is for use in the treatment of infection with, or disease caused by a bacterium selected from Enterococcus faecalis, Enterococcus faecium and the Neisseria genus; more preferably for use in the treatment of infection with, or disease caused by a bacterium selected from the Neisseria genus; and most preferably for use in the treatment of infection with, or disease caused by the bacterium Neisseria gonorrhoeae.

65. A compound according to claim 55, wherein Ar¹ is selected from any one of the following groups: 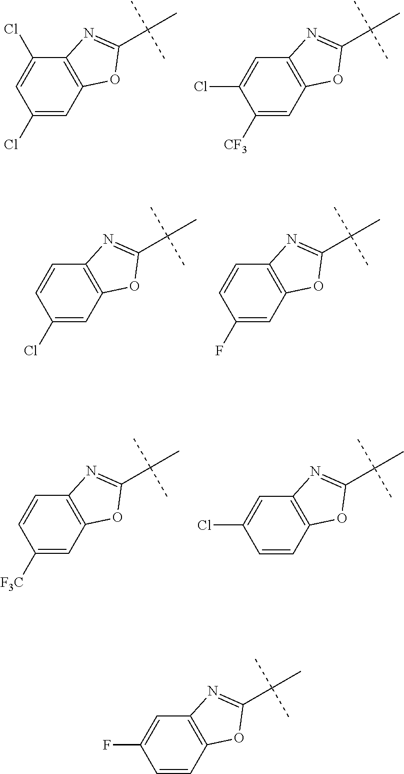 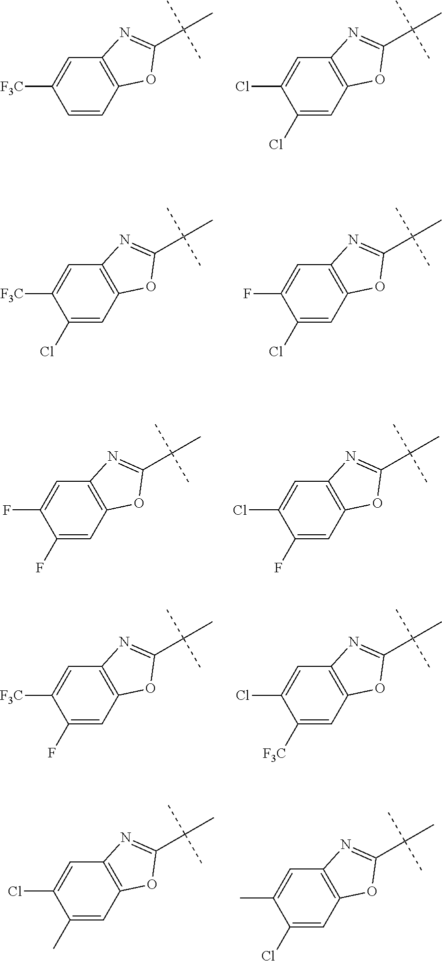 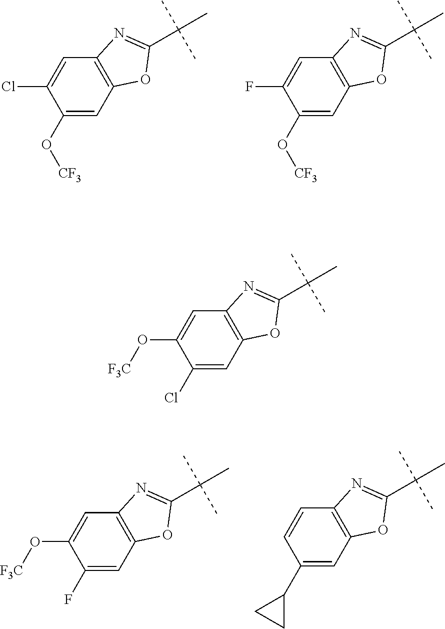
R¹ is H and Ar² is the following group: 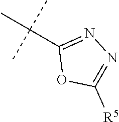
wherein R⁵ is C₁₋₄alkyl such as methyl, isopropyl, tert-butyl, cyclopropyl, —CONR^{5A}R^{5B} and —CH₂NR^{5A}R^{5B}.

66. A method for the treatment of a bacterial infection and/or a disease caused by a bacterial infection, which comprises administering to a mammal suffering such an infection or disease an effective amount of a compound as claimed in claim 38.
